(19) United States
(12) Patent Application Publication
(10) Pub. No.: US 2023/0159445 A1
(43) Pub. Date: May 25, 2023

(54) STAT INHIBITORY COMPOUNDS AND COMPOSITIONS

(71) Applicant: JANPIX LIMITED, Cambridge, MA (US)

(72) Inventors: Roman FLECK, Boston, MA (US); John PROUDFOOT, Newtown, CT (US); Jeff OMEARA, Georgetown (CA); Krishna Kumar GNANASEKARAN, Mississauga (CA); Patrick Thomas GUNNING, Mississauga (CA); Alford Antoine JOHN, Brampton (CA); Angel Sampedro PALERM, Milton (CA); Alla DARWISH, Kitchener (CA); Dziyana KRASKOUSKAYA, Mississauga (CA); Ahmed Magdy Ahmed Mohamed ALI, Cambridge, MA (US)

(21) Appl. No.: 17/909,153

(22) PCT Filed: Mar. 5, 2021
(86) PCT No.: PCT/US2021/021222

§ 371 Date: Sep. 2, 2022

Related U.S. Application Data
(60) Provisional application No. 62/985,678, filed on Mar. 5, 2020.

(51) Int. Cl. C07C 311/19

(52) U.S. Cl. CPC C07C 311/19

(57) ABSTRACT

Provided herein are compounds and pharmaceutical compositions comprising said compounds that are useful for the inhibition of Signal Transducer and Activator of Transcription 5a and 5b (STAT5). Furthermore, the subject compounds and compositions are useful for the treatment of cancer, such as, for example, breast cancer and pancreatic cancer.

### CROSS-REFERENCE TO RELATED APPLICATION

This application claims the benefit of U.S. Provisional Application No. 62/985,678 filed on Mar. 5, 2020, which is hereby incorporated by reference in its entirety.

### BACKGROUND

The Signal Transducer and Activator of Transcription (“STAT”) proteins constitute a family of cytoplasmic transcription factors that play a fundamental role in cell signaling. The STAT protein family consists of 7 members, STAT1 to STAT6, including STAT5 and STAT3. STAT5 can transduce intracellular and extracellular signals to the nucleus and control the expression of genes responsible for multiple physiological processes. STAT proteins are ideal targets for anti-cancer therapy because cancer cells are more dependent on STAT activity than their normal counterparts. Therefore, a need exists in the medicinal arts for compounds, formulation, and methods of STAT5 modulation.

### SUMMARY

Provided herein are compounds and pharmaceutical compositions comprising said compounds that are useful for the inhibition of Signal Transducer and Activator of Transcription, for example STAT 5a and 5b (STAT5). Furthermore, the subject compounds and compositions are useful for the treatment of cancer, such as, for example, breast cancer and pancreatic cancer.

One aspect of the disclosure provides a compound having the structure of Formula (I), or a pharmaceutically acceptable salt or solvate thereof:

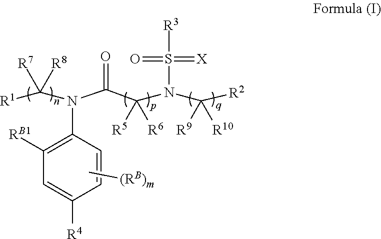

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
   provided that p is 1 and q is 1;
each of R^{B} and R^{B1} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, or 3.

In some embodiments of a compound of Formula (I), or a pharmaceutically acceptable salt or solvate thereof:
each of R⁵ and R⁶ is independently selected from hydrogen, F, —CN, —OR¹¹, —SR¹¹, —N(R¹¹)², substituted or unsubstituted C₁-C₆ alkyl, substituted or unsubstituted C₁-C₆haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring.

In some embodiments, the disclosure provides a compound having the structure of Formula (II), or a pharmaceutically acceptable salt or solvate thereof:

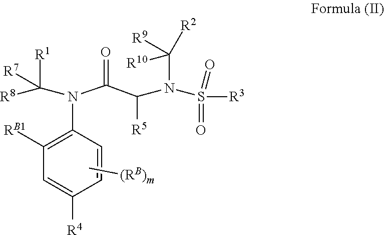

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹C(═O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In some embodiments of a compound of Formula (II), or a pharmaceutically acceptable salt or solvate thereof:
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆heteroalkyl, or R⁹ and R¹⁰, taken together with the carbon to which they are attached form a substituted or unsubstituted 3, 4, 5, or 6-membered ring.

In some embodiments, the disclosure provides a compound having the structure of Formula (IIa), or a pharmaceutically acceptable salt or solvate thereof:

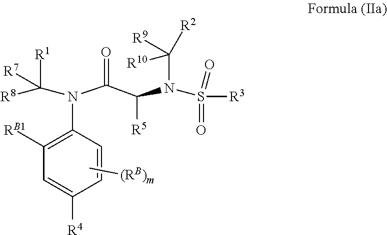

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆haloalkyl, and substituted or unsubstituted C₁-C₆heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆haloalkyl, substituted or unsubstituted C₁-C₆heteroalkyl, substituted or unsubstituted C₃-C₈ cycloalkyl, and substituted or unsubstituted C₃-C₇ heterocycloalkyl,
   wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C1-C6heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
   m is 0, 1, 2, or 3.

In some embodiments, the disclosure provides a compound having the structure of Formula (IIb), or a pharmaceutically acceptable salt or solvate thereof:

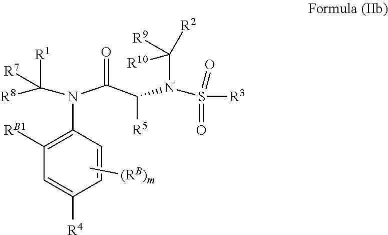

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, NR¹¹C(═O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In some embodiments, the disclosure provides a compound having the structure of Formula (III), or a pharmaceutically acceptable salt or solvate thereof:

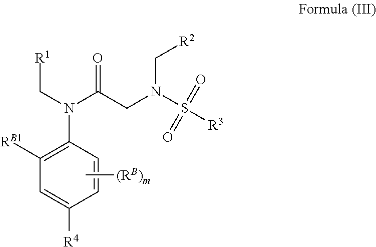

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{B} and R^{B1} is independently halogen, D, —CN, —NO₂, —OR¹¹, —SR¹¹, —N(R¹¹)₂, —NR¹¹C(=O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In another aspect of the disclosure, the disclosure provides a compound having the structure of Formula (IV), or a pharmaceutically acceptable salt or solvate thereof:

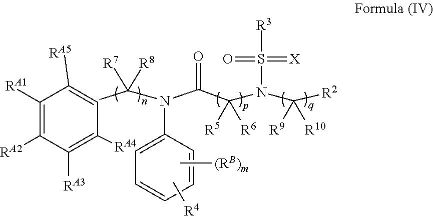

wherein,
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl is optionally substituted with hydroxy, amino, or methoxy, or R⁷ and R⁸, taken together with the carbon to which they are attached form a substituted or unsubstituted 3, 4, 5, or 6-membered ring; each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
   provided that p is 1 and q is 1;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl, wherein
   at least one of R^{A1} and R^{A3} is substituted or unsubstituted —C₃-C₈ cycloalkyl, substituted or unsubstituted —C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, 3, or 4;
provided that when (i)

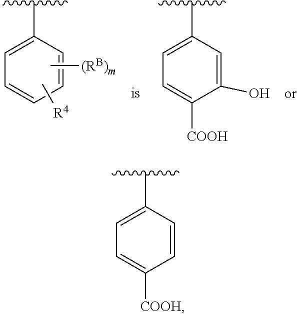

(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other is hydrogen or unsubstituted t-butyl,
then R^{A4} and R^{A5} are not the same.

In some embodiments of a compound of Formula (IV), or a pharmaceutically acceptable salt or solvate thereof:
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆haloalkyl, substituted or unsubstituted C₁-C₆heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁹ and R¹⁰, taken together with the carbon to which they are attached form a substituted or unsubstituted 3, 4, 5, or 6-membered ring.

In some embodiments, the disclosure provides a compound having the structure of Formula (V), or a pharmaceutically acceptable salt or solvate thereof:

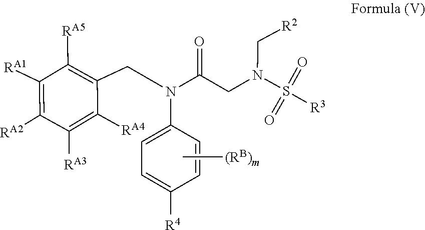

wherein,
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl, wherein
   at least one of R^{A1} and R^{A3} is substituted or unsubstituted —C₃-C₈ cycloalkyl, substituted or unsubstituted —C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(—O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, 3, or 4;
provided that when (i)

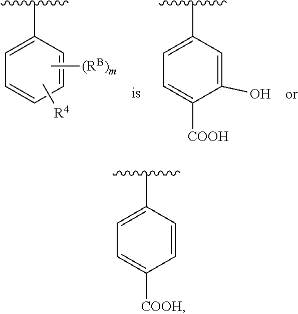

(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other is hydrogen or unsubstituted t-butyl,
then R^{A4} and R^{A5} are not the same.

In one aspect, described herein is a compound selected from Table 1 or Table 2, or a pharmaceutically acceptable salt or solvate thereof. Also described herein is a pharmaceutical composition comprising a compound selected from Table 1 or Table 2, or a pharmaceutically acceptable salt or solvate thereof, and a pharmaceutically acceptable excipient or carrier.

Another aspect of the disclosure provides a pharmaceutical composition comprising a compound of Formula (VI), (I), (II), (IIa), (IIb), (III), (IV), or (V), or a pharmaceutically acceptable salt or solvate thereof, and a pharmaceutically acceptable excipient or carrier.

Another aspect of the disclosure provides a method of making the compounds and compositions described herein.

Another aspect of the disclosure provides a method of modulating signal transducer and activator of transcription 5a and 5b (STAT5) proteins in a subject in need thereof, comprising administering to a subject a therapeutically effective amount a compound of Formula (VI), (I), (II), (IIa), (IIb), (III), (IV), or (V), or a pharmaceutically acceptable salt or solvate thereof

In yet another aspect of the disclosure, the disclosure provides a method comprising administering to a subject with cancer a therapeutically effective amount of a compound of Formula (I), (II), (IIa), (IIb), (III), (IV), or (V), or a pharmaceutically acceptable salt or solvate thereof. In some embodiments, the cancer is a solid tumor or hematologic cancer. In some embodiments, the cancer is breast cancer, head and neck squamous cell carcinoma, non-small cell lung cancer, hepatocellular cancer, colorectal cancer, gastric adenocarcinoma, melanoma, or advanced cancer.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure may be obtained by reference to the following detailed description that sets forth illustrative embodiments, in which the principles of the disclosure are utilized, and the accompanying drawings of which:

FIG. 1A-F shows an exemplary STAT5 inhibitory compound of Formula (I), Formula (II), Formula (IIa), Formula (IIb), Formula (III), Formula (IV), or Formula (V).

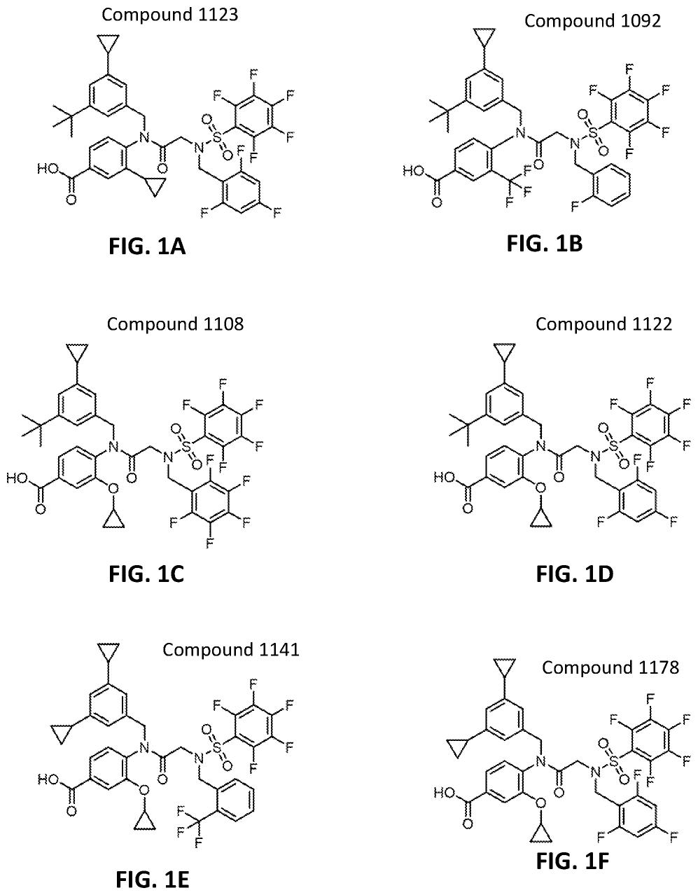

FIG. 2A-F shows an exemplary STAT5 inhibitory compound of Formula (I), Formula (II), Formula (IIa), Formula (IIb), Formula (III), Formula (IV), or Formula (V).

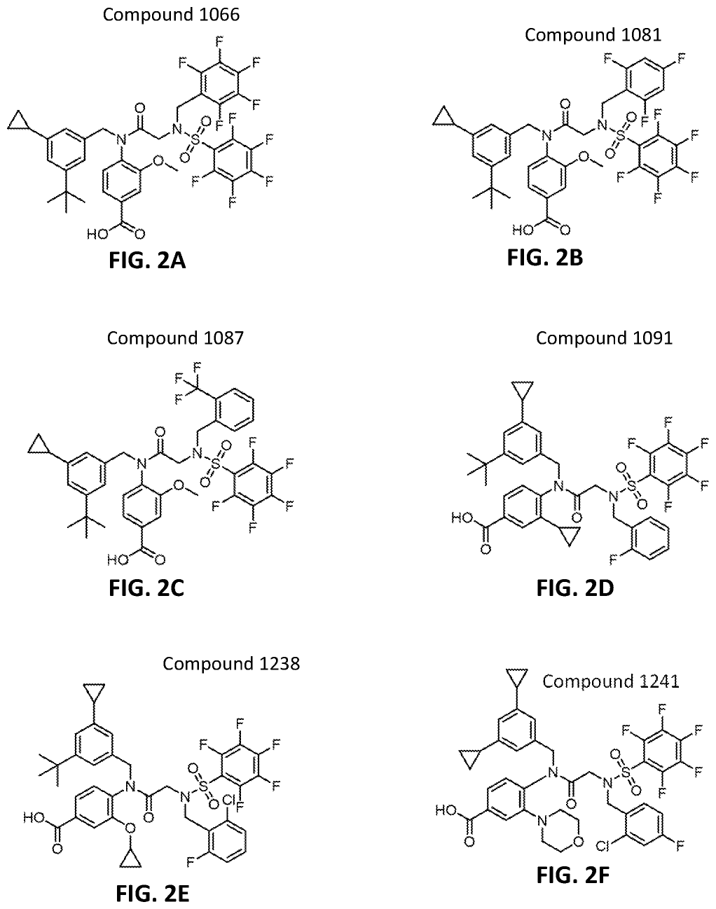

FIG. 3A-F shows an exemplary STAT5 inhibitory compound of Formula (I), Formula (II), Formula (IIa), Formula (IIb), Formula (III), Formula (IV), or Formula (V).

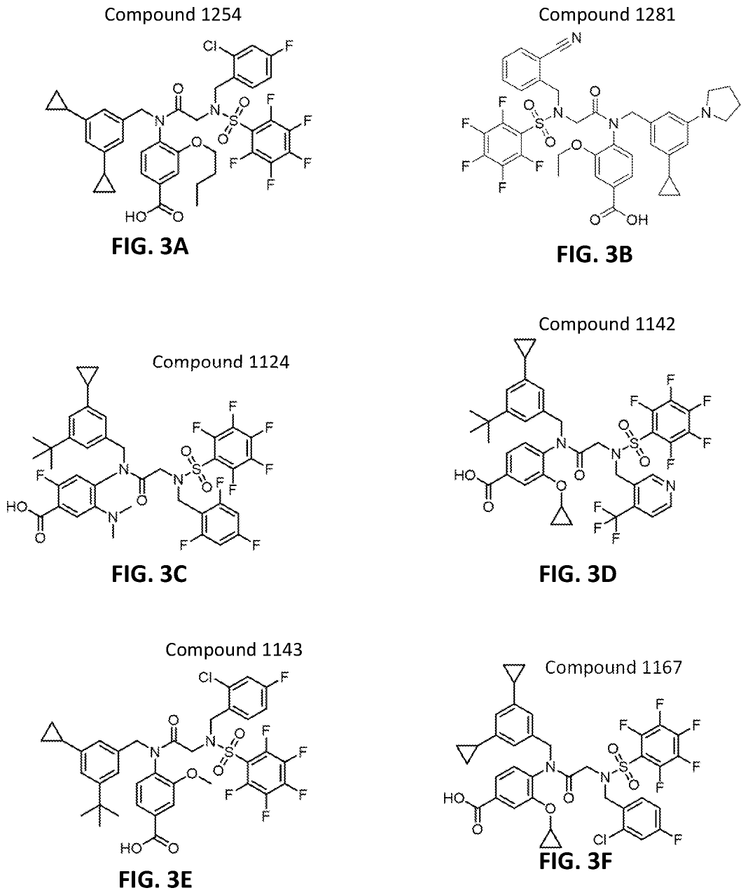

### DETAILED DESCRIPTION

The present disclosure relates to STAT5 inhibitory compounds, pharmaceutical compositions comprising said compounds, and methods of making and/or using the compounds.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Methoxyl” refers to the —O-Me radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Hydroxyamino” refers to the —NH—OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Acyl-sulfonamide” refers to a monovalent radical where the carbon atom of a carbonyl is bound to a sulfonamide group. Exemplary acyl-sulfonamides include —C(O)NR^{a}S(O)₂R^{a}, —C(O)NR^{a}S(O)₂N(R^{a})₂, —NR^{a}S(O)₂C(O)R^{a}, —NR^{a}S(O)₂C(O)N(R^{a})₂, —C(O)NR^{a}S(O)₂C(O)N(R^{a})₂, —NR^{a}S(O)₂NR^{a}C(O)N(R^{a})₂, —C(O)NR^{a}S(O)₂NR^{a}C(O)N(R^{a})₂, and —C(O)S(O)₂N(R^{a})₂, where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO2. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, ——NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkylamino” refers to a radical of the formula —N(Rₐ)₂ where Rₐ is an alkyl radical as defined, or two Rₐ, taken together with the nitrogen atom, can form a substituted or unsubstituted C₂-C₇ heterocyloalkyl ring such as:

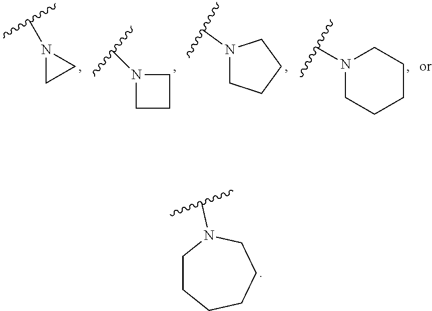

Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising at least one aromatic ring. In some embodiments, an aryl comprises hydrogens and 6 to 30 carbon atoms. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆ alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocyclyl,” “heterocycle,” or “heterocyclic” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused, bridged, or spirocyclic ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—CN, —R^{b}—O—R^{e}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and Re is a straight or branched alkylene or alkenylene chain, and where each of the above substituents is unsubstituted unless otherwise indicated.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms that selected from the group consisting of nitrogen, oxygen, phosphorous, and sulfur, and at least one aromatic ring. In some embodiments, a heteroaryl is a 5- to 14-membered ring system radical comprising one to thirteen carbon atoms, one to six heteroatoms selected from the group consisting of nitrogen, oxygen, phosphorous, and sulfur. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, b enzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “spiro” or “spirocyclic” refers to a compound or moiety having one atom as the only common member of two rings.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof. As used herein, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention,” that is, reducing the probability of developing a disease or condition in a subject, who does not have, but is at risk of or susceptible to developing a disease or condition.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The present disclosure also provides compounds that bear a sulfonyl moiety, a suloximinyl moiety, a sulfinyl moiety, or a combination thereof. For example, a compound of the disclosure can bear the divalent radical

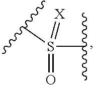

where X is O, NR^{Z}, or absent, and R^{z} is alkyl, cycloalkyl, heteroalkyl, or cycloheteroalkyl, any of which is substituted or unsubstituted, or hydrogen. In some embodiments, a compound of the disclosure can bear the monovalent radical

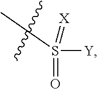

where Y is a substituted or unsubstituted 5-membered or 6-membered ring optionally comprising 1-3 hetero ring atoms selected from O, N, and S; and X is O, NR^{Z}, or absent, where R^{Z} is H, alkyl, cycloalkyl, heteroalkyl, or cycloheteroalkyl, any of which is substituted or unsubstituted, or hydrogen. It shall be understood that when X is “absent,” the monovalent radical

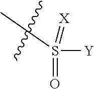

shall be equivalent to

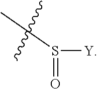

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In some aspects of the disclosed methods, the subject has been diagnosed with a need for treatment of one or more oncological disorders or cancers prior to the administering step. In some aspects of the disclosed method, the subject has been diagnosed with a need for inhibition or negative modulation of STAT5 prior to the administering step. In some aspects of the disclosed method, the subject has been diagnosed with a need for treatment of one or more oncological disorders or cancers associated with STAT5 dysfunction prior to the administering step. In some embodiments, the subject is suspected of having a condition or disease.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “substituted,” “substituent” or the like, unless otherwise indicated, can refer to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted.

The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

### II. STAT5 Inhibitory Compounds.

Provided herein are STAT5 inhibitory compounds and pharmaceutical compositions comprising said compounds. The subject compounds and compositions are useful for inhibiting signal transducer and activator of transcription 5a and 5b (STAT5) proteins and for the treatment of a cell proliferative disease such as cancer.

Described herein is a compound having the structure of Formula (VI), or a pharmaceutically acceptable salt or solvate thereof:

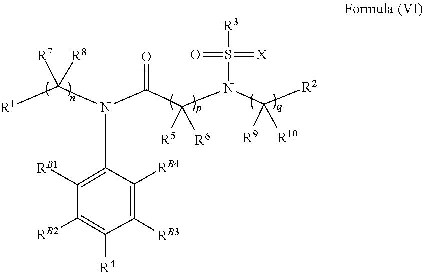

wherein
R¹ is substituted or unsubstituted alkyl, substituted or unsubstituted aryl, substituted or unsubstituted cycloalkyl, substituted or unsubstituted heteroaryl, or substituted or unsubstituted heterocycloalkyl, wherein the heteroaryl or heterocycloalkyl contains 1 to 4 heteroatoms selected from O, N, and S;
R² is substituted or unsubstituted C₃-C₈ cycloalkyl, substituted or unsubstituted C₂-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, CN, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, CN, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl,
   provided that p is 1 and q is 1;
each of R^{B1}, R^{B2}, R^{B3}, and R^{B4} is independently H or R^{B}, wherein each R^{B} is independently D, halogen, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3; and
p is 1, 2, or 3.

In some embodiments of a compound of Formula (VI), or a pharmaceutically acceptable salt or solvate thereof, R¹ is substituted or unsubstituted alkyl, substituted or unsubstituted aryl, substituted or unsubstituted cycloalkyl, substituted or unsubstituted heteroaryl, or substituted or unsubstituted heterocycloalkyl, wherein the aryl, cycloalkyl, heteroaryl, or heterocycloalkyl is mono- or bi-cyclic. In some embodiments, R¹ is substituted or unsubstituted mono- or bi-cyclic aryl, substituted or unsubstituted C₃-C₈ cycloalkyl, substituted or unsubstituted mono- or bi-cyclic heteroaryl, or substituted or unsubstituted C₃-C₇ heterocycloalkyl.

In some embodiments of a compound of Formula (VI), or a pharmaceutically acceptable salt or solvate thereof, R¹ is substituted or unsubstituted C₁-C₆ alkyl, substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S. In some embodiments, R¹ is methyl.

In some embodiments of a compound of Formula (VI), or a pharmaceutically acceptable salt or solvate thereof, R^{B1}, R^{B3}, and R^{B4} are H. In some embodiments of a compound of Formula (VI), or a pharmaceutically acceptable salt or solvate thereof, R^{B1} is R^{B}. In some embodiments of a compound of Formula (VI), or a pharmaceutically acceptable salt or solvate thereof, R^{B2} is OH. In some embodiments of a compound of Formula (VI), or a pharmaceutically acceptable salt or solvate thereof, R^{B1}, R^{B2}, R^{B3}, and R^{B4} are H.

In some embodiments of a compound of Formula (VI), or a pharmaceutically acceptable salt or solvate thereof, R¹ is

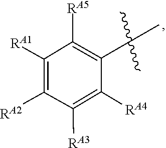

wherein each of R^{A1}, R^{A2}, R^{A3}, R^{A4}, and R^{A5} is independently R^{A}, and wherein each R^{A} is independently H, halogen, —CN, —NO₂, —OR¹¹, —N(R¹¹)₂, substituted or unsubstituted C₁-C₆ alkyl, substituted or unsubstituted C₁-C₆haloalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, R^{A1} is D. In some embodiments, R^{A2} is D. In some embodiments, R^{A3} is D. In some embodiments, R^{A4} is D.

In some embodiments, a compound of Formula (VI) has a structure of Formula (I), as described in this disclosure.

One aspect of the disclosure provides a compound having the structure of Formula (I), or a pharmaceutically acceptable salt or solvate thereof:

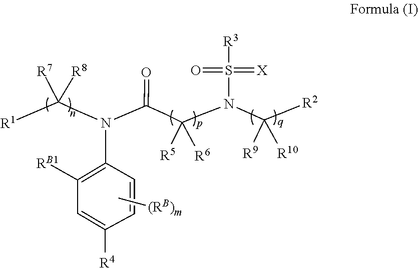

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
   provided that p is 1 and q is 1;
each of R^{B} and R^{B1} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, or 3.

In some embodiments of a compound of Formula (I), or a pharmaceutically acceptable salt or solvate thereof:
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring.

In some embodiments of a compound of Formula (VI) or (I), or a pharmaceutically acceptable salt or solvate thereof, R⁶ is independently selected from H, F, —CN, substituted or unsubstituted C₁-C₆ alkyl, substituted or unsubstituted C₁-C₆ haloalkyl, substituted or unsubstituted C₁-C₆ heteroalkyl, and substituted or unsubstituted C₁-C₆ alkoxy. In some embodiments, each R⁶ is independently selected from H, F, methyl, ethyl, propyl, iso-propyl, n-butyl, iso-butyl, sec-butyl, t-butyl, —CF₃, —CH₂CF₃, —CH₂CH₂F, —OCF₃, —OH, —OCH₃, —OCH₂CH₃, —OCH₂OMe, and —OCH₂CH₂OH. In some embodiments, each R⁶ is H. In some embodiments, R⁶ is D.

In some embodiments of a compound of Formula (VI) or (I), or a pharmaceutically acceptable salt or solvate thereof, R⁵ and R⁶ taken together form an oxo.

In some embodiments of a compound of Formula (VI) or (I), or a pharmaceutically acceptable salt or solvate thereof, R⁵ and R⁶ taken together with the carbon to which they are attached form a substituted or unsubstituted 4, 5, or 6 membered heterocyclic ring. In some embodiments, R⁵ and R⁶ taken together with the carbon to which they are attached form an oxetane, azetidine, tetrahydrofuran, or morpholine ring.

In some embodiments of a compound of Formula (VI) or (I), or a pharmaceutically acceptable salt or solvate thereof, R⁵ and R⁶ taken together with the carbon to which they are attached form a substituted or unsubstituted 3, 4, 5, or 6 membered cycloalkyl ring. In some embodiments, R⁵ and R⁶ taken together with the carbon to which they are attached form a substituted or unsubstituted cyclobutane, cyclopentane, or cyclohexane.

In some embodiments of a compound of Formula (VI) or (I), or a pharmaceutically acceptable salt or solvate thereof, X is O. In some embodiments, X is NR¹¹. In some embodiments, X is NH. In some embodiments, X is N-alkyl. In some embodiments, X is absent.

In some embodiments of a compound of Formula (I), or a pharmaceutically acceptable salt or solvate thereof, the compound has the structure of Formula (II):

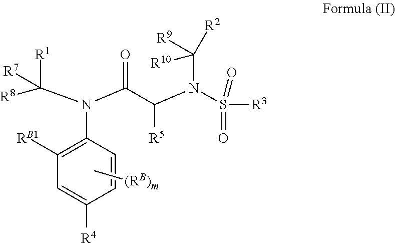

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹C(═O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In some embodiments of a compound of Formula (II), or a pharmaceutically acceptable salt or solvate thereof:
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring.

In some embodiments of a compound of Formula (II), or a pharmaceutically acceptable salt or solvate thereof, the compound has the structure of Formula (IIa):

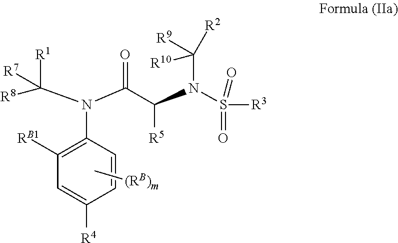

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In some embodiments of a compound of Formula (II), or a pharmaceutically acceptable salt or solvate thereof, the compound has the structure of Formula (IIb):

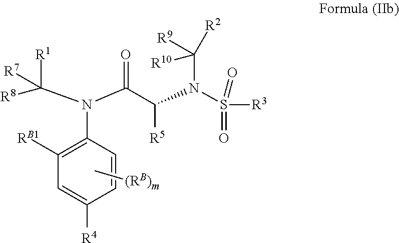

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹C(═O)R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
m is 0, 1, 2, or 3.

In some embodiments of a compound of Formula (VI) or (I), or a pharmaceutically acceptable salt or solvate thereof, each of R⁵ is independently selected from the group consisting of H, F, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl. In some embodiments, each of R⁵ is independently selected from the group consisting of H, F, —CN, —NH(CH₃), —NH₂, —N(CH₃)₂ , —NHR¹¹, methyl, ethyl, propyl, iso-propyl, n-butyl, iso-butyl, sec-butyl, t-butyl, linear or branched pentyl, linear or branched hexyl, cyclopropyl, cyclobutyl, cyclopentyl, cyclohexyl, cycloheptyl, cyclooctyl, —CF₃, —CH₂CF₃, —CH₂CH₂F, —OCF₃, —OH, —SH, —OCH₃, —OCH₂CH₃, —OCH₂OMe, and —OCH₂CH₂OH.

In some embodiments of a compound of Formula (VI), (I), (II), (IIa), or (IIb), or a pharmaceutically acceptable salt or solvate thereof, each of R⁵ is independently H, methyl, ethyl, propyl, butyl, pentyl, or hexyl, wherein the methyl, ethyl, propyl, butyl, pentyl, or hexyl is linear or branched, substituted or unsubstituted. In some embodiments, each of R⁵ is independently H, methyl, ethyl, propyl, butyl, pentyl, or hexyl, wherein the methyl, ethyl, propyl, butyl, pentyl, or hexyl is linear or branched, and optionally substituted with 1 to 3 F, methoxy, hydroxy, or amino. In some embodiments, each of R⁵ is independently H, CH₃, CF₃, or C₂F. In some embodiments, R⁵ is D.

In some embodiments of a compound of Formula (VI) or (I), or a pharmaceutically acceptable salt or solvate thereof, each of R⁷, R⁸, R⁹, and R¹⁰ is independently selected from the group consisting of H, amino, F, substituted or unsubstituted C₁-C₆ alkoxy, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy. In some embodiments, each of R⁷, R⁸, R⁹, and R¹⁰ is independently selected from the group consisting of H, amino, F, substituted or unsubstituted C₁-C₆ alkoxy, substituted or unsubstituted C₁-C₆ alkyl, substituted or unsubstituted C₁-C₆ haloalkyl, and substituted or unsubstituted C₁-C₆ heteroalkyl. In some embodiments, each of R⁷, R⁸, R⁹, and R¹⁰ is independently selected from the group consisting of H, amino, F, substituted or unsubstituted C₁-C₆ alkoxy, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy. In some embodiments, each of R⁷, R⁸, R⁹, and R¹⁰ is independently selected from the group consisting of H, F, —NH(CH₃), —NH₂, —N(CH₃)₂, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, and —OC₂C₂OH.

In some embodiments of a compound of Formula (VI), (I), (II), (IIa), or (IIb), or a pharmaceutically acceptable salt or solvate thereof, each of R⁷, R⁸, R⁹, and R¹⁰ is independently selected from the group consisting of H, F, substituted or unsubstituted C₁-C₆ alkyl, substituted or unsubstituted C₁-C₆ fluoroalkyl, and substituted or unsubstituted C₁-C₆ heteroalkyl, wherein the alkyl, fluoroalkyl or heteroalkyl is optionally substituted with hydroxy, amino, or methoxy. In some embodiments, each of R⁷, R⁸, R⁹, and R¹⁰ is independently H, F, methyl, ethyl, propyl, —CF₃, or —CH₂CF₃. In some embodiments, each of R⁷, R⁸, R⁹, and R¹⁰ is H. In some embodiments, R⁷ is D. In some embodiments, R⁸ is D. In some embodiments, R⁹ is D. In some embodiments, R¹⁰ is D.

In some embodiments of a compound of Formula (VI), (I), (II), (IIa), or (IIb), or a pharmaceutically acceptable salt or solvate thereof, R⁵ and R⁹, taken together with the intervening atoms to which they are attached form a 4, 5 or 6-membered substituted or unsubstituted cycloalkyl or heterocycloalkyl ring.

In some embodiments of a compound of Formula (VI), (I), (II), (IIa), or (IIb), or a pharmaceutically acceptable salt or solvate thereof, R⁷ and R⁸, taken together form a substituted or unsubstituted 3, 4, 5, or 6-membered cycloalkyl or heterocycloalkyl ring.

In some embodiments of a compound of Formula (VI), (I), (II), (IIa), or (IIb), or a pharmaceutically acceptable salt or solvate thereof, R⁹ and R¹⁰, taken together form a substituted or unsubstituted 3, 4, 5, or 6-membered cycloalkyl or heterocycloalkyl ring.

In some embodiments of a compound of Formula (I), or a pharmaceutically acceptable salt or solvate thereof, the compound has the structure of Formula (III):

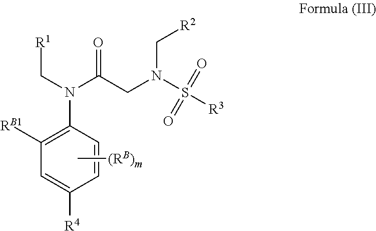

wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is a pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{B} and R^{B1} is independently halogen, D, —CN, —NO₂, —OR¹¹, —SR¹¹, —N(R¹¹)₂, NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

In some embodiments of a compound of Formula (VI), (I), (II), (IIa), (IIb), or (III), or a pharmaceutically acceptable salt or solvate thereof, R^{B1} is halogen. In some embodiments, R^{B1} is F or Cl.

In some embodiments of a compound of Formula (VI), (I), (II), (IIa), (IIb), or (III), or a pharmaceutically acceptable salt or solvate thereof, R^{B1} is a linear or branched, substituted or unsubstituted C₁-C₆ alkyl. In some embodiments, R^{B1} is methyl, ethyl, propyl, iso-propyl, n-butyl, iso-butyl, sec-butyl, t-butyl, linear or branched pentyl, linear or branched hexyl, —CF₃, —C₂NH₂, —CH₂CF₃, —CH₂CHNH₂, —CH₂C₂F, —CH₂OH, or —CH₂C₂OH. In some embodiments, R^{B1} is substituted or unsubstituted —C₀₋₃ alkylene-C₃₋₈ cycloalkyl, or substituted or unsubstituted —C₀₋₃ alkylene-C₃₋₇ heterocycloalkyl.

In some embodiments of a compound of Formula (VI), (I), (II), (IIa), (IIb), or (III), or a pharmaceutically acceptable salt or solvate thereof, R^{B1} is C₃₋₆ cycloalkyl, —CH₂-C₃₋₆ cycloalkyl, —(C₂)₂—C₃₋₆ cycloalkyl, —(C₂)₃—C₃₋₆ cycloalkyl, C₃₋₅ heterocycloalkyl, —CH₂—C₃₋₅ heterocycloalkyl, —(C₂)₂—C₃₋₅ heterocycloalkyl, or —(C₂)₃—C₃₋₅ heterocycloalkyl, wherein the cycloalkyl and heterocycloalkyl is substituted or unsubstituted. In some embodiments, the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur. In some embodiments, R^{B1} is

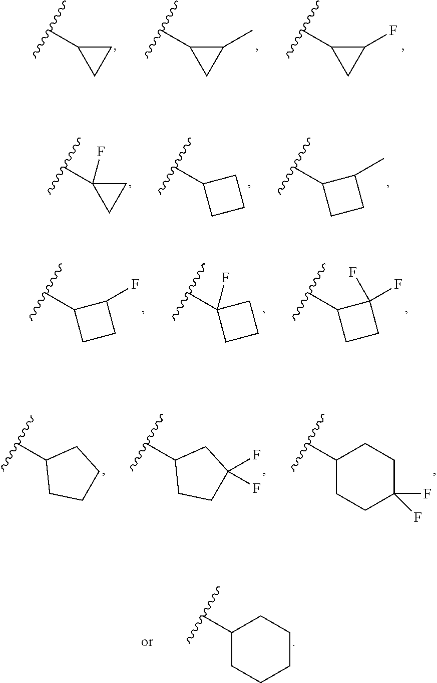

In some embodiments, R^{B1} is

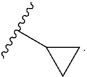 [SMILES: *C1CC1]

In some embodiments, R^{B1} is

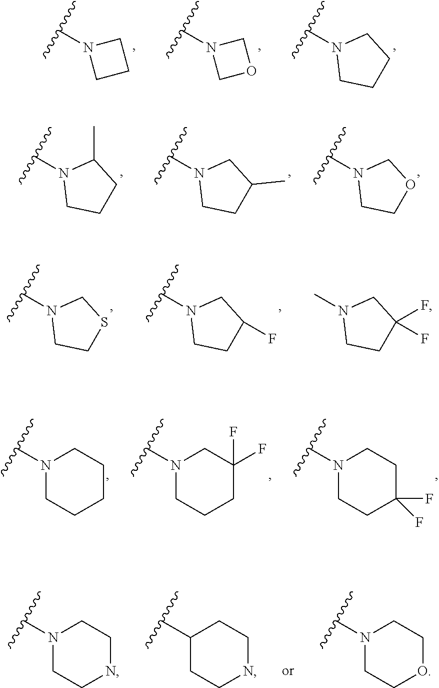

In some embodiments of a compound of Formula (VI), (I), (II), (IIa), (IIb), or (III), or a pharmaceutically acceptable salt or solvate thereof, R^{B1} is —OR¹¹. In some embodiments, R^{B1} is OH. In some embodiments, R^{B1} is substituted or unsubstituted —O—C₁-C₆ alkyl. In some embodiments, R^{B1} is

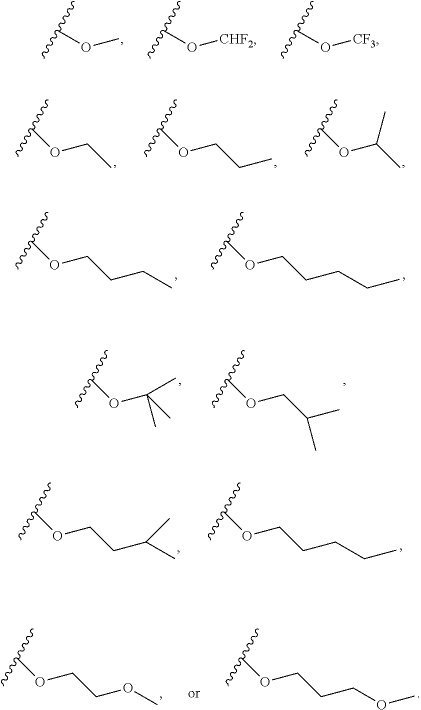

In some embodiments, R^{B1} is

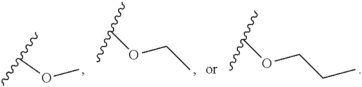

In some embodiments, R^{B1} is

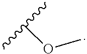 [SMILES: *OC]

In some embodiments, R^{B1} is

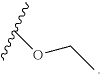 [SMILES: *OCC]

In some embodiments, R^{B1} is

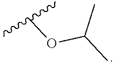 [SMILES: *OC(C)C]

In some embodiments of a compound of Formula (VI), (I), (II), (IIa), (IIb), or (III), or a pharmaceutically acceptable salt or solvate thereof, R^{B1} is substituted or unsubstituted alkylene-C₃₋₈ cycloalkyl, or substituted or unsubstituted alkylene-C₃₋₇ heterocycloalkyl. In some embodiments, R^{B1} is substituted or unsubstituted alkylene-C₃₋₆ cycloalkyl, or substituted or unsubstituted alkylene-C₃₋₆ heterocycloalkyl. In some embodiments, R^{B1} is

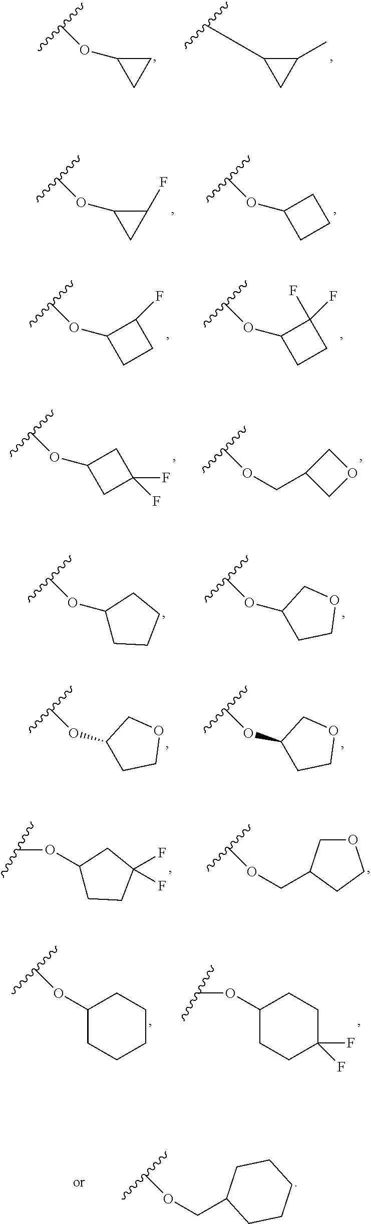

In some embodiments, R^{B1} is

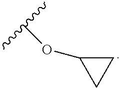 [SMILES: *OC1CC1]

In some embodiments of a compound of Formula (VI), (I), (II), (IIa), (IIb), or (III), or a pharmaceutically acceptable salt or solvate thereof, R^{B1} is -N(R¹¹)2. In some embodiments, R^{B1} is —N(CH₃)₂, —NHCH₃, —N(C₂CH₃)₂, —NHC₂CH₃, or —N(C₂C₂CH₃)₂.

In some embodiments of a compound of Formula (VI), (I), (II), (IIa), (IIb), or (III), or a pharmaceutically acceptable salt or solvate thereof, R^{B1} is —NR¹¹S(═O)₂R¹¹. In some embodiments, R^{B1} is —NCH₃S(═O)₂CH₃.

In some embodiments of a compound of Formula (VI), (I), (II), (IIa), (IIb), or (III), or a pharmaceutically acceptable salt or solvate thereof, m is 0 or 1.

In some embodiments of a compound of Formula (I), (II), (IIa), (IIb), or (III), or a pharmaceutically acceptable salt or solvate thereof,

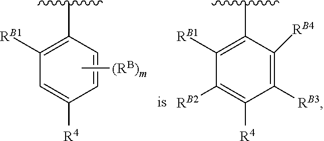

wherein each of R^{B2}, R^{B3}, and R^{B4} is independently H or R^{B}.

In some embodiments of a compound of Formula (VI), (I), (II), (IIa), (IIb), or (III), or a pharmaceutically acceptable salt or solvate thereof, each R^{B} is independently halogen, D, —CN, —OR¹¹, —SR¹¹, —N(R¹¹)₂, —NR¹¹S(—O)₂R¹¹, substituted or unsubstituted C₁-C₆ alkyl, substituted or unsubstituted —C₀₋₃ alkylene-C₃₋₆ cycloalkyl, or substituted or unsubstituted —C₀₋₃ alkylene-C₃₋₅ heterocycloalkyl. In some embodiments, each R^{B} is independently a halogen selected from F and Cl. In some embodiments, each R^{B} is independently linear or branched, substituted or unsubstituted C₁-C₆ alkyl. In some embodiments, each C₁-C₆ alkyl is independently methyl, ethyl, propyl, iso-propyl, n-butyl, iso-butyl, sec-butyl, t-butyl, linear or branched pentyl, linear or branched hexyl, —CF₃, —CH₂NH₂, —CH₂CF₃, —CH₂CHNH₂, —CH₂C₂F, —CH₂OH, or —C₂C₂OH.

In some embodiments of a compound of Formula (VI), (I), (II), (IIa), (IIb), or (III), or a pharmaceutically acceptable salt or solvate thereof, at least one R^{B} is NR11C(═O)R¹¹. In some embodiments, at least one R^{B} is —NHCOCH₃ or —N(CH₃)COCH₃.

In some embodiments of a compound of Formula (VI), (I), (II), (IIa), (IIb), or (III), or a pharmaceutically acceptable salt or solvate thereof, each of R^{B} is independently substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each of R^{B} is independently substituted or unsubstituted —C₀₋₆ alkylene-C₃₋₈ cycloalkyl. In some embodiments, each of R^{B} is independently substituted or unsubstituted —C₀₋₆ alkylene-C₃₋₇ heterocycloalkyl. In some embodiments, each of R^{B} is independently substituted or unsubstituted —C₀₋₃ alkylene-C₃₋₈ cycloalkyl, or substituted or unsubstituted —C₀₋₃ alkylene-C₃₋₇ heterocycloalkyl. In some embodiments, each of R^{B} is independently C₃₋₆ cycloalkyl, —CH₂-C₃₋₆ cycloalkyl, —(C₂)₂—C₃₋₆ cycloalkyl, —(C₂)₃—C₃₋₆ cycloalkyl, C₃₋₅ heterocycloalkyl, —CH₂-C₃₋₅ heterocycloalkyl, —(C₂)₂—C₃₋₅ heterocycloalkyl, or —(C₂)₃—C₃₋₅ heterocycloalkyl, wherein the cycloalkyl and heterocycloalkyl is substituted or unsubstituted. In some embodiments, the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

In some embodiments of a compound of Formula (VI), (I), (II), (IIa), (IIb), or (III), or a pharmaceutically acceptable salt or solvate thereof, each of R^{B} is independently substituted or unsubstituted —C₀₋₆ alkylene-C₃₋₈ cycloalkyl, or substituted or unsubstituted —C₀₋₆ alkylene-C₃₋₇ heterocycloalkyl. In some embodiments each of the cycloalkyl is independently

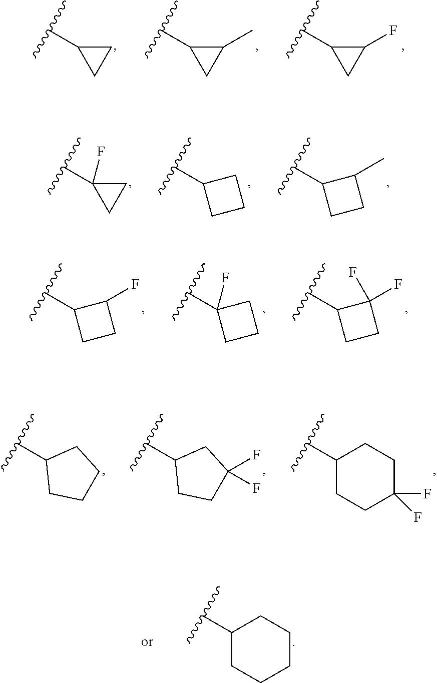

In some embodiments, each of the heterocycloalkyl is independently

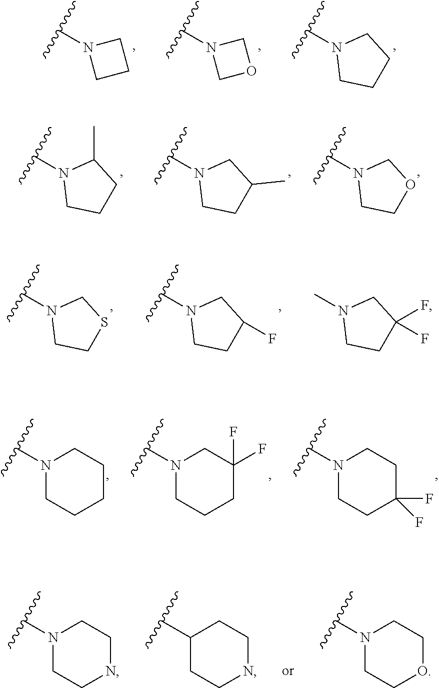

In some embodiments of a compound of Formula (VI), (I), (II), (IIa), (IIb), or (III), or a pharmaceutically acceptable salt or solvate thereof, each R^{B} is independently —OR¹¹. In some embodiments, each —OR¹¹ is independently OH, —O—C₁-C₆ alkyl, —O—C₁-C₆ haloalkyl, —O—C₁-C₆ heteroalkyl, —O—C₀₋₆ alkylene-C₃₋₈ cycloalkyl, or —O—C₀₋₆ alkylene-C₃₋₇ heterocycloalkyl, wherein the alkyl, haloalkyl, heteroalkyl, cycloalkyl, and heterocycloalkyl is substituted or unsubstituted. In some embodiments, each R^{B} is independently substituted or unsubstituted —O— C₁-C₆ alkyl. In some embodiments, each R^{B} is independently

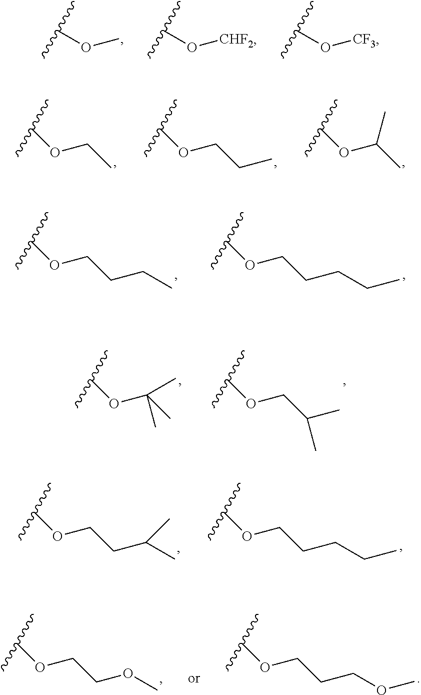

In some embodiments, each R^{B} is

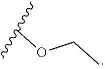 [SMILES: *OCC]

In some embodiments, each R^{B} is OH.

In some embodiments of a compound of Formula (VI), (I), (II), (IIa), (IIb), or (III), or a pharmaceutically acceptable salt or solvate thereof, each R^{B} is independently substituted or unsubstituted —O—C₀₋₆ alkylene-C₃₋₈ cycloalkyl, or substituted or unsubstituted —O—C₀₋₆ alkylene-C₃₋₇ heterocycloalkyl. In some embodiments, each R^{B} is independently

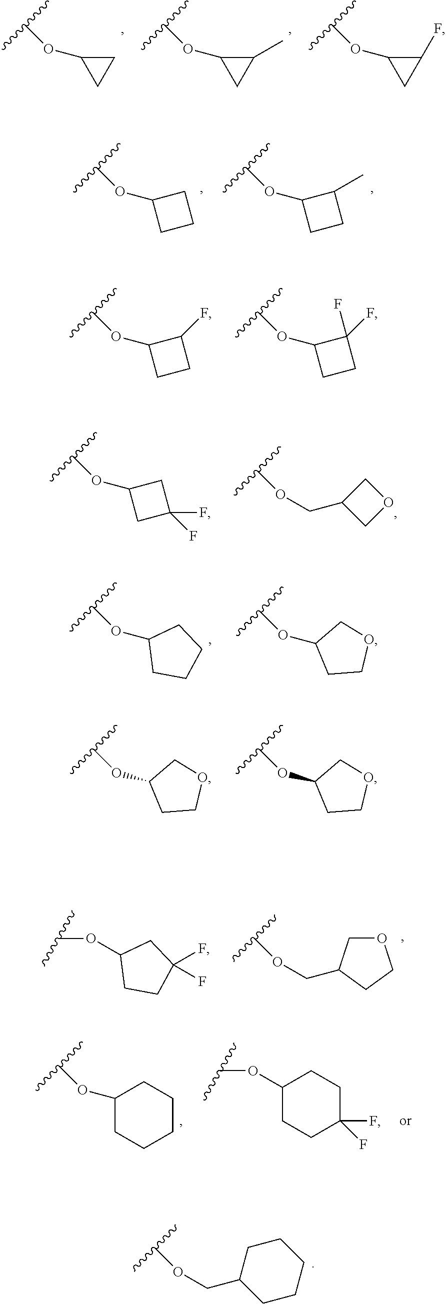

In some embodiments, each R^{B} is

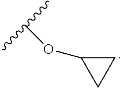 [SMILES: *OC1CC1]

In some embodiments of a compound of Formula (VI), (I), (II), (IIa), (IIb), or (III), or a pharmaceutically acceptable salt or solvate thereof, each R^{B} is independently —N(R¹¹)₂. In some embodiments, each R^{B} is independently —N(CH₃)₂, —NHCH₃, —N(C₂CH₃)₂, —NHC₂CH₃, or —N(C₂C₂CH₃)₂.

In some embodiments of a compound of Formula (VI), (I), (II), (IIa), (IIb), or (III), or a pharmaceutically acceptable salt or solvate thereof, at least one R^{B} is —NR¹¹S(═O)₂R¹¹. In some embodiments, at least one R^{B} is —NR¹¹S(═O)₂R¹¹, wherein each R¹¹ is independently H or C₁-C₃ alkyl. In some embodiments, the —NR¹¹S(═O)₂R^{1l} is —NCH₃S(═O)₂CH₃.

In some embodiments of a compound of Formula (VI), (I), (II), (IIa), (IIb), or (III), or a pharmaceutically acceptable salt or solvate thereof, R⁴ is COOH or an isostere thereof. In some embodiments, R⁴ is SO₂H.

In some embodiments of a compound of Formula (VI), (I), (II), (IIa), (IIb), or (III), or a pharmaceutically acceptable salt or solvate thereof, R⁴ is C(O)N(R¹¹)₂, C(O)OR¹¹, or S(O)₂N(R¹¹)₂. In some embodiments, R⁴ is C(O)N(R¹¹)₂, C(O)OR¹¹, or S(O)₂N(R¹¹)₂, wherein each R¹¹ is independent H or C₁-C₆ alkyl. In some embodiments, R⁴ is C(O)N(Me)₂, C(O)NH₂, C(O)NHCH₃, C(O)OMe, S(O)₂N(Me)₂, S(O)₂NH₂, or S(O)₂NHCH₃.

In some embodiments of a compound of Formula (VI), (I), (II), (IIa), (IIb), or (III), or a pharmaceutically acceptable salt or solvate thereof, R⁴ is —OR¹¹. In some embodiments, R⁴ is

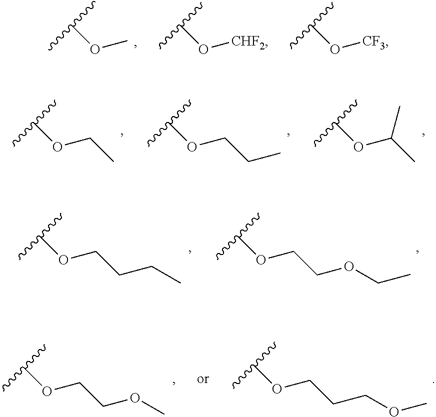

In some embodiments of a compound of Formula (VI), (I), (II), (IIa), (IIb), or (III), or a pharmaceutically acceptable salt or solvate thereof, R⁴ is —C₀₋₆ alkylene-R⁴¹ or —C₂₋₄ alkylene-R⁴¹. In some embodiments,

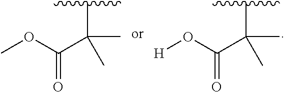

In some embodiments of a compound of Formula (I), (II), (IIa), (IIb), or (III), or a pharmaceutically acceptable salt or solvate thereof,

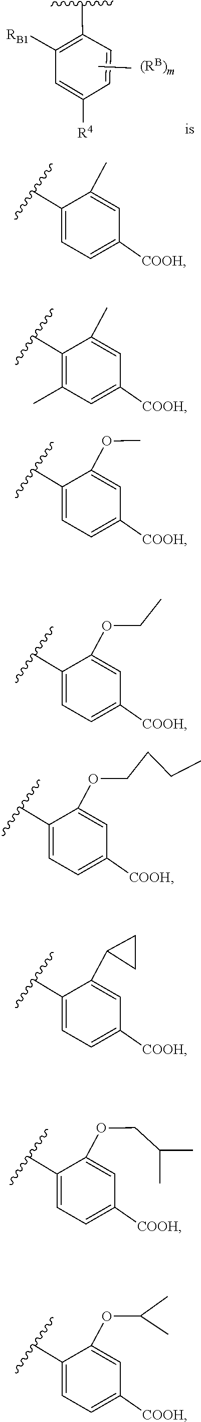

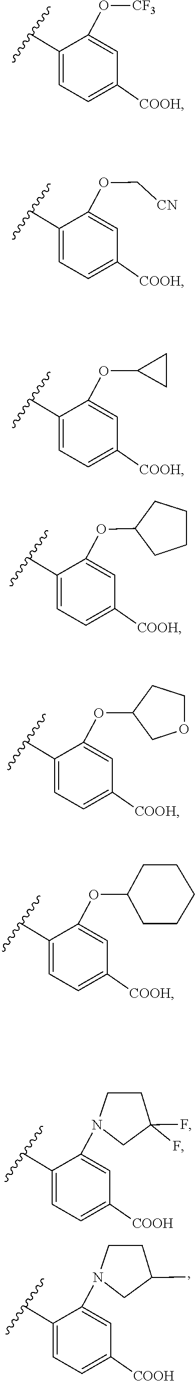

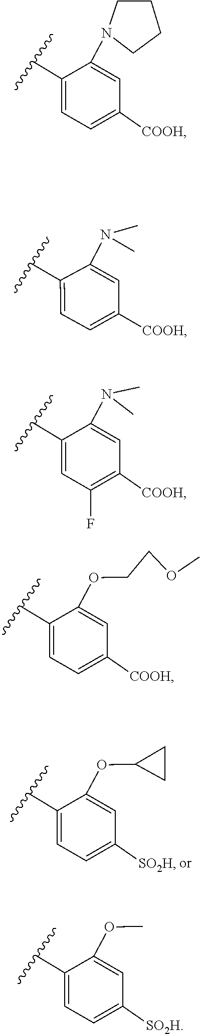

In some embodiments of a compound of Formula (I), (II), (IIa), (IIb), or (III), or a pharmaceutically acceptable salt or solvate thereof,

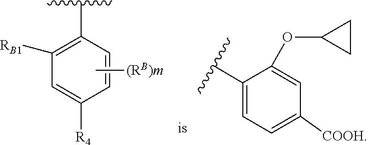

In some embodiments of a compound of Formula (I), (II), (IIa), (IIb), or (III), or a pharmaceutically acceptable salt or solvate thereof,

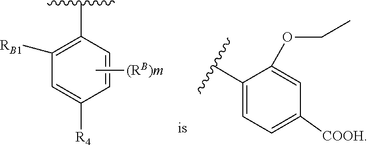

In some embodiments of a compound of Formula (VI), (I), (II), (IIa), (IIb), or (III), or a pharmaceutically acceptable salt or solvate thereof, R¹ is substituted or unsubstituted phenyl. In some embodiments, R¹ is substituted phenyl, and wherein the phenyl is substituted with 1 to 5 substituents independently selected from halogen, D, —CN, —NO₂, —OR¹¹, —N(R¹¹)₂, substituted or unsubstituted C₁-C₆ alkyl, substituted or unsubstituted C₁-C₆haloalkyl, substituted or unsubstituted —C₀₋₆ alkylene-C₃₋₈ cycloalkyl, and substituted or unsubstituted —C₀₋₆ alkylene-C₃₋₇ heterocycloalkyl. In some embodiments, R¹ is substituted phenyl, and wherein the phenyl is substituted with F or Cl. In some embodiments, R¹ is substituted phenyl, wherein the phenyl is substituted with —O—C₁-C₆ alkyl, and wherein the alkyl is substituted or unsubstituted. In some embodiments, R¹ is substituted phenyl, and wherein the phenyl is substituted with one or two C₁-C₆ alkyl, and wherein the alkyl is linear or branched, substituted or unsubstituted. In some embodiments, R¹ is substituted phenyl, and wherein the phenyl is substituted with one or two C₃₋₈ cycloalkyl, and wherein the cycloalkyl is substituted or unsubstituted. In some embodiments, R¹ is substituted phenyl, wherein the phenyl is substituted with one C₃₋₈ cycloalkyl and one C₁-C₆ alkyl, and wherein the cycloalkyl and alkyl is substituted or unsubstituted.

In some embodiments of a compound of Formula (VI), (I), (II), (IIa), (IIb), or (III), or a pharmaceutically acceptable salt or solvate thereof, R¹ is substituted phenyl, wherein the phenyl is substituted with 1, 2, or 3 R^{A}, and wherein each R^{A} is independently halogen, D, —CN, —NO₂, —OR¹¹, substituted or unsubstituted C₁-C₆ alkyl, substituted or unsubstituted C₁-C₆haloalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments of a compound of Formula (VI), (I), (II), (IIa), (IIb), or (III), or a pharmaceutically acceptable salt or solvate thereof, each R^{A} is independently halogen, —OR¹¹, substituted or unsubstituted C₁-C₆ alkyl, substituted or unsubstituted C₁-C₆ haloalkyl, substituted or unsubstituted —C₀₋₃ alkylene-C₃₋₆ cycloalkyl, or substituted or unsubstituted —C₀₋₃ alkylene-C₃₋₅ heterocycloalkyl. In some embodiments, each R^{A} is independently halogen, C₁-C₆ alkyl, —C₀₋₃ alkylene-C₃₋₆ cycloalkyl, —C₀₋₃ alkylene-C₃₋₅ heterocycloalkyl, —O—C₁-C₆ alkyl, -0—C₀₋₃ alkylene-C₃₋₆ cycloalkyl, or —O—C₀₋₃ alkylene-C₃₋₅ heterocycloalkyl, and wherein the alkyl, cycloalkyl, and heterocycloalkyl is substituted or unsubstituted. In some embodiments, each R^{A} is independently F, Cl, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

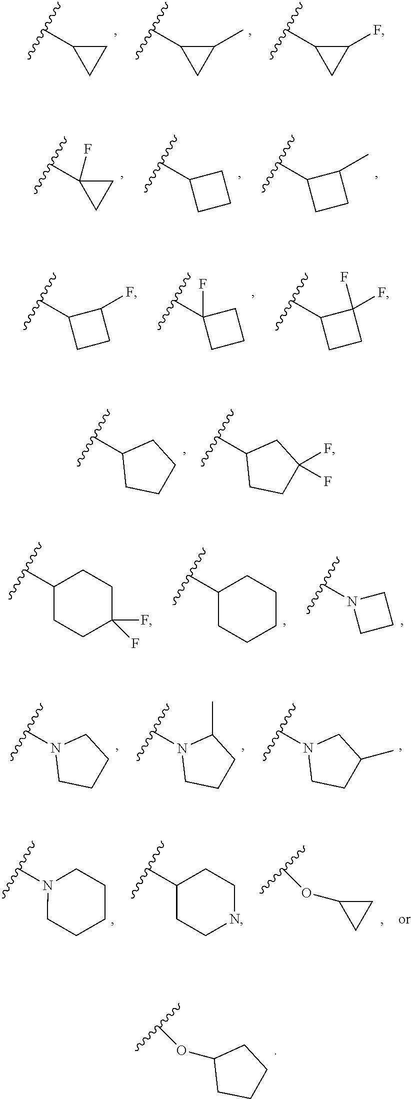

In some embodiments of a compound of Formula (VI), (I), (II), (IIa), (IIb), or (III), or a pharmaceutically acceptable salt or solvate thereof, each R^{A} is

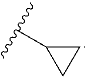 [SMILES: *C1CC1]

In some embodiments of a compound of Formula (VI), (I), (II), (IIa), (IIb), or (III), or a pharmaceutically acceptable salt or solvate thereof, R¹ is

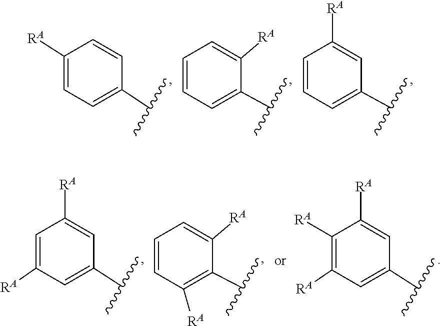

In some embodiments of a compound of Formula (VI), (I), (II), (IIa), (IIb), or (III), or a pharmaceutically acceptable salt or solvate thereof, R¹ is substituted phenyl, wherein the phenyl is substituted with 1, 2, or 3 R^{A}, and wherein two R^{A}, taken together with the intervening atoms to which they are attached form a 4, 5, or 6 membered ring. In some embodiments, the 4, 5, or 6 membered ring comprises 1 to 3 heteroatoms selected from N, O, and S. In some embodiments, R¹ is

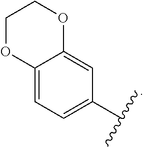 [SMILES: *c1ccc2c(c1)OCCO2]

In some embodiments of a compound of Formula (VI), (I), (II), (IIa), (IIb), or (III), or a pharmaceutically acceptable salt or solvate thereof, R¹ is

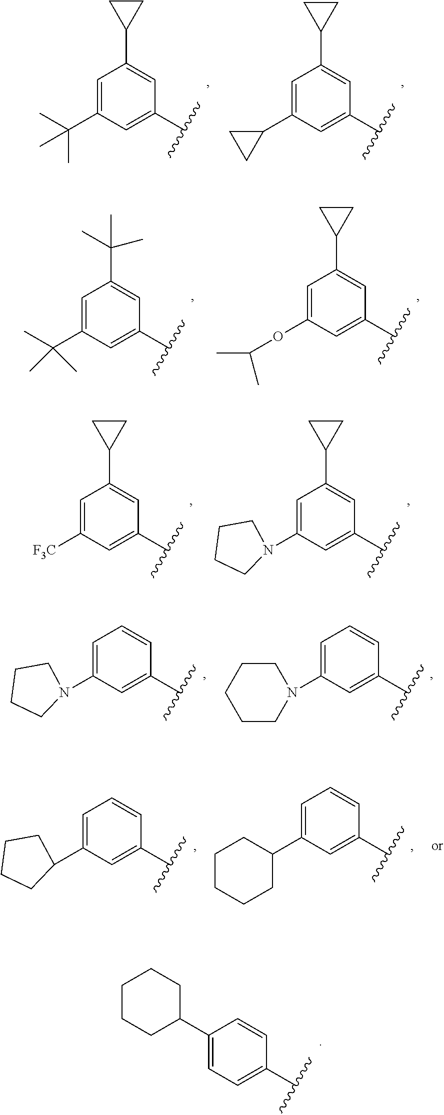

In some embodiments, R¹ is

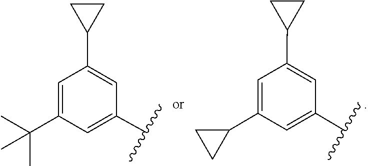 [SMILES: *c1cc(C2CC2)cc(C(C)(C)C)c1 ; *c1cc(C2CC2)cc(C2CC2)c1]

In some embodiments of a compound of Formula (VI), (I), (II), (IIa), (IIb), or (III), or a pharmaceutically acceptable salt or solvate thereof, R¹ is naphthyl.

In some embodiments of a compound of Formula (VI), (I), (II), (IIa), (IIb), or (III), or a pharmaceutically acceptable salt or solvate thereof, R¹ is substituted or unsubstituted C₃-C₈ cycloalkyl.

In some embodiments of a compound of Formula (VI), (I), (II), (IIa), (IIb), or (III), or a pharmaceutically acceptable salt or solvate thereof, R¹ is substituted or unsubstituted C₄-C₆ cycloalkyl. In some embodiments, R¹ is

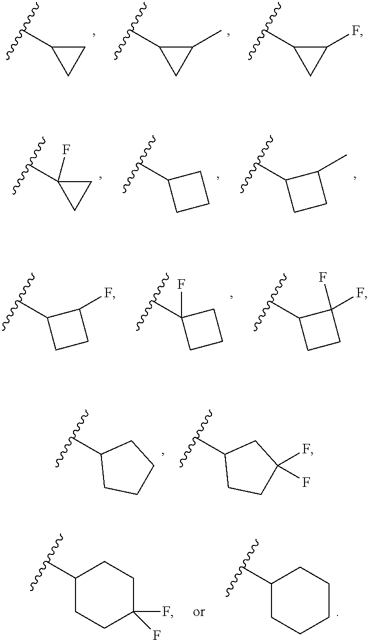

In some embodiments of a compound of Formula (VI), (I), (II), (IIa), (IIb), or (III), or a pharmaceutically acceptable salt or solvate thereof, R¹ is substituted or unsubstituted monocyclic heteroaryl containing 1, 2, or 3 nitrogens. In some embodiments, R¹ is substituted or unsubstituted pyridinyl, pyridazinyl, or pyrimidinyl. In some embodiments, R¹ is

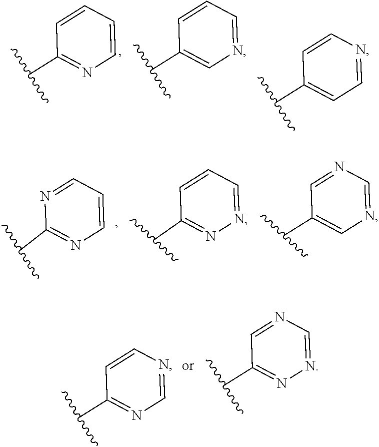

In some embodiments, R¹ is

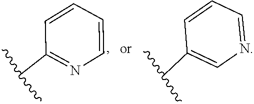 [SMILES: *c1ccccn1 ; *c1cccnc1]

In some embodiments of a compound of Formula (VI), (I), (II), (IIa), (IIb), or (III), or a pharmaceutically acceptable salt or solvate thereof, R¹ is substituted or unsubstituted bicyclic heteroaryl comprising 1 to 2 N.

In some embodiments of a compound of Formula (VI), (I), (II), (IIa), (IIb), or (III), or a pharmaceutically acceptable salt or solvate thereof, R¹ is substituted or unsubstituted 5-6, 6-6, or 6-5 fused bicyclic heteroaryl containing 1-3 hetero ring atoms selected from O, N and S. In some embodiments, R¹ is

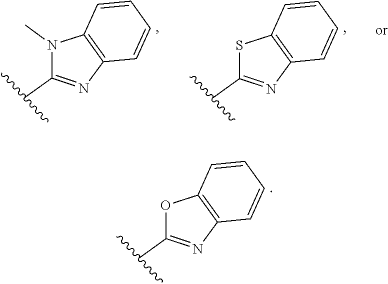 [SMILES: *c1nc2ccccc2n1C ; *c1nc2ccccc2s1 ; *c1nc2ccccc2o1]

In some embodiments of a compound of Formula (VI), (I), (II), (IIa), (IIb), or (III), or a pharmaceutically acceptable salt or solvate thereof, R² is phenyl or substituted phenyl. In some embodiments, R² is phenyl substituted with 1 to 5 R^{c}, and wherein each R^{c} is independently D, halogen, —OR¹¹, —SR¹¹, —N(R¹¹)₂, —CN, —NO₂, substituted or unsubstituted C₁-C₆ alkyl, substituted or unsubstituted C₁-C₆haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, R² is phenyl substituted with 1 to 5 R^{c}, and wherein each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —CH₂C₂F, -OCF₃, —OH, —OCH₃, —OC₂CH₃, —OC₂OMe, —OC₂C₂OH, —OC(CH₃)₃, —OC₂C₂OCH₃,

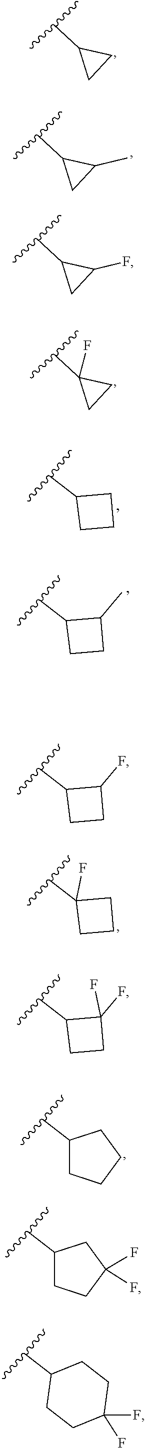

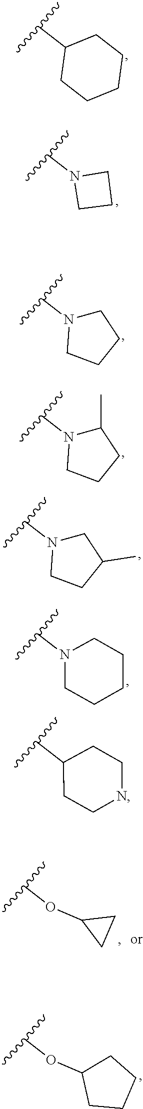

In some embodiments, R² is

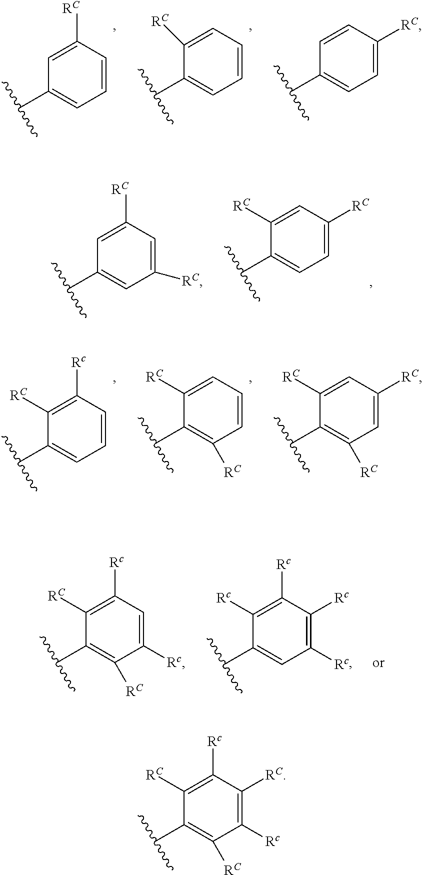

In some embodiments, R² is

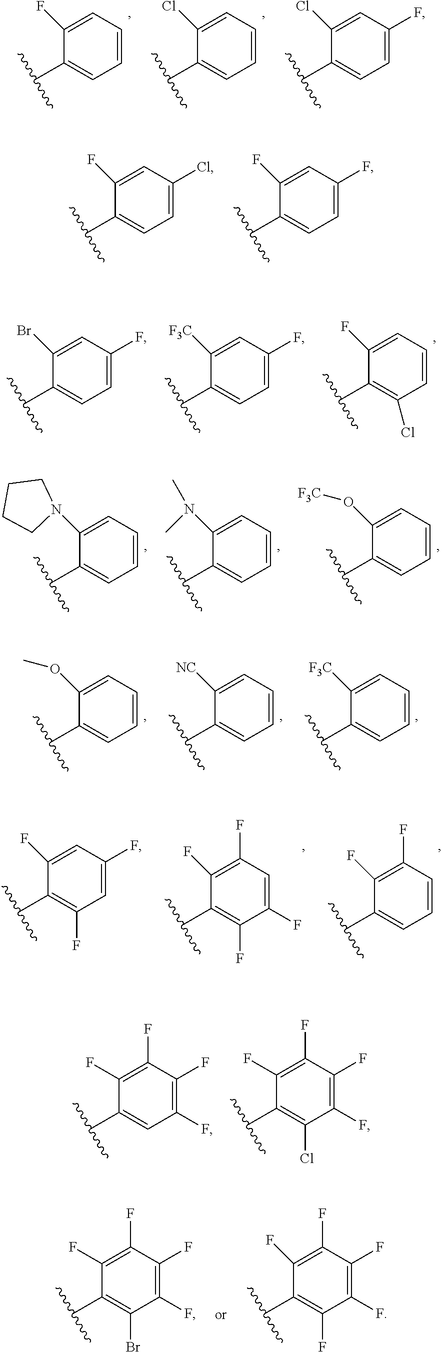

In some embodiments, R² is

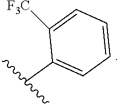 [SMILES: *c1ccccc1C(F)(F)F]

In some embodiments, R² is

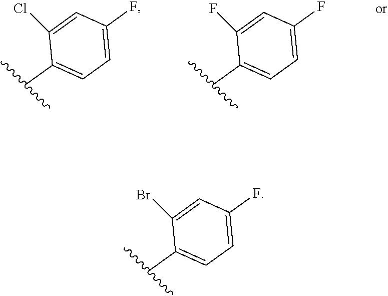 [SMILES: *c1ccc(F)cc1Cl ; *c1ccc(F)cc1F ; *c1ccc(F)cc1Br]

In some embodiments, R² is

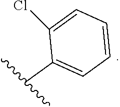 [SMILES: *c1ccccc1Cl]

In some embodiments, R² is

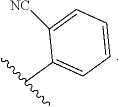 [SMILES: *c1ccccc1C#N]

In some embodiments, R² is

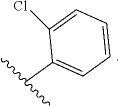 [SMILES: *c1ccccc1Cl]

In some embodiments, R² is

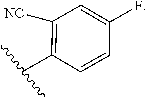 [SMILES: *c1ccc(F)cc1C#N]

In some embodiments of a compound of Formula (VI), (I), (II), (IIa), (IIb), or (III), or a pharmaceutically acceptable salt or solvate thereof, wherein R² is substituted or unsubstituted 5-membered or 6-membered monocyclic heteroaryl. In some embodiments, R² is pyridinyl, pyridazinyl, pyrimidinyl, triazinyl, wherein the pyridinyl, pyridazinyl, pyrimidinyl, or triazinyl is substituted with 1 to 4 r^{c}, and wherein each R^{c} is independently D, halogen, —OR¹¹, —SR¹¹, —N(R¹¹)₂, —CN, —NO₂, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

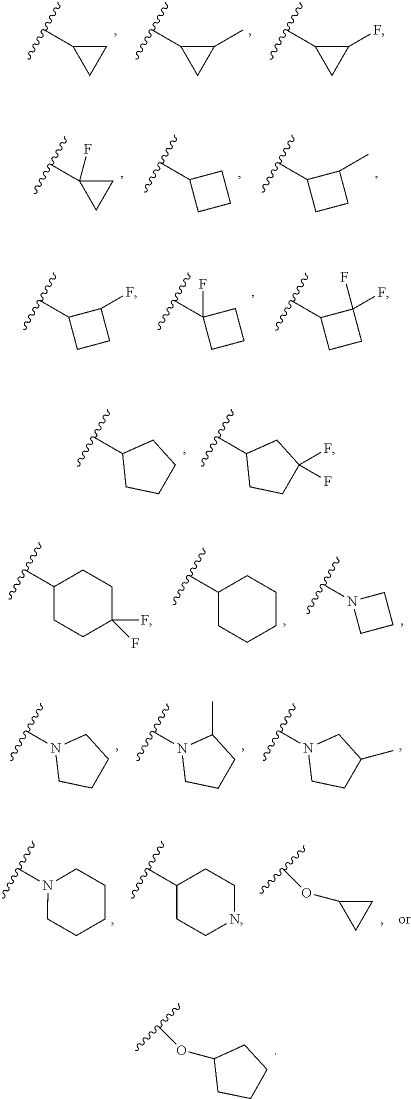

In some embodiments of a compound of Formula (VI), (I), (II), (IIa), (IIb), or (III), or a pharmaceutically acceptable salt or solvate thereof, R² is

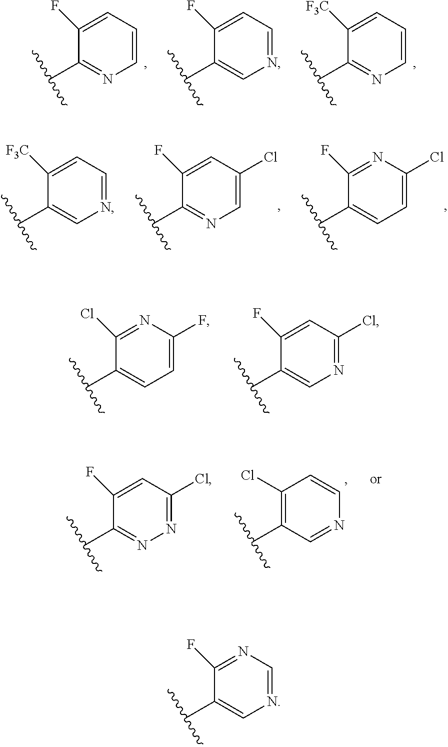

In some embodiments of a compound of Formula (VI), (I), (II), (IIa), (IIb), or (III), or a pharmaceutically acceptable salt or solvate thereof, R² is substituted or unsubstituted 5-6, 6-6, or 6-5 fused bicyclic heteroaryl containing 1-3 hetero ring atoms selected from O, N and S.

In some embodiments of a compound of Formula (VI), (I), (II), (IIa), (IIb), or (III), or a pharmaceutically acceptable salt or solvate thereof, R² is substituted or unsubstituted bicyclic C₅-C₈ cycloalkyl. In some embodiments, R² is bicyclo(1.1.1)pentane.

In some embodiments of a compound of Formula (VI), (I), (II), (IIa), (IIb), or (III), or a pharmaceutically acceptable salt or solvate thereof, R³ is substituted heteroaryl. In some embodiments, R³ is 5 or 6-membered substituted heteroaryl. In some embodiments, R³ is pyridinyl.

In some embodiments of a compound of Formula (VI), (I), (II), (IIa), (IIb), or (III), or a pharmaceutically acceptable salt or solvate thereof, R³ is pentafluorophenyl.

In some embodiments of a compound of Formula (VI), (I), (II), (IIa), (IIb), or (III), or a pharmaceutically acceptable salt or solvate thereof, each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl, wherein the alkyl, haloalkyl, heteroalkyl, cycloalkyl, or heterocycloalkyl is optionally substituted with hydroxy, amino, or methoxy. In some embodiments, each R¹¹ is independently H, substituted or unsubstituted C₁-C₃ alkyl, substituted or unsubstituted C₁-C₃ haloalkyl, substituted or unsubstituted C₁-C₃ heteroalkyl, substituted or unsubstituted —C₀₋₃ alkylene-C₃₋₆ cycloalkyl, or substituted or unsubstituted —C₀₋₃ alkylene-C₃₋₆ heterocycloalkyl. In some embodiments, each R¹¹ is independently H, methyl, ethyl, propyl, iso-propyl, n-butyl, iso-butyl, sec-butyl, t-butyl, linear or branched pentyl, linear or branched hexyl, cyclopropyl, cyclobutyl, cyclopentyl, cyclohexyl, —CF₃, —CH₂OCH₃, —CH₂NHCH₃, or —CH₂C₂F. In some embodiments, each R¹¹ is independently H or methyl.

In one aspect, the disclosure provides a compound having a structure of Formula (IV), or a pharmaceutically acceptable salt or solvate thereof:

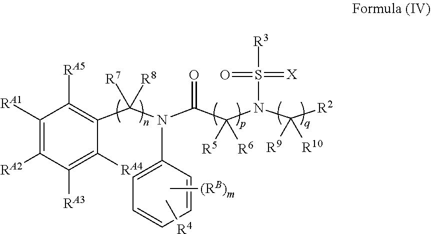

wherein
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰ , taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
   provided that p is 1 and q is 1;

each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl,
wherein at least one of ^{RA1} and R^{A3} is substituted or unsubstituted —C₃-C₈ cycloalkyl, substituted or unsubstituted heterocycloalkyl, substituted or unsubstituted alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, -N(R¹¹)₂, —-NR¹¹S(—O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, 3, or 4.

In some embodiments of a compound of Formula (IV) or (V), or a pharmaceutically acceptable salt or solvate thereof, R² is a phenyl substituted with at least one group that is not hydrogen or halogen. In some embodiments, when

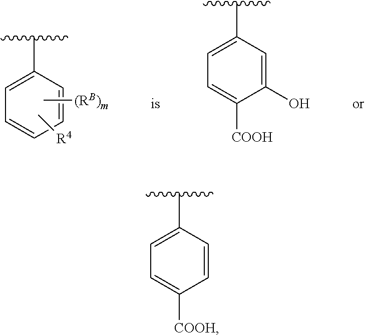

R² is a phenyl substituted with at least one group that is not hydrogen or halogen. In some embodiments, when (i)

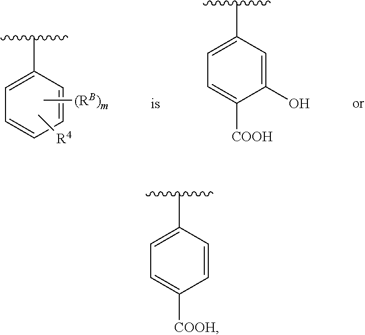

and (ii) one of R^{A1} and R^{A3} is cyclopentyl, then the other of R^{A1} and R^{A3} is not H, cyclopropyl, or t-butyl.

In some embodiments of a compound of Formula (IV), or a pharmaceutically acceptable salt or solvate thereof:
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
   wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰ , taken together form a substituted or unsubstituted 3, 4, 5, or 6-membered ring; or
R⁵ and R⁹, taken together with the intervening atoms to which they are attached form a 4, 5 or 6-membered ring,
   wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
   wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
   provided that p is 1 and q is 1;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl, wherein at least one of R^{A1} and R^{A3} is substituted or unsubstituted —C₃-C₈ cycloalkyl, substituted or unsubstituted —C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(═O)₂R¹¹, NR¹¹C(═O)R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, 3, or 4,
provided that when (i)

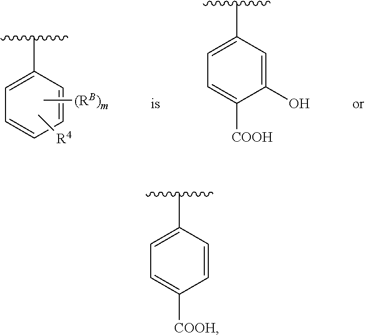

(ii) each of n, p, and q is 1, (iii)
R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other of R^{Al} and R^{A3} is hydrogen or unsubstituted t-butyl,
then R^{A4} differs from R^{A5}.

In some embodiments of a compound of Formula (IV), or a pharmaceutically acceptable salt or solvate thereof:
each of R⁵ and R⁶ is independently selected from hydrogen, F, —CN, —OR¹¹, —SR¹¹, —N(R¹¹)², substituted or unsubstituted C₁-C₆ alkyl, substituted or unsubstituted C₁-C₆haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁷ and R⁸, taken together with the carbon to which they are attached form a substituted or unsubstituted 3, 4, 5, or 6-membered ring; and
each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁹ and R¹⁰, taken together with the carbon to which they are attached form a substituted or unsubstituted 3, 4, 5, or 6-membered ring.

In some embodiments of a compound of Formula (IV), or a pharmaceutically acceptable salt or solvate thereof, each of R⁵ and R⁶ is independently selected from the group consisting of H, F, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆heteroalkyl, substituted or unsubstituted C₃-C₈ cycloalkyl, and substituted or unsubstituted C₃-C₇ heterocycloalkyl. In some embodiments, each of R⁵ and R⁶ is independently selected from the group consisting of H, F, —CN, —NH(CH₃), —NH₂, —N(CH₃)₂, —NHR¹¹, methyl, ethyl, propyl, iso-propyl, n-butyl, iso-butyl, sec-butyl, t-butyl, linear or branched pentyl, linear or branched hexyl, cyclopropyl, cyclobutyl, cyclopentyl, cyclohexyl, cycloheptyl, cyclooctyl, —CF₃, —CH₂CF₃, —CH₂C₂F, -OCF₃, —OH, —SH, —OCH₃, —OC₂CH₃, —OC₂OMe, and —OC₂C₂OH. In some embodiments, each of R⁵ and R⁶ is independently H, methyl, ethyl, propyl, butyl, pentyl, or hexyl, wherein the methyl, ethyl, propyl, butyl, pentyl, or hexyl is linear or branched, substituted or unsubstituted. In some embodiments, each of R⁵ and R⁶ is independently H, methyl, ethyl, propyl, butyl, pentyl, or hexyl, wherein the methyl, ethyl, propyl, butyl, pentyl, or hexyl is linear or branched, and optionally substituted with 1 to 3 F, methoxy, hydroxy, or amino. In some embodiments, each of R⁵ and R⁶ is independently H, CH₃, CF₃, or C₂F. In some embodiments, R⁵ is D. In some embodiments, R⁶ is D.

In some embodiments of a compound of Formula (IV), or a pharmaceutically acceptable salt or solvate thereof, R⁵ and R⁶ taken together with the carbon to which they are attached form a substituted or unsubstituted 4, 5, or 6 membered heterocyclic ring. In some embodiments, R⁵ and R⁶ taken together with the carbon to which they are attached form an oxetane, azetidine, tetrahydrofuran, or morpholine ring. In some embodiments of a compound of Formula (IV), or a pharmaceutically acceptable salt or solvate thereof, R⁵ and R⁶ taken together form an oxo.

In some embodiments of a compound of Formula (IV), or a pharmaceutically acceptable salt or solvate thereof, R⁵ and R⁶ taken together with the carbon to which they are attached form a substituted or unsubstituted 3, 4, 5, or 6 membered cycloalkyl ring. In some embodiments, R⁵ and R⁶ taken together with the carbon to which they are attached form a substituted or unsubstituted cyclobutane, cyclopentane, or cyclohexane.

In some embodiments of a compound of Formula (IV), or a pharmaceutically acceptable salt or solvate thereof, each of R⁷, R⁸, R⁹, and R¹⁰ is independently selected from the group consisting of H, amino, F, substituted or unsubstituted C₁-C₆ alkoxy, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy. In some embodiments, each of R⁷, R⁸, R⁹, and R¹⁰ is independently selected from the group consisting of H, amino, F, substituted or unsubstituted C₁-C₆ alkoxy, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy. In some embodiments, each of R⁷, R⁸, R⁹, and R¹⁰ is independently selected from the group consisting of H, F, —NH(CH₃), —NH₂, —N(CH₃)₂, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, and —OC₂C₂OH. In some embodiments, R⁷ is D. In some embodiments, R⁸ is D. In some embodiments, R⁹ is D. In some embodiments, R¹⁰ is D.

In some embodiments of a compound of Formula (IV), or a pharmaceutically acceptable salt or solvate thereof, each of R⁷, R⁸, R⁹, and R¹⁰ is independently selected from the group consisting of H, F, substituted or unsubstituted C₁-C₆ alkyl, substituted or unsubstituted C₁-C₆ fluoroalkyl, and substituted or unsubstituted C₁-C₆ heteroalkyl, wherein the alkyl, fluoroalkyl or heteroalkyl is optionally substituted with hydroxy, amino, or methoxy. In some embodiments, each of R⁷, R⁸, R⁹, and R¹⁰ is independently H, F, methyl, ethyl, propyl, —CF₃, or —CH₂CF₃. In some embodiments, each of R⁷, R⁸, R⁹, and R¹⁰ is H.

In some embodiments of a compound of Formula (IV), or a pharmaceutically acceptable salt or solvate thereof, R⁵ and R⁹, taken together with the intervening atoms to which they are attached form a 4, 5 or 6-membered cycloalkyl or heterocycloalkyl ring, wherein R⁶ is independently selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy.

In some embodiments of a compound of Formula (IV), or a pharmaceutically acceptable salt or solvate thereof, R⁷ and R⁸, taken together form a substituted or unsubstituted 3, 4, 5, or 6-membered cycloalkyl or heterocycloalkyl ring, wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy.

In some embodiments of a compound of Formula (IV), or a pharmaceutically acceptable salt or solvate thereof, R⁹ and R¹⁰ , taken together form a substituted or unsubstituted 3, 4, 5, or 6-membered cycloalkyl or heterocycloalkyl ring, wherein each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy.

In some embodiments of a compound of Formula (IV), or a pharmaceutically acceptable salt or solvate thereof, R³ is pentafluorophenyl.

In some embodiments of a compound of Formula (IV), or a pharmaceutically acceptable salt or solvate thereof, X is O. In some embodiments, X is NR¹¹. In some embodiments, X is NH. In some embodiments, X is N-alkyl. In some embodiments, X is absent.

In some embodiments of a compound of Formula (IV), or a pharmaceutically acceptable salt or solvate thereof, the compound has the structure of Formula (V):

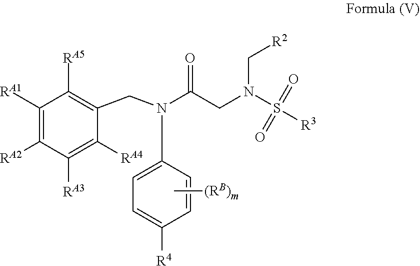

wherein
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl,
   wherein at least one of R^{A1} and R^{A3} is substituted or unsubstituted —C₃-C₈ cycloalkyl, substituted or unsubstituted heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(═O)₂R¹¹, NR¹¹C(═O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
each of m and k is independently 0, 1, 2, 3, or 4.

In some embodiments of a compound of Formula (IV) or (V), or a pharmaceutically acceptable salt, solvate, ester, or polymorph thereof, when (i)

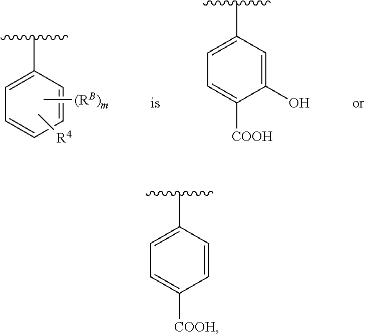

(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other of R^{A1} and R^{A3} is hydrogen or unsubstituted t-butyl, then R^{A4} differs from R^{A5}.

In some embodiments of a compound of Formula (IV) or (V), or a pharmaceutically acceptable salt or solvate thereof:
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆haloalkyl, substituted or unsubstituted —C₀₋₆ alkylene-C₃₋₈ cycloalkyl, and substituted or unsubstituted —C₀₋₆ alkylene-C₃₋₇ heterocycloalkyl, wherein at least one of R^{Al} and R^{A3} is substituted or unsubstituted —C₃-C₈ cycloalkyl, substituted or unsubstituted —C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, -N(R¹¹)₂, —NR¹¹S(═O)₂R¹¹, NR¹¹C(═O)R¹¹ substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, 3, or 4,
provided that when (i)

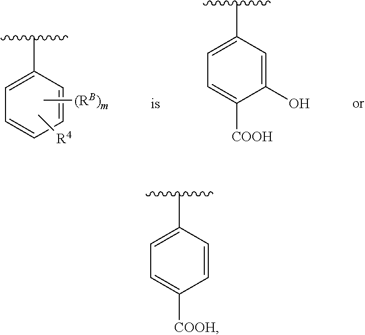

(ii) each of n, p, and q is 1, (iii) R² is phenyl substituted with 1-5 halogens, and (iv) R^{A1} and R^{A3} are both C₃-C₈ unsubstituted cycloalkyl, or one of R^{A1} and R^{A3} is C₃-C₅ unsubstituted cycloalkyl and the other of R^{A1} and R^{A3} is hydrogen or unsubstituted t-butyl,
then R^{A4} and R^{A5} are not the same.

In some embodiments of a compound of Formula (IV) or (V), or a pharmaceutically acceptable salt or solvate thereof,
when (i)

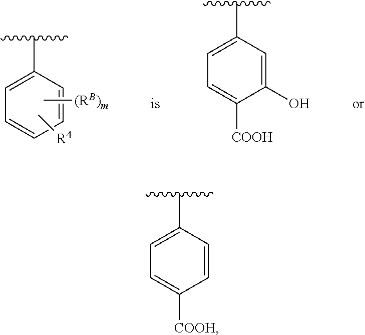

(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other is hydrogen or unsubstituted t-butyl, then at least one of R^{A4} and R^{A5} is not hydrogen.

In some embodiments of a compound of Formula (IV) or (V), or a pharmaceutically acceptable salt or solvate thereof,
when (i)

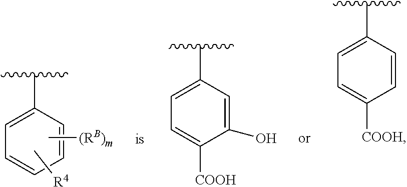

(ii) each of n, p, and q is 1, (iii) R² is phenyl substituted with 1-5 halogens, and (iv) R^{A1} and R^{A3} are both selected from cyclopropyl and cyclopentyl, or one of R^{A1} and R^{A3} is selected from cyclopropyl and cyclopentyl and the other is hydrogen or unsubstituted t-butyl, then at least one of R^{A4} and R^{A5} is not hydrogen.

In some embodiments of a compound of Formula (IV) or (V), or a pharmaceutically acceptable salt or solvate thereof,

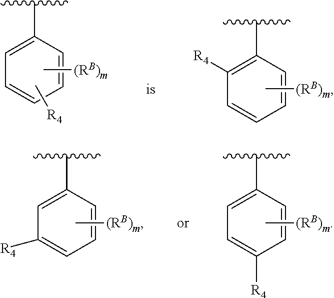

In some embodiments, m is 0, 1, or 2. In some embodiments,

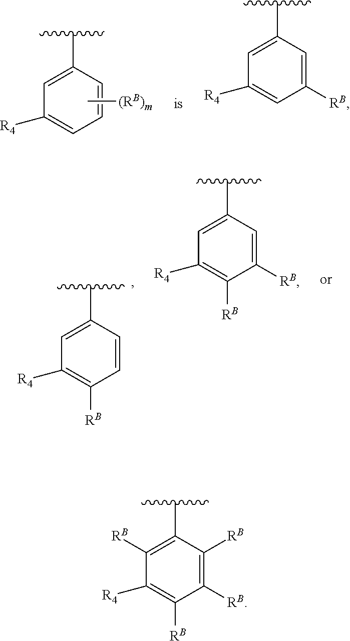

In some embodiments,

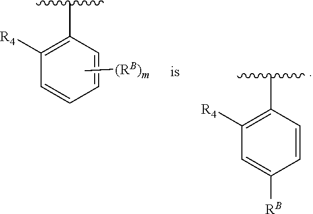

In some embodiments,

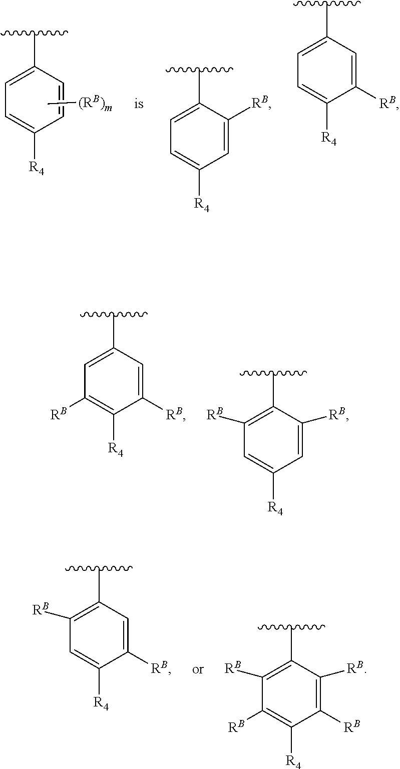

In some embodiments,

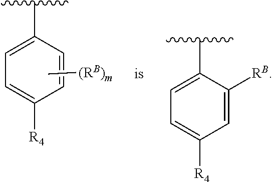

In some embodiments of a compound of Formula (IV) or (V), or a pharmaceutically acceptable salt or solvate thereof, R⁴ is COOH.

In some embodiments of a compound of Formula (IV) or (V), or a pharmaceutically acceptable salt or solvate thereof, R⁴ is SO₂H.

In some embodiments of a compound of Formula (IV) or (V), or a pharmaceutically acceptable salt or solvate thereof, R⁴ is C(O)N(R¹¹)₂, C(O)OR¹¹, or S(O)₂N(R¹¹)₂. In some embodiments, R⁴ is C(O)N(R¹¹)₂, C(O)OR¹¹, or S(O)₂N(R¹¹)₂, wherein each R¹¹ is independent H or C₁-C₆ alkyl. In some embodiments, R⁴ is C(O)N(Me)₂, C(O)NH₂, C(O)NHCH₃, C(O)OMe, S(O)₂N(Me)₂, S(O)₂NH₂, or S(O)₂NHCH₃.

In some embodiments of a compound of Formula (IV) or (V), or a pharmaceutically acceptable salt or solvate thereof, R⁴ is —OR¹¹. In some embodiments, R⁴ is

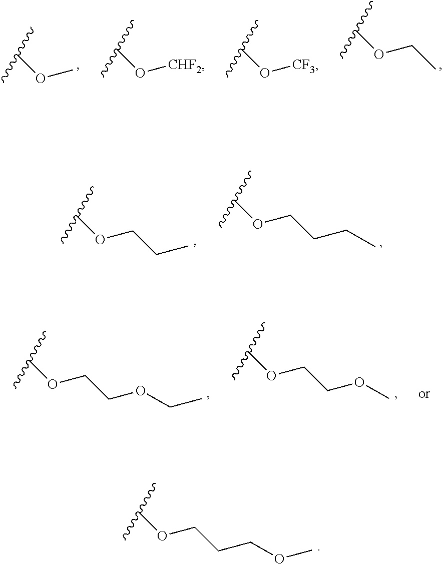

In some embodiments of a compound of Formula (IV) or (V), or a pharmaceutically acceptable salt or solvate thereof, R⁴ is —C₀₋₆ alkylene-R⁴¹ or —C₂₋₄ alkylene-R⁴¹. In some embodiments,

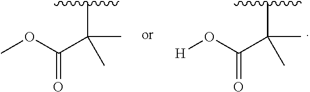

In some embodiments of a compound of Formula (IV) or (V), or a pharmaceutically acceptable salt or solvate thereof, each R^{B} is independently halogen, D, —CN, —OR¹¹, —SR¹¹, —N(R¹¹)₂, —NR¹¹S(═O)₂R¹¹, substituted or unsubstituted C₁-C₆ alkyl, substituted or unsubstituted —C₀₋₃ alkylene-C₃₋₆ cycloalkyl, or substituted or unsubstituted —C₀₋₃ alkylene-C₃₋₅ heterocycloalkyl. In some embodiments, at least one R^{B} is a halogen selected from F and Cl. In some embodiments, each R^{B} is independently a halogen selected from F and Cl. In some embodiments, at least one R^{B} is a linear or branched, substituted or unsubstituted C₁-C₆ alkyl. In some embodiments, each R^{B} is independently linear or branched, substituted or unsubstituted C₁-C₆ alkyl. In some embodiments, each C₁-C₆ alkyl is independently methyl, ethyl, propyl, iso-propyl, n-butyl, iso-butyl, sec-butyl, t-butyl, linear or branched pentyl, linear or branched hexyl, —CF₃, —CH₂NH₂, —CH₂CF₃, —CH₂CHNH₂, —CH₂C₂F, —CH₂OH, or —CH₂C₂OH.

In some embodiments of a compound of Formula (IV) or (V), or a pharmaceutically acceptable salt or solvate thereof, at least one R^{B} is substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, at least one R^{B} is substituted or unsubstituted —C₀₋₆ alkylene-C₃₋₈ cycloalkyl. In some embodiments, at least one R^{B} is substituted or unsubstituted —C₀₋₆ alkylene-C₃₋₇ heterocycloalkyl. In some embodiments, at least one R^{B} is substituted or unsubstituted —C₀₋₃ alkylene-C₃₋₈ cycloalkyl, or substituted or unsubstituted —C₀₋₃ alkylene-C₃₋₇ heterocycloalkyl. In some embodiments, at least one R^{B} is C₃₋₆ cycloalkyl, —CH₂-C₃₋₆ cycloalkyl, —(C₂)2-C₃₋₆ cycloalkyl, —(C₂)₃—C₃₋₆ cycloalkyl, C₃₋₅ heterocycloalkyl, —CH₂-C₃₋₅ heterocycloalkyl, —(C₂)₂—C₃₋₅ heterocycloalkyl, or —(C₂)₃—C₃₋₅ heterocycloalkyl, wherein the cycloalkyl and heterocycloalkyl is substituted or unsubstituted. In some embodiments, each of R^{B} is independently substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each of R^{B} is independently substituted or unsubstituted —C₀₋₆ alkylene-C₃₋₈ cycloalkyl. In some embodiments, each of R^{B} is independently substituted or unsubstituted —C₀₋₆ alkylene-C₃₋₇ heterocycloalkyl. In some embodiments, each of R^{B} is independently substituted or unsubstituted —C₀₋₃ alkylene-C₃₋₈ cycloalkyl, or substituted or unsubstituted —C₀₋₃ alkylene-C₃₋₇ heterocycloalkyl. In some embodiments, the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

In some embodiments of a compound of Formula (IV) or (V), or a pharmaceutically acceptable salt or solvate thereof, each of R^{B} is independently C₃₋₆ cycloalkyl, —CH₂—C₃₋₆ cycloalkyl, —(C₂)₂—C₃₋₆ cycloalkyl, —(C₂)₃—C₃₋₆ cycloalkyl, C₃₋₅ heterocycloalkyl, —CH₂—C₃₋₅ heterocycloalkyl, —(C₂)₂—C₃₋₅ heterocycloalkyl, or —(C₂)₃—C₃₋₅ heterocycloalkyl, wherein the cycloalkyl and heterocycloalkyl is substituted or unsubstituted. In some embodiments, the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

In some embodiments of a compound of Formula (IV) or (V), or a pharmaceutically acceptable salt or solvate thereof, at least one R^{B} is substituted or unsubstituted —C₀₋₆ alkylene-C₃₋₈ cycloalkyl, or substituted or unsubstituted —C₀₋₆ alkylene-C₃₋₇ heterocycloalkyl, and each of the cycloalkyl is independently

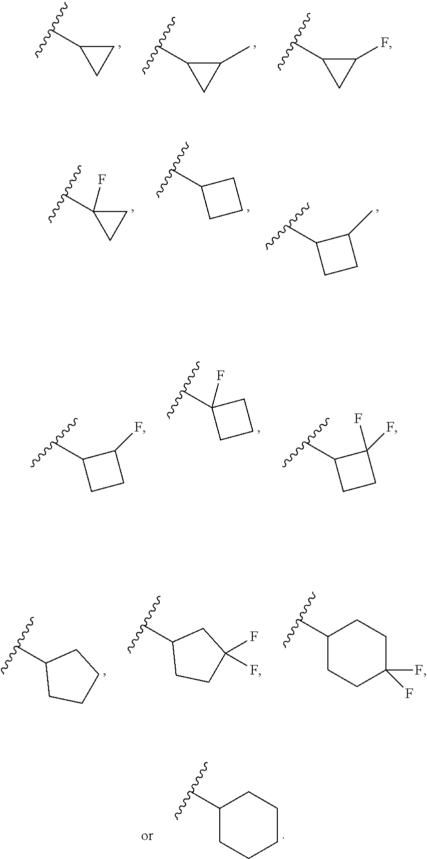

In some embodiments of a compound of Formula (IV) or (V), or a pharmaceutically acceptable salt or solvate thereof, at least one R^{B} is substituted or unsubstituted —C₀₋₆ alkylene-C₃₋₈ cycloalkyl, or substituted or unsubstituted —C₀₋₆ alkylene-C₃₋₇ heterocycloalkyl, and each of the heterocycloalkyl is independently

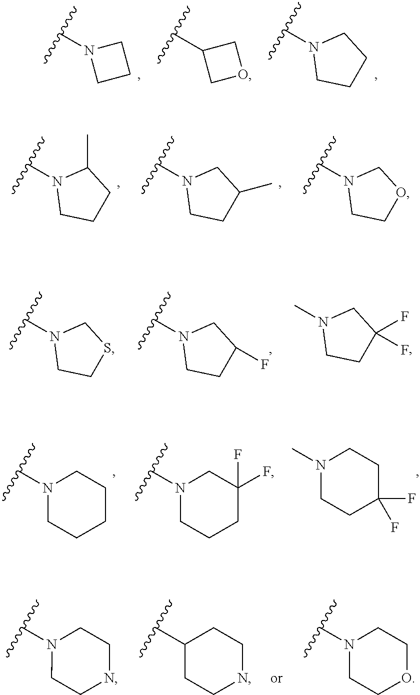

In some embodiments of a compound of Formula (IV) or (V), or a pharmaceutically acceptable salt or solvate thereof, at least one R^{B} is -OR″. In some embodiments, each R^{B} is independently —OR¹¹. In some embodiments, each —OR¹¹ is independently OH, —O—C₁-C₆ alkyl, —O—C₁-C₆ haloalkyl, —O—C₁-C₆ heteroalkyl, —O—C₀₋₆ alkylene-C₃₋₈ cycloalkyl, or —O—C₀₋₆ alkylene-C₃₋₇ heterocycloalkyl, wherein the alkyl, haloalkyl, heteroalkyl, cycloalkyl, and heterocycloalkyl is substituted or unsubstituted.

In some embodiments of a compound of Formula (IV) or (V), or a pharmaceutically acceptable salt or solvate thereof, at least one R^{B} is substituted or unsubstituted —O—C₁-C₆ alkyl. In some embodiments, at least one R^{B} is

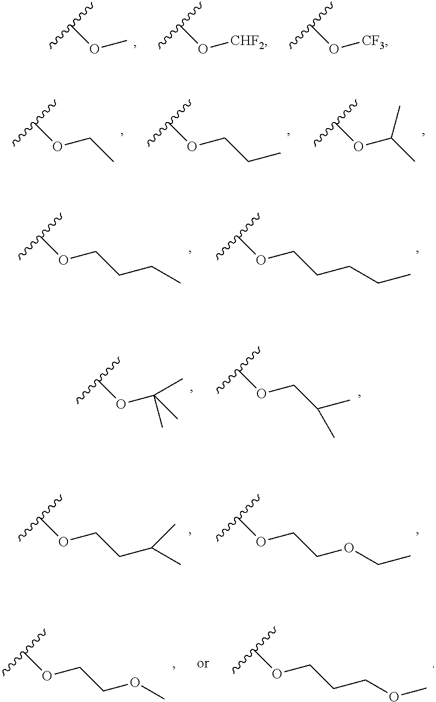

In some embodiments of a compound of Formula (IV) or (V), or a pharmaceutically acceptable salt or solvate thereof, each R^{B} is independently substituted or unsubstituted —O—C₁-C₆ alkyl. In some embodiments, each R^{B} is independently

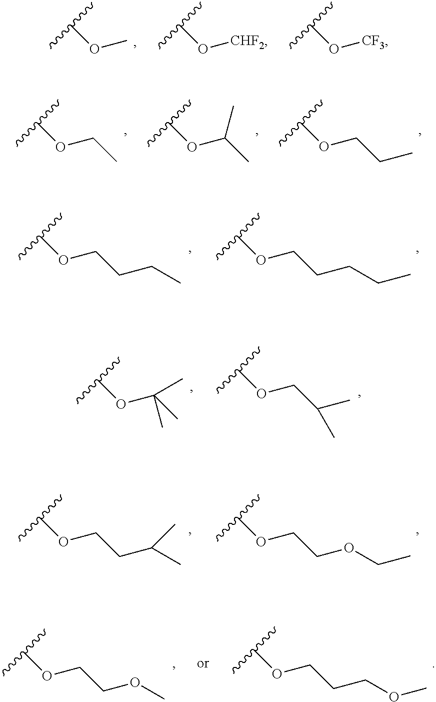

In some embodiments, each R^{B} is

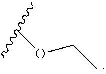 [SMILES: *OCC]

In some embodiments of a compound of Formula (IV) or (V), or a pharmaceutically acceptable salt or solvate thereof, at least one R^{B} is OH. In some embodiments, each R^{B} is OH.

In some embodiments of a compound of Formula (IV) or (V), or a pharmaceutically acceptable salt or solvate thereof, at least one R^{B} is substituted or unsubstituted —O—C₀₋₆ alkylene-C₃₋₈ cycloalkyl, or substituted or unsubstituted —O—C₀₋₆ alkylene-C₃₋₇ heterocycloalkyl. In some embodiments. at least one R^{B} is

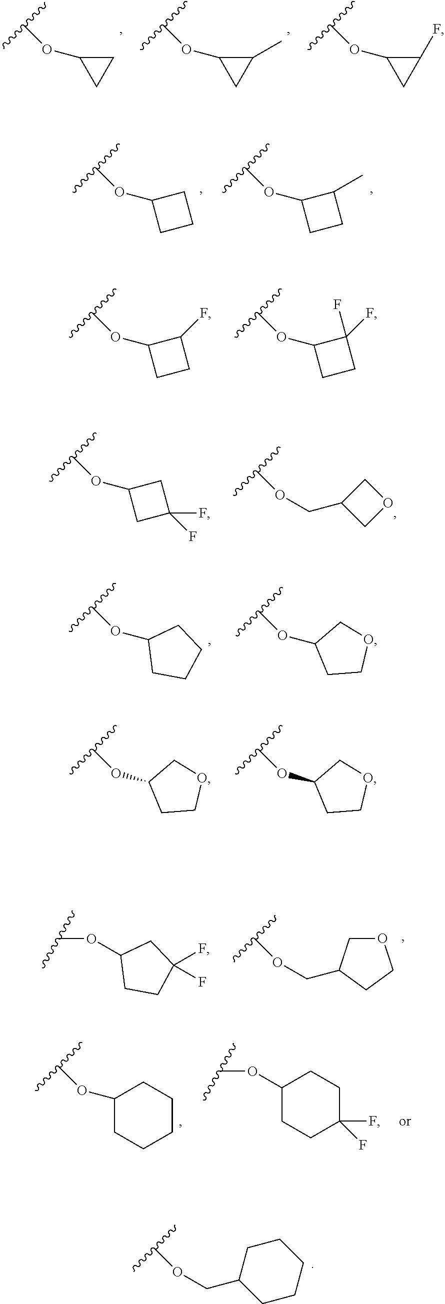

In some embodiments of a compound of Formula (IV) or (V), or a pharmaceutically acceptable salt or solvate thereof, at least one R^{B} is

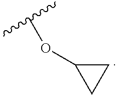 [SMILES: *OC1CC1]

In some embodiments of a compound of Formula (IV) or (V), or a pharmaceutically acceptable salt or solvate thereof, each R^{B} is independently substituted or unsubstituted —O—C₀₋₆ alkylene-C₃₋₈ cycloalkyl, or substituted or unsubstituted —O—C₀₋₆ alkylene-C₃₋₇ heterocycloalkyl. In some embodiments, each R^{B} is independently

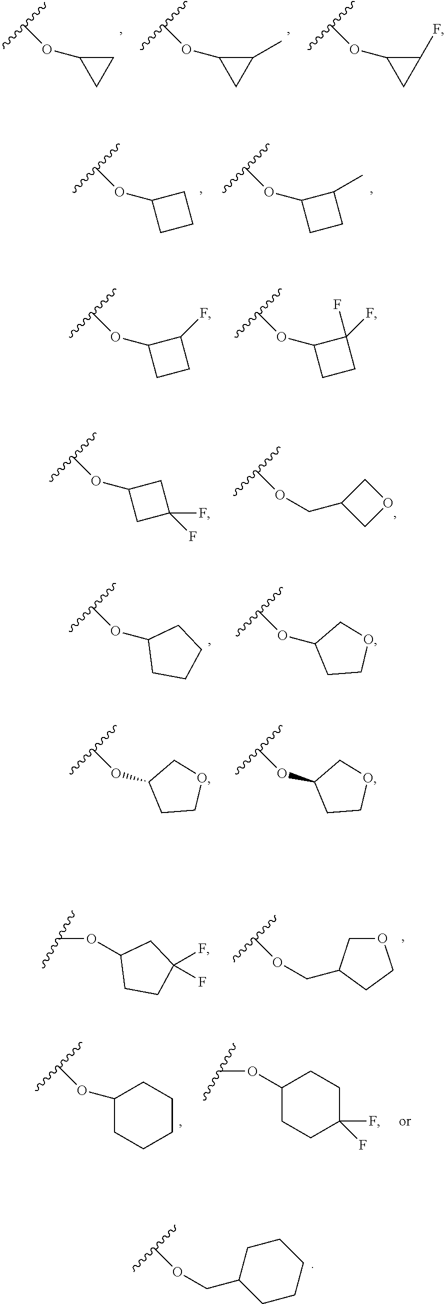

In some embodiments, each R^{B} is

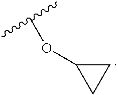 [SMILES: *OC1CC1]

In some embodiments of a compound of Formula (IV) or (V), or a pharmaceutically acceptable salt or solvate thereof, at least one R^{B} is —N(R¹¹)₂. In some embodiments, at least one R^{B} is —N(CH₃)₂, —NHCH₃, —N(C₂CH₃)₂, —NHC₂CH₃, or —N(C₂C₂CH₃)₂. In some embodiments, each R^{B} is independently —N(R¹¹)₂. In some embodiments, each R^{B} is independently —N(CH₃)₂, —NHCH₃, —N(C₂CH₃)₂, —NHC₂CH₃, or —N(C₂C₂CH₃)₂.

In some embodiments of a compound of Formula (IV) or (V), or a pharmaceutically acceptable salt or solvate thereof, at least one R^{B} is —NR¹¹S(═O)₂R¹¹, wherein each R¹¹ is independently H or C₁-C₃ alkyl. In some embodiments, the —NR¹¹S(═O)₂R¹¹ is —NCH₃S(═O)₂CH₃.

In some embodiments of a compound of Formula (IV) or (V), or a pharmaceutically acceptable salt or solvate thereof,

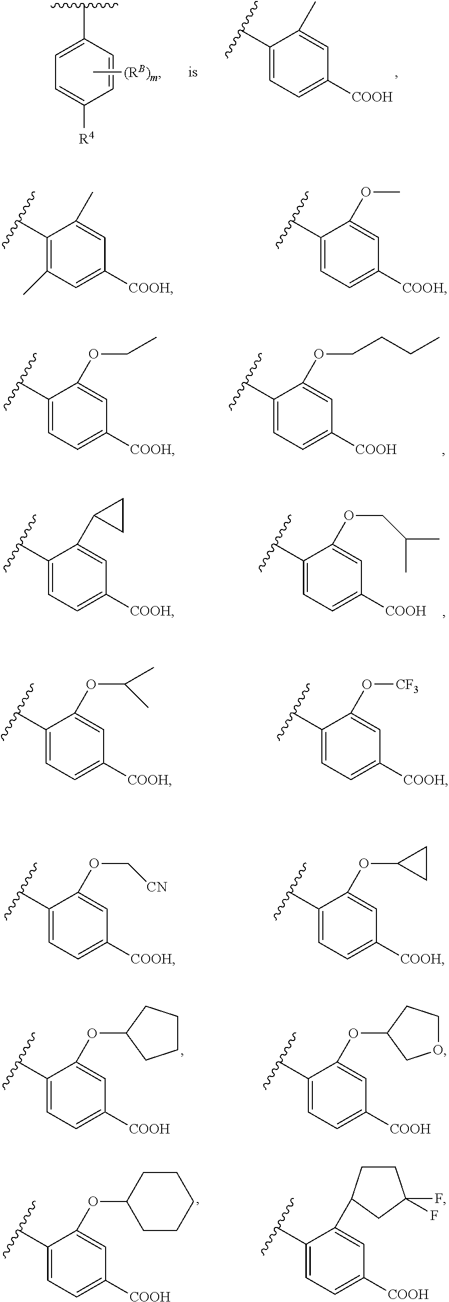

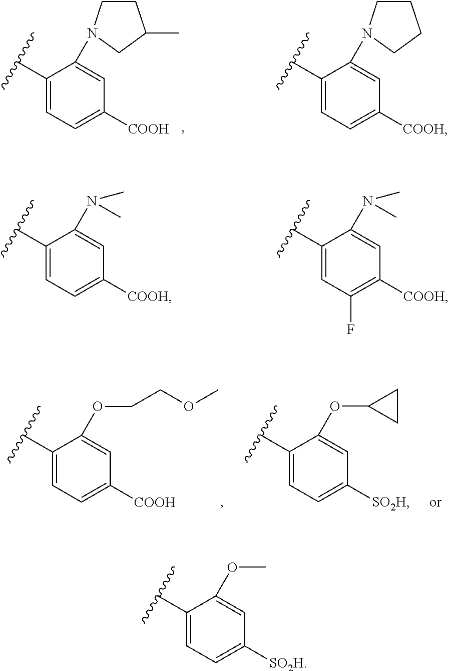

In some embodiments of a compound of Formula (IV) or (V), or a pharmaceutically acceptable salt or solvate thereof,

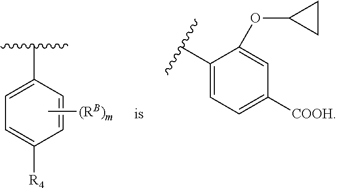

In some embodiments of a compound of Formula (IV) or (V), or a pharmaceutically acceptable salt or solvate thereof,

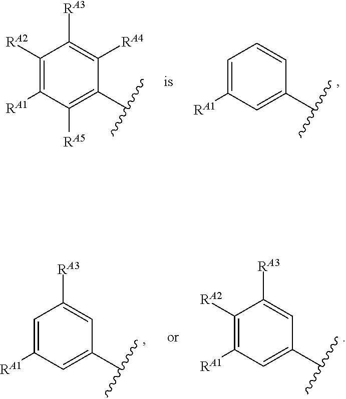

In some embodiments of a compound of Formula (IV) or (V), or a pharmaceutically acceptable salt or solvate thereof, at least one of R^{A1} and R^{A3} is substituted or unsubstituted —C₃-C₆ cycloalkyl. In some embodiments, at least one of R^{A1} and R^{A3} is

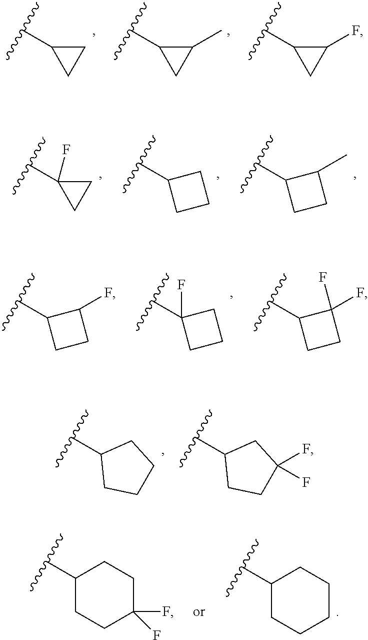

In some embodiments, R^{A1} is

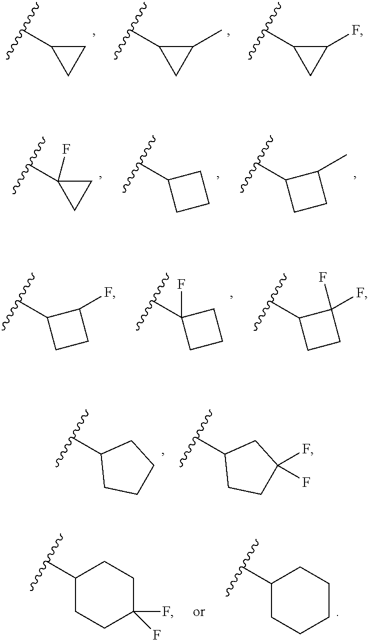

In some embodiments of a compound of Formula (IV) or (V), or a pharmaceutically acceptable salt or solvate thereof, at least one of R^{A1} and R^{A3} is substituted or unsubstituted —C₃-C₅ heterocycloalkyl. In some embodiments, at least one of R^{A1} and R^{A3} is

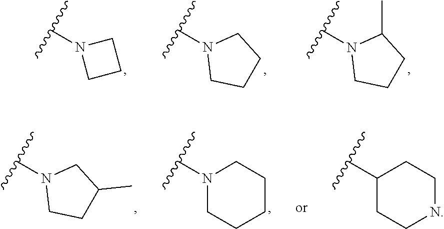

In some embodiments, R^{A1} is

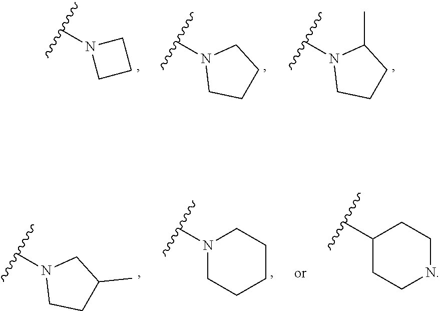

In some embodiments of a compound of Formula (IV) or (V), or a pharmaceutically acceptable salt or solvate thereof, at least one of R^{A1} and R^{A3} is substituted or unsubstituted —O—C₀₋₃ alkylene-C₃₋₆ cycloalkyl, or substituted or unsubstituted —O—C₀₋₃ alkylene-C₃₋₅ heterocycloalkyl. In some embodiments, R^{A1} is substituted or unsubstituted —O—C₀₋₃ alkylene-C₃₋₆ cycloalkyl, or substituted or unsubstituted —O—C₀₋₃ alkylene-C₃₋₅ heterocycloalkyl.

In some embodiments of a compound of Formula (IV) or (V), or a pharmaceutically acceptable salt or solvate thereof, each of R^{A2}, R^{A3}, R^{A4}, and R^{A5} is independently halogen, —OR¹¹, substituted or unsubstituted C₁-C₆ alkyl, substituted or unsubstituted C₁-C₆ haloalkyl, substituted or unsubstituted —C₀₋₃ alkylene-C₃₋₆ cycloalkyl, or substituted or unsubstituted —C₀₋₃ alkylene-C₃₋₅ heterocycloalkyl.

In some embodiments of a compound of Formula (IV) or (V), or a pharmaceutically acceptable salt or solvate thereof, R^{A1} is D. In some embodiments, R^{A2} is D. In some embodiments, R^{A3} is D. In some embodiments, R^{A4} is D. In some embodiments, R^{A5} is D.

In some embodiments of a compound of Formula (IV) or (V), or a pharmaceutically acceptable salt or solvate thereof, each of R^{A2}, R^{A3}, R^{A4}, and R^{A5} is independently halogen, C₁-C₆ alkyl, —C₀₋₃ alkylene-C₃₋₆ cycloalkyl, —C₀₋₃ alkylene-C₃₋₅ heterocycloalkyl, -0-C₁-C₆ alkyl, —O—C₀₋₃ alkylene-C₃₋₆ cycloalkyl, or —O—C₀₋₃ alkylene-C₃₋₅ heterocycloalkyl, and wherein the alkyl, cycloalkyl, and heterocycloalkyl is substituted or unsubstituted. In some embodiments, each of R^{A2}, R^{A3}, R^{A4}, and R^{A5} is independently F, Cl, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

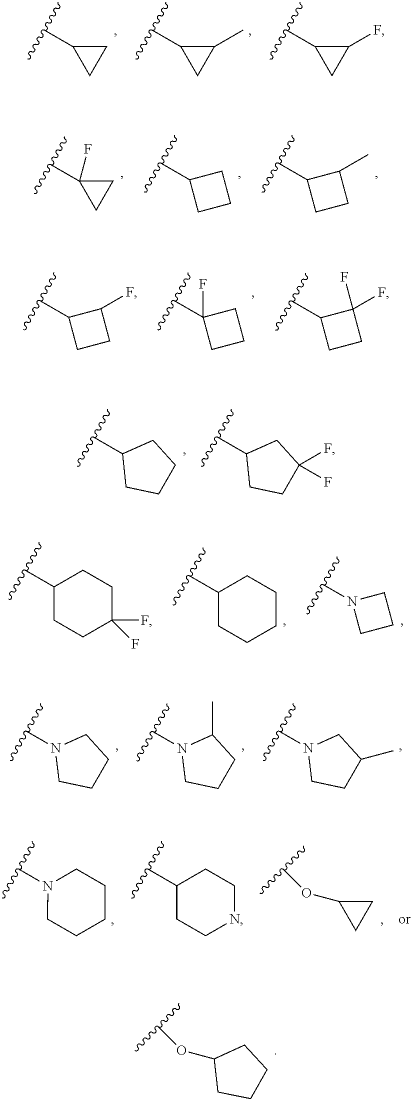

In some embodiments of a compound of Formula (IV) or (V), or a pharmaceutically acceptable salt or solvate thereof,

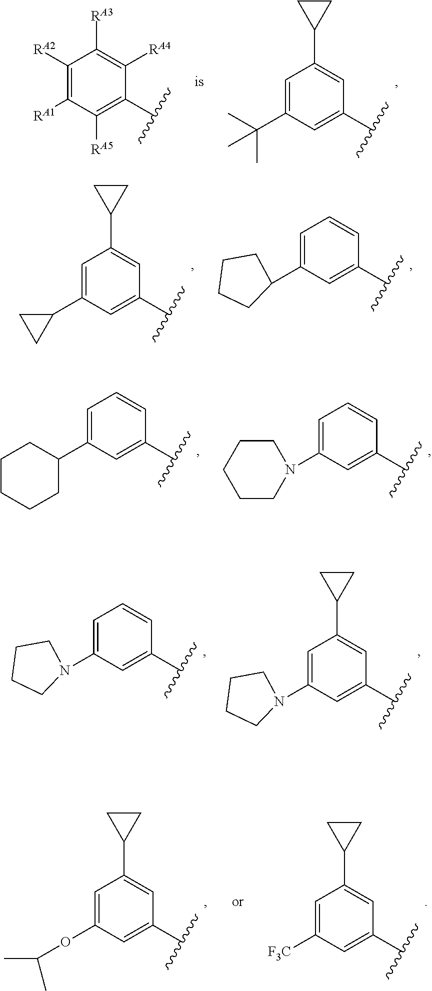

In some embodiments of a compound of Formula (IV) or (V), or a pharmaceutically acceptable salt or solvate thereof, R² is phenyl or substituted phenyl. In some embodiments, R² is phenyl substituted with 1 to 5 R^{c}, and wherein each R^{c} is independently D, halogen, —OR¹¹, —S¹¹, —N(R¹¹)₂, —CN, —NO₂, substituted or unsubstituted C₁-C₆ alkyl, substituted or unsubstituted C₁-C₆haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, R² is phenyl substituted with 1 to 5 R^{c}, and wherein each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

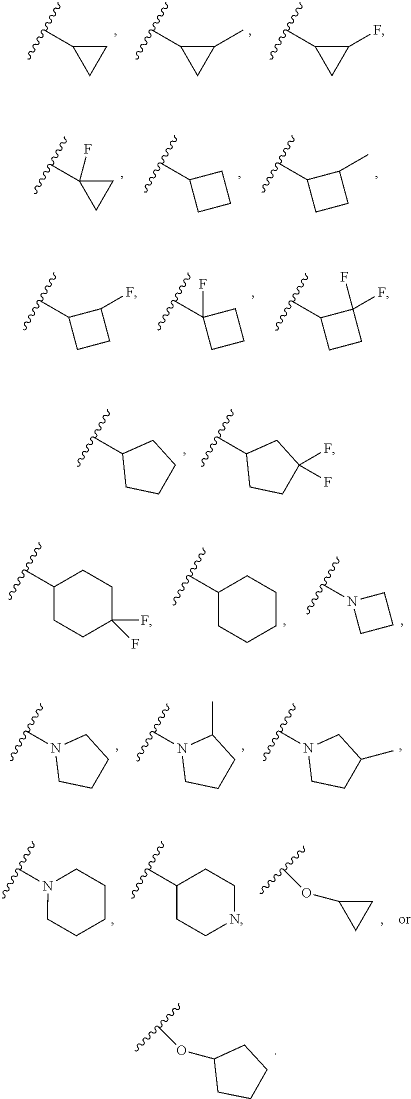

In some embodiments, R² is

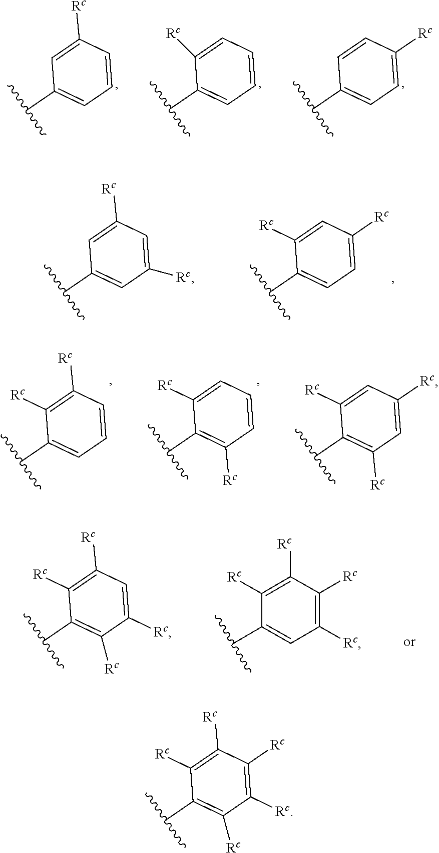

In some embodiments, R² is

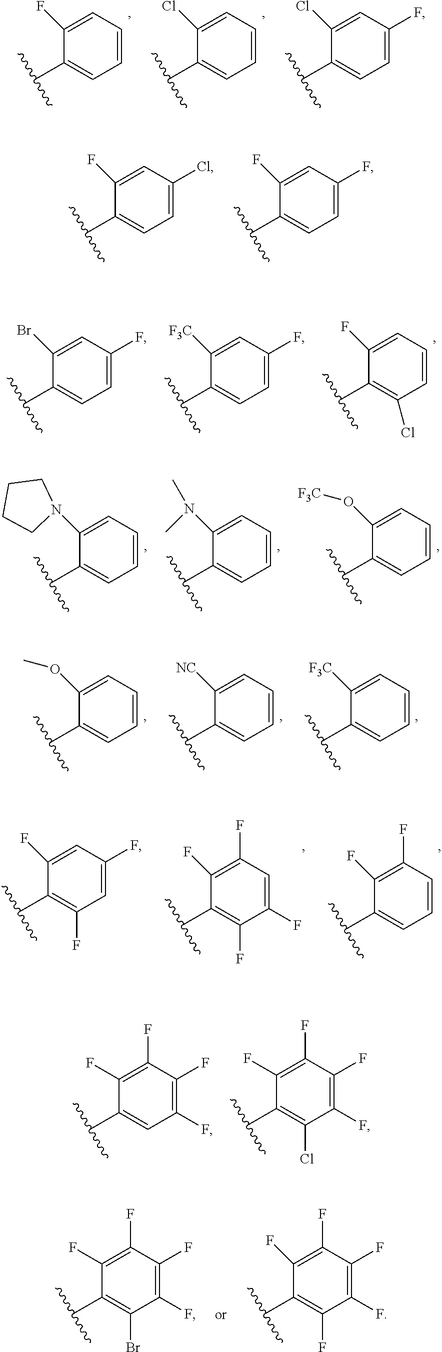

In some embodiments of a compound of Formula (IV) or (V), or a pharmaceutically acceptable salt or solvate thereof, wherein R² is substituted or unsubstituted 5-membered or 6-membered monocyclic heteroaryl. In some embodiments, R² is pyridinyl, pyridazinyl, pyrimidinyl, triazinyl, wherein the pyridinyl, pyridazinyl, pyrimidinyl, or triazinyl is substituted with 1 to 4 R^{c}, and wherein each R^{c} is independently D, halogen, —OR¹¹, —SR¹¹, —N(R¹¹)₂, —CN, —NO₂, substituted or unsubstituted C₁-C₆ alkyl, substituted or unsubstituted C₁-C₆haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

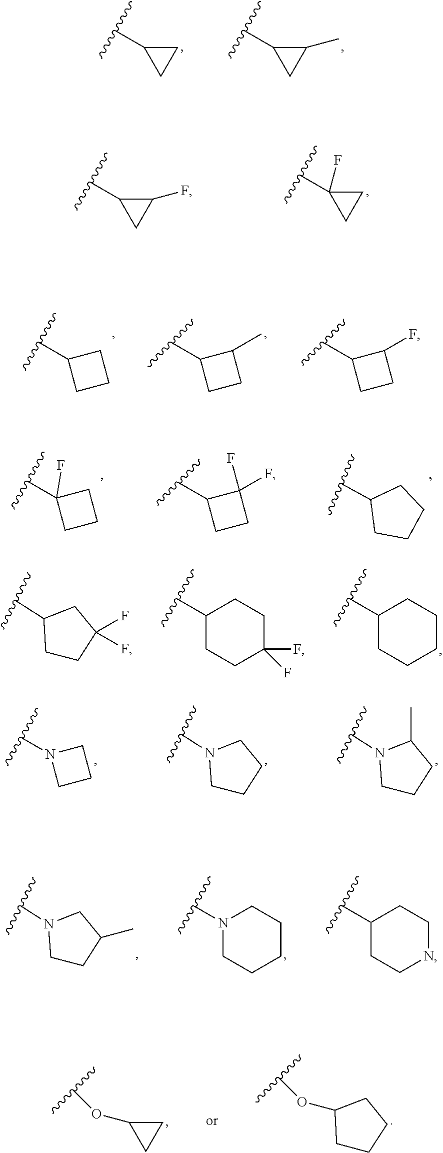

In some embodiments, each R^{c} is Cl or F. In some embodiments, each R^{c} is —CF₃.

In some embodiments of a compound of Formula (IV) or (V), or a pharmaceutically acceptable salt or solvate thereof, R² is

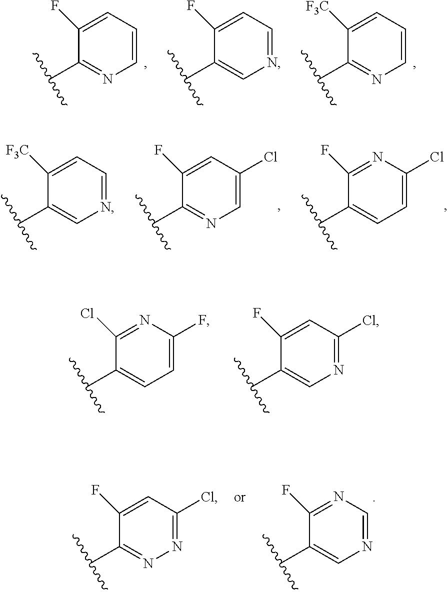

In some embodiments of a compound of Formula (IV) or (V), or a pharmaceutically acceptable salt or solvate thereof, R² is substituted or unsubstituted 5-6, 6-6, or 6-5 fused bicyclic heteroaryl containing 1-3 hetero ring atoms selected from O, N and S.

In some embodiments of a compound of Formula (IV) or (V), or a pharmaceutically acceptable salt or solvate thereof, R² is substituted or unsubstituted bicyclic C₅-C₈ cycloalkyl. In some embodiments, R² is bicyclo(1.1.1)pentane.

In some embodiments of a compound of Formula (IV) or (V), or a pharmaceutically acceptable salt or solvate thereof, R³ is substituted heteroaryl. In some embodiments, R³ is 5 or 6-membered substituted heteroaryl. In some embodiments, R³ is pyridinyl.

In some embodiments of a compound of Formula (IV) or (V), or a pharmaceutically acceptable salt or solvate thereof, R³ is pentafluorophenyl.

In some embodiments of a compound of Formula (IV) or (V), or a pharmaceutically acceptable salt or solvate thereof, each R¹¹ is independently H, substituted or unsubstituted C₁-C₆ alkyl, substituted or unsubstituted C₁-C₆ haloalkyl, substituted or unsubstituted C1-C6 heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl. In some embodiments, each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl, wherein the alkyl, haloalkyl, heteroalkyl, cycloalkyl, or heterocycloalkyl is optionally substituted with hydroxy, amino, or methoxy. In some embodiments, each R¹¹ is independently H, substituted or unsubstituted C₁-C₃ alkyl, substituted or unsubstituted C₁-C₃ haloalkyl, substituted or unsubstituted C₁-C₃ heteroalkyl, substituted or unsubstituted —C₀₋₃ alkylene-C₃₋₆ cycloalkyl, or substituted or unsubstituted —C₀₋₃ alkylene-C₃₋₆ heterocycloalkyl. In some embodiments, each R¹¹ is independently H, methyl, ethyl, propyl, iso-propyl, n-butyl, iso-butyl, sec-butyl, t-butyl, linear or branched pentyl, linear or branched hexyl, cyclopropyl, cyclobutyl, cyclopentyl, cyclohexyl, —CF₃, —CH₂OCH₃, —CH₂NHCH₃, or —CH₂C₂F.

In one aspect, provided herein is an ester of a compound of Formula (VI), (I), (II), (IIa), (IIb), (III), (IV), or (V), or a pharmaceutically acceptable salt or solvate thereof. In some embodiments, the ester is a reaction product of an acid group of the described compound with an alcohol. In some embodiments, the ester is a reaction product of an alcohol with R⁴ group in the described compounds. In some embodiments, the ester is a C₁-C₆ alkyl ester, C₁-C₆ heteroalkyl ester or C₂-C₆ alkenyl ester, and wherein the alkyl, heteroalkyl, and alkenyl is substituted or unsubstituted. In some embodiments, the alcohol that forms an ester with a described compound has a structure of R²⁰OH, wherein R²⁰ is substituted or unsubstituted alkyl, substituted or unsubstituted haloalkyl, or substituted or unsubstituted heteroalkyl. In some embodiments, the alcohol that forms ester with a described compound has a structure of R²⁰OH, wherein R²⁰ is substituted or unsubstituted C₁-C₁₂ alkyl, substituted or unsubstituted C₁-C₁₂ haloalkyl, or substituted or unsubstituted C₁-C₁₂ heteroalkyl.

In one aspect, provided herein is an amide of a compound of Formula (VI), (I), (II), (IIa), (IIb), (III), (IV), or (V), or a pharmaceutically acceptable salt or solvate thereof. In some embodiments, the amide is a reaction product of an acid group of the described compound with an amine. In some embodiments, the amide is a reaction product of an amine with R⁴ group in the described compounds. In some embodiments, the amide results from reacting the compound with a sulfonamide, NH₃, mono-C₁-C₆ alkylamino, or di-C₁-C₆ alkylamino. In some embodiments, the amide is a sulfonamide or a phosphoramide. In some embodiments, the amide comprises a —NC(═O)— moiety. In some embodiments, the amine that forms an amide with a described compound has a structure of NH(R²¹)₂, wherein each R²¹ is independently H, substituted or unsubstituted C₁-C₁₂ alkyl, substituted or unsubstituted C₁-C₁₂ haloalkyl, or substituted or unsubstituted C₁-C₁₂ heteroalkyl.

In some embodiments of a compound of Formula (VI), (I), (II), (IIa), (IIb), (III), (IV), or (V), or a pharmaceutically acceptable salt or solvate thereof, the abundance of deuterium in each of R⁵, R⁶, R⁷, R⁸, R⁹, and/or R¹⁰ is independently at least 1%, at least 10%, 20%, at least 30%, at least 40%, at least 50%, at least 60%, at least 70%, at least 80%, at least 90%, or 100% of a total number of hydrogen and deuterium.

In some embodiments of a compound of Formula (VI), (I), (II), (IIa), (IIb), (III), (IV), or (V), or a pharmaceutically acceptable salt or solvate thereof, one or more of R¹ to R¹¹ groups comprise deuterium at a percentage higher than the natural abundance of deuterium. In some embodiments, R¹ comprises deuterium at a percentage higher than the natural abundance of deuterium. In some embodiments, R² comprises deuterium at a percentage higher than the natural abundance of deuterium. In some embodiments, R³ comprises deuterium at a percentage higher than the natural abundance of deuterium. In some embodiments, R⁴ comprises deuterium at a percentage higher than the natural abundance of deuterium. In some embodiments, R⁵ comprises deuterium at a percentage higher than the natural abundance of deuterium. In some embodiments, R⁶ comprises deuterium at a percentage higher than the natural abundance of deuterium. In some embodiments, R⁷ comprises deuterium at a percentage higher than the natural abundance of deuterium. In some embodiments, R⁸ comprises deuterium at a percentage higher than the natural abundance of deuterium. In some embodiments, R⁹ comprises deuterium at a percentage higher than the natural abundance of deuterium. In some embodiments, R¹⁰ comprises deuterium at a percentage higher than the natural abundance of deuterium. In some embodiments, R¹¹ comprises deuterium at a percentage higher than the natural abundance of deuterium. In some embodiments, the percentage of deuterium is at least 1%, at least 10%, 20%, at least 30%, at least 40%, at least 50%, at least 60%, at least 70%, at least 80%, at least 90%, at least 99%, or 100%.

In some embodiments of a compound of Formula (VI), (I), (II), (IIa), (IIb), (III), (IV), or (V), or a pharmaceutically acceptable salt or solvate thereof, the abundance of deuterium in the compound is higher than the natural abundance of deuterium. In some embodiments, the percentage of deuterium is at least 1%, at least 10%, 20%, at least 30%, at least 40%, at least 50%, at least 60%, at least 70%, at least 80%, at least 90%, at least 99%, or 100%.

In some embodiments, described herein is a compound selected from TABLE 1, or a pharmaceutically acceptable salt or solvate thereof.

TABLE 1
Exemplary Compounds

| Compound | | | |
|---|---|---|---|
| No. | Structure | IUPAC name | MW |
| 1008 | 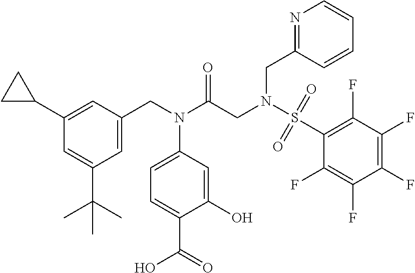 [SMILES: CC(C)(C)c1cc(CN(C(=O)CN(Cc2ccccn2)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(pyridin-2- ylmethyl)-(2,3,4,5,6- pentafluoro phenyl)sulfonamido) acetamido)-2- hydroxybenzoic acid | 717.7 |
| 1009 | 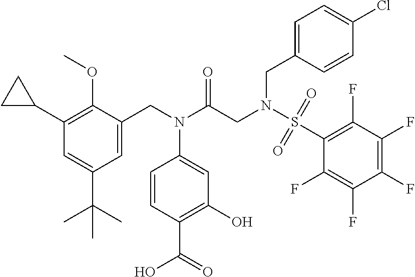 [SMILES: COc1c(CN(C(=O)CN(Cc2ccc(Cl)cc2)S(=O)(=O)c2c(F)c(F)c(F)c(F)c2F)c2ccc(C(=O)O)c(O)c2)cc(C(C)(C)C)cc1C1CC1] | 4-(N-(5-(tert-butyl)-3- cyclopropyl-2- methoxybenzyl)-2-(N- (4-chlorobenzyl)- (2,3,4,5,6- pentafluorophenyl)sulfon- amido)acetamido)-2- hydroxybenzoic acid | 781.2 |
| 1010 | 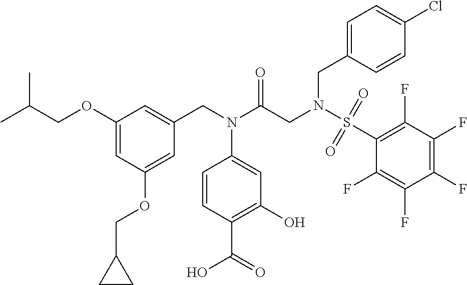 [SMILES: CC(C)COc1cc(CN(C(=O)CN(Cc2ccc(Cl)cc2)S(=O)(=O)c2c(F)c(F)c(F)c(F)c2F)c2ccc(C(=O)O)c(O)c2)cc(OCC2CC2)c1] | 4-(2-(N-(4- chlorobenzyl)- (2,3,4,5,6- pentafluorophenyl) sulfonamido)-N-(3- (cyclopropylmethoxy)- 5- isobutoxybenzyl) acetamido)- 2-hydroxybenzoic acid | 797.2 |
| 1011 | 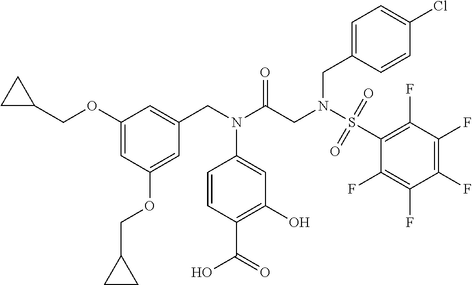 [SMILES: O=C(O)c1ccc(N(Cc2cc(OCC3CC3)cc(OCC3CC3)c2)C(=O)CN(Cc2ccc(Cl)cc2)S(=O)(=O)c2c(F)c(F)c(F)c(F)c2F)cc1O] | 4-(N-(3,5- bis(cyclopropylmethoxy) benzyl)-2-(N-(4- chlorobenzyl)- (2,3,4,5,6- pentafluorophenyl)sulfon- amido)acetamido)-2- hydroxybenzoic acid | 795.2 |
| 1013 | 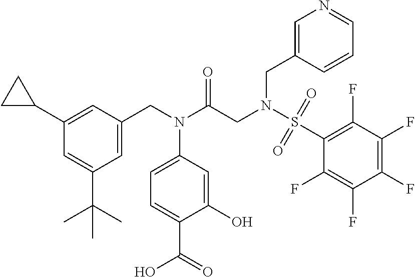 [SMILES: CC(C)(C)c1cc(CN(C(=O)CN(Cc2cccnc2)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(pyridin-3- ylmethyl)-(2,3,4,5,6- pentafluoro- phenyl)sulfonamido) acetamido)-2- hydroxybenzoic acid | 717.7 |
| 1015 | 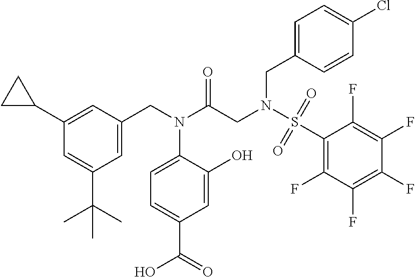 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)cc2O)cc(C2CC2)c1] | 4-(N-(3-(tert-butyl)-5- cyclopropylbenzyl)-2- (N-(4-chlorobenzyl)- (2,3,4,5,6- pentafluorophenyl)sulfon- amido)acetamido)-3- hydroxybenzoic acid | 751.2 |
| 1016 | 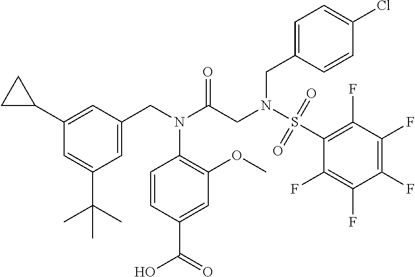 [SMILES: COc1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | 4-(N-(3-(tert-butyl)-5- cyclopropylbenzyl)-2- (N-(4-chlorobenzyl)- (2,3,4,5,6- pentafluorophenyl)sulfon- amido)acetamido)-3- methoxybenzoic acid | 765.2 |
| 1017 | 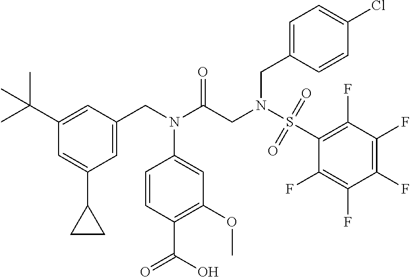 [SMILES: COc1cc(N(Cc2cc(C3CC3)cc(C(C)(C)C)c2)C(=O)CN(Cc2ccc(Cl)cc2)S(=O)(=O)c2c(F)c(F)c(F)c(F)c2F)ccc1C(=O)O] | 4-(N-(3-(tert-butyl)-5- cyclopropylbenzyl)-2- (N-(4-chlorobenzyl)- (2,3,4,5,6- pentafluorophenyl)sulfon- amido)acetamido)-2- methoxybenzoic acid | 765.2 |
| 1018 | 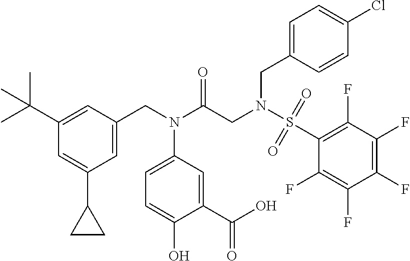 [SMILES: CC(C)(C)c1cc(CN(C(=O)CN(Cc2ccc(Cl)cc2)S(=O)(=O)c2c(F)c(F)c(F)c(F)c2F)c2ccc(O)c(C(=O)O)c2)cc(C2CC2)c1] | 5-(N-(3-(tert-butyl)-5- cyclopropylbenzyl)-2- (N-(4-chlorobenzyl)- (2,3,4,5,6- pentafluorophenyl)sulfon- amido)acetamido)-2- hydroxybenzoic acid | 751.2 |
| 1019 | 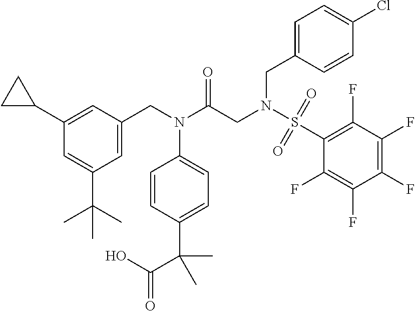 [SMILES: CC(C)(C)c1cc(CN(C(=O)CN(Cc2ccc(Cl)cc2)S(=O)(=O)c2c(F)c(F)c(F)c(F)c2F)c2ccc(C(C)(C)C(=O)O)cc2)cc(C2CC2)c1] | 2-(4-(N-(3-(tert-butyl)- 5-cyclopropylbenzyl)-2- (N-(4-chlorobenzyl)- (2,3,4,5,6- pentafluorophenyl)sulfon- amido)acetamido)phenyl)- 2-methylpropanoic acid | 777.2 |
| 1020 | 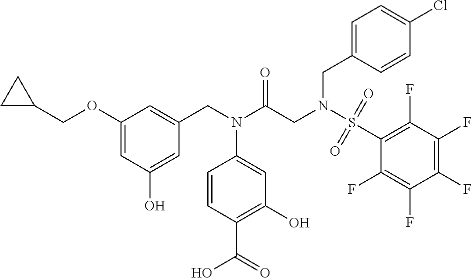 [SMILES: O=C(O)c1ccc(N(Cc2cc(O)cc(OCC3CC3)c2)C(=O)CN(Cc2ccc(Cl)cc2)S(=O)(=O)c2c(F)c(F)c(F)c(F)c2F)cc1O] | 4-(2-(N-(4- chlorobenzyl)- (2,3,4,5,6- pentafluorophenyl)sulfon- amido)-N-(3- (cyclopropylmethoxy)- 5- hydroxybenzyl)acetamido)- 2-hydroxybenzoic acid | 741.1 |
| 1021 | 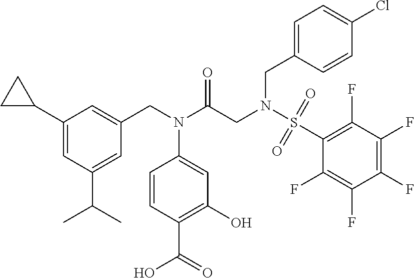 [SMILES: CC(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CC2)c1] | 4-(2-(N-(4- chlorobenzyl)- (2,3,4,5,6- pentafluorophenyl)sulfon- amido)-N-(3- cyclopropyl-5- isopropylbenzyl) acetamido)- 2-hydroxybenzoic acid | 737.1 |
| 1026 | 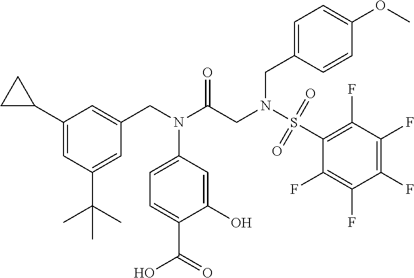 [SMILES: COc1ccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)cc1] | 4-(N-(3-(tert-butyl)-5- cyclopropylbenzyl)-2- (N-(4-methoxybenzyl)- (2,3,4,5,6-pentafluoro- phenyl)sulfonamido) acetamido)-2- hydroxybenzoic acid | 746.7 |
| 1027 | 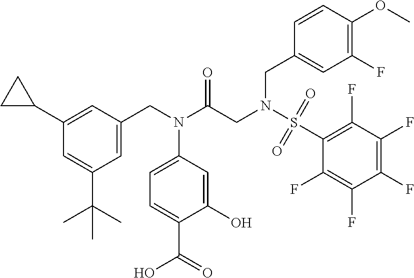 [SMILES: COc1ccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)cc1F] | 4-(N-(3-(tert-butyl)-5- cyclopropylbenzyl)-2- (N-(3-fluoro-4- methoxybenzyl)- (2,3,4,5,6-pentafluoro- phenyl)sulfonamido) acetamido)-2- hydroxybenzoic acid | 764.7 |
| 1028 | 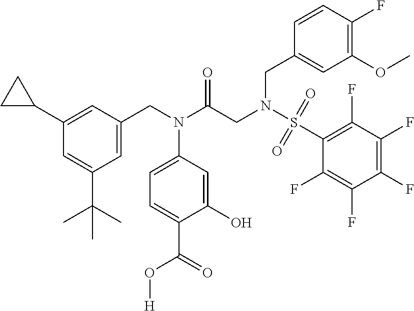 [SMILES: COc1cc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)ccc1F] | 4-(N-(3-(tert-butyl)-5- cyclopropylbenzyl)-2- (N-(4-fluoro-3- methoxybenzyl)- (2,3,4,5,6-pentafluoro- phenyl)sulfonamido) acetamido)-2- hydroxybenzoic acid | 764.7 |
| 1029 | 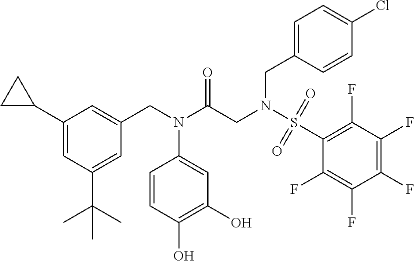 [SMILES: CC(C)(C)c1cc(CN(C(=O)CN(Cc2ccc(Cl)cc2)S(=O)(=O)c2c(F)c(F)c(F)c(F)c2F)c2ccc(O)c(O)c2)cc(C2CC2)c1] | N-(3-(tert-butyl)-5- cyclopropylbenzyl)-2- (N-(4-chlorobenzyl)- (2,3,4,5,6- pentafluorophenyl) sulfonamido)-N-(3,4- dihydroxyphenyl) acetamide | 723.2 |
| 1030 | 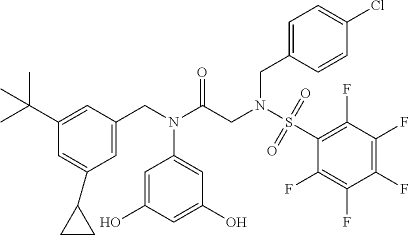 [SMILES: CC(C)(C)c1cc(CN(C(=O)CN(Cc2ccc(Cl)cc2)S(=O)(=O)c2c(F)c(F)c(F)c(F)c2F)c2cc(O)cc(O)c2)cc(C2CC2)c1] | N-(3-(tert-butyl)-5- cyclopropylbenzyl)-2- (N-(4-chlorobenzyl)- (2,3,4,5,6- pentafluorophenyl) sulfonamido)-N-(3,5- dihydroxyphenyl) acetamide | 723.2 |
| 1031 | 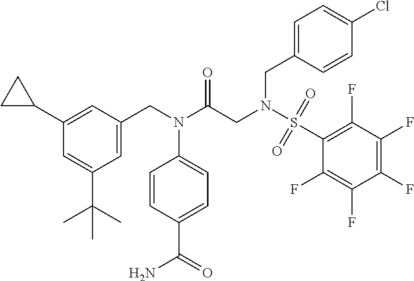 [SMILES: CC(C)(C)c1cc(CN(C(=O)CN(Cc2ccc(Cl)cc2)S(=O)(=O)c2c(F)c(F)c(F)c(F)c2F)c2ccc(C(N)=O)cc2)cc(C2CC2)c1] | 4-(N-(3-(tert-butyl)-5- cyclopropylbenzyl)-2- (N-(4-chlorobenzyl)- (2,3,4,5,6- pentafluorophenyl)sulfon- amido)acetamido) benzamide | 734.2 |
| 1032 | 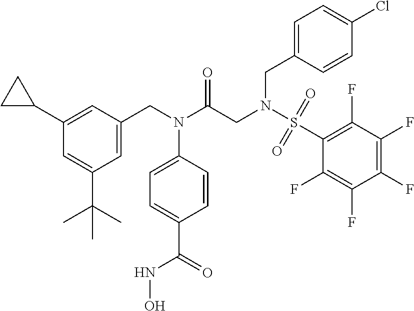 [SMILES: CC(C)(C)c1cc(CN(C(=O)CN(Cc2ccc(Cl)cc2)S(=O)(=O)c2c(F)c(F)c(F)c(F)c2F)c2ccc(C(=O)NO)cc2)cc(C2CC2)c1] | 4-(N-(3-(tert-butyl)-5- cyclopropylbenzyl)-2- (N-(4-chlorobenzyl)- (2,3,4,5,6- pentafluorophenyl)sulfon- amido)acetamido)-N- hydroxybenzamide | 750.2 |
| 1033 | 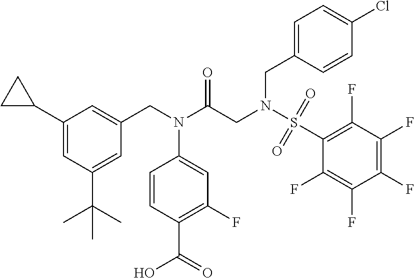 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(F)c2)cc(C2CC2)c1] | 4-(N-(3-(tert-butyl)-5- cyclopropylbenzyl)-2- (N-(4-chlorobenzyl)- (2,3,4,5,6- pentafluorophenyl)sulfon- amido)acetamido)-2- fluorobenzoic acid | 753.2 |
| 1034 | 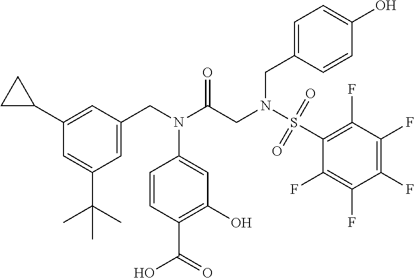 [SMILES: CC(C)(C)c1cc(CN(C(=O)CN(Cc2ccc(O)cc2)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(4-hydroxybenzyl)- (2,3,4,5,6-pentafluoro- phenyl)sulfonamido) acetamido)-2- hydroxybenzoic acid | 732.7 |
| 1035 | 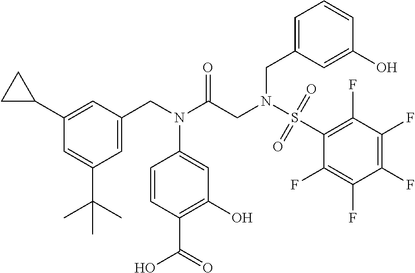 [SMILES: CC(C)(C)c1cc(CN(C(=O)CN(Cc2cccc(O)c2)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(3-hydroxybenzyl)- (2,3,4,5,6-pentafluoro- phenyl)sulfonamido) acetamido)-2- hydroxybenzoic acid | 732.7 |
| 1036 | 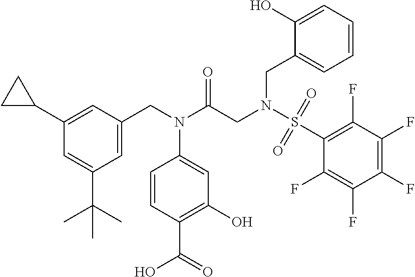 [SMILES: CC(C)(C)c1cc(CN(C(=O)CN(Cc2ccccc2O)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(2-hydroxybenzyl)- (2,3,4,5,6-pentafluoro- phenyl)sulfonamido) acetamido)-2- hydroxybenzoic acid | 732.7 |
| 1037 | 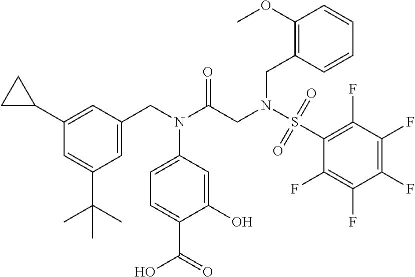 [SMILES: COc1ccccc1CN(CC(=O)N(Cc1cc(C2CC2)cc(C(C)(C)C)c1)c1ccc(C(=O)O)c(O)c1)S(=O)(=O)c1c(F)c(F)c(F)c(F)c1F] | 4-(N-(3-(tert-butyl)-5- cyclopropylbenzyl)-2- (N-(2-methoxybenzyl)- (2,3,4,5,6-pentafluoro- phenyl)sulfonamido) acetamido)-2- hydroxybenzoic acid | 746.7 |
| 1038 | 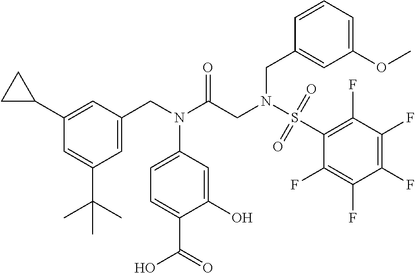 [SMILES: COc1cccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)c1] | 4-(N-(3-(tert-butyl)-5- cyclopropylbenzyl)-2- (N-(3-methoxybenzyl)- (2,3,4,5,6-pentafluoro- phenyl)sulfonamido) acetamido)-2- hydroxybenzoic acid | 746.7 |
| 1039 | 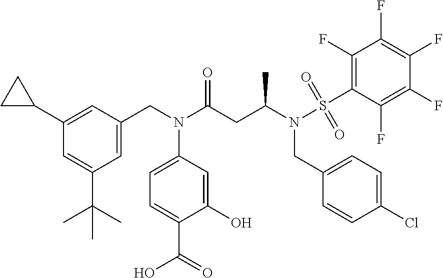 [SMILES: C[C@H](CC(=O)N(Cc1cc(C2CC2)cc(C(C)(C)C)c1)c1ccc(C(=O)O)c(O)c1)N(Cc1ccc(Cl)cc1)S(=O)(=O)c1c(F)c(F)c(F)c(F)c1F] | (R)-4-(N-(3-(tert-butyl)- 5-cyclopropylbenzyl)-3- (N-(4-chlorobenzyl)- (2,3,4,5,6- pentafluorophenyl)sulfon- amido)butanamido)-2- hydroxybenzoic acid | 779.2 |
| 1040 | 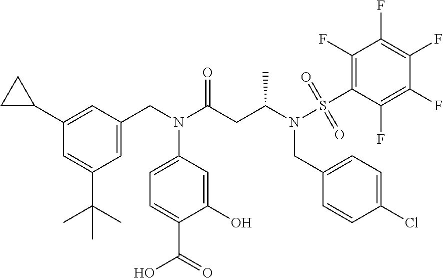 [SMILES: C[C@@H](CC(=O)N(Cc1cc(C2CC2)cc(C(C)(C)C)c1)c1ccc(C(=O)O)c(O)c1)N(Cc1ccc(Cl)cc1)S(=O)(=O)c1c(F)c(F)c(F)c(F)c1F] | (S)-4-(N-(3-(tert-butyl)- 5-cyclopropylbenzyl)-3- (N-(4-chlorobenzyl)- (2,3,4,5,6- pentafluorophenyl)sulfon- amido)butanamido)-2- hydroxybenzoic acid | 779.2 |
| 1041 | 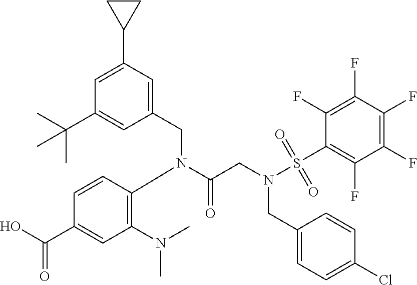 [SMILES: CN(C)c1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | 4-(N-(3-(tert-butyl)-5- cyclopropylbenzyl)-2- (N-(4-chlorobenzyl)- (2,3,4,5,6- pentafluorophenyl)sulfon- amido)acetamido)-3- (dimethylamino)benzoic acid | 778.2 |
| 1042 | 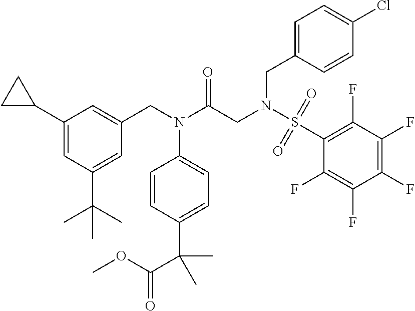 [SMILES: COC(=O)C(C)(C)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1] | methyl 2-(4-(N-(3-(tert- butyl)-5- cyclopropylbenzyl)-2- (N-(4-chlorobenzyl)- (2,3,4,5,6- pentafluorophenyl)sulfon- amido)acetamido)phenyl)- 2-methylpropanoate | 791.3 |
| 1048 | 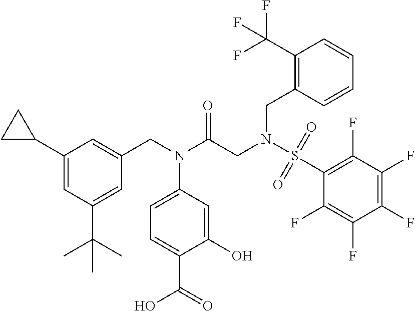 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-2- hydroxybenzoic acid | 784.7 |
| 1050 | 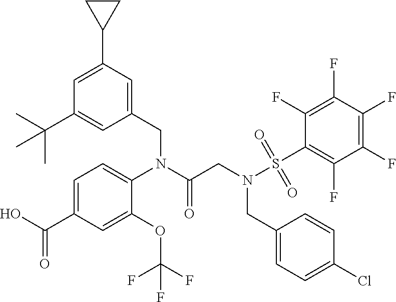 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)cc2OC(F)(F)F)cc(C2CC2)c1] | 4-(N-(3-(tert-butyl)-5- cyclopropylbenzyl)-2- (N-(4-chlorobenzyl)- (2,3,4,5,6- pentafluorophenyl)sulfon- amido)acetamido)-3- (trifluoromethoxy) benzoic acid | 819.2 |
| 1051 | 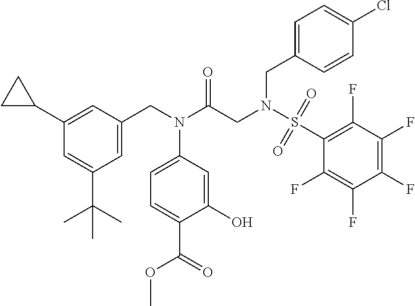 [SMILES: COC(=O)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1O] | methyl 4-(N-(3-(tert- butyl)-5- cyclopropylbenzyl)-2- (N-(4-chlorobenzyl)- (2,3,4,5,6- pentafluorophenyl)sulfon- amido)acetamido)-2- hydroxybenzoate | 765.2 |
| 1052 | 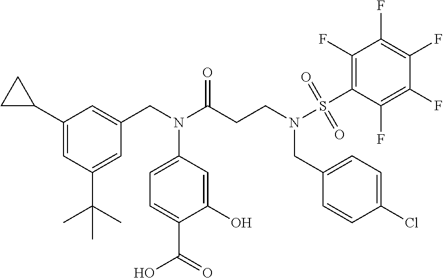 [SMILES: CC(C)(C)c1cc(CN(C(=O)CCN(Cc2ccc(Cl)cc2)S(=O)(=O)c2c(F)c(F)c(F)c(F)c2F)c2ccc(C(=O)O)c(O)c2)cc(C2CC2)c1] | 4-(N-(3-(tert-butyl)-5- cyclopropylbenzyl)-3- (N-(4-chlorobenzyl)- (2,3,4,5,6- pentafluorophenyl)sulfon- amido)propanamido)- 2-hydroxybenzoic acid | 765.2 |
| 1054 | 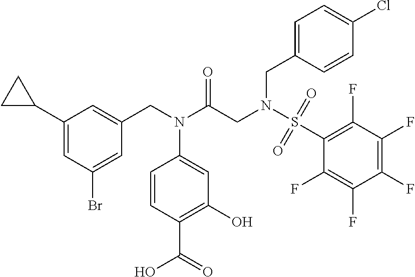 [SMILES: O=C(O)c1ccc(N(Cc2cc(Br)cc(C3CC3)c2)C(=O)CN(Cc2ccc(Cl)cc2)S(=O)(=O)c2c(F)c(F)c(F)c(F)c2F)cc1O] | 4-(N-(3-bromo-5- cyclopropylbenzyl)-2- (N-(4-chlorobenzyl)- (2,3,4,5,6- pentafluorophenyl)sulfon- amido)acetamido)-2- hydroxybenzoic acid | 774 |
| 1055 | 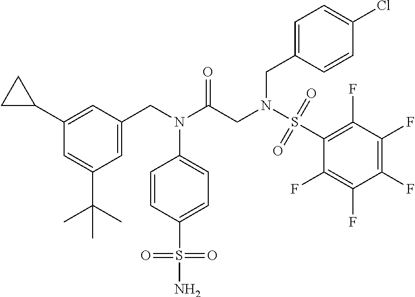 [SMILES: CC(C)(C)c1cc(CN(C(=O)CN(Cc2ccc(Cl)cc2)S(=O)(=O)c2c(F)c(F)c(F)c(F)c2F)c2ccc(S(N)(=O)=O)cc2)cc(C2CC2)c1] | N-(3-(tert-butyl)-5- cyclopropylbenzyl)-2- (N-(4-chlorobenzyl)- (2,3,4,5,6- pentafluorophenyl)sulfon- amido)-N-(4- sulfamoylphenyl) acetamide | 770.2 |
| 1056 | 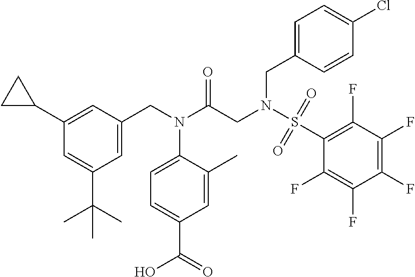 [SMILES: Cc1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | 4-(N-(3-(tert-butyl)-5- cyclopropylbenzyl)-2- (N-(4-chlorobenzyl)- (2,3,4,5,6- pentafluorophenyl)sulfon- amido)acetamido)-3- methylbenzoic acid | 749.2 |
| 1058 | 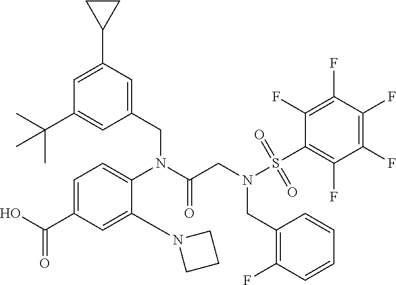 [SMILES: CC(C)(C)c1cc(CN(C(=O)CN(Cc2ccccc2F)S(=O)(=O)c2c(F)c(F)c(F)c(F)c2F)c2ccc(C(=O)O)cc2N2CCC2)cc(C2CC2)c1] | 3-(azetidin-1-yl)-4-(N- (3-(tert-butyl)-5- cyclopropylbenzyl)-2- (N-(2-fluorobenzyl)- (2,3,4,5,6-pentafluoro- phenyl)sulfonamido) acetamido)benzoic acid | 773.8 |
| 1059 | 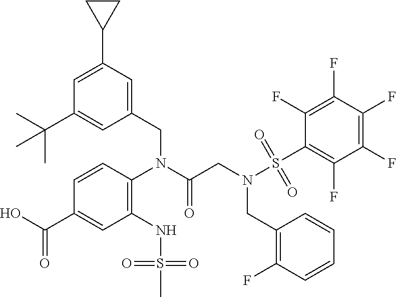 [SMILES: CC(C)(C)c1cc(CN(C(=O)CN(Cc2ccccc2F)S(=O)(=O)c2c(F)c(F)c(F)c(F)c2F)c2ccc(C(=O)O)cc2NS(C)(=O)=O)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- (methylsulfonamido) benzoic acid | 811.8 |
| 1060 | 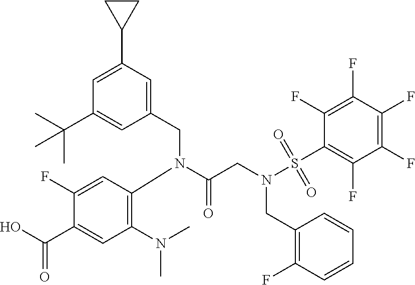 [SMILES: CN(C)c1cc(C(=O)O)c(F)cc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-5- (dimethylamino)-2- fluorobenzoic acid | 779.8 |
| 1061 | 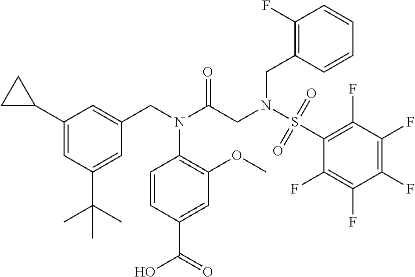 [SMILES: COc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- methoxybenzoic acid | 748.7 |
| 1062 | 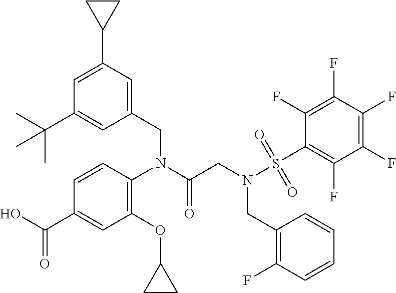 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- cyclopropoxybenzoic acid | 774.8 |
| 1064 | 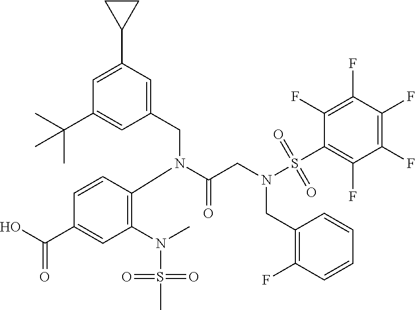 [SMILES: CN(c1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F)S(C)(=O)=O] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3-(N- methylmethylsulfonamido) benzoic acid | 825.8 |
| 1065 | 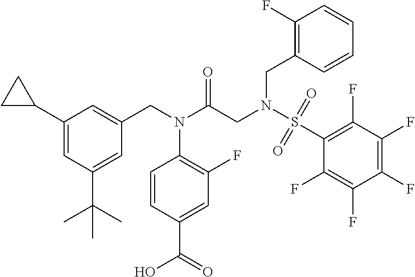 [SMILES: CC(C)(C)c1cc(CN(C(=O)CN(Cc2ccccc2F)S(=O)(=O)c2c(F)c(F)c(F)c(F)c2F)c2ccc(C(=O)O)cc2F)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- fluorobenzoic acid | 736.7 |
| 1066 | 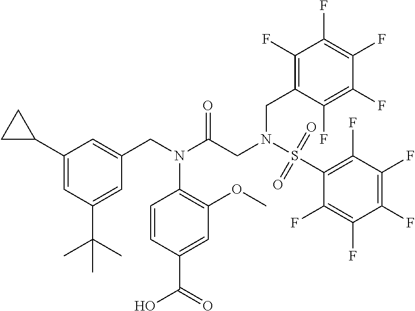 [SMILES: COc1cc(C(=O)O)ccc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | 4-(N-(3-(tert-butyl)-5- cyclopropylbenzyl)-2- (N- ((perfluorophenyl) methyl)-(2,3,4,5,6- pentafluoro- phenyl)sulfonamido) acetamido)-3- methoxybenzoic acid | 820.7 |
| 1067 | 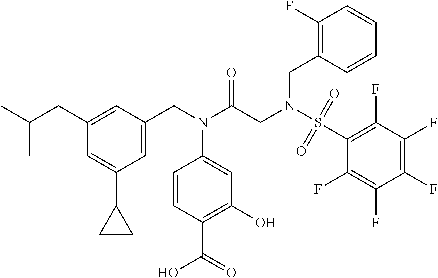 [SMILES: CC(C)Cc1cc(CN(C(=O)CN(Cc2ccccc2F)S(=O)(=O)c2c(F)c(F)c(F)c(F)c2F)c2ccc(C(=O)O)c(O)c2)cc(C2CC2)c1] | 4-(N-(3-cyclopropyl-5- isobutylbenzyl)-2-(N- (2-fluorobenzyl)- (2,3,4,5,6-pentafluoro- phenyl)sulfonamido) acetamido)-2- hydroxybenzoic acid | 734.7 |
| 1069 | 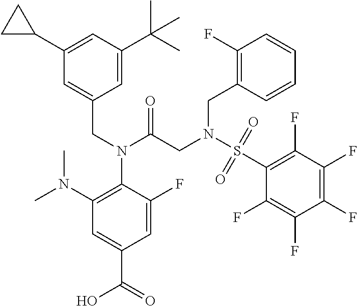 [SMILES: CN(C)c1cc(C(=O)O)cc(F)c1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- (dimethylamino)-5- fluorobenzoic acid | 779.8 |
| 1070 | 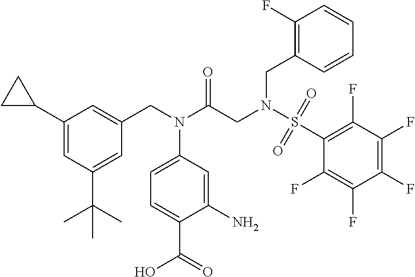 [SMILES: CC(C)(C)c1cc(CN(C(=O)CN(Cc2ccccc2F)S(=O)(=O)c2c(F)c(F)c(F)c(F)c2F)c2ccc(C(=O)O)c(N)c2)cc(C2CC2)c1] | 2-amino-4-(N-(3-(tert- butyl)-5- cyclopropylbenzyl)-2- (N-(2-fluorobenzyl)- (2,3,4,5,6-pentafluoro- phenyl)sulfonamido) acetamido)benzoic acid | 733.7 |
| 1071 | 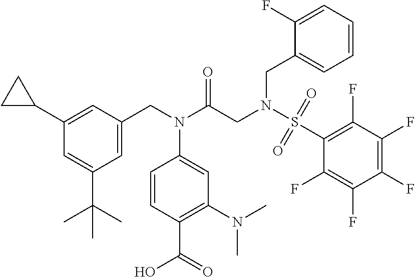 [SMILES: CN(C)c1cc(N(Cc2cc(C3CC3)cc(C(C)(C)C)c2)C(=O)CN(Cc2ccccc2F)S(=O)(=O)c2c(F)c(F)c(F)c(F)c2F)ccc1C(=O)O] | 4-(N-(3-(tert-butyl)-5- cyclopropylbenzyl)-2- (N-(2-fluorobenzyl)- (2,3,4,5,6-pentafluoro- phenyl)sulfonamido) acetamido)-2- (dimethylamino) benzoic acid | 761.8 |
| 1072 | 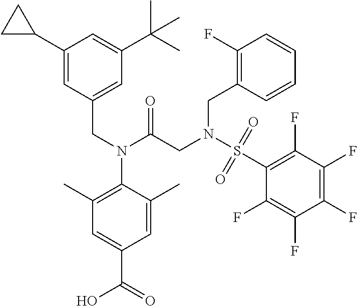 [SMILES: Cc1cc(C(=O)O)cc(C)c1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3,5- dimethylbenzoic acid | 746.8 |
| 1073 | 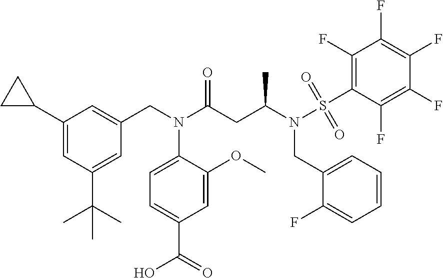 [SMILES: COc1cc(C(=O)O)ccc1N(Cc1cc(C2CC2)cc(C(C)(C)C)c1)C(=O)C[C@@H](C)N(Cc1ccccc1F)S(=O)(=O)c1c(F)c(F)c(F)c(F)c1F] | (R)-4-(N-(3-tert-butyl)- 5-cyclopropylbenzyl)-3- (N-(2-fluorobenzyl)- (2,3,4,5,6-pentafluoro- phenyl)sulfonamido) butanamido)-3- methoxybenzoic acid | 776.8 |
| 1074 | 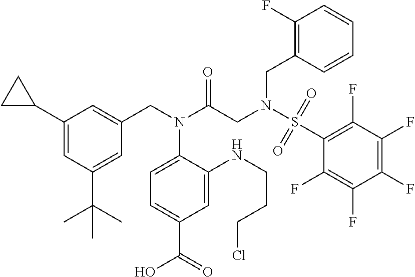 [SMILES: CC(C)(C)c1cc(CN(C(=O)CN(Cc2ccccc2F)S(=O)(=O)c2c(F)c(F)c(F)c(F)c2F)c2ccc(C(=O)O)cc2NCCCCl)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3-((3- chloropropyl)amino) benzoic acid | 810.2 |
| 1075 | 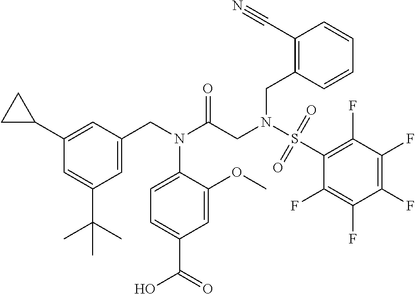 [SMILES: COc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | 4-(N-(3-(tert-butyl)-5- cyclopropylbenzyl)-2- (N-(2-cyanobenzyl)- (2,3,4,5,6- pentafluorophenyl)sulfon- amido)acetamido)-3- methoxybenzoic acid | 755.8 |
| 1076 | 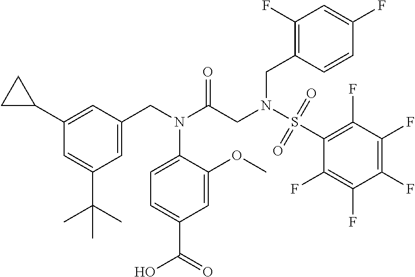 [SMILES: COc1cc(C(=O)O)ccc1N(Cc1cc(C2CC2)cc(C(C)(C)C)c1)C(=O)CN(Cc1ccc(F)cc1F)S(=O)(=O)c1c(F)c(F)c(F)c(F)c1F] | 4-(N-(3-(tert-butyl)-5- cyclopropylbenzyl)-2- (N-(2,4- difluorobenzyl)- (2,3,4,5,6- pentafluorophenyl)sulfon- amido)acetamido)-3- methoxybenzoic acid | 766.7 |
| 1077 | 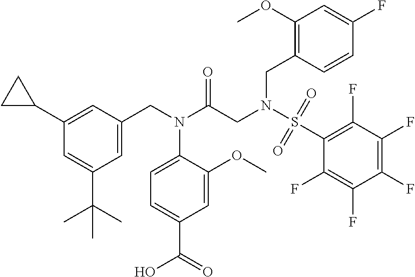 [SMILES: COc1cc(F)ccc1CN(CC(=O)N(Cc1cc(C2CC2)cc(C(C)(C)C)c1)c1ccc(C(=O)O)cc1OC)S(=O)(=O)c1c(F)c(F)c(F)c(F)c1F] | 4-(N-(3-(tert-butyl)-5- cyclopropylbenzyl)-2- (N-(4-fluoro-2- methoxybenzyl)- (2,3,4,5,6-pentafluoro- phenyl)sulfonamido) acetamido)-3- methoxybenzoic acid | 778.8 |
| 1078 | 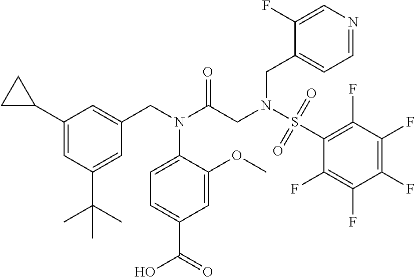 [SMILES: COc1cc(C(=O)O)ccc1N(Cc1cc(C2CC2)cc(C(C)(C)C)c1)C(=O)CN(Cc1ccncc1F)S(=O)(=O)c1c(F)c(F)c(F)c(F)c1F] | 4-(N-(3-(tert-butyl)-5- cyclopropylbenzyl)-2- (N-((3-fluoropyridin-4- yl)methyl)-(2,3,4,5,6- pentafluoro- phenyl)sulfonamido) acetamido)-3- methoxybenzoic acid | 749.7 |
| 1079 | 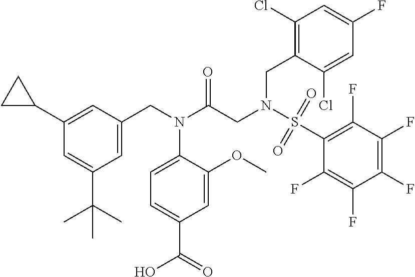 [SMILES: COc1cc(C(=O)O)ccc1N(Cc1cc(C2CC2)cc(C(C)(C)C)c1)C(=O)CN(Cc1c(Cl)cc(F)cc1Cl)S(=O)(=O)c1c(F)c(F)c(F)c(F)c1F] | 4-(N-(3-(tert-butyl)-5- cyclopropylbenzyl)-2- (N-(2,6-dichloro-4- fluorobenzyl)- (2,3,4,5,6- pentafluorophenyl)sulfon- amido)acetamido)-3- methoxybenzoic acid | 817.6 |
| 1080 | 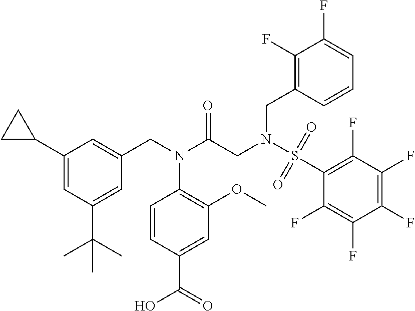 [SMILES: COc1cc(C(=O)O)ccc1N(Cc1cc(C2CC2)cc(C(C)(C)C)c1)C(=O)CN(Cc1cccc(F)c1F)S(=O)(=O)c1c(F)c(F)c(F)c(F)c1F] | 4-(N-(3-(tert-butyl)-5- cyclopropylbenzyl)-2- (N-(2,3- difluorobenzyl)- (2,3,4,5,6- pentafluorophenyl)sulfo namido)acetamido)-3- methoxybenzoic acid | 766.7 |
| 1081 | 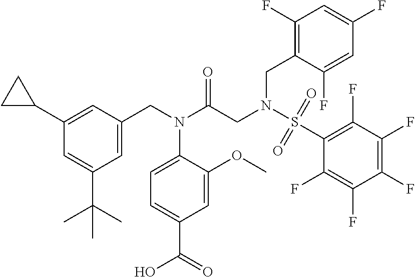 [SMILES: COc1cc(C(=O)O)ccc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | 4-(N-(3-(tert-butyl)-5- trifluorobenzyl)- (2,3,4,5,6-pentafluoro- phenyl)sulfonamido) acetamido)-3- methoxybenzoic acid | 784.7 |
| 1082 | 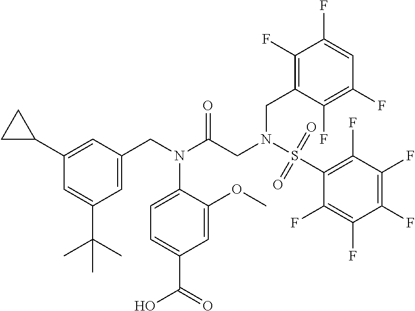 [SMILES: COc1cc(C(=O)O)ccc1N(Cc1cc(C2CC2)cc(C(C)(C)C)c1)C(=O)CN(Cc1c(F)c(F)cc(F)c1F)S(=O)(=O)c1c(F)c(F)c(F)c(F)c1F] | 4-(N-(3-(tert-butyl)-5- cyclopropylbenzyl)-2- (N-(2,3,5,6- tetrafluorobenzyl)- (2,3,4,5,6-pentafluoro- phenyl)sulfonamido) acetamido)-3- methoxybenzoic acid | 802.7 |
| 1083 | 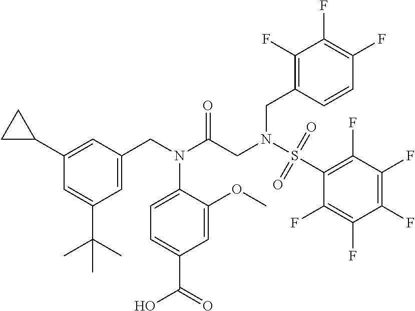 [SMILES: COc1cc(C(=O)O)ccc1N(Cc1cc(C2CC2)cc(C(C)(C)C)c1)C(=O)CN(Cc1ccc(F)c(F)c1F)S(=O)(=O)c1c(F)c(F)c(F)c(F)c1F] | 4-(N-(3-(tert-butyl)-5- cyclopropylbenzyl)-2- (N-(2,3,4- trifluorobenzyl)- (2,3,4,5,6-pentafluoro- phenyl)sulfonamido) acetamido)-3- methoxybenzoic acid | 784.7 |
| 1084 | 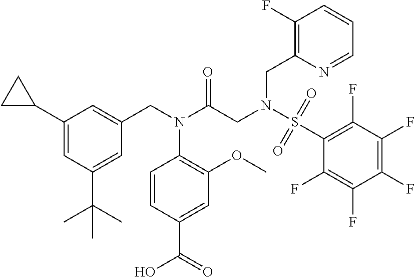 [SMILES: COc1cc(C(=O)O)ccc1N(Cc1cc(C2CC2)cc(C(C)(C)C)c1)C(=O)CN(Cc1ncccc1F)S(=O)(=O)c1c(F)c(F)c(F)c(F)c1F] | 4-(N-(3-(tert-butyl)-5- cyclopropylbenzyl)-2- (N-((3-fluoropyridin-2- yl)methyl)-(2,3,4,5,6- pentafluoro- phenyl)sulfonamido) acetamido)-3- methoxybenzoic acid | 749.7 |
| 1085 | 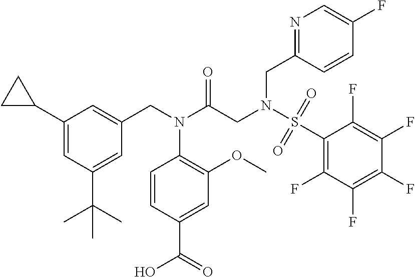 [SMILES: COc1cc(C(=O)O)ccc1N(Cc1cc(C2CC2)cc(C(C)(C)C)c1)C(=O)CN(Cc1ccc(F)cn1)S(=O)(=O)c1c(F)c(F)c(F)c(F)c1F] | 4-(N-(3-(tert-butyl)-5- cyclopropylbenzyl)-2- (N-((5-fluoropyridin-2- yl)methyl)-(2,3,4,5,6- pentafluoro- phenyl)sulfonamido) acetamido)-3- methoxybenzoic acid | 749.7 |
| 1086 | 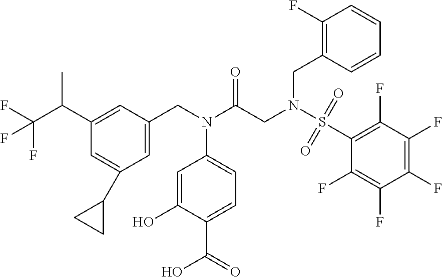 [SMILES: CC(c1cc(CN(C(=O)CN(Cc2ccccc2F)S(=O)(=O)c2c(F)c(F)c(F)c(F)c2F)c2ccc(C(=O)O)c(O)c2)cc(C2CC2)c1)C(F)(F)F] | 4-(N-(3-cyclopropyl-5- (1,1,1-trifluoropropan- 2-yl)benzyl)-2-(N-(2- fluorobenzyl)- (2,3,4,5,6-pentafluoro- phenyl)sulfonamido) acetamido)-2- hydroxybenzoic acid | 774.7 |
| 1087 | 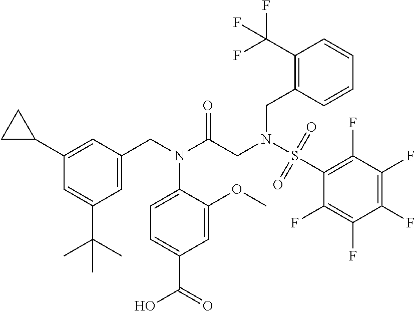 [SMILES: COc1cc(C(=O)O)ccc1N(Cc1cc(C2CC2)cc(C(C)(C)C)c1)C(=O)CN(Cc1ccccc1C(F)(F)F)S(=O)(=O)c1c(F)c(F)c(F)c(F)c1F] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-3- methoxybenzoic acid | 798.7 |
| 1088 | 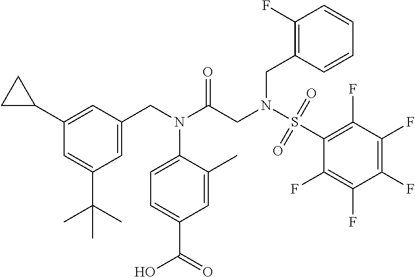 [SMILES: Cc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- methylbenzoic acid | 732.7 |
| 1089 | 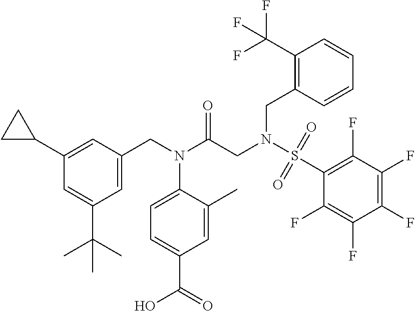 [SMILES: Cc1cc(C(=O)O)ccc1N(Cc1cc(C2CC2)cc(C(C)(C)C)c1)C(=O)CN(Cc1ccccc1C(F)(F)F)S(=O)(=O)c1c(F)c(F)c(F)c(F)c1F] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-3- methylbenzoic acid | 782.7 |
| 1090 | 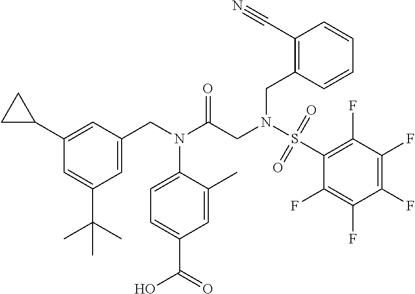 [SMILES: Cc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | 4-(N-(3-(tert-butyl)-5- cyclopropylbenzyl)-2- (N-(2-cyanobenzyl)- (2,3,4,5,6- pentafluorophenyl)sulfon- amido)acetamido)-3- methylbenzoic acid | 739.8 |
| 1091 | 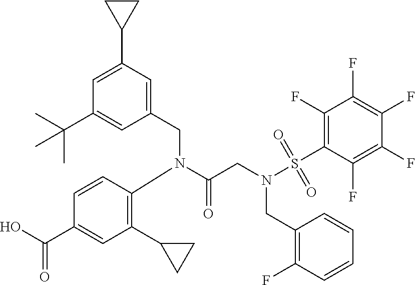 [SMILES: CC(C)(C)c1cc(CN(C(=O)CN(Cc2ccccc2F)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- cyclopropylbenzoic acid | 758.8 |
| 1092 | 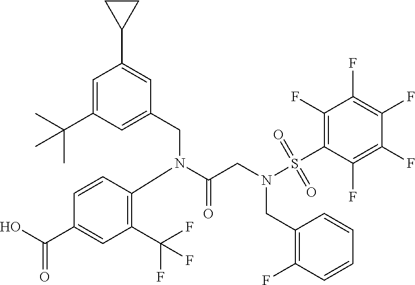 [SMILES: CC(C)(C)c1cc(CN(C(=O)CN(Cc2ccccc2F)S(=O)(=O)c2c(F)c(F)c(F)c(F)c2F)c2ccc(C(=O)O)cc2C(F)(F)F)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- (trifluoromethyl)benzoic acid | 786.7 |
| 1093 | 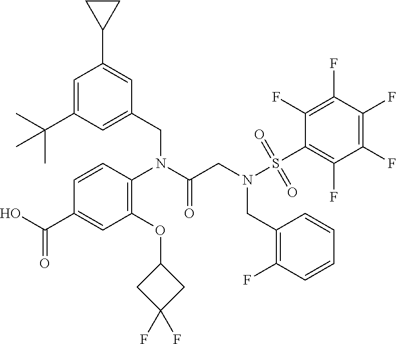 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CC(F)(F)C2)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3-(3,3- difluorocyclobutoxy) benzoic acid | 824.8 |
| 1094 | 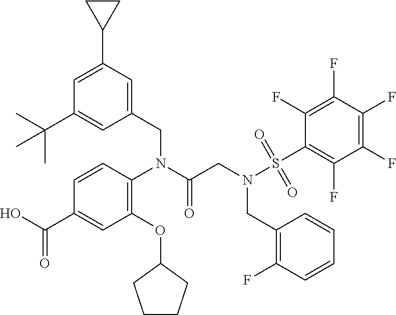 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CCCC2)cc(C2CC2)c1] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- (cyclopentyloxy)benzoic acid | 802.8 |
| 1095 | 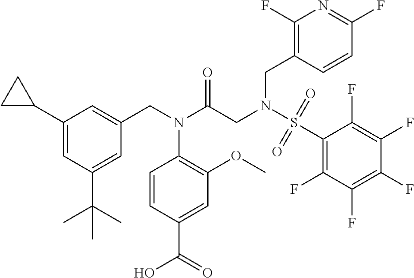 [SMILES: COc1cc(C(=O)O)ccc1N(Cc1cc(C2CC2)cc(C(C)(C)C)c1)C(=O)CN(Cc1ccc(F)nc1F)S(=O)(=O)c1c(F)c(F)c(F)c(F)c1F] | 4-(N-(3-(tert-butyl)-5- cyclopropylbenzyl)-2- (N-((2,6- difluoropyridin-3- yl)methyl)-(2,3,4,5,6- pentafluorophenyl)sulfon- amido)acetamido)-3- methoxybenzoic acid | 767.7 |
| 1096 | 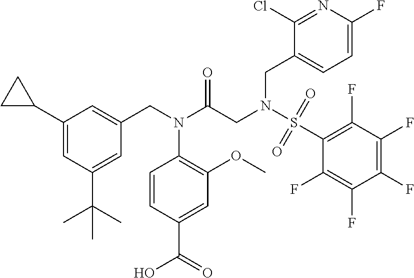 [SMILES: COc1cc(C(=O)O)ccc1N(Cc1cc(C2CC2)cc(C(C)(C)C)c1)C(=O)CN(Cc1ccc(F)nc1Cl)S(=O)(=O)c1c(F)c(F)c(F)c(F)c1F] | 4-(N-(3-(tert-butyl)-5- cyclopropylbenzyl)-2- (N-((2-chloro-6- fluoropyridin-3- yl)methyl)-(2,3,4,5,6- pentafluorophenyl)sulfon- amido)acetamido)-3- methoxybenzoic acid | 784.2 |
| 1097 | 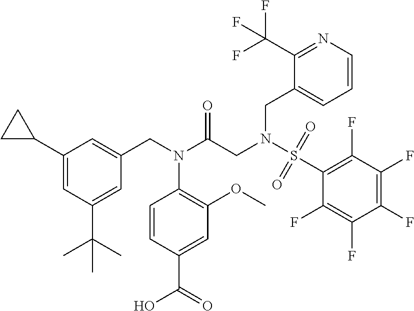 [SMILES: COc1cc(C(=O)O)ccc1N(Cc1cc(C2CC2)cc(C(C)(C)C)c1)C(=O)CN(Cc1cccnc1C(F)(F)F)S(=O)(=O)c1c(F)c(F)c(F)c(F)c1F] | 4-(N-(3-(tert-butyl)-5- cyclopropylbenzyl)-2- (N-((2- (trifluoromethyl)pyridin- 3-yl)methyl)- (2,3,4,5,6-pentafluoro- phenyl)sulfonamido) acetamido)-3- methoxybenzoic acid | 799.7 |
| 1098 | 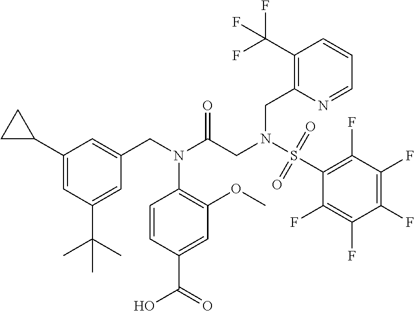 [SMILES: COc1cc(C(=O)O)ccc1N(Cc1cc(C2CC2)cc(C(C)(C)C)c1)C(=O)CN(Cc1ncccc1C(F)(F)F)S(=O)(=O)c1c(F)c(F)c(F)c(F)c1F] | 4-(N-(3-(tert-butyl)-5- cyclopropylbenzyl)-2- (N-((3- (trifluoromethyl)pyridin- 2-yl)methyl)- (2,3,4,5,6-pentafluoro- phenyl)sulfonamido) acetamido)-3- methoxybenzoic acid | 799.7 |
| 1099 | 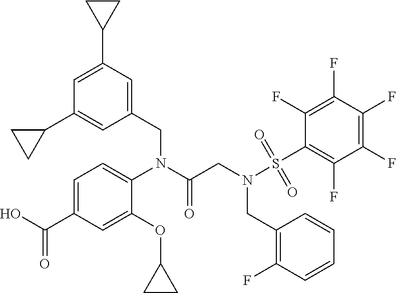 [SMILES: O=C(O)c1ccc(N(Cc2cc(C3CC3)cc(C3CC3)c2)C(=O)CN(Cc2ccccc2F)S(=O)(=O)c2c(F)c(F)c(F)c(F)c2F)c(OC2CC2)c1] | 3-cyclopropoxy-4-(N- (3,5- dicyclopropylbenzyl)-2- (N-(2-fluorobenzyl)- (2,3,4,5,6-pentafluoro- phenyl)sulfonamido) acetamido)benzoic acid | 758.7 |
| 1100 | 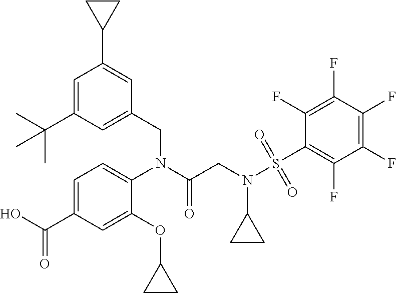 [SMILES: CC(C)(C)c1cc(CN(C(=O)CN(C2CC2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-cyclopropyl- (2,3,4,5,6- pentafluorophenyl)sulfon- amido)acetamido)-3- cyclopropoxybenzoic acid | 706.7 |
| 1101 | 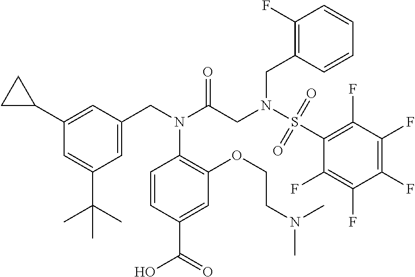 [SMILES: CN(C)CCOc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl) sulfonamido) taceamido)-3-(2- (dimethylamino)ethoxy) benzoic acid | 805.8 |
| 1105 | 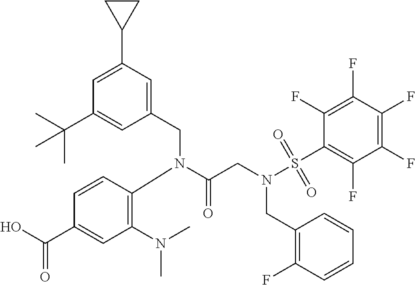 [SMILES: CN(C)c1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- (dimethylamino)benzoic acid | 761.8 |
| 1106 | 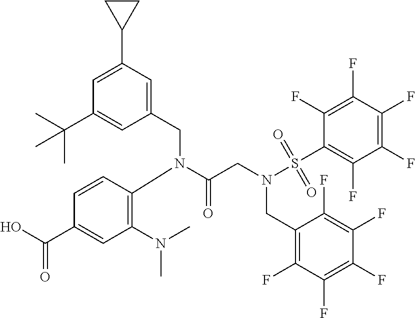 [SMILES: CN(C)c1cc(C(=O)O)ccc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | 4-(N-(3-(tert-butyl)-5- cyclopropylbenzyl)-2- (N- ((perfluorophenyl) methyl)-(2,3,4,5,6- pentafluoro phenyl)sulfonamido) acetamido)-3- (dimethylamino)benzoic acid | 833.7 |
| 1107 | 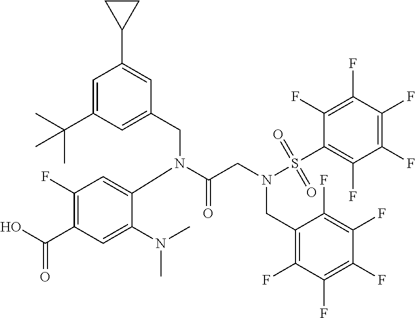 [SMILES: CN(C)c1cc(C(=O)O)c(F)cc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | 4-(N-(3-(tert-butyl)-5- cyclopropylbenzyl)-2- (N- ((perfluorophenyl) methyl)-(2,3,4,5,6- pentafluoro- phenyl)sulfonamido) acetamido)-5- (dimethylamino)-2- fluorobenzoic acid | 851.7 |
| 1108 | 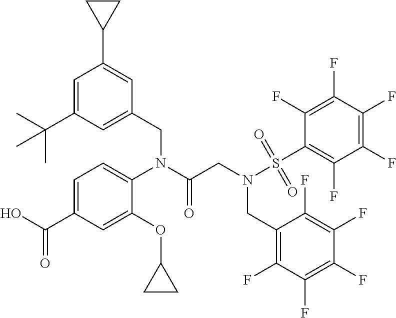 [SMILES: CC(C)(C)c1cc(CN(C(=O)CN(Cc2c(F)c(F)c(F)c(F)c2F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N- ((perfluorophenyl) methyl)-(2,3,4,5,6- pentafluoro phenyl)sulfonamido) acetamido)-3- cyclopropoxybenzoic acid | 846.7 |
| 1110 | 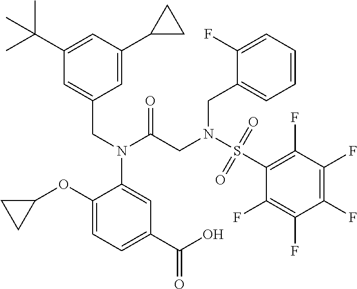 [SMILES: CC(C)(C)c1cc(CN(C(=O)CN(Cc2ccccc2F)S(=O)(=O)c2c(F)c(F)c(F)c(F)c2F)c2cc(C(=O)O)ccc2OC2CC2)cc(C2CC2)c1] | 3-(N-(3-(tert-butyl)-5- cyclopropylbenzyl)-2- (N-(2-fluorobenzyl)- (2,3,4,5,6-pentafluoro- phenyl)sulfonamido) acetamido)-4- cyclopropoxybenzoic acid | 774.8 |
| 1111 | 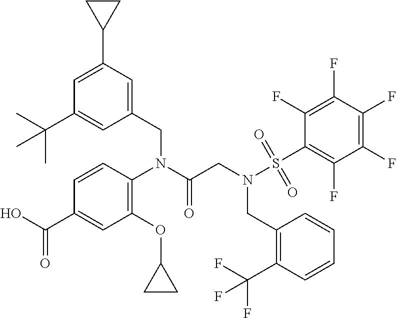 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-3- cyclopropoxybenzoic acid | 824.8 |
| 1112 | 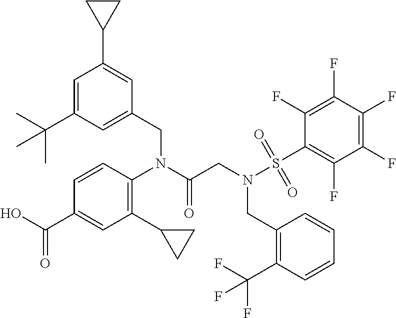 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-3- cyclopropylbenzoic acid | 808.8 |
| 1113 | 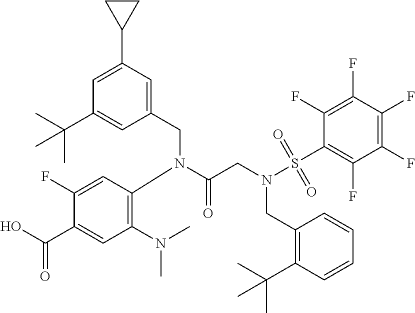 [SMILES: CN(C)c1cc(C(=O)O)c(F)cc1N(Cc1cc(C2CC2)cc(C(C)(C)C)c1)C(=O)CN(Cc1ccccc1C(C)(C)C)S(=O)(=O)c1c(F)c(F)c(F)c(F)c1F] | 4-(N-(3-(tert-butyl)-5- cyclopropylbenzyl)-2- (N-(2- (trifluoromethyl)benzyl)- (2,3,4,5,6-pentafluoro- phenyl)sulfonamido) acetamido)-5- (dimethylamino)-2- fluorobenzoic acid | 829.8 |
| 1114 | 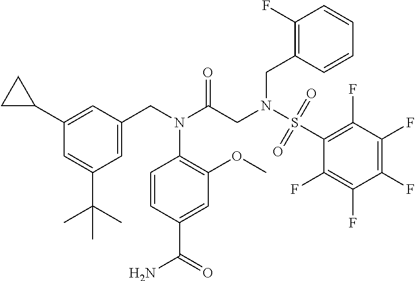 [SMILES: COc1cc(C(N)=O)ccc1N(Cc1cc(C2CC2)cc(C(C)(C)C)c1)C(=O)CN(Cc1ccccc1F)S(=O)(=O)c1c(F)c(F)c(F)c(F)c1F] | 4-(N-(3-(tert-butyl)-5- cyclopropylbenzyl)-2- (N-(2-fluorobenzyl)- (2,3,4,5,6-pentafluoro- phenyl)sulfonamido) acetamido)-3- methoxybenzamide | 747.8 |
| 1115 | 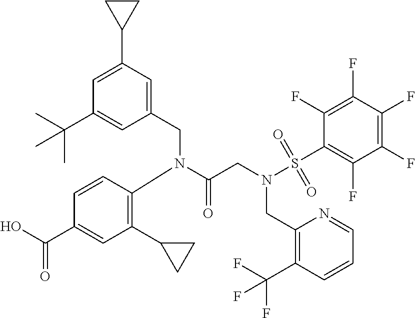 [SMILES: CC(C)(C)c1cc(CN(C(=O)CN(Cc2ncccc2C(F)(F)F)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-((3- (trifluoromethyl)pyridin- 2-yl)methyl)- (2,3,4,5,6-pentafluoro- phenyl)sulfonamido) acetamido)-3- cyclopropylbenzoic acid | 809.8 |
| 1116 | 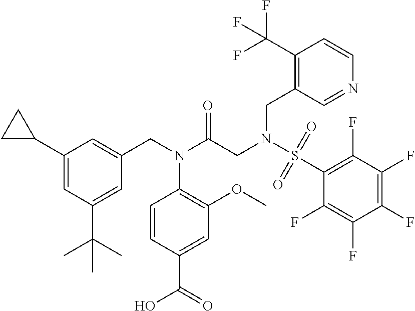 [SMILES: COc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoic acid | 799.7 |
| 1117 | 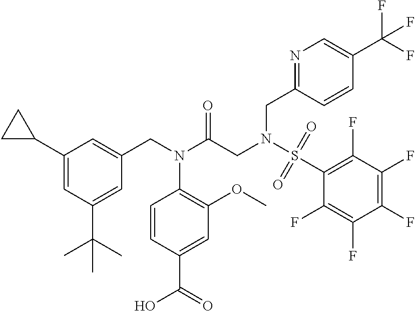 [SMILES: COc1cc(C(=O)O)ccc1N(Cc1cc(C2CC2)cc(C(C)(C)C)c1)C(=O)CN(Cc1ccc(C(F)(F)F)cn1)S(=O)(=O)c1c(F)c(F)c(F)c(F)c1F] | 4-(N-(3-(tert-butyl)-5- cyclopropylbenzyl)-2- (N-((5- (trifluoromethyl)pyridin- 2-yl)methyl)- (2,3,4,5,6-pentafluoro- phenyl)sulfonamido) acetamido)-3- methoxybenzoic acid | 799.7 |
| 1118 | 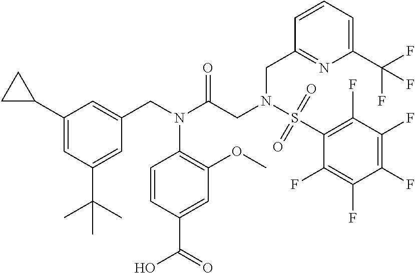 [SMILES: COc1cc(C(=O)O)ccc1N(Cc1cc(C2CC2)cc(C(C)(C)C)c1)C(=O)CN(Cc1cccc(C(F)(F)F)n1)S(=O)(=O)c1c(F)c(F)c(F)c(F)c1F] | 4-(N-(3-(tert-butyl)-5- cyclopropylbenzyl)-2- (N-((6- (trifluoromethyl)pyridin- 2-yl)methyl)- (2,3,4,5,6-pentafluoro- phenyl)sulfonamido) acetamido)-3- methoxybenzoic acid | 799.7 |
| 1119 | 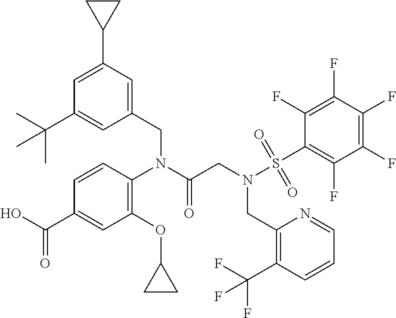 [SMILES: CC(C)(C)c1cc(CN(C(=O)CN(Cc2ncccc2C(F)(F)F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-((3- (trifluoromethyl)pyridin- 2-yl)methyl)- (2,3,4,5,6-pentafluoro- phenyl)sulfonamido) acetamido)-3- cyclopropoxybenzoic acid | 825.8 |
| 1120 | 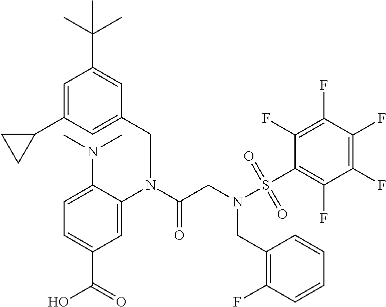 [SMILES: CN(C)c1ccc(C(=O)O)cc1N(Cc1cc(C2CC2)cc(C(C)(C)C)c1)C(=O)CN(Cc1ccccc1F)S(=O)(=O)c1c(F)c(F)c(F)c(F)c1F] | 3-(N-(3-(tert-butyl)-5- cyclopropylbenzyl)-2- (N-(2-fluorobenzyl)- (2,3,4,5,6-pentafluoro- phenyl)sulfonamido) acetamido)-4- (dimethylamino) benzoic acid | 761.8 |
| 1122 | 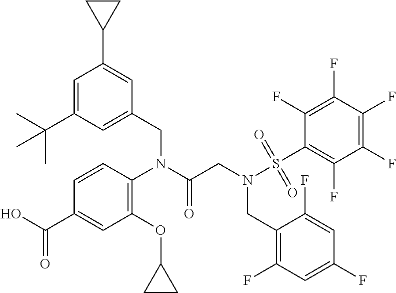 [SMILES: CC(C)(C)c1cc(CN(C(=O)CN(Cc2c(F)cc(F)cc2F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2,4,6- trifluorobenzyl)- (2,3,4,5,6-pentafluoro- phenyl)sulfonamido) acetamido)-3- cyclopropoxybenzoic acid | 810.8 |
| 1123 | 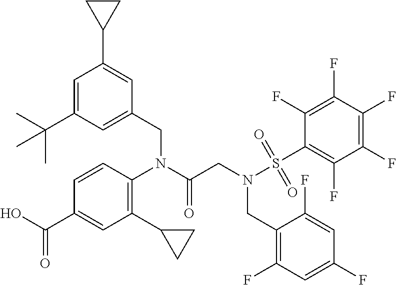 [SMILES: CC(C)(C)c1cc(CN(C(=O)CN(Cc2c(F)cc(F)cc2F)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-(2,4,6- trifluorobenzyl)- (2,3,4,5,6-pentafluoro- phenyl)sulfonamido) acetamido)-3- cyclopropylbenzoic acid | 794.8 |
| 1124 | 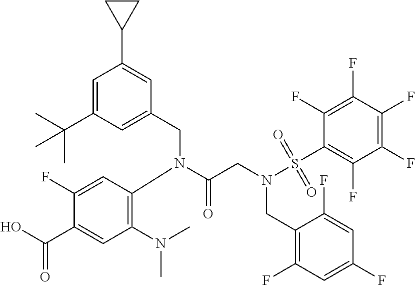 [SMILES: CN(C)c1cc(C(=O)O)c(F)cc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | 4-(N-(3-(tert-butyl)-5- cyclopropylbenzyl)-2- (N-(2,4,6- trifluorobenzyl)- (2,3,4,5,6-pentafluoro- phenyl)sulfonamido) acetamido)-5- (dimethylamino)-2- fluorobenzoic acid | 815.7 |
| 1125 | 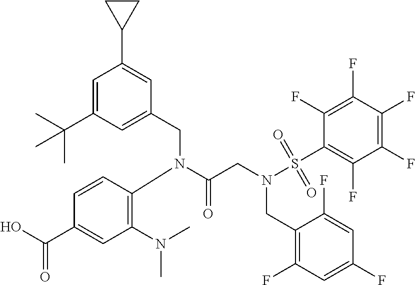 [SMILES: CN(C)c1cc(C(=O)O)ccc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | 4-(N-(3 -(tert-butyl)-5 - cyclopropylbenzyl)-2- (N-(2,4,6- trifluorobenzyl)- (2,3,4,5,6-pentafluoro- phenyl)sulfonamido) acetamido)-3- (dimethylamino) benzoic acid | 797.8 |
| 1126 | 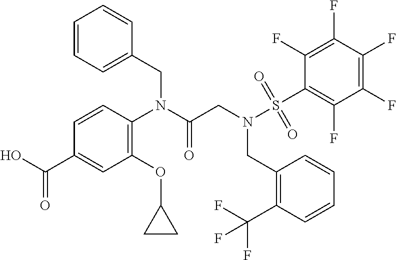 [SMILES: O=C(O)c1ccc(N(Cc2ccccc2)C(=O)CN(Cc2ccccc2C(F)(F)F)S(=O)(=O)c2c(F)c(F)c(F)c(F)c2F)c(OC2CC2)c1] | 4-(N-benzyl-2-(N-(2- (trifluoromethyl)benzyl)- (2,3,4,5,6-pentafluoro- phenyl)sulfonamido) acetamido)-3- cyclopropoxybenzoic acid | 728.6 |
| 1127 | 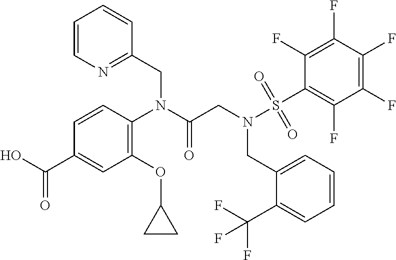 [SMILES: O=C(O)c1ccc(N(Cc2ccccn2)C(=O)CN(Cc2ccccc2C(F)(F)F)S(=O)(=O)c2c(F)c(F)c(F)c(F)c2F)c(OC2CC2)c1] | 3-cyclopropoxy-4-(2- (N-(2- (trifluoromethyl)benzyl)- (2,3,4,5,6-pentafluoro- phenyl)sulfonamido)-N- (pyridin-2- ylmethyl)acetamido) benzoic acid | 729.6 |
| 1128 | 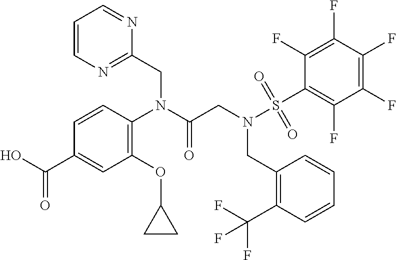 [SMILES: O=C(O)c1ccc(N(Cc2ncccn2)C(=O)CN(Cc2ccccc2C(F)(F)F)S(=O)(=O)c2c(F)c(F)c(F)c(F)c2F)c(OC2CC2)c1] | 3-cyclopropoxy-4-(2- (N-(2- (trifluoromethyl)benzyl)- (2,3,4,5,6-pentafluoro- phenyl)sulfonamido)-N- (pyrimidin-2- ylmethyl)acetamido) benzoic acid | 730.6 |
| 1129 | 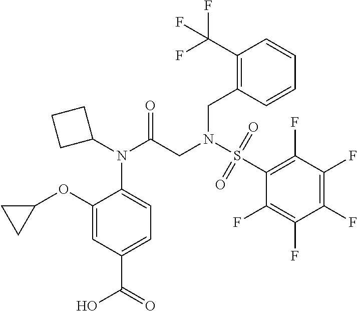 [SMILES: O=C(O)c1ccc(N(C(=O)CN(Cc2ccccc2C(F)(F)F)S(=O)(=O)c2c(F)c(F)c(F)c(F)c2F)C2CCC2)c(OC2CC2)c1] | 4-(N-cyclobutyl-2-(N- (2- (trifluoromethyl)benzyl)- (2,3,4,5,6-pentafluoro- phenyl)sulfonamido) acetamido)-3- cyclopropoxybenzoic acid | 692.6 |
| 1130 | 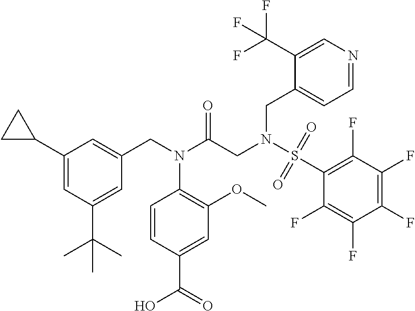 [SMILES: COc1cc(C(=O)O)ccc1N(Cc1cc(C2CC2)cc(C(C)(C)C)c1)C(=O)CN(Cc1ccncc1C(F)(F)F)S(=O)(=O)c1c(F)c(F)c(F)c(F)c1F] | 4-(N-(3-(tert-butyl)-5- cyclopropylbenzyl)-2- (N-((3- (trifluoromethyl)pyridin- 4-yl)methyl)- (2,3,4,5,6-pentafluoro- phenyl)sulfonamido) acetamido)-3- methoxybenzoic acid | 799.7 |
| 1131 | 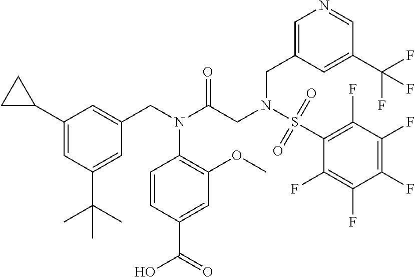 [SMILES: COc1cc(C(=O)O)ccc1N(Cc1cc(C2CC2)cc(C(C)(C)C)c1)C(=O)CN(Cc1cncc(C(F)(F)F)c1)S(=O)(=O)c1c(F)c(F)c(F)c(F)c1F] | 4-(N-(3-(tert-butyl)-5- cyclopropylbenzyl)-2- (N-((5- (trifluoromethyl)pyridin- 3-yl)methyl)- (2,3,4,5,6-pentafluoro- phenyl)sulfonamido) acetamido)-3- methoxybenzoic acid | 799.7 |
| 1132 | 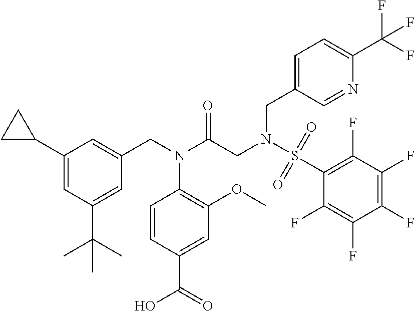 [SMILES: COc1cc(C(=O)O)ccc1N(Cc1cc(C2CC2)cc(C(C)(C)C)c1)C(=O)CN(Cc1ccc(C(F)(F)F)nc1)S(=O)(=O)c1c(F)c(F)c(F)c(F)c1F] | 4-(N-(3-(tert-butyl)-5- cyclopropylbenzyl)-2- (N-((6- (trifluoromethyl)pyridin- 3-yl)methyl)- (2,3,4,5,6-pentafluoro- phenyl)sulfonamido) acetamido)-3- methoxybenzoic acid | 799.7 |
| 1133 | 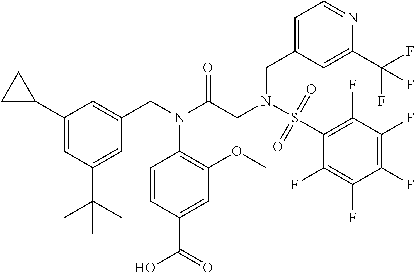 [SMILES: COc1cc(C(=O)O)ccc1N(Cc1cc(C2CC2)cc(C(C)(C)C)c1)C(=O)CN(Cc1ccnc(C(F)(F)F)c1)S(=O)(=O)c1c(F)c(F)c(F)c(F)c1F] | 4-(N-(3-(tert-butyl)-5- cyclopropylbenzyl)-2- (N-((2- (trifluoromethyl)pyridin- 4-yl)methyl)- (2,3,4,5,6-pentafluoro- phenyl)sulfonamido) acetamido)-3- methoxybenzoic acid | 799.7 |
| 1134 | 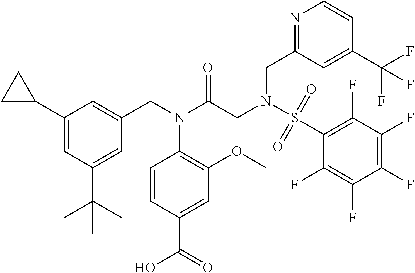 [SMILES: COc1cc(C(=O)O)ccc1N(Cc1cc(C2CC2)cc(C(C)(C)C)c1)C(=O)CN(Cc1cc(C(F)(F)F)ccn1)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 2-yl)methyl)- (2,3,4,5,6-pentafluoro- phenyl)sulfonamido) acetamido)-3- methoxybenzoic acid | 799.7 |
| 1135 | 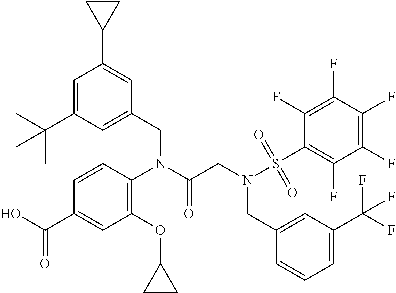 [SMILES: CC(C)(C)c1cc(CN(C(=O)CN(Cc2cccc(C(F)(F)F)c2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(3- (trifluoromethyl)benzyl)- (2,3,4,5,6-pentafluoro- phenyl)sulfonamido) acetamido)-3- cyclopropoxybenzoic acid | 824.8 |
| 1136 | 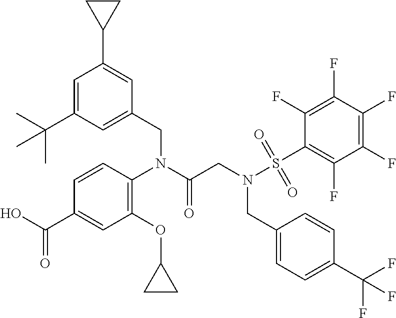 [SMILES: CC(C)(C)c1cc(CN(C(=O)CN(Cc2ccc(C(F)(F)F)cc2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(4- (trifluoromethyl)benzyl)- (2,3,4,5,6-pentafluoro- phenyl)sulfonamido) acetamido)-3- cyclopropoxybenzoic acid | 824.8 |
| 1137 | 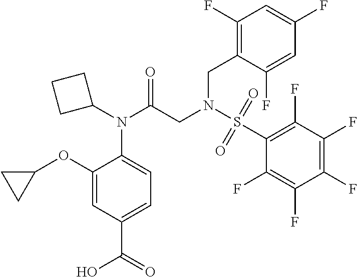 [SMILES: O=C(O)c1ccc(N(C(=O)CN(Cc2c(F)cc(F)cc2F)S(=O)(=O)c2c(F)c(F)c(F)c(F)c2F)C2CCC2)c(OC2CC2)c1] | 4-(N-cyclobutyl-2-(N- (2,4,6-trifluorobenzyl)- (2,3,4,5,6-pentafluoro- phenyl)sulfonamido) acetamido)-3- cyclopropoxybenzoic acid | 678.6 |
| 1138 | 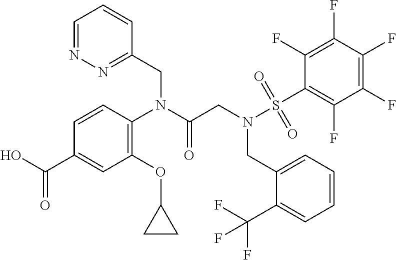 [SMILES: O=C(O)c1ccc(N(Cc2cccnn2)C(=O)CN(Cc2ccccc2C(F)(F)F)S(=O)(=O)c2c(F)c(F)c(F)c(F)c2F)c(OC2CC2)c1] | 3-cyclopropoxy-4-(2- (N-(2- (trifluoromethyl)benzyl)- (2,3,4,5,6-pentafluoro- phenyl)sulfonamido)-N- (pyridazin-3- ylmethyl)acetamido) benzoic acid | 730.6 |
| 1139 | 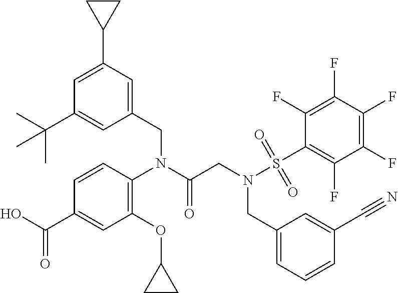 [SMILES: CC(C)(C)c1cc(CN(C(=O)CN(Cc2cccc(C#N)c2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(3-cyanobenzyl)- (2,3,4,5,6- pentafluorophenyl)sulfon- amido)acetamido)-3- cyclopropoxybenzoic acid | 781.8 |
| 1140 | 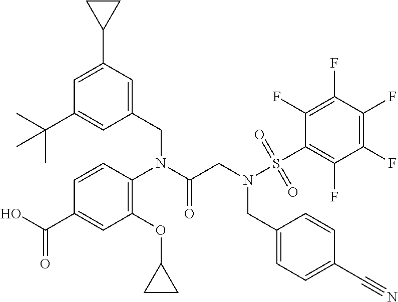 [SMILES: CC(C)(C)c1cc(CN(C(=O)CN(Cc2ccc(C#N)cc2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(4-cyanobenzyl)- (2,3,4,5,6- pentafluorophenyl)sulfon- amido)acetamido)-3- cyclopropoxybenzoic acid | 781.8 |
| 1141 | 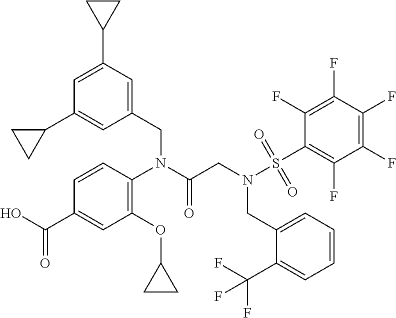 [SMILES: O=C(O)c1ccc(N(Cc2cc(C3CC3)cc(C3CC3)c2)C(=O)CN(Cc2ccccc2C(F)(F)F)S(=O)(=O)c2c(F)c(F)c(F)c(F)c2F)c(OC2CC2)c1] | 3-cyclopropoxy-4-(N- (3,5- dicyclopropylbenzyl)-2- (N-(2- (trifluoromethyl)benzyl)- (2,3,4,5,6-pentafluoro- phenyl)sulfonamido) acetamido)benzoic acid | 808.7 |
| 1142 | 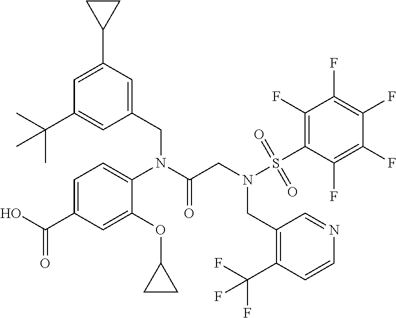 [SMILES: CC(C)(C)c1cc(CN(C(=O)CN(Cc2cnccc2C(F)(F)F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- cyclopropoxybenzoic acid | 825.8 |
| 1143 | 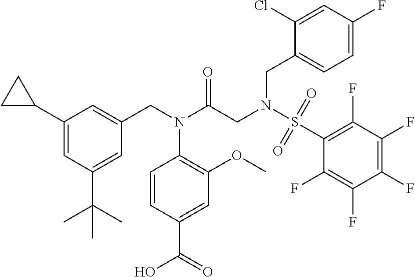 [SMILES: COc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- methoxybenzoic acid | 783.2 |
| 1144 | 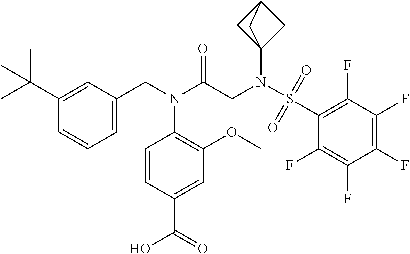 [SMILES: COc1cc(C(=O)O)ccc1N(Cc1cccc(C(C)(C)C)c1)C(=O)CN(C12CC(C1)C2)S(=O)(=O)c1c(F)c(F)c(F)c(F)c1F] | 4-(2-(N- (bicyclo[1.1.1]pentan-1- yl)-(2,3,4,5,6- pentafluorophenyl)sulfon- amido)-N-(3-(tert- butyl)benzyl)acetamido)- 3-methoxybenzoic acid | 666.7 |
| 1145 | 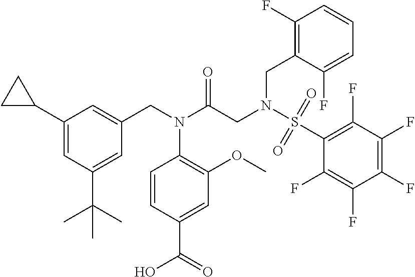 [SMILES: COc1cc(C(=O)O)ccc1N(Cc1cc(C2CC2)cc(C(C)(C)C)c1)C(=O)CN(Cc1c(F)cccc1F)S(=O)(=O)c1c(F)c(F)c(F)c(F)c1F] | 4-(N-(3-(tert-butyl)-5- cyclopropylbenzyl)-2- (N-(2,6- difluorobenzyl)- (2,3,4,5,6- pentafluorophenyl)sulfon- amido)acetamido)-3- methoxybenzoic acid | 766.7 |
| 1146 | 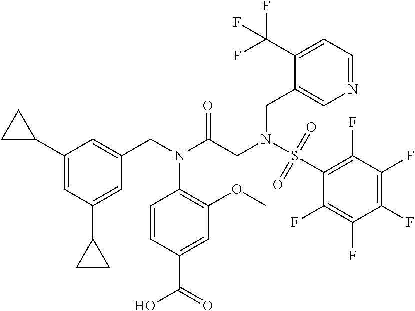 [SMILES: COc1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | 4-(N-(3,5- dicyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoic acid | 783.7 |
| 1147 | 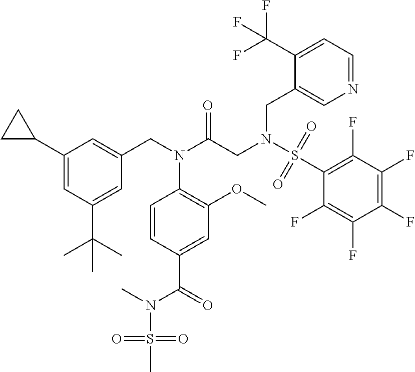 [SMILES: COc1cc(C(=O)N(C)S(C)(=O)=O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3-methoxy-N- methyl-N- (methylsulfonyl) benzamide | 890.9 |
| 1148 | 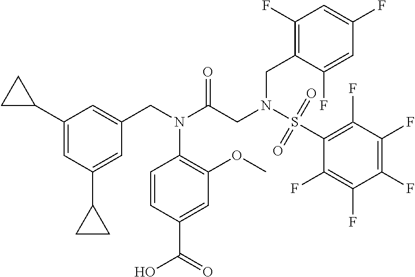 [SMILES: COc1cc(C(=O)O)ccc1N(Cc1cc(C2CC2)cc(C2CC2)c1)C(=O)CN(Cc1c(F)cc(F)cc1F)S(=O)(=O)c1c(F)c(F)c(F)c(F)c1F] | 4-(N-(3,5- dicyclopropylbenzyl)-2- (N-(2,4,6- trifluorobenzyl)- (2,3,4,5,6-pentafluoro- phenyl)sulfonamido) acetamido)-3- methoxybenzoic acid | 768.7 |
| 1149 | 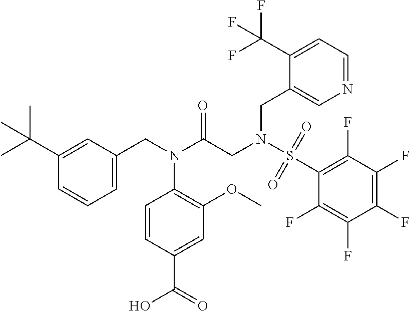 [SMILES: COc1cc(C(=O)O)ccc1N(Cc1cccc(C(C)(C)C)c1)C(=O)CN(Cc1cnccc1C(F)(F)F)S(=O)(=O)c1c(F)c(F)c(F)c(F)c1F] | 4-(N-(3-(tert- butyl)benzyl)-2-(N-((4- (trifluoromethyl)pyridin- 3-yl)methyl)- (2,3,4,5,6-pentafluoro- phenyl)sulfonamido) acetamido)-3- methoxybenzoic acid | 759.7 |
| 1150 | 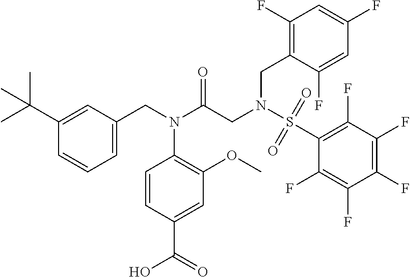 [SMILES: COc1cc(C(=O)O)ccc1N(Cc1cccc(C(C)(C)C)c1)C(=O)CN(Cc1c(F)cc(F)cc1F)S(=O)(=O)c1c(F)c(F)c(F)c(F)c1F] | 4-(N-(3-(tert- butyl)benzyl)-2-(N- (2,4,6-trifluorobenzyl)- (2,3,4,5,6-pentafluoro- phenyl)sulfonamido) acetamido)-3- methoxybenzoic acid | 744.7 |
| 1151 | 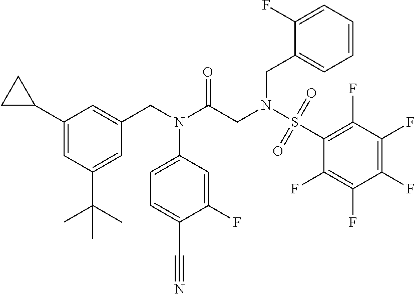 [SMILES: CC(C)(C)c1cc(CN(C(=O)CN(Cc2ccccc2F)S(=O)(=O)c2c(F)c(F)c(F)c(F)c2F)c2ccc(C#N)c(F)c2)cc(C2CC2)c1] | N-(3-(tert-butyl)-5- cyclopropylbenzyl)-N- (4-cyano-3- fluorophenyl)-2-(N-(2- fluorobenzyl)- (2,3,4,5,6-pentafluoro- phenyl)sulfonamido) acetamide | 717.7 |
| 1152 | 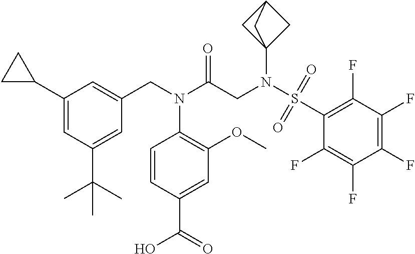 [SMILES: COc1cc(C(=O)O)ccc1N(Cc1cc(C2CC2)cc(C(C)(C)C)c1)C(=O)CN(C12CC(C1)C2)S(=O)(=O)c1c(F)c(F)c(F)c(F)c1F] | 4-(2-(N- (bicyclo[1.1.1]pentan-1- yl)-(2,3,4,5,6- pentafluorophenyl)sulfon- amido)-N-(3-(tert- butyl)-5- cyclopropylbenzyl) acetamido)-3- methoxybenzoic acid | 706.7 |
| 1153 | 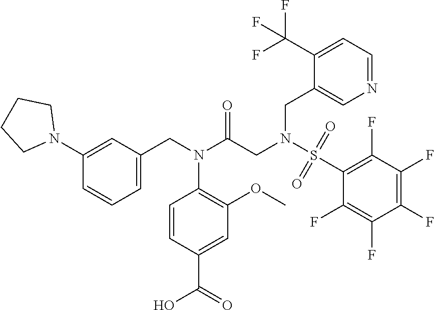 [SMILES: COc1cc(C(=O)O)ccc1N(Cc1cccc(N2CCCC2)c1)C(=O)CN(Cc1cnccc1C(F)(F)F)S(=O)(=O)c1c(F)c(F)c(F)c(F)c1F] | 3-methoxy-4-(2-(N-((4- (trifluoromethyl)pyridin- 3-yl)methyl)- (2,3,4,5,6-pentafluoro- phenyl)sulfonamido)-N- (3-(pyrrolidin-1- yl)benzyl)acetamido) benzoic acid | 772.7 |
| 1154 | 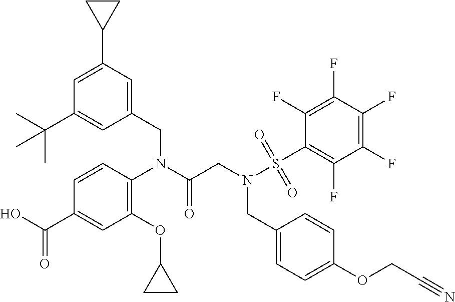 [SMILES: CC(C)(C)c1cc(CN(C(=O)CN(Cc2ccc(OCC#N)cc2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(4- (cyanomethoxy)benzyl)- (2,3,4,5,6- pentafluorophenyl)sulfon- amido)acetamido)-3- cyclopropoxybenzoic acid | 811.8 |
| 1155 | 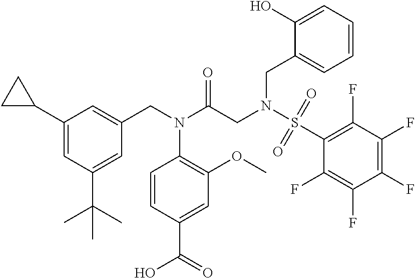 [SMILES: COc1cc(C(=O)O)ccc1N(Cc1cc(C2CC2)cc(C(C)(C)C)c1)C(=O)CN(Cc1ccccc1O)S(=O)(=O)c1c(F)c(F)c(F)c(F)c1F] | 4-(N-(3-(tert-butyl)-5- cyclopropylbenzyl)-2- (N-(2-hydroxybenzyl)- (2,3,4,5,6-pentafluoro- phenyl)sulfonamido) acetamido)-3- methoxybenzoic acid | 746.7 |
| 1156 | 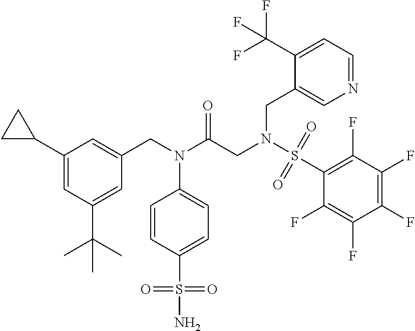 [SMILES: CC(C)(C)c1cc(CN(C(=O)CN(Cc2cnccc2C(F)(F)F)S(=O)(=O)c2c(F)c(F)c(F)c(F)c2F)c2ccc(S(N)(=O)=O)cc2)cc(C2CC2)c1] | N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido)-N- (4- sulfamoylphenyl) acetamide | 804.8 |
| 1157 | 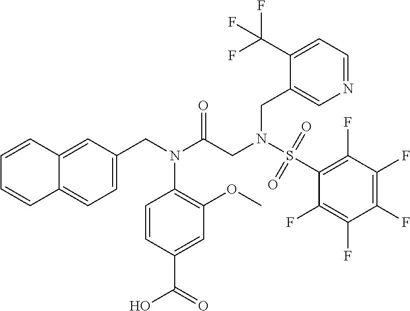 [SMILES: COc1cc(C(=O)O)ccc1N(Cc1ccc2ccccc2c1)C(=O)CN(Cc1cnccc1C(F)(F)F)S(=O)(=O)c1c(F)c(F)c(F)c(F)c1F] | 3-methoxy-4-(N- (naphthalen-2- ylmethyl)-2-(N-((4- (trifluoromethyl)pyridin- 3-yl)methyl)- (2,3,4,5,6-pentafluoro- phenyl)sulfonamido) acetamido)benzoic acid | 753.6 |
| 1158 | 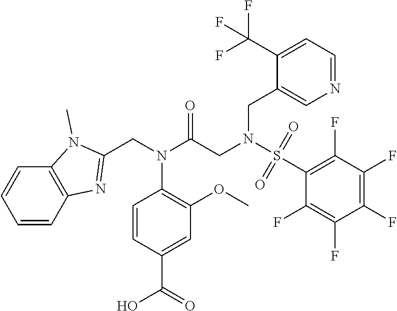 [SMILES: COc1cc(C(=O)O)ccc1N(Cc1nc2ccccc2n1C)C(=O)CN(Cc1cnccc1C(F)(F)F)S(=O)(=O)c1c(F)c(F)c(F)c(F)c1F] | 3-methoxy-4-(N-((1- methyl-1H- benzo[d]imidazol-2- yl)methyl)-2-(N-((4- (trifluoromethyl)pyridin- 3-yl)methyl)- (2,3,4,5,6-pentafluoro- phenyl)sulfonamido) acetamido)benzoic acid | 757.6 |
| 1159 | 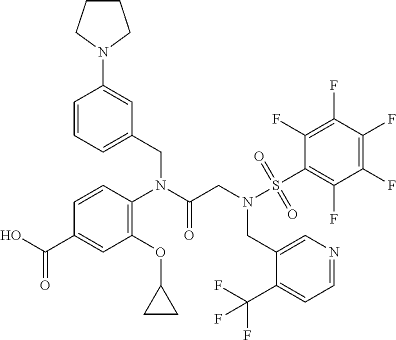 [SMILES: O=C(O)c1ccc(N(Cc2cccc(N3CCCC3)c2)C(=O)CN(Cc2cnccc2C(F)(F)F)S(=O)(=O)c2c(F)c(F)c(F)c(F)c2F)c(OC2CC2)c1] | 3-cyclopropoxy-4-(2- (N-((4- (trifluoromethyl)pyridin- 3-yl)methyl)- (2,3,4,5,6-pentafluoro- phenyl)sulfonamido)-N- (3-(pyrrolidin-1- yl)benzyl)acetamido) benzoic acid | 798.7 |
| 1160 | 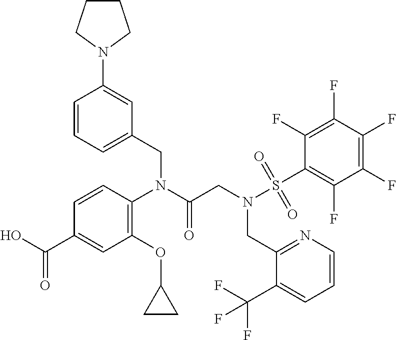 [SMILES: O=C(O)c1ccc(N(Cc2cccc(N3CCCC3)c2)C(=O)CN(Cc2ncccc2C(F)(F)F)S(=O)(=O)c2c(F)c(F)c(F)c(F)c2F)c(OC2CC2)c1] | 3-cyclopropoxy-4-(2- (N-((3- (trifluoromethyl)pyridin- 2-yl)methyl)- (2,3,4,5,6-pentafluoro- phenyl)sulfonamido)-N- (3-(pyrrolidin-1- yl)benzyl)acetamido) benzoic acid | 798.7 |
| 1161 | 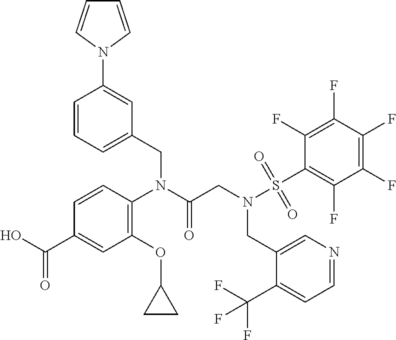 [SMILES: O=C(O)c1ccc(N(Cc2cccc(-n3cccc3)c2)C(=O)CN(Cc2cnccc2C(F)(F)F)S(=O)(=O)c2c(F)c(F)c(F)c(F)c2F)c(OC2CC2)c1] | 4-(N-(3-(1H-pyrrol-1- yl)benzyl)-2-(N-((4- (trifluoromethyl)pyridin- 3-yl)methyl)- (2,3,4,5,6-pentafluoro- phenyl)sulfonamido) acetamido)-3- cyclopropoxybenzoic acid | 794.7 |
| 1162 | 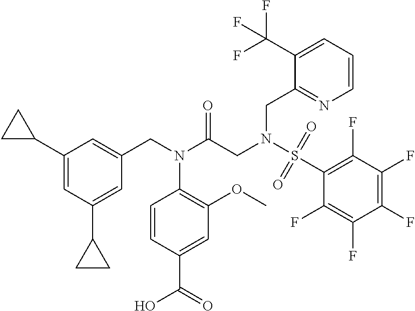 [SMILES: COc1cc(C(=O)O)ccc1N(Cc1cc(C2CC2)cc(C2CC2)c1)C(=O)CN(Cc1ncccc1C(F)(F)F)S(=O)(=O)c1c(F)c(F)c(F)c(F)c1F] | 4-(N-(3,5- dicyclopropylbenzyl)-2- (N-((3- (trifluoromethyl)pyridin- 2-yl)methyl)- (2,3,4,5,6-pentafluoro- phenyl)sulfonamido) acetamido)-3- methoxybenzoic acid | 783.7 |
| 1163 | 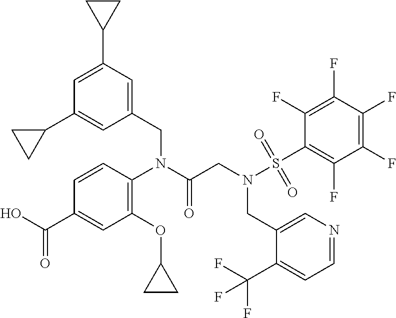 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(OC2CC2)c1] | 3-cyclopropoxy-4-(N- (3,5- dicyclopropylbenzyl)-2- (N-((4- (trifluoromethyl)pyridin- 3-yl)methyl)- (2,3,4,5,6-pentafluoro- phenyl)sulfonamido) acetamido)benzoic acid | 809.7 |
| 1164 | 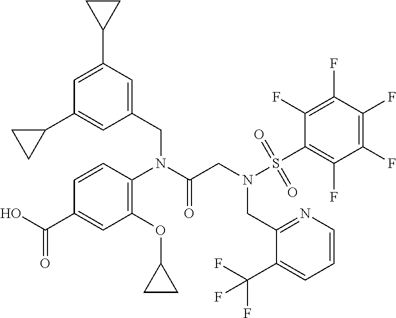 [SMILES: O=C(O)c1ccc(N(Cc2cc(C3CC3)cc(C3CC3)c2)C(=O)CN(Cc2ncccc2C(F)(F)F)S(=O)(=O)c2c(F)c(F)c(F)c(F)c2F)c(OC2CC2)c1] | 3-cyclopropoxy-4-(N- (3,5- dicyclopropylbenzyl)-2- (N-((3- (trifluoromethyl)pyridin- 2-yl)methyl)- (2,3,4,5,6-pentafluoro- phenyl)sulfonamido) acetamido)benzoic acid | 809.7 |
| 1165 | 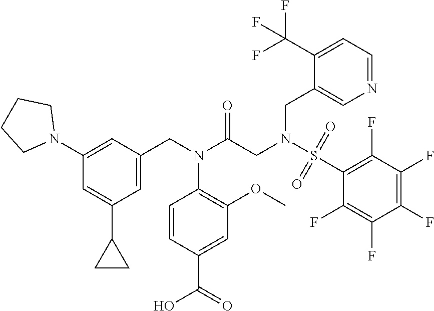 [SMILES: COc1cc(C(=O)O)ccc1N(Cc1cc(C2CC2)cc(N2CCCC2)c1)C(=O)CN(Cc1cnccc1C(F)(F)F)S(=O)(=O)c1c(F)c(F)c(F)c(F)c1F] | 4-(N-(3-cyclopropyl-5- (pyrrolidin-1- yl)benzyl)-2-(N-((4- (trifluoromethyl)pyridin- 3-yl)methyl)- (2,3,4,5,6-pentafluoro- phenyl)sulfonamido) acetamido)-3- methoxybenzoic acid | 812.7 |
| 1166 | 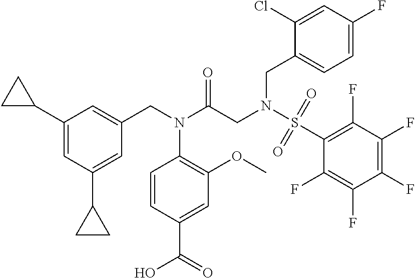 [SMILES: COc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- methoxybenzoic acid | 767.1 |
| 1167 | 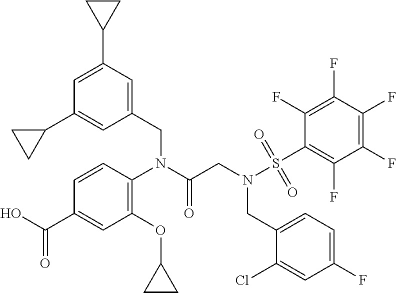 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- cyclopropoxybenzoic acid | 793.2 |
| 1168 | 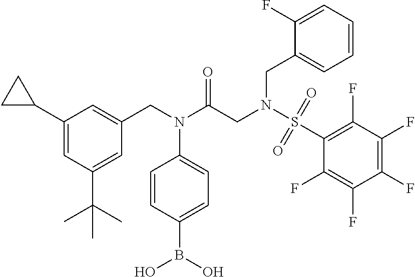 [SMILES: CC(C)(C)c1cc(CN(C(=O)CN(Cc2ccccc2F)S(=O)(=O)c2c(F)c(F)c(F)c(F)c2F)c2ccc(B(O)O)cc2)cc(C2CC2)c1] | (4-(N-(3-(tert-butyl)-5- cyclopropylbenzyl)-2- (N-(2-fluorobenzyl)- (2,3,4,5,6-pentafluoro- phenyl)sulfonamido) acetamido)phenyl)boronic acid | 718.5 |
| 1169 | 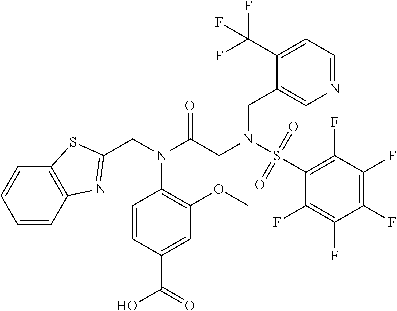 [SMILES: COc1cc(C(=O)O)ccc1N(Cc1nc2ccccc2s1)C(=O)CN(Cc1cnccc1C(F)(F)F)S(=O)(=O)c1c(F)c(F)c(F)c(F)c1F] | 4-(N-(benzo[d]thiazol- 2-ylmethyl)-2-(N-((4- (trifluoromethyl)pyridin- 3-yl)methyl)- (2,3,4,5,6-pentafluoro- phenyl)sulfonamido) acetamido)-3- methoxybenzoic acid | 760.6 |
| 1170 | 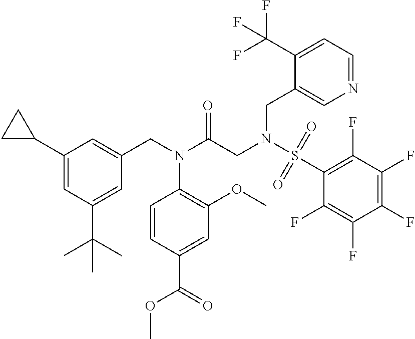 [SMILES: COC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | methyl 4-(N-(3-(tert- butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoate | 813.8 |
| 1171 | 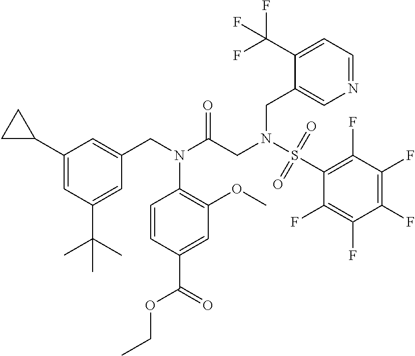 [SMILES: CCOC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | ethyl 4-(N-(3-(tert- butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoate | 827.8 |
| 1172 | 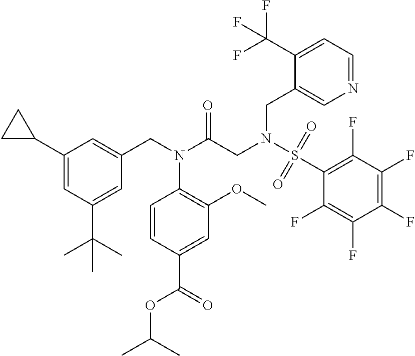 [SMILES: COc1cc(C(=O)OC(C)C)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | isopropyl 4-(N-(3-(tert- butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoate | 841.8 |
| 1173 | 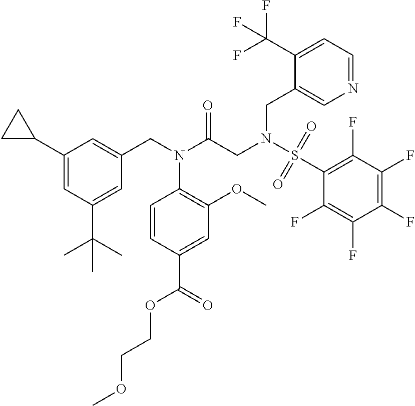 [SMILES: COCCOC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | 2-methoxyethyl 4-(N- (3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoate | 857.8 |
| 1174 | 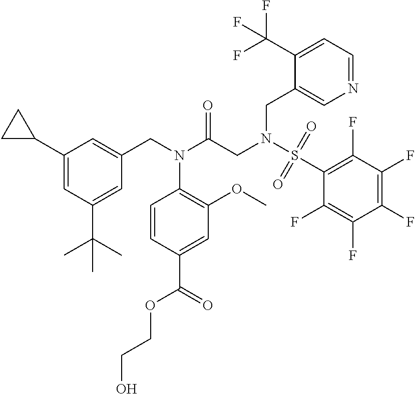 [SMILES: COc1cc(C(=O)OCCO)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 2-hydroxyethyl 4-(N- (3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methoxybenzoate | 843.8 |
| 1175 | 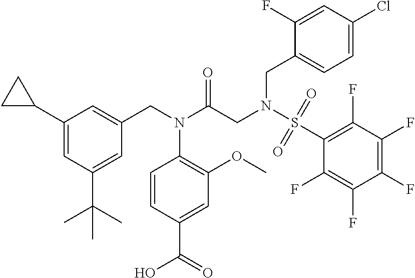 [SMILES: COc1cc(C(=O)O)ccc1N(Cc1cc(C2CC2)cc(C(C)(C)C)c1)C(=O)CN(Cc1ccc(Cl)cc1F)S(=O)(=O)c1c(F)c(F)c(F)c(F)c1F] | 4-(N-(3-(tert-butyl)-5- cyclopropylbenzyl)-2- (N-(4-chloro-2- fluorobenzyl)- (2,3,4,5,6- pentafluorophenyl)sulfon- amido)acetamido)-3- methoxybenzoic acid | 783.2 |
| 1176 | 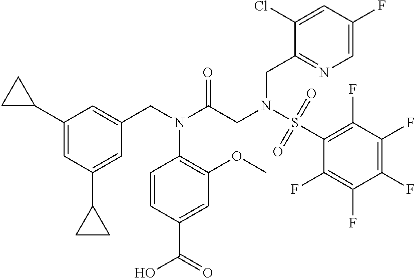 [SMILES: COc1cc(C(=O)O)ccc1N(Cc1cc(C2CC2)cc(C2CC2)c1)C(=O)CN(Cc1ncc(F)cc1Cl)S(=O)(=O)c1c(F)c(F)c(F)c(F)c1F] | 4-(2-(N-((3-chloro-5- fluoropyridin-2- yl)methyl)-(2,3,4,5,6- pentafluorophenyl)sulfon- amido)-N-(3,5- dicyclopropylbenzyl) acetamido)-3- methoxybenzoic acid | 768.1 |
| 1177 | 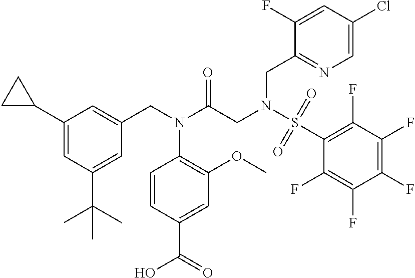 [SMILES: COc1cc(C(=O)O)ccc1N(Cc1cc(C2CC2)cc(C(C)(C)C)c1)C(=O)CN(Cc1ncc(Cl)cc1F)S(=O)(=O)c1c(F)c(F)c(F)c(F)c1F] | 4-(N-(3-(tert-butyl)-5- cyclopropylbenzyl)-2- (N-((5-chloro-3- fluoropyridin-2- yl)methyl)-(2,3,4,5,6- pentafluorophenyl)sulfon- amido)acetamido)-3- methoxybenzoic acid | 784.2 |
| 1178 | 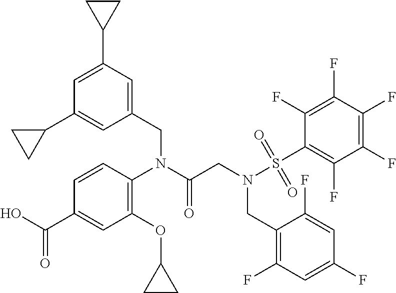 [SMILES: O=C(O)c1ccc(N(Cc2cc(C3CC3)cc(C3CC3)c2)C(=O)CN(Cc2c(F)cc(F)cc2F)S(=O)(=O)c2c(F)c(F)c(F)c(F)c2F)c(OC2CC2)c1] | 3-cyclopropoxy-4-(N- (3,5- dicyclopropylbenzyl)-2- (N-(2,4,6- trifluorobenzyl)- (2,3,4,5,6-pentafluoro- phenyl)sulfonamido) acetamido)benzoic acid | 794.7 |
| 1179 | 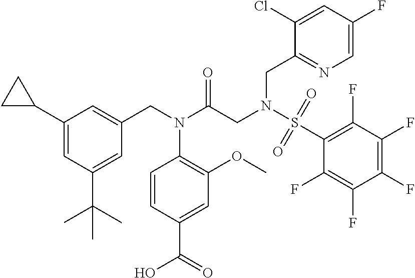 [SMILES: COc1cc(C(=O)O)ccc1N(Cc1cc(C2CC2)cc(C(C)(C)C)c1)C(=O)CN(Cc1ncc(F)cc1Cl)S(=O)(=O)c1c(F)c(F)c(F)c(F)c1F] | 4-(N-(3-(tert-butyl)-5- cyclopropylbenzyl)-2- (N-((3-chloro-5- fluoropyridin-2- yl)methyl)-(2,3,4,5,6- pentafluorophenyl)sulfon- amido)acetamido)-3- methoxybenzoic acid | 784.2 |
| 1180 | 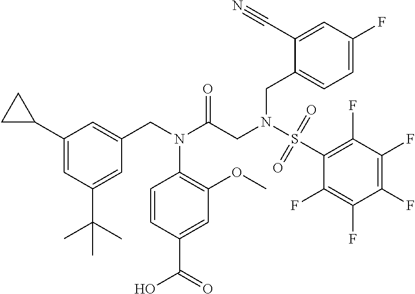 [SMILES: COc1cc(C(=O)O)ccc1N(Cc1cc(C2CC2)cc(C(C)(C)C)c1)C(=O)CN(Cc1ccc(F)cc1C#N)S(=O)(=O)c1c(F)c(F)c(F)c(F)c1F] | 4-(N-(3-(tert-butyl)-5- cyclopropylbenzyl)-2- (N-(2-cyano-4- fluorobenzyl)- (2,3,4,5,6- pentafluorophenyl)sulfon- amido)acetamido)-3- methoxybenzoic acid | 773.7 |
| 1181 | 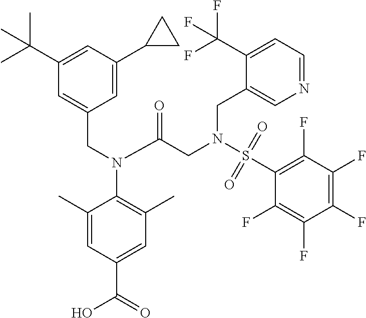 [SMILES: Cc1cc(C(=O)O)cc(C)c1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3,5- dimethylbenzoic acid | 797.8 |
| 1182 | 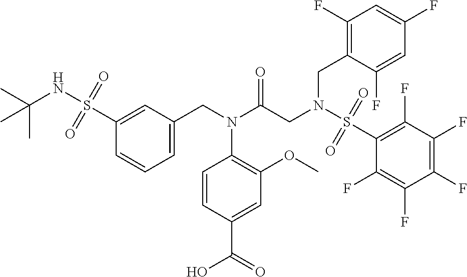 [SMILES: COc1cc(C(=O)O)ccc1N(Cc1cccc(S(=O)(=O)NC(C)(C)C)c1)C(=O)CN(Cc1c(F)cc(F)cc1F)S(=O)(=O)c1c(F)c(F)c(F)c(F)c1F] | 4-(N-(3-(N-(tert- butyl)sulfamoyl)benzyl)- 2-(N-(2,4,6- trifluorobenzyl)- (2,3,4,5,6-pentafluoro- phenyl)sulfonamido) acetamido)-3- methoxybenzoic acid | 823.7 |
| 1183 | 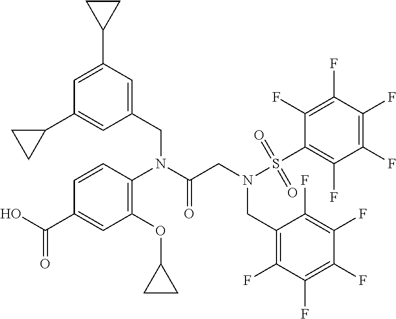 [SMILES: O=C(O)c1ccc(N(Cc2cc(C3CC3)cc(C3CC3)c2)C(=O)CN(Cc2c(F)c(F)c(F)c(F)c2F)S(=O)(=O)c2c(F)c(F)c(F)c(F)c2F)c(OC2CC2)c1] | 3-cyclopropoxy-4-(N- (3,5- dicyclopropylbenzyl)-2- (N- ((perfluorophenyl) methyl)-(2,3,4,5,6- pentafluoro- phenyl)sulfonamido) acetamido)benzoic acid | 830.7 |
| 1186 | 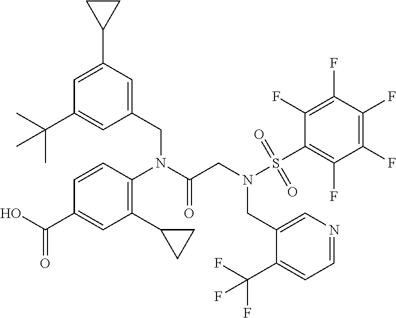 [SMILES: CC(C)(C)c1cc(CN(C(=O)CN(Cc2cnccc2C(F)(F)F)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- cyclopropylbenzoic acid | 809.8 |
| 1187 | 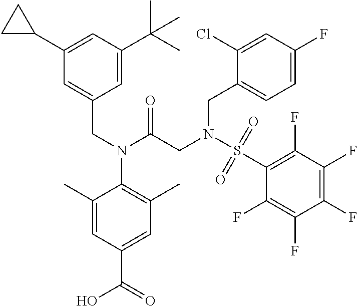 [SMILES: Cc1cc(C(=O)O)cc(C)c1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3,5- dimethylbenzoic acid | 781.2 |
| 1188 | 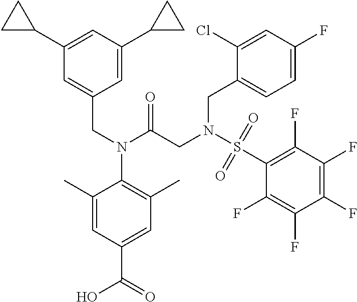 [SMILES: Cc1cc(C(=O)O)cc(C)c1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3,5- dimethylbenzoic acid | 765.2 |
| 1189 | 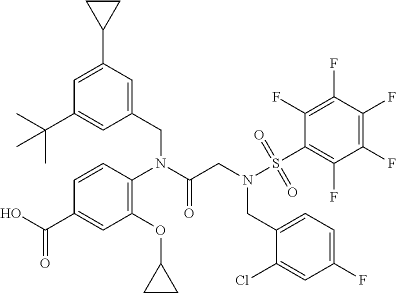 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- cyclopropoxybenzoic acid | 809.2 |
| 1190 | 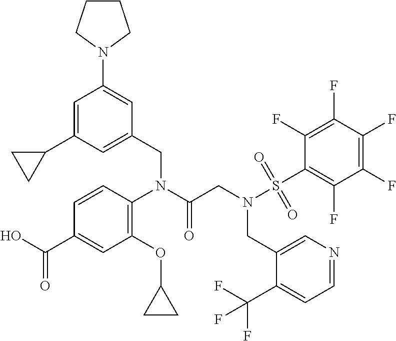 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(OC2CC2)c1] | 3-cyclopropoxy-4-(N- (3-cyclopropyl-5- (pyrrolidin-1- yl)benzyl)-2-(N-((4- (trifluoromethyl)pyridin- 3-yl)methyl)- (2,3,4,5,6-pentafluoro- phenyl)sulfonamido) acetamido)benzoic acid | 838.8 |
| 1191 | 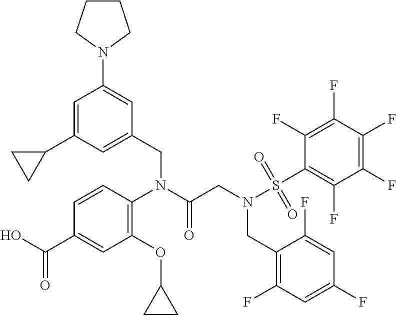 [SMILES: O=C(O)c1ccc(N(Cc2cc(C3CC3)cc(N3CCCC3)c2)C(=O)CN(Cc2c(F)cc(F)cc2F)S(=O)(=O)c2c(F)c(F)c(F)c(F)c2F)c(OC2CC2)c1] | 3-cyclopropoxy-4-(N- (3-cyclopropyl-5- (pyrrolidin-1- yl)benzyl)-2-(N-(2,4,6- trifluorobenzyl)- (2,3,4,5,6-pentafluoro- phenyl)sulfonamido) acetamido)benzoic acid | 823.8 |
| 1192 | 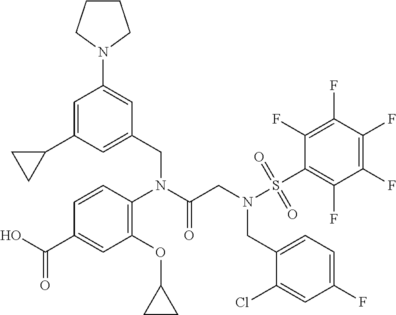 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(OC2CC2)c1] | 4-(2-(N-(2-chloro-4- fluorobenzyl)- (2,3,4,5,6- pentafluorophenyl)sulfon- amido)-N-(3- cyclopropyl-5- (pyrrolidin-1- yl)benzyl)acetamido)-3- cyclopropoxybenzoic acid | 822.2 |
| 1193 | 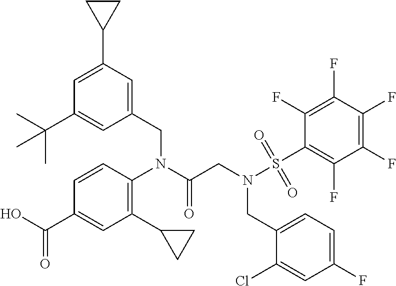 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2C2CC2)cc(C2CC2)c1] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- cyclopropylbenzoic acid | 793.2 |
| 1194 | 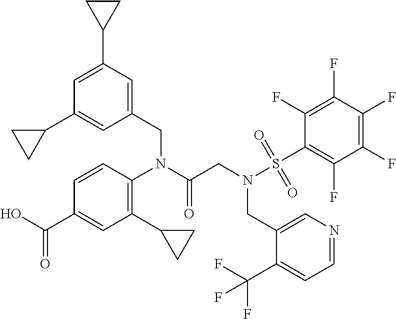 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(C2CC2)c1] | 3-cyclopropyl-4-(N- (3,5- dicyclopropylbenzyl)-2- (N-((4- (trifluoromethyl)pyridin- 3-yl)methyl)- (2,3,4,5,6-pentafluoro- phenyl)sulfonamido) acetamido)benzoic acid | 793.7 |
| 1195 | 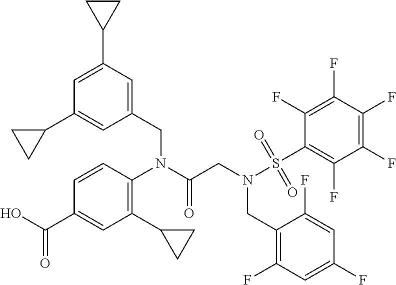 [SMILES: O=C(O)c1ccc(N(Cc2cc(C3CC3)cc(C3CC3)c2)C(=O)CN(Cc2c(F)cc(F)cc2F)S(=O)(=O)c2c(F)c(F)c(F)c(F)c2F)c(C2CC2)c1] | 3-cyclopropyl-4-(N- (3,5- dicyclopropylbenzyl)-2- (N-(2,4,6- trifluorobenzyl)- (2,3,4,5,6-pentafluoro- phenyl)sulfonamido) acetamido)benzoic acid | 778.7 |
| 1196 | 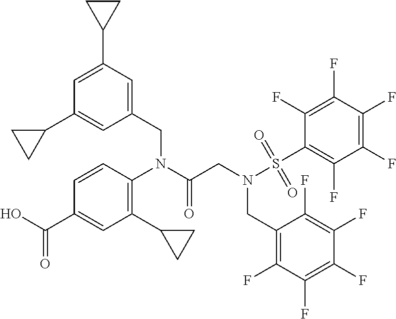 [SMILES: O=C(O)c1ccc(N(Cc2cc(C3CC3)cc(C3CC3)c2)C(=O)CN(Cc2c(F)c(F)c(F)c(F)c2F)S(=O)(=O)c2c(F)c(F)c(F)c(F)c2F)c(C2CC2)c1] | 3-cyclopropyl-4-(N- (3,5- dicyclopropylbenzyl)-2- (N- ((perfluorophenyl) methyl)-(2,3,4,5,6- pentafluoro- phenyl)sulfonamido) acetamido)benzoic acid | 814.7 |
| 1197 | 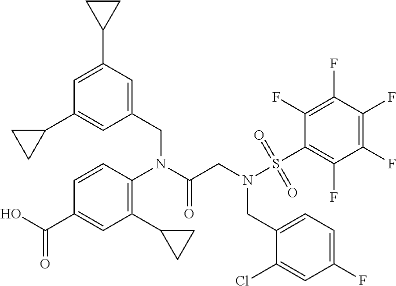 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(C2CC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- cyclopropylbenzoic acid | 777.2 |
| 1198 | 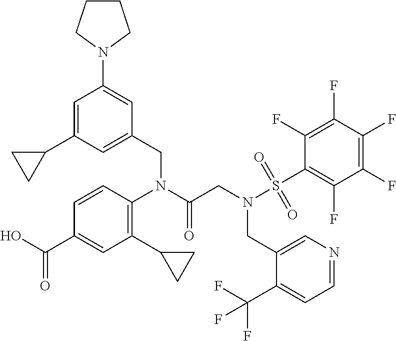 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(C2CC2)c1] | 3-cyclopropyl-4-(N-(3- cyclopropyl-5- (pyrrolidin-1- yl)benzyl)-2-(N-((4- (trifluoromethyl)pyridin- 3-yl)methyl)- (2,3,4,5,6-pentafluoro- phenyl)sulfonamido) acetamido)benzoic acid | 822.8 |
| 1199 | 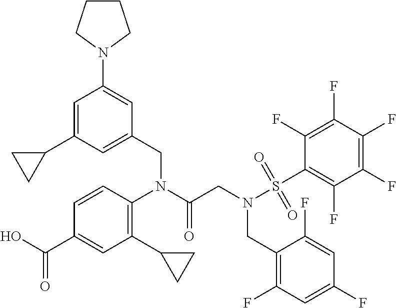 [SMILES: O=C(O)c1ccc(N(Cc2cc(C3CC3)cc(N3CCCC3)c2)C(=O)CN(Cc2c(F)cc(F)cc2F)S(=O)(=O)c2c(F)c(F)c(F)c(F)c2F)c(C2CC2)c1] | 3-cyclopropyl-4-(N-(3- cyclopropyl-5- (pyrrolidin-1- yl)benzyl)-2-(N-(2,4,6- trifluorobenzyl)- (2,3,4,5,6-pentafluoro- phenyl)sulfonamido) acetamido)benzoic acid | 807.8 |
| 1200 | 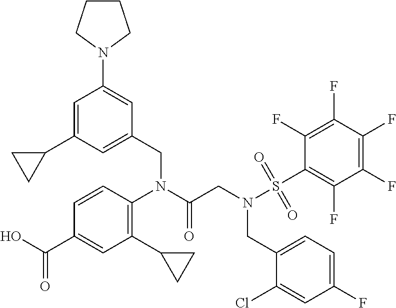 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(C2CC2)c1] | 4-(2-(N-(2-chloro-4- fluorobenzyl)- (2,3,4,5,6- pentafluorophenyl)sulfon- amido)-N-(3- cyclopropyl-5- (pyrrolidin-1- yl)benzyl)acetamido)-3- cyclopropylbenzoic acid | 806.2 |
| 1201 | 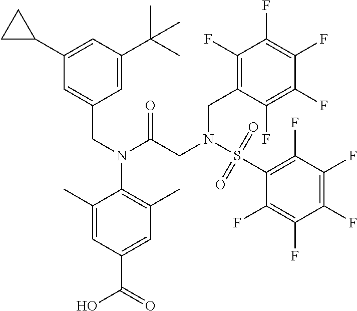 [SMILES: Cc1cc(C(=O)O)cc(C)c1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | 4-(N-(3-(tert-butyl)-5- cyclopropylbenzyl)-2- (N- ((perfluorophenyl) methyl)-(2,3,4,5,6- pentafluoro phenyl)sulfonamido) acetamido)-3,5- dimethylbenzoic acid | 818.7 |
| 1202 | 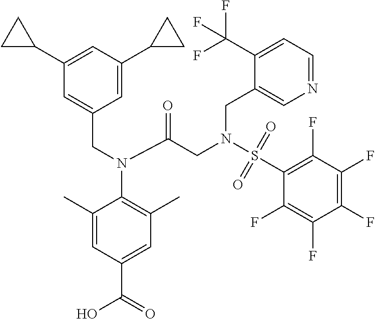 [SMILES: Cc1cc(C(=O)O)cc(C)c1N(Cc1cc(C2CC2)cc(C2CC2)c1)C(=O)CN(Cc1cnccc1C(F)(F)F)S(=O)(=O)c1c(F)c(F)c(F)c(F)c1F] | 4-(N-(3,5- dicyclopropylbenzyl)-2- (N-((4- (trifluoromethyl)pyridin- 3-yl)methyl)- (2,3,4,5,6-pentafluoro- phenyl)sulfonamido) acetamido)-3,5- dimethylbenzoic acid | 781.7 |
| 1203 | 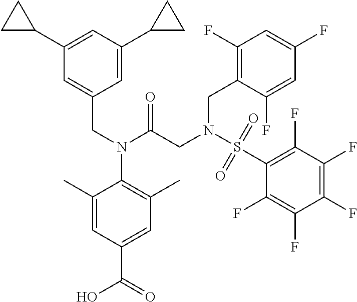 [SMILES: Cc1cc(C(=O)O)cc(C)c1N(Cc1cc(C2CC2)cc(C2CC2)c1)C(=O)CN(Cc1c(F)cc(F)cc1F)S(=O)(=O)c1c(F)c(F)c(F)c(F)c1F] | 4-(N-(3,5- dicyclopropylbenzyl)-2- (N-(2,4,6- trifluorobenzyl)- (2,3,4,5,6-pentafluoro- phenyl) sulfonamido) acetamido)-3,5- dimethylbenzoic acid | 766.7 |
| 1204 | 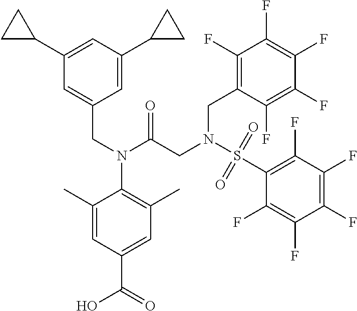 [SMILES: Cc1cc(C(=O)O)cc(C)c1N(Cc1cc(C2CC2)cc(C2CC2)c1)C(=O)CN(Cc1c(F)c(F)c(F)c(F)c1F)S(=O)(=O)c1c(F)c(F)c(F)c(F)c1F] | 4-(N-(3,5- dicyclopropylbenzyl)-2- (N- ((perfluorophenyl) methyl)-(2,3,4,5,6- pentafluoro- phenyl)sulfonamido) acetamido)-3,5- dimethylbenzoic acid | 802.7 |
| 1205 | 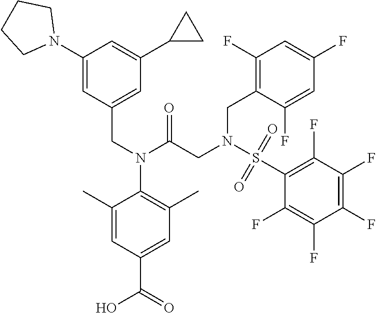 [SMILES: Cc1cc(C(=O)O)cc(C)c1N(Cc1cc(C2CC2)cc(N2CCCC2)c1)C(=O)CN(Cc1c(F)cc(F)cc1F)S(=O)(=O)c1c(F)c(F)c(F)c(F)c1F] | 4-(N-(3-cyclopropyl-5- (pyrrolidin-1- yl)benzyl)-2-(N-(2,4,6- trifluorobenzyl)- (2,3,4,5,6-pentafluoro- phenyl)sulfonamido) acetamido)-3,5- dimethylbenzoic acid | 795.7 |
| 1206 | 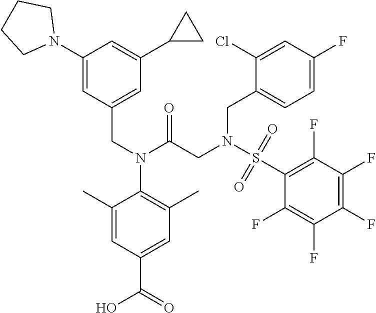 [SMILES: Cc1cc(C(=O)O)cc(C)c1N(Cc1cc(C2CC2)cc(N2CCCC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3- cyclopropyl-5- (pyrrolidin-1- yl)benzyl)acetamido)- 3,5-dimethylbenzoic acid | 794.2 |
| 1207 | 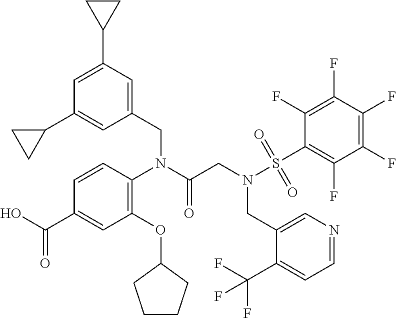 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(OC2CCCC2)c1] | 3-(cyclopentyloxy)-4- (N-(3,5- dicyclopropylbenzyl)-2- (N-((4- (trifluoromethyl)pyridin- 3-yl)methyl)- (2,3,4,5,6-pentafluoro- phenyl)sulfonamido) acetamido)benzoic acid | 837.8 |
| 1208 | 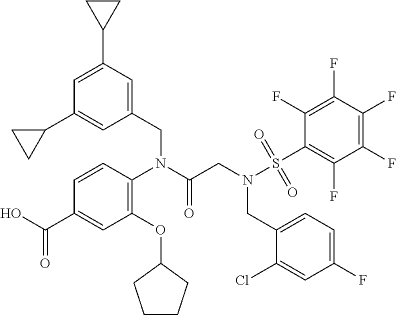 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCCC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- (cyclopentyloxy) benzoic acid | 821.2 |
| 1209 | 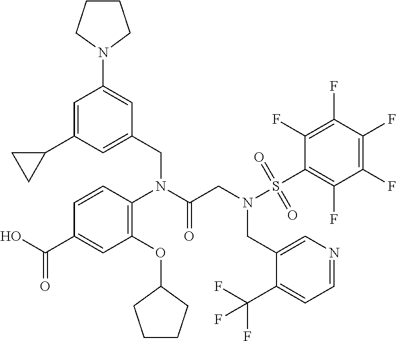 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(OC2CCCC2)c1] | 3-(cyclopentyloxy)-4- (N-(3-cyclopropyl-5- (pyrrolidin-1- yl)benzyl)-2-(N-((4- (trifluoromethyl)pyridin- 3-yl)methyl)- (2,3,4,5,6-pentafluoro- phenyl)sulfonamido) acetamido)benzoic acid | 866.8 |
| 1210 | 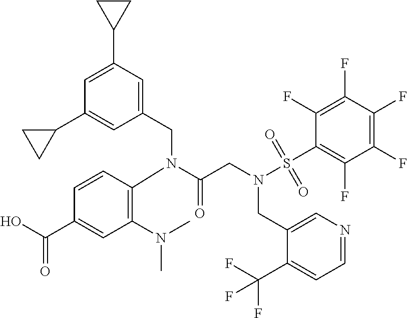 [SMILES: CN(C)c1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | 4-(N-(3,5- dicyclopropylbenzyl)-2- (N-((4- (trifluoromethyl)pyridin- 3-yl)methyl)- (2,3,4,5,6-pentafluoro- phenyl)sulfonamido) acetamido)-3- (dimethylamino) benzoic acid | 796.7 |
| 1211 | 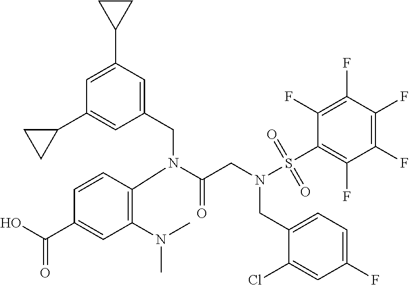 [SMILES: CN(C)c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- (dimethylamino) benzoic acid | 780.2 |
| 1212 | 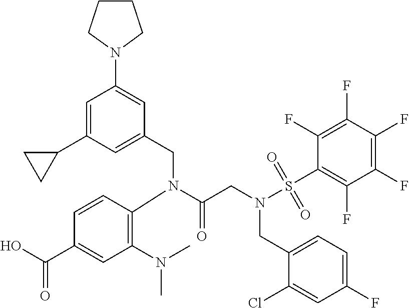 [SMILES: CN(C)c1cc(C(=O)O)ccc1N(Cc1cc(C2CC2)cc(N2CCCC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3- cyclopropyl-5- (pyrrolidin-1- yl)benzyl)acetamido)-3- (dimethylamino) benzoic acid | 809.2 |
| 1213 | 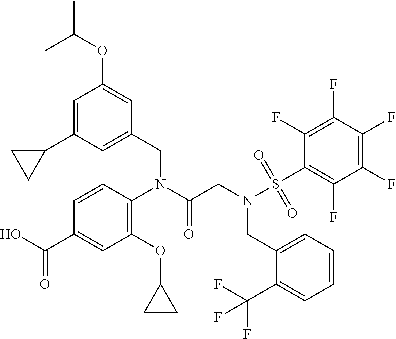 [SMILES: CC(C)Oc1cc(CN(C(=O)CN(Cc2ccccc2C(F)(F)F)S(=O)(=O)c2c(F)c(F)c(F)c(F)c2F)c2ccc(C(=O)O)cc2OC2CC2)cc(C2CC2)c1] | 3-cyclopropoxy-4-(N- (3-cyclopropyl-5- isopropoxybenzyl)-2- (N-(2- (trifluoromethyl)benzyl)- (2,3,4,5,6-pentafluoro- phenyl)sulfonamido) acetamido)benzoic acid | 826.8 |
| 1214 | 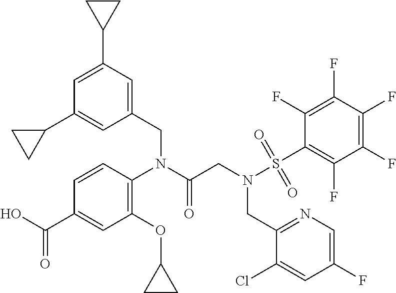 [SMILES: O=C(O)c1ccc(N(Cc2cc(C3CC3)cc(C3CC3)c2)C(=O)CN(Cc2ncc(F)cc2Cl)S(=O)(=O)c2c(F)c(F)c(F)c(F)c2F)c(OC2CC2)c1] | 4-(2-(N-((3-chloro-5- fluoropyridin-2- yl)methyl)-(2,3,4,5,6- pentafluorophenyl)sulfon- amido)-N-(3,5- dicyclopropylbenzyl) acetamido)-3- cyclopropoxybenzoic acid | 794.2 |
| 1215 | 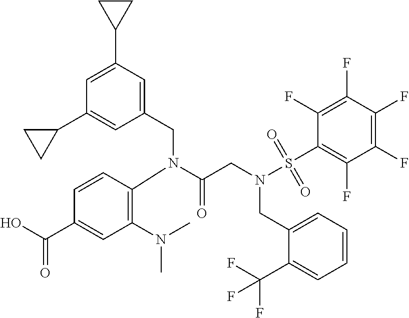 [SMILES: CN(C)c1cc(C(=O)O)ccc1N(Cc1cc(C2CC2)cc(C2CC2)c1)C(=O)CN(Cc1ccccc1C(F)(F)F)S(=O)(=O)c1c(F)c(F)c(F)c(F)c1F] | 4-(N-(3,5- dicyclopropylbenzyl)-2- (N-(2- (trifluoromethyl)benzyl)- (2,3,4,5,6-pentafluoro- phenyl)sulfonamido) acetamido)-3- (dimethylamino) benzoic acid | 795.7 |
| 1216 | 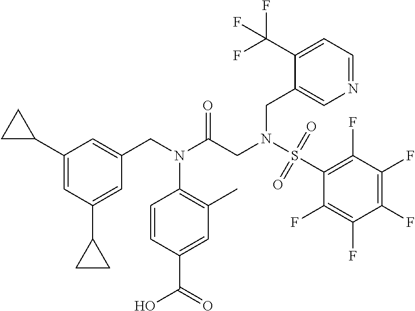 [SMILES: Cc1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | 4-(N-(3,5- dicyclopropylbenzyl)-2- (N-((4- (trifluoromethyl)pyridin- 3-yl)methyl)- (2,3,4,5,6-pentafluoro- phenyl)sulfonamido) acetamido)-3- methylbenzoic acid | 767.7 |
| 1217 | 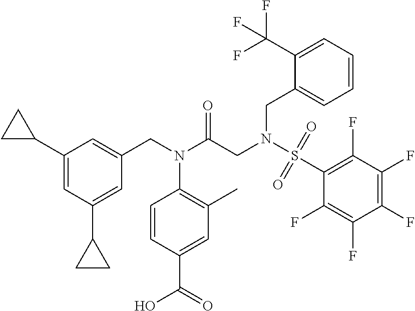 [SMILES: Cc1cc(C(=O)O)ccc1N(Cc1cc(C2CC2)cc(C2CC2)c1)C(=O)CN(Cc1ccccc1C(F)(F)F)S(=O)(=O)c1c(F)c(F)c(F)c(F)c1F] | 4-(N-(3,5- dicyclopropylbenzyl)-2- (N-(2- (trifluoromethyl)benzyl)- (2,3,4,5,6-pentafluoro- phenyl)sulfonamido) acetamido)-3- methylbenzoic acid | 766.7 |
| 1218 | 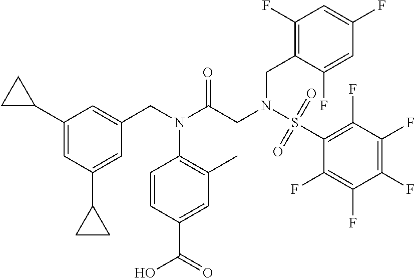 [SMILES: Cc1cc(C(=O)O)ccc1N(Cc1cc(C2CC2)cc(C2CC2)c1)C(=O)CN(Cc1c(F)cc(F)cc1F)S(=O)(=O)c1c(F)c(F)c(F)c(F)c1F] | 4-(N-(3,5- dicyclopropylbenzyl)-2- (N-(2,4,6- trifluorobenzyl)- (2,3,4,5,6-pentafluoro- phenyl)sulfonamido) acetamido)-3- methylbenzoic acid | 752.7 |
| 1219 | 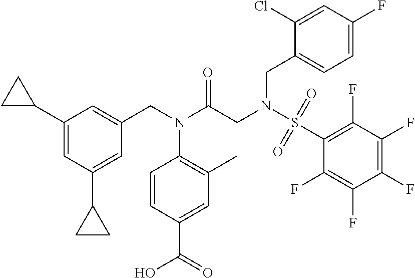 [SMILES: Cc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- methylbenzoic acid | 751.1 |
| 1220 | 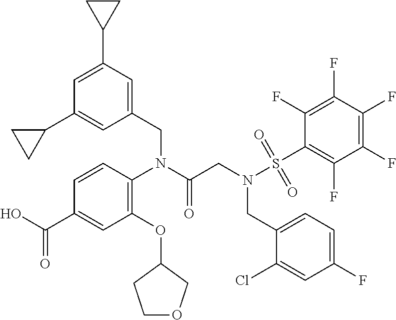 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCOC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- ((tetrahydrofuran-3- yl)oxy)benzoic acid | 823.2 |
| 1221 | 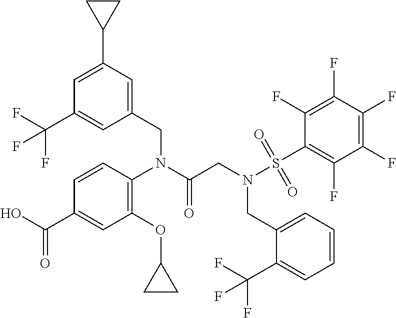 [SMILES: O=C(O)c1ccc(N(Cc2cc(C3CC3)cc(C(F)(F)F)c2)C(=O)CN(Cc2ccccc2C(F)(F)F)S(=O)(=O)c2c(F)c(F)c(F)c(F)c2F)c(OC2CC2)c1] | 3-cyclopropoxy-4-(N- (3-cyclopropyl-5- (trifluoromethyl)benzyl)- 2-(N-(2- (trifluoromethyl)benzyl)- (2,3,4,5,6-pentafluoro- phenyl)sulfonamido) acetamido)benzoic acid | 836.7 |
| 1222 | 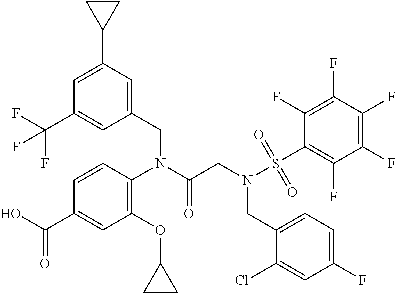 [SMILES: O=C(O)c1ccc(N(Cc2cc(C3CC3)cc(C(F)(F)F)c2)C(=O)CN(Cc2ccc(F)cc2Cl)S(=O)(=O)c2c(F)c(F)c(F)c(F)c2F)c(OC2CC2)c1] | 4-(2-(N-(2-chloro-4- fluorobenzyl)- (2,3,4,5,6- pentafluorophenyl)sulfon- amido)-N-(3- cyclopropyl-5- (trifluoromethyl)benzyl) acetamido)-3- cyclopropoxybenzoic acid | 821.1 |
| 1223 | 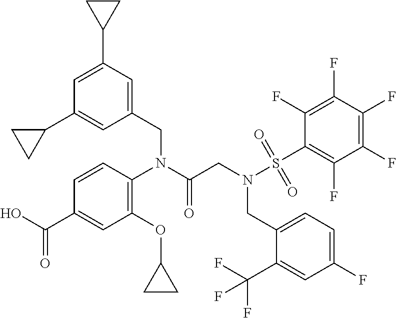 [SMILES: O=C(O)c1ccc(N(Cc2cc(C3CC3)cc(C3CC3)c2)C(=O)CN(Cc2ccc(F)cc2C(F)(F)F)S(=O)(=O)c2c(F)c(F)c(F)c(F)c2F)c(OC2CC2)c1] | 3-cyclopropoxy-4-(N- (3,5- dicyclopropylbenzyl)-2- (N-(4-fluoro-2- (trifluoromethyl)benzyl)- (2,3,4,5,6-pentafluoro- phenyl)sulfonamido) acetamido)benzoic acid | 826.7 |
| 1224 | 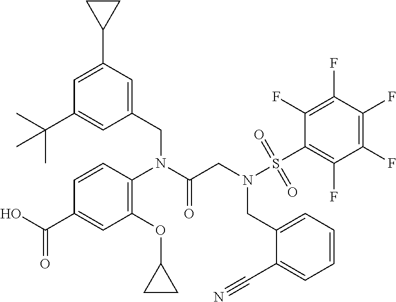 [SMILES: CC(C)(C)c1cc(CN(C(=O)CN(Cc2ccccc2C#N)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2-cyanobenzyl)- (2,3,4,5,6- pentafluorophenyl)sulfon- amido)acetamido)-3- cyclopropoxybenzoic acid | 781.8 |
| 1225 | 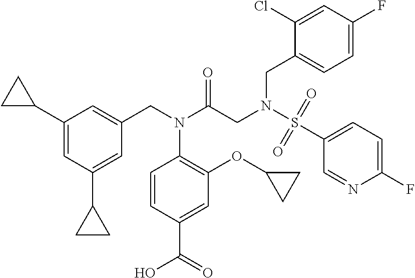 [SMILES: O=C(O)c1ccc(N(Cc2cc(C3CC3)cc(C3CC3)c2)C(=O)CN(Cc2ccc(F)cc2Cl)S(=O)(=O)c2ccc(F)nc2)c(OC2CC2)c1] | 4-(2-(N-(2-chloro-4- fluorobenzyl)-(6- fluoropyridine)-3- sulfonamido)-N-(3,5- dicyclopropylbenzyl) acetamido)-3- cyclopropoxybenzoic acid | 722.2 |
| 1226 | 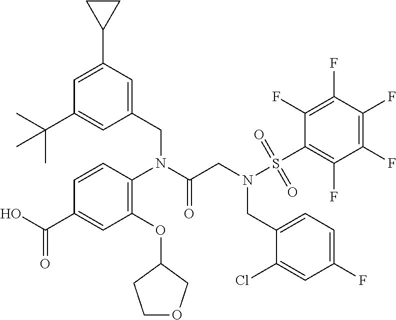 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2OC2CCOC2)cc(C2CC2)c1] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- ((tetrahydrofuran-3- yl)oxy)benzoic acid | 839.2 |
| 1227 | 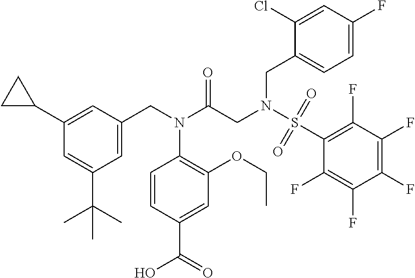 [SMILES: CCOc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- ethoxybenzoic acid | 797.2 |
| 1228 | 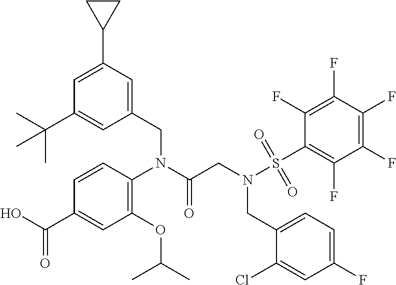 [SMILES: CC(C)Oc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- isopropoxybenzoic acid | 811.2 |
| 1229 | 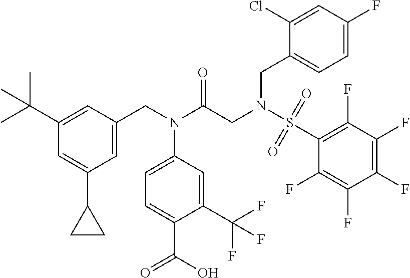 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)c(C(F)(F)F)c2)cc(C2CC2)c1] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-2- (trifluoromethyl) benzoic acid | 821.2 |
| 1230 | 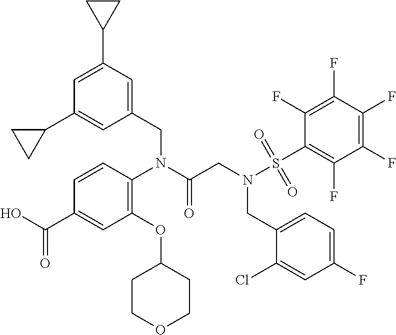 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCOCC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- ((tetrahydro-2H-pyran- 4-yl)oxy)benzoic acid | 837.2 |
| 1231 | 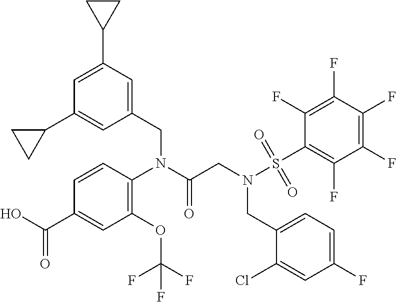 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC(F)(F)F)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- (trifluoromethoxy) benzoic acid | 821.1 |
| 1232 | 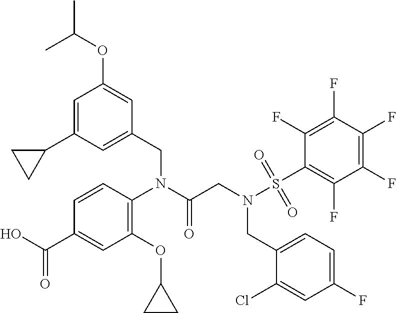 [SMILES: CC(C)Oc1cc(CN(C(=O)CN(Cc2ccc(F)cc2Cl)S(=O)(=O)c2c(F)c(F)c(F)c(F)c2F)c2ccc(C(=O)O)cc2OC2CC2)cc(C2CC2)c1] | 4-(2-(N-(2-chloro-4- fluorobenzyl)- (2,3,4,5,6- pentafluorophenyl)sulfon- amido)-N-(3- cyclopropyl-5- isopropoxybenzyl) acetamido)-3- cyclopropoxybenzoic acid | 811.2 |
| 1234 | 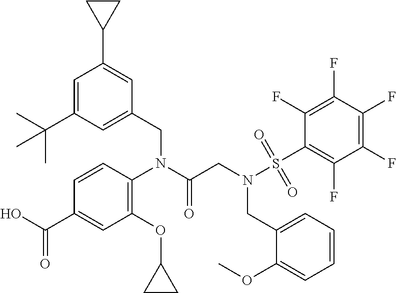 [SMILES: COc1ccccc1CN(CC(=O)N(Cc1cc(C2CC2)cc(C(C)(C)C)c1)c1ccc(C(=O)O)cc1OC1CC1)S(=O)(=O)c1c(F)c(F)c(F)c(F)c1F] | 4-(N-(3-(tert-butyl)-5- cyclopropylbenzyl)-2- (N-(2-methoxybenzyl)- (2,3,4,5,6-pentafluoro- phenyl)sulfonamido) acetamido)-3- cyclopropoxybenzoic acid | 786.8 |
| 1235 | 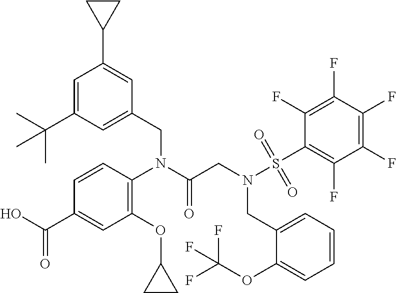 [SMILES: CC(C)(C)c1cc(CN(C(=O)CN(Cc2ccccc2OC(F)(F)F)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2- (trifluoromethoxy) benzyl)-(2,3,4,5,6- pentafluoro- phenyl)sulfonamido) acetamido)-3- cyclopropoxybenzoic acid | 840.8 |
| 1236 | 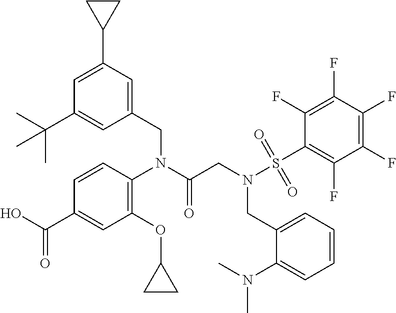 [SMILES: CN(C)c1ccccc1CN(CC(=O)N(Cc1cc(C2CC2)cc(C(C)(C)C)c1)c1ccc(C(=O)O)cc1OC1CC1)S(=O)(=O)c1c(F)c(F)c(F)c(F)c1F] | 4-(N-(3-(tert-butyl)-5- cyclopropylbenzyl)-2- (N-(2- (dimethylamino)benzyl)- (2,3,4,5,6- pentafluorophenyl)sulfon- amido)acetamido)-3- cyclopropoxybenzoic acid | 799.8 |
| 1237 | 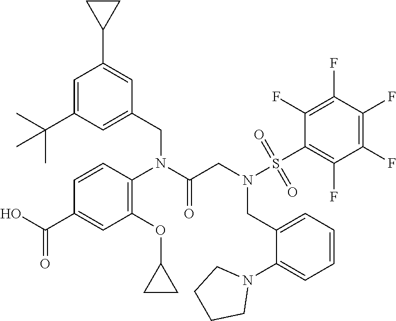 [SMILES: CC(C)(C)c1cc(CN(C(=O)CN(Cc2ccccc2N2CCCC2)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2-(pyrrolidin-1- yl)benzyl)-(2,3,4,5,6- pentafluoro- phenyl)sulfonamido) acetamido)-3- cyclopropoxybenzoic acid | 825.9 |
| 1238 | 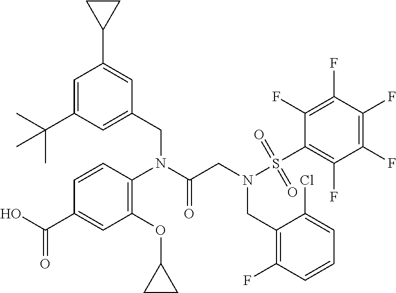 [SMILES: CC(C)(C)c1cc(CN(C(=O)CN(Cc2c(F)cccc2Cl)S(=O)(=O)c2c(F)c(F)c(F)c(F)c2F)c2ccc(C(=O)O)cc2OC2CC2)cc(C2CC2)c1] | 4-(N-(3-(tert-butyl)-5- cyclopropylbenzyl)-2- (N-(2-chloro-6- fluorobenzyl)- (2,3,4,5,6- pentafluorophenyl)sulfon- amido)acetamido)-3- cyclopropoxybenzoic acid | 809.2 |
| 1239 | 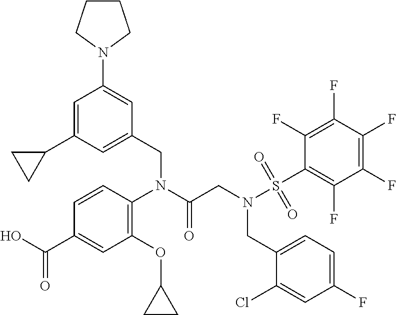 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(OC2CC2)c1] | 4-(2-(N-(2-chloro-4- fluorobenzyl)- (2,3,4,5,6- pentafluorophenyl)sulfon- amido)-N-(3- cyclopropyl-5- (pyrrolidin-1- yl)benzyl)acetamido)-3- (cyclopentyloxy)benzoic acid | 850.3 |
| 1240 | 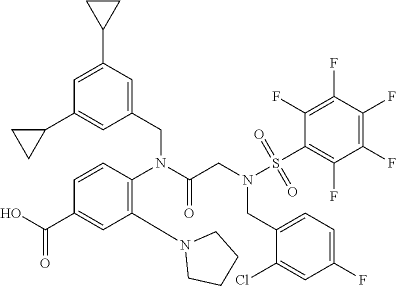 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCCC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3-(pyrrolidin- 1-yl)benzoic acid | 806.2 |
| 1241 | 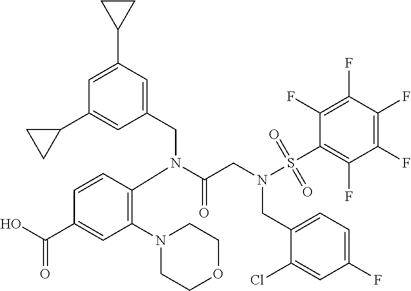 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCOCC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- morpholinobenzoic acid | 822.2 |
| 1242 | 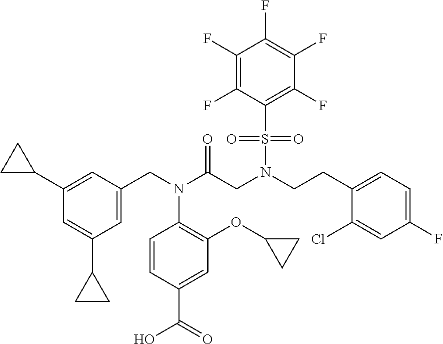 [SMILES: O=C(O)c1ccc(N(Cc2cc(C3CC3)cc(C3CC3)c2)C(=O)CN(CCc2ccc(F)cc2Cl)S(=O)(=O)c2c(F)c(F)c(F)c(F)c2F)c(OC2CC2)c1] | 4-(2-(N-(2-chloro-4- fluorophenethyl)- (2,3,4,5,6- pentafluorophenyl)sulfon- amido)-N-(3,5- dicyclopropylbenzyl) acetamido)-3- cyclopropoxybenzoic acid | 807.2 |
| 1243 | 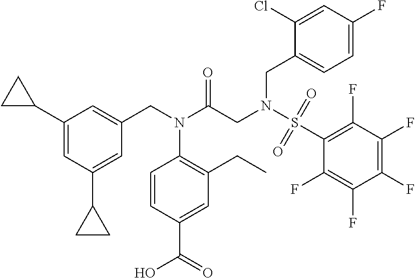 [SMILES: CCc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- ethylbenzoic acid | 765.2 |
| 1244 | 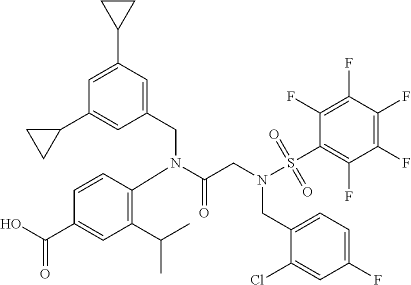 [SMILES: CC(C)c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- isopropylbenzoic acid | 779.2 |
| 1245 | 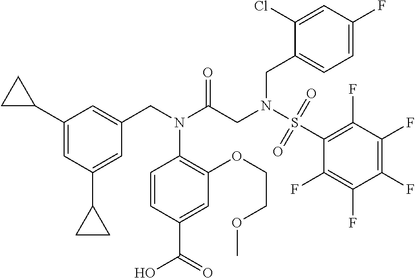 [SMILES: COCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3-(2- methoxyethoxy)benzoic acid | 811.2 |
| 1246 | 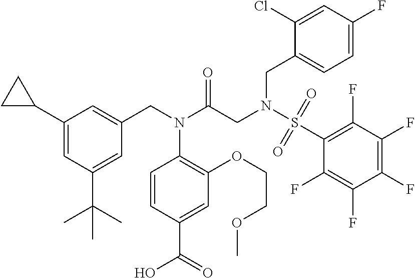 [SMILES: COCCOc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- (2- methoxyethoxy)benzoic acid | 827.2 |
| 1247 | 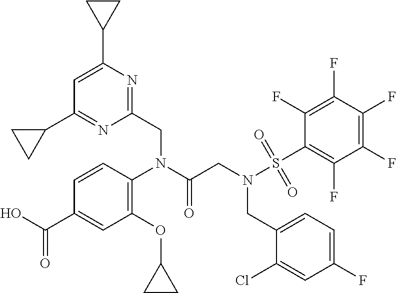 [SMILES: O=C(O)c1ccc(N(Cc2nc(C3CC3)cc(C3CC3)n2)C(=O)CN(Cc2ccc(F)cc2Cl)S(=O)(=O)c2c(F)c(F)c(F)c(F)c2F)c(OC2CC2)c1] | 4-(2-(N-(2-chloro-4- fluorobenzyl)- (2,3,4,5,6- pentafluorophenyl)sulfon- amido)-N-((4,6- dicyclopropylpyrimidin- 2- yl)methyl)acetamido)- 3-cyclopropoxybenzoic acid | 795.2 |
| 1248 | 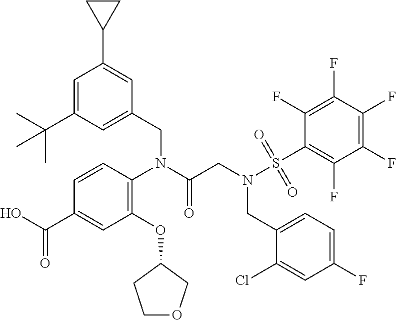 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2O[C@H]2CCOC2)cc(C2CC2)c1] | (S)-4-(N-(3-(tert-butyl)- 5-cyclopropylbenzyl)-2- (N-(2-chloro-4- fluorobenzyl)- (2,3,4,5,6- pentafluorophenyl)sulfon- amido)acetamido)-3- ((tetrahydrofuran-3- yl)oxy)benzoic acid | 839.2 |
| 1249 | 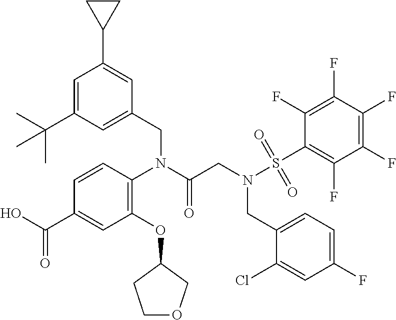 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2O[C@@H]2CCOC2)cc(C2CC2)c1] | (R)-4-(N-(3-(tert-butyl)- 5-cyclopropylbenzyl)-2- (N-(2-chloro-4- fluorobenzyl)- (2,3,4,5,6- pentafluorophenyl)sulfon- amido)acetamido)-3- ((tetrahydrofuran-3- yl)oxy)benzoic acid | 839.2 |
| 1250 | 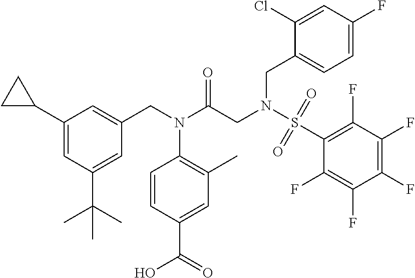 [SMILES: Cc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | 4-(N-(3-(tert-butyl)-5- cyclopropylbenzyl)-2- (N-(2-chloro-4- fluorobenzyl)- (2,3,4,5,6- pentafluorophenyl)sulfon- amido)acetamido)-3- methylbenzoic acid | 767.2 |
| 1251 | 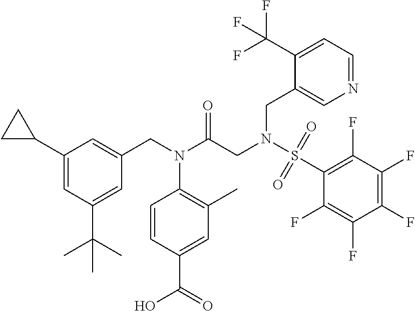 [SMILES: Cc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- methylbenzoic acid | 783.7 |
| 1252 | 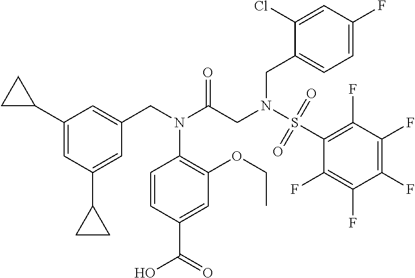 [SMILES: CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- ethoxybenzoic acid | 781.2 |
| 1253 | 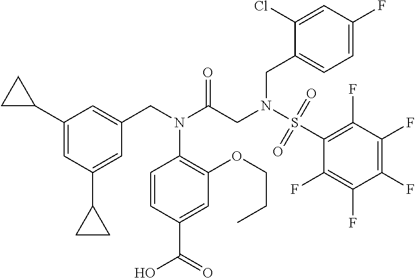 [SMILES: CCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- propoxybenzoic acid | 795.2 |
| 1254 | 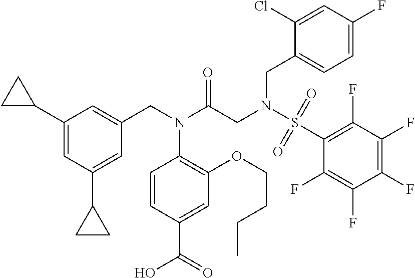 [SMILES: CCCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 3-butoxy-4-(2-(N-(2- chloro-4-fluorobenzyl)- (2,3,4,5,6- pentafluorophenyl)sulfon- amido)-N-(3,5- dicyclopropylbenzyl) acetamido)benzoic acid | 809.2 |
| 1255 | 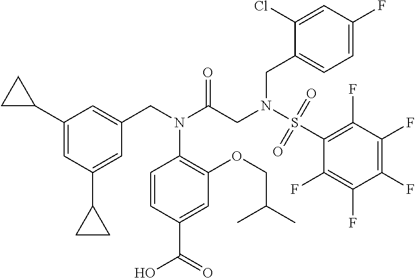 [SMILES: CC(C)COc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3- isobutoxybenzoic acid | 809.2 |
| 1256 | 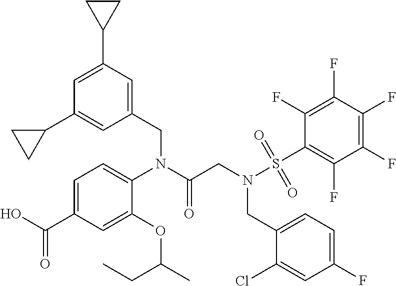 [SMILES: CCC(C)Oc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 3-(sec-butoxy)-4-(2-(N- (2-chloro-4- fluorobenzyl)- (2,3,4,5,6- pentafluorophenyl)sulfon- amido)-N-(3,5- dicyclopropylbenzyl) acetamido)benzoic acid | 809.2 |
| 1257 | 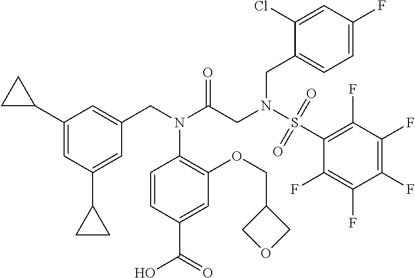 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OCC2COC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3-(oxetan-3- ylmethoxy)benzoic acid | 823.2 |
| 1258 | 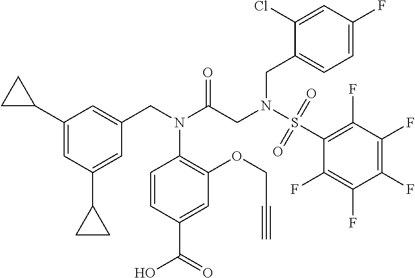 [SMILES: C#CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3-(prop-2-yn- 1-yloxy)benzoic acid | 791.2 |
| 1260 | 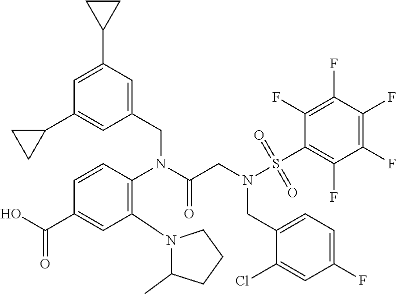 [SMILES: CC1CCCN1c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3-(2- methylpyrrolidin-1- yl)benzoic acid | 820.2 |
| 1261 | 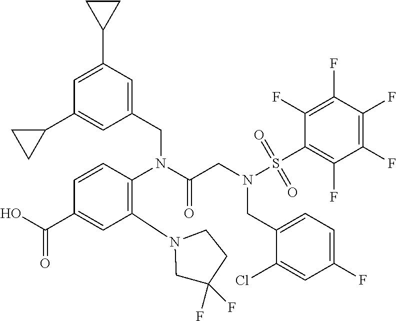 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCC(F)(F)C2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3-(3,3- difluoropyrrolidin-1- yl)benzoic acid | 842.2 |
| 1262 | 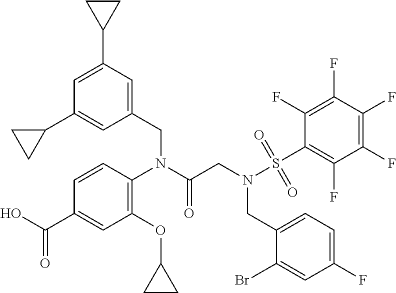 [SMILES: O=C(O)c1ccc(N(Cc2cc(C3CC3)cc(C3CC3)c2)C(=O)CN(Cc2ccc(F)cc2Br)S(=O)(=O)c2c(F)c(F)c(F)c(F)c2F)c(OC2CC2)c1] | 4-(2-(N-(2-bromo-4- fluorobenzyl)- (2,3,4,5,6- pentafluorophenyl)sulfon- amido)-N-(3,5- dicyclopropylbenzyl) acetamido)-3- cyclopropoxybenzoic acid | 837.6 |
| 1263 | 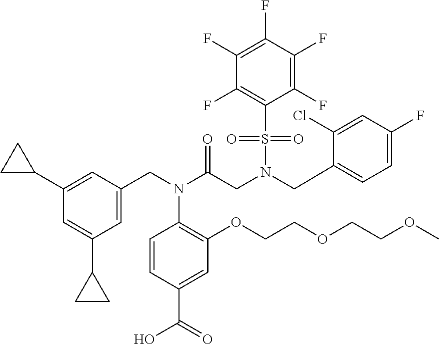 [SMILES: COCCOCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-(N-(2-chloro-4- fluorobenzyl)- (2,3,4,5,6- pentafluorophenyl)sulfon- amido)-N-(3,5- dicyclopropylbenzyl) acetamido)-3-(2-(2- methoxyethoxy)ethoxy) benzoic acid | 855.2 |
| 1264 | 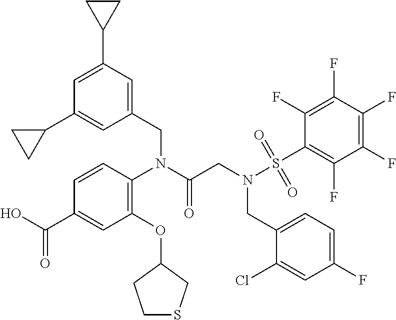 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCSC2)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- ((tetrahydrothiophen-3- yl)oxy)benzoic acid | 839.3 |
| 1267 | 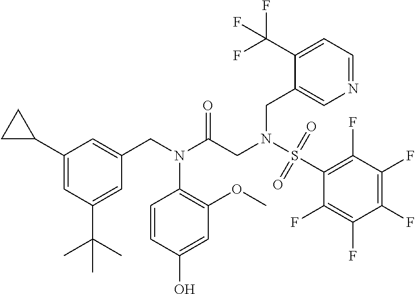 [SMILES: COc1cc(O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | N-(3-(tert-butyl)-5- cyclopropylbenzyl)-N- (4-hydroxy-2- methoxyphenyl)-2-(N- ((4- (trifluoromethyl)pyridin- 3-yl)methyl)- (2,3,4,5,6-pentafluoro- phenyl)sulfonamido) acetamide | 771.7 |
| 1268 | 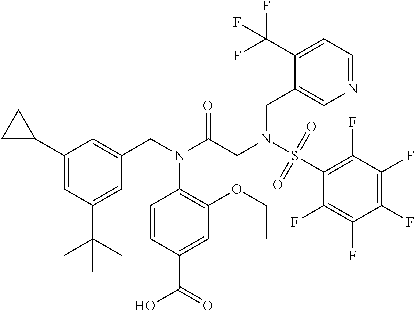 [SMILES: CCOc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | 4-(N-(3-(tert-butyl)-5- cyclopropylbenzyl)-2- (N-((4- (trifluoromethyl)pyridin- 3-yl)methyl)- (2,3,4,5,6-pentafluoro- phenyl)sulfonamido) acetamido)-3- ethoxybenzoic acid | 813.8 |
| 1270 | 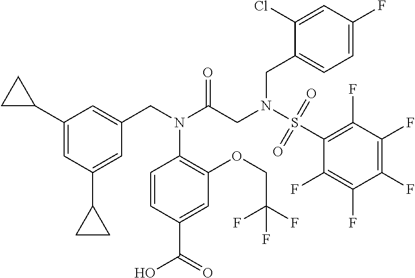 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OCC(F)(F)F)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3-(2,2,2- trifluoroethoxy)benzoic acid | 835.1 |
| 1271 | 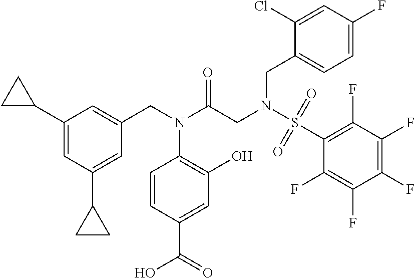 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(O)c1] | 4-(2-(N-(2-chloro-4- fluorobenzyl)- (2,3,4,5,6- pentafluorophenyl)sulfon- amido)-N-(3,5- dicyclopropylbenzyl) acetamido)-3- hydroxybenzoic acid | 753.1 |
| 1272 | 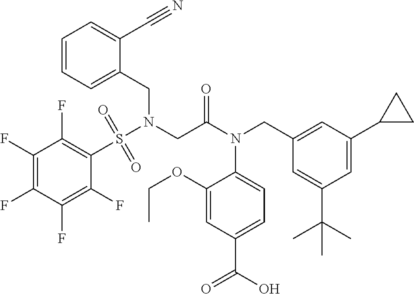 [SMILES: CCOc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | 4-(N-(3-(tert-butyl)-5- cyclopropylbenzyl)-2- ((N-(2-cyanobenzyl)- 2,3,4,5,6- pentafluorophenyl)sulfon- amido)acetamido)-3- ethoxybenzoic acid | 769.8 |
| 1273 | 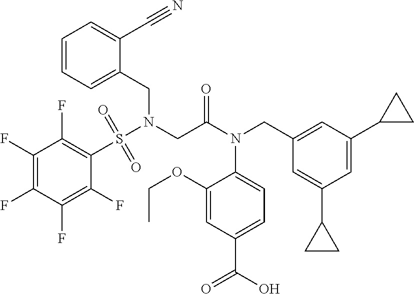 [SMILES: CCOc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F] | 4-(2-((N-(2- cyanobenzyl)-2,3,4,5,6- pentafluorophenyl)sulfon- amido)-N-(3,5- dicyclopropylbenzyl) acetamido)-3- ethoxybenzoic acid | 753.7 |
| 1274 | 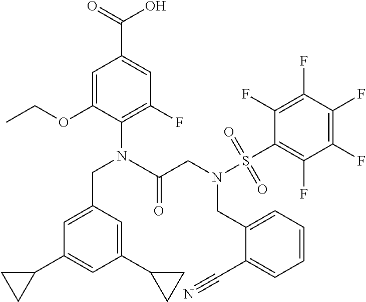 [SMILES: CCOc1cc(C(=O)O)cc(F)c1N(Cc1cc(C2CC2)cc(C2CC2)c1)C(=O)CN(Cc1ccccc1C#N)S(=O)(=O)c1c(F)c(F)c(F)c(F)c1F] | 4-(2-((N-(2- cyanobenzyl)-2,3,4,5,6- pentafluorophenyl)sulfon- amido)-N-(3,5- dicyclopropylbenzyl) acetamido)-3-ethoxy-5- fluorobenzoic acid | 771.7 |
| 1275 | 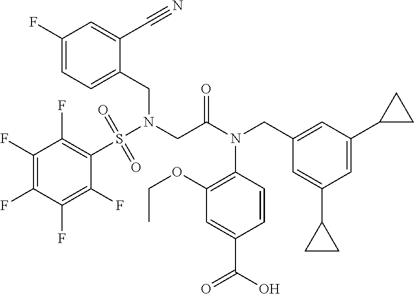 [SMILES: CCOc1cc(C(=O)O)ccc1N(Cc1cc(C2CC2)cc(C2CC2)c1)C(=O)CN(Cc1ccc(F)cc1C#N)S(=O)(=O)c1c(F)c(F)c(F)c(F)c1F] | 4-(2-((N-(2-cyano-4- fluorobenzyl)-2,3,4,5,6- pentafluorophenyl)sulfon- amido)-N-(3,5- dicyclopropylbenzyl) acetamido)-3- ethoxybenzoic acid | 771.7 |
| 1276 | 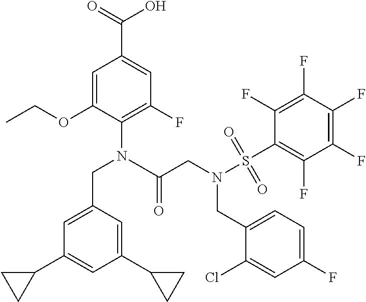 [SMILES: CCOc1cc(C(=O)O)cc(F)c1N(Cc1cc(C2CC2)cc(C2CC2)c1)C(=O)CN(Cc1ccc(F)cc1Cl)S(=O)(=O)c1c(F)c(F)c(F)c(F)c1F] | 4-(2-((N-(2-chloro-4- fluorobenzyl)-2,3,4,5,6- pentafluorophenyl)sulfon- amido)-N-(3,5- dicyclopropylbenzyl) acetamido)-3-ethoxy-5- fluorobenzoic acid | 799.2 |
| 1277 | 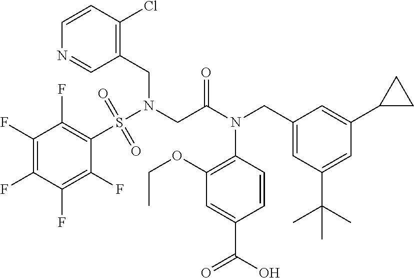 [SMILES: CCOc1cc(C(=O)O)ccc1N(Cc1cc(C2CC2)cc(C(C)(C)C)c1)C(=O)CN(Cc1cnccc1Cl)S(=O)(=O)c1c(F)c(F)c(F)c(F)c1F] | 4-(N-(3-(tert-butyl)-5- cyclopropylbenzyl)-2- ((N-((4-chloropyridin- 3-yl)methyl)-2,3,4,5,6- pentafluorophenyl)sulfon- amido)acetamido)-3- ethoxybenzoic acid | 780.2 |
| 1278 | 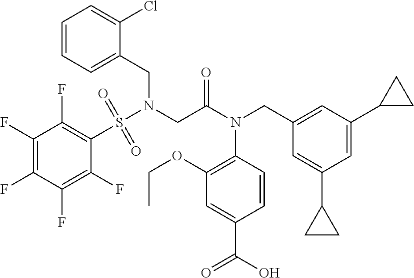 [SMILES: CCOc1cc(C(=O)O)ccc1N(Cc1cc(C2CC2)cc(C2CC2)c1)C(=O)CN(Cc1ccccc1Cl)S(=O)(=O)c1c(F)c(F)c(F)c(F)c1F] | 4-(2-((N-(2- chlorobenzyl)-2,3,4,5,6- pentafluorophenyl)sulfon- amido)-N-(3,5- dicyclopropylbenzyl) acetamido)-3- ethoxybenzoic acid | 763.2 |
| 1279 | 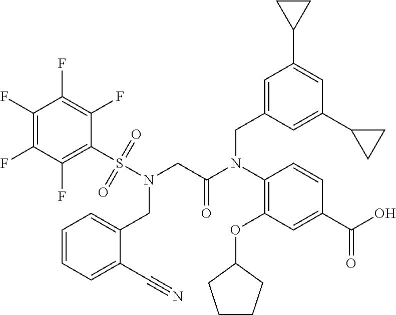 [SMILES: N#Cc1ccccc1CN(CC(=O)N(Cc1cc(C2CC2)cc(C2CC2)c1)c1ccc(C(=O)O)cc1OC1CCCC1)S(=O)(=O)c1c(F)c(F)c(F)c(F)c1F] | 4-(2-((N-(2- cyanobenzyl)-2,3,4,5,6- pentafluorophenyl)sulfon- amido)-N-(3,5- dicyclopropylbenzyl) acetamido)-3- (cyclopentyloxy) benzoic acid | 793.8 |
| 1280 | 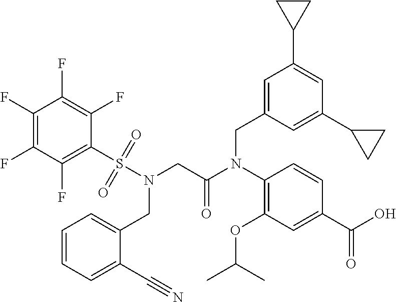 [SMILES: CC(C)Oc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F] | 4-(2-((N-(2- cyanobenzyl)-2,3,4,5,6- pentafluorophenyl)sulfon- amido)-N-(3,5- dicyclopropylbenzyl) acetamido)-3- isopropoxybenzoic acid | 767.8 |
| 1281 | 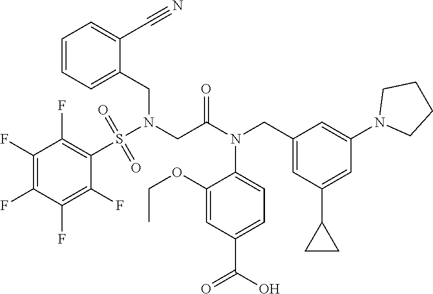 [SMILES: CCOc1cc(C(=O)O)ccc1N(Cc1cc(C2CC2)cc(N2CCCC2)c1)C(=O)CN(Cc1ccccc1C#N)S(=O)(=O)c1c(F)c(F)c(F)c(F)c1F] | 4-(2-((N-(2- cyanobenzyl)-2,3,4,5,6- pentafluorophenyl)sulfon- amido)-N-(3- cyclopropyl-5- (pyrrolidin-1- yl)benzyl)acetamido)-3- ethoxybenzoic acid | 782.8 |

In some embodiments, described herein is a compound selected from Table 2, or a pharmaceutically acceptable salt or solvate thereof.

TABLE 2
Exemplary Compounds

| Compound | | | |
|---|---|---|---|
| No. | Structure | IUPAC name | MW |
| 2001 | 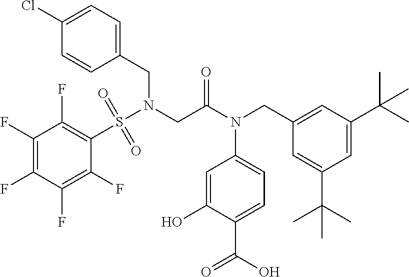 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C(C)(C)C)c1] | 4-(2-(N-(4-chlorobenzyl)- (2,3,4,5,6- pentafluorophenyl)sulfon- amido)-N-(3,5-di-tert- butylbenzyl)acetamido)-2- hydroxybenzoic acid | 767.2 |
| 2002 | 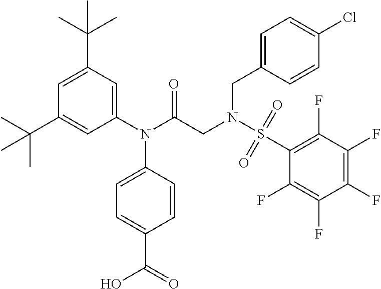 [SMILES: CC(C)(C)c1cc(N(C(=O)CN(Cc2ccc(Cl)cc2)S(=O)(=O)c2c(F)c(F)c(F)c(F)c2F)c2ccc(C(=O)O)cc2)cc(C(C)(C)C)c1] | 4-(2-(N-(4-chlorobenzyl)- (2,3,4,5,6- pentafluorophenyl)sulfon- amido)-N-(3,5-di-tert- butylphenyl)acetamido)ben- zoic acid | 737.2 |
| 2003 | 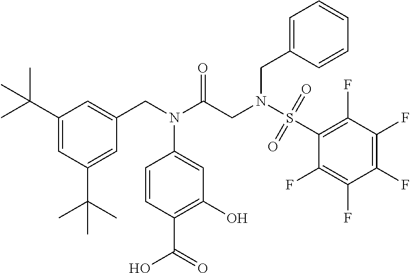 [SMILES: CC(C)(C)c1cc(CN(C(=O)CN(Cc2ccccc2)S(=O)(=O)c2c(F)c(F)c(F)c(F)c2F)c2ccc(C(=O)O)c(O)c2)cc(C(C)(C)C)c1] | 4-(2-(N-benzyl-(2,3,4,5,6- pentafluorophenyl)sulfon- amido)-N-(3,5-di-tert- butylbenzyl)acetamido)-2- hydroxybenzoic acid | 732.8 |
| 2004 | 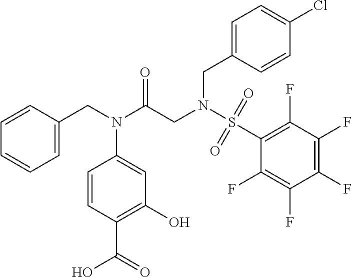 [SMILES: O=C(O)c1ccc(N(Cc2ccccc2)C(=O)CN(Cc2ccc(Cl)cc2)S(=O)(=O)c2c(F)c(F)c(F)c(F)c2F)cc1O] | 4-(N-benzyl-2-(N-(4- chlorobenzyl)-(2,3,4,5,6- pentafluorophenyl)sulfon- amido)acetamido)-2- hydroxybenzoic acid | 655 |
| 2005 | 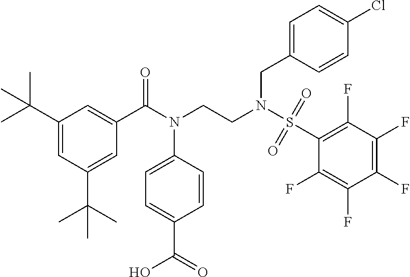 [SMILES: CC(C)(C)c1cc(C(=O)N(CCN(Cc2ccc(Cl)cc2)S(=O)(=O)c2c(F)c(F)c(F)c(F)c2F)c2ccc(C(=O)O)cc2)cc(C(C)(C)C)c1] | 4-(3,5-di-tert-butyl-N-(2- (N-(4-chlorobenzyl)- (2,3,4,5,6- pentafluorophenyl)sulfon- amido)ethyl)benzamido)ben- zoic acid | 751.2 |
| 2006 | 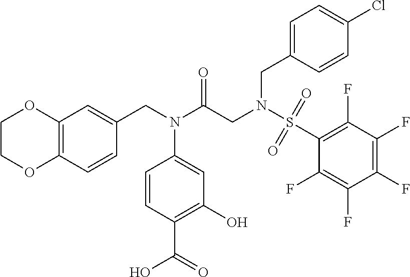 [SMILES: O=C(O)c1ccc(N(Cc2ccc3c(c2)OCCO3)C(=O)CN(Cc2ccc(Cl)cc2)S(=O)(=O)c2c(F)c(F)c(F)c(F)c2F)cc1O] | 4-(2-(N-(4-chlorobenzyl)- (2,3,4,5,6- pentafluorophenyl)sulfon- amido)-N-((2,3- dihydrobenzo[b][1,4]dioxin- 6-yl)methyl)acetamido)-2- hydroxybenzoic acid | 713 |
| 2007 | 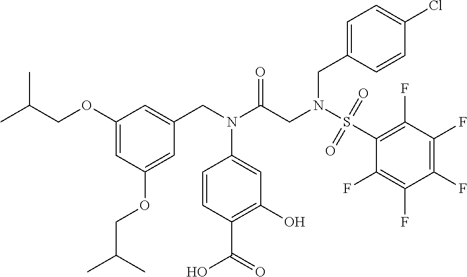 [SMILES: CC(C)COc1cc(CN(C(=O)CN(Cc2ccc(Cl)cc2)S(=O)(=O)c2c(F)c(F)c(F)c(F)c2F)c2ccc(C(=O)O)c(O)c2)cc(OCC(C)C)c1] | 4-(2-(N-(4-chlorobenzyl)- (2,3,4,5,6- pentafluorophenyl)sulfon- amido)-N-(3,5- diisobutoxybenzyl)acetami- do)-2-hydroxybenzoic acid | 799.2 |
| 2008 | 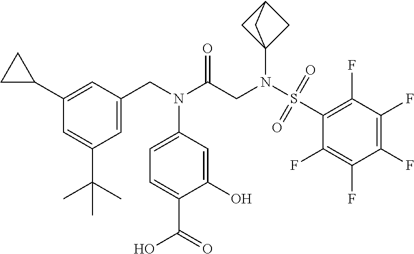 [SMILES: CC(C)(C)c1cc(CN(C(=O)CN(C23CC(C2)C3)S(=O)(=O)c2c(F)c(F)c(F)c(F)c2F)c2ccc(C(=O)O)c(O)c2)cc(C2CC2)c1] | 4-(2-(N- (bicyclo[1.1.1]pentan-1-yl)- (2,3,4,5,6- pentafluorophenyl)sulfon- amido)-N-(3-(tert-butyl)-5- cyclopropylbenzyl)acetami- do)-2-hydroxybenzoic acid | 692.7 |
| 2009 | 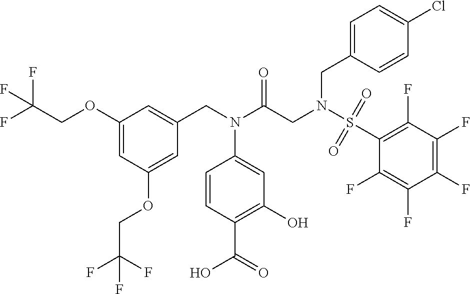 [SMILES: O=C(O)c1ccc(N(Cc2cc(OCC(F)(F)F)cc(OCC(F)(F)F)c2)C(=O)CN(Cc2ccc(Cl)cc2)S(=O)(=O)c2c(F)c(F)c(F)c(F)c2F)cc1O] | 4-(N-(3,5-bis(2,2,2- trifluoroethoxy)benzyl)-2- (N-(4-chlorobenzyl)- (2,3,4,5,6- pentafluorophenyl)sulfon- amido)acetamido)-2- hydroxybenzoic acid | 851 |
| 2010 | 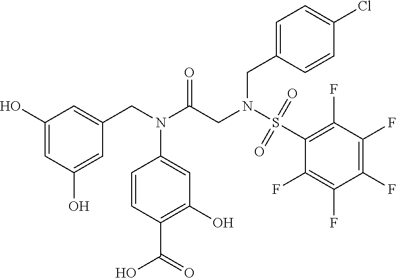 [SMILES: O=C(O)c1ccc(N(Cc2cc(O)cc(O)c2)C(=O)CN(Cc2ccc(Cl)cc2)S(=O)(=O)c2c(F)c(F)c(F)c(F)c2F)cc1O] | 4-(2-(N-(4-chlorobenzyl)- (2,3,4,5,6- pentafluorophenyl)sulfon- amido)-N-(3,5- dihydroxybenzyl)acetamido)- 2-hydroxybenzoic acid | 687 |
| 2011 | 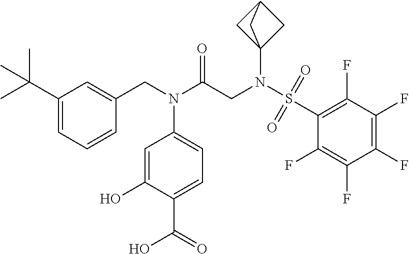 [SMILES: CC(C)(C)c1cccc(CN(C(=O)CN(C23CC(C2)C3)S(=O)(=O)c2c(F)c(F)c(F)c(F)c2F)c2ccc(C(=O)O)c(O)c2)c1] | 4-(2-(N- (bicyclo[1.1.1]pentan-1-yl)- (2,3,4,5,6- pentafluorophenyl)sulfon- amido)-N-(3-(tert- butyl)benzyl)acetamido)-2- hydroxybenzoic acid | 652.6 |
| 2012 | 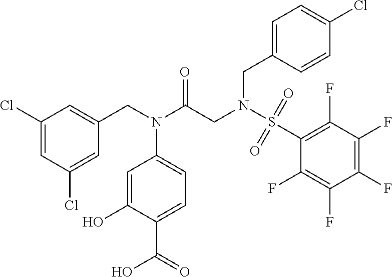 [SMILES: O=C(O)c1ccc(N(Cc2cc(Cl)cc(Cl)c2)C(=O)CN(Cc2ccc(Cl)cc2)S(=O)(=O)c2c(F)c(F)c(F)c(F)c2F)cc1O] | 4-(2-(N-(4-chlorobenzyl)- (2,3,4,5,6- pentafluorophenyl)sulfon- amido)-N-(3,5- dichlorobenzyl)acetamido)- 2-hydroxybenzoic acid | 723.9 |
| 2013 | 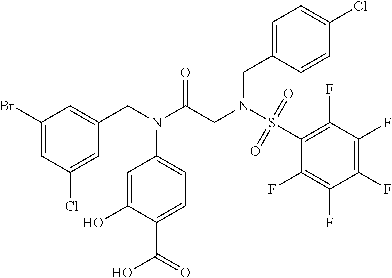 [SMILES: O=C(O)c1ccc(N(Cc2cc(Cl)cc(Br)c2)C(=O)CN(Cc2ccc(Cl)cc2)S(=O)(=O)c2c(F)c(F)c(F)c(F)c2F)cc1O] | 4-(N-(3-bromo-5- chlorobenzyl)-2-(N-(4- chlorobenzyl)-(2,3,4,5,6- pentafluorophenyl)sulfon- amido)acetamido)-2- hydroxybenzoic acid | 768.3 |
| 2014 | 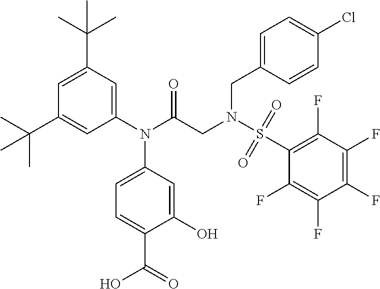 [SMILES: CC(C)(C)c1cc(N(C(=O)CN(Cc2ccc(Cl)cc2)S(=O)(=O)c2c(F)c(F)c(F)c(F)c2F)c2ccc(C(=O)O)c(O)c2)cc(C(C)(C)C)c1] | 4-(2-(N-(4-chlorobenzyl)- (2,3,4,5,6- pentafluorophenyl)sulfon- amido)-N-(3,5-di-tert- butylphenyl)acetamido)-2- hydroxybenzoic acid | 753.2 |
| 2015 | 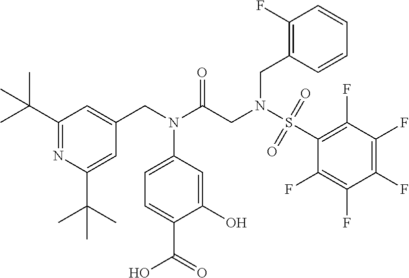 [SMILES: CC(C)(C)c1cc(CN(C(=O)CN(Cc2ccccc2F)S(=O)(=O)c2c(F)c(F)c(F)c(F)c2F)c2ccc(C(=O)O)c(O)c2)cc(C(C)(C)C)n1] | 4-(N-((2,6-di-tert- butylpyridin-4-yl)methyl)- 2-(N-(2-fluorobenzyl)- (2,3,4,5,6-pentafluoro- phenyl)sulfonamido)acetami- do)-2-hydroxybenzoic acid | 751.7 |
| 2016 | 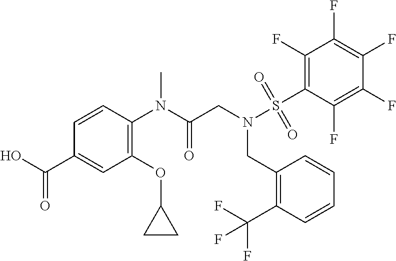 [SMILES: CN(C(=O)CN(Cc1ccccc1C(F)(F)F)S(=O)(=O)c1c(F)c(F)c(F)c(F)c1F)c1ccc(C(=O)O)cc1OC1CC1] | 3-cyclopropoxy-4-(N- methyl-2-(N-(2- (trifluoromethyl)benzyl)- (2,3,4,5,6-pentafluoro- phenyl)sulfonamido)acetami- do)benzoic acid | 652.5 |
| 2017 | 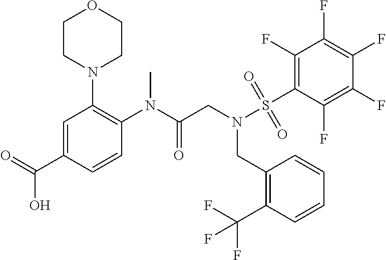 [SMILES: CN(C(=O)CN(Cc1ccccc1C(F)(F)F)S(=O)(=O)c1c(F)c(F)c(F)c(F)c1F)c1ccc(C(=O)O)cc1N1CCOCC1] | 4-(N-methyl-2-(N-(2- (trifluoromethyl)benzyl)- (2,3,4,5,6-pentafluoro- phenyl)sulfonamido)acetami- do)-3-morpholinobenzoic acid | 681.6 |
| 2018 | 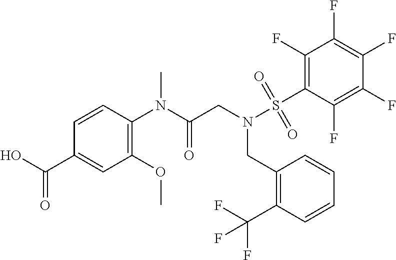 [SMILES: COc1cc(C(=O)O)ccc1N(C)C(=O)CN(Cc1ccccc1C(F)(F)F)S(=O)(=O)c1c(F)c(F)c(F)c(F)c1F] | 3-methoxy-4-(N-methyl-2- (N-(2- (trifluoromethyl)benzyl)- (2,3,4,5,6-pentafluoro- phenyl)sulfonamido)acetami- do)benzoic acid | 626.5 |
| 2020 | 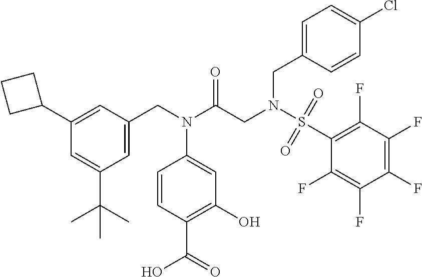 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CCC2)c1] | 4-(N-(3-(tert-butyl)-5- cyclobutylbenzyl)-2-(N-(4- chlorobenzyl)-(2,3,4,5,6- pentafluorophenyl)sulfon- amido)acetamido)-2- hydroxybenzoic acid | 765.2 |
| 2022 | 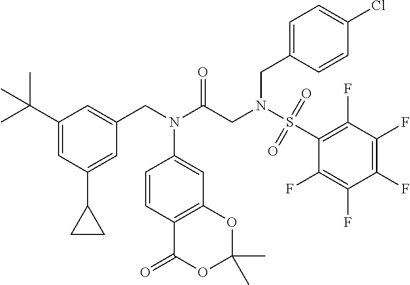 [SMILES: CC1(C)OC(=O)c2ccc(N(Cc3cc(C4CC4)cc(C(C)(C)C)c3)C(=O)CN(Cc3ccc(Cl)cc3)S(=O)(=O)c3c(F)c(F)c(F)c(F)c3F)cc2O1] | N-(3-(tert-butyl)-5- cyclopropylbenzyl)-2-(N- (4-chlorobenzyl)-(2,3,4,5,6- pentafluorophenyl)sulfon- amido)-N-(2,2-dimethyl-4- oxo-4H- benzo[d][1,3]dioxin-7- yl)acetamide | 791.2 |

Described herein are compounds, or pharmaceutically acceptable salts or solvates thereof, that are active STAT5 inhibitors. In some embodiments, a compound described herein, or a pharmaceutically acceptable salt or solvate thereof, has an IC₅₀ value that is below 50 μM, below 25 μM, below 20 μM, below 15 μM, below 10 μM, below 5 μM, below 4 μM, below 3 μM, below 2.5 μM, below 2 μM, below 1.9 μM, below 1.8 μM, below 1.7 μM, below 1.6 μM, below 1.5 μM, below 1.4 μM, below 1.3 μM, below 1.2 μM, below 1.1 μM, below 1.0 μM, below 0.9 μM, below 0.8 μM, below 0.7 μM, below 0.6 μM, below 0.5 μM, below 0.4 μM, below 0.3 μM, below 0.2 μM, below 0.1 μM, or below 0.01 μM as determined in a cell cytotoxicity assay. In some embodiments, the IC₅₀ value is determined accordingly to EXAMPLE 1B or EXAMPLE 2B. In some embodiments, a compound described herein, or a pharmaceutically acceptable salt or solvate thereof, has an IC₅₀ value from about 0.001 μM to about 0.5 μM. In some embodiments, a compound described herein, or a pharmaceutically acceptable salt or solvate thereof, has an IC₅₀ value within a range of from about 0.001 μM, 0.01 μM, 0.05 μM, or 0.1 μM to about 0.15 μM, 0.2 μM, 0.25 μM, 0.30 μM, or 0.50 μM. In some embodiments, the IC₅₀ value is determined using MV4-11 cells, wherein the compound and a vehicle control (0.5% DMSO) are added to the cell solution and incubated for 72 h at 37° C. in 5% CO₂. In some embodiments, the IC₅₀ value is determined using normal human fibroblast (NHF) cells, wherein the compound and a vehicle control (0.5% DMSO) are added to the cell solution and incubated for 72 h at 37° C. in 5% CO₂.

In some embodiments, a compound described herein, or a pharmaceutically acceptable salt or solvate thereof, has a stability such as an in vivo or ex vivo stability as measured by its reactivity profiling with glutathione. In some embodiments, the reactivity profiling is determined according to EXAMPLE B3. In some embodiments, a compound described herein, or a pharmaceutically acceptable salt or solvate thereof, has a T_{1/2} that is that is higher than 5 minutes, higher than 10 minutes, higher than 30 minutes, higher than 60 minutes, higher than 90 minutes, higher than 120 minutes, higher than 180 minutes, higher than 240 minutes, higher than 300 minutes, higher than 360 minutes, higher than 420 minutes, higher than 480 minutes, higher than 540 minutes, higher than 600 minutes, higher than 700 minutes, higher than 800 minutes, higher than 900 minutes, higher than 1000 minutes, higher than 1100 minutes, higher than 1200 minutes, higher than 1300 minutes, higher than 1400 minutes, or higher than 1500 minutes. In some embodiments, the T_{1/2} is determined in a glutathione (GSH) environment. In some embodiments, the T_{1/2}is determined according to EXAMPLE B3. In some embodiments, the T_{1/2} is determined using 5 μM of the compound with 0.5% DMSO in the presence of GSH (5 mM) and PBS buffer (pH 7.4) after incubation at 25° C. at 600 rpm, and quenched with 600 μL solution of acetonitrile at 0, 30, 60 and 120 minutes.

In some embodiments, a compound described herein, or a pharmaceutically acceptable salt or solvate thereof, has a cell permeability. In some embodiments, the cell permeability is measured in a parallel artificial membrane permeability assay (PAMPA). In some embodiments, the cell permeability is measured in a PAMPA assay according to EXAMPLE B4. In some embodiments, a compound described herein, or a pharmaceutically acceptable salt or solvate thereof, has a permeability of at least 1, at least 2, at least 3, at least 4, at least 5, at least 5.5, at least 6, at least 6.5, or at least 7 as expressed in Log Pe and determined in PAMPA. In some embodiments, a compound described herein, or a pharmaceutically acceptable salt or solvate thereof, has a permeability of at most 20, at most 10, at most 8, at most 7, at most 6.5, at most 5.5, at most 5.5, at most 5, or at most 4 as expressed in Log Pe and determined in PAMPA. In some embodiments, a compound described herein, or a pharmaceutically acceptable salt or solvate thereof, has a permeability within a range of from about 4 or 5 to about 6 or 7 as expressed in Log Pe and determined in PAMPA. In some embodiments, the PAMPA assay is performed using a PVDF (Polyvinylidene fluoride) artificial membrane between a donor compartment and an acceptor compartment with an incubation condition of about 25° C. and 60 rpm for 16 hours. In some embodiments, a starting concentration of the described compound in the donor compartment is 10 μM. In some embodiments, the acceptor compartment comprises 5 μL lecithin in dodecane solution (1.8% solution w/v) and 300 μL PBS buffer at pH 7.4. In some embodiments, the PAMPA assay is performed using a PVDF artificial membrane between a donor compartment and an acceptor compartment with an incubation condition of about 25° C. and 60 rpm for 16 hours, wherein the donor compartment comprises about 300 μL solution comprising the compound at a starting concentration of 10 μM and wherein the acceptor compartment comprises about 5 μL lecithin in dodecane solution (1.8% solution w/v) and 300 μL PBS buffer at pH 7.4. In some embodiments, the concentrations of the compound are determined by LC/MS/MS.

### Isosteres.

As used herein, “carboxylic acid or an isostere thereof” refers to a carboxylic acid moiety, or a functional group or moiety that exhibits similar physical, biological and/or chemical properties as a carboxylic acid moiety. Examples of carboxylic acid bioisosteres include, but are not limited to, hydroxamic acids, hydroxamic esters, sulfinic acids, sulfonic acids, sulfonamides, acyl-sulfonamides, sulfonylureas, acylureas, tetrazole, thiazolidine diones, oxozolidine diones, oxadiazol-5(4H)-one, oxothiadiazole-2-oxide, oxadiazol-5(4H)-thione, isoxazole, tetramic acid, cyclopentane 1,3-diones, cyclopentane 1,2-diones, phosphoric acids, phosphinic acids, and halogenated phenols. For example, a carboxylic acid isostere can be: —B(OH)₂, —S(O)₂NH₂,

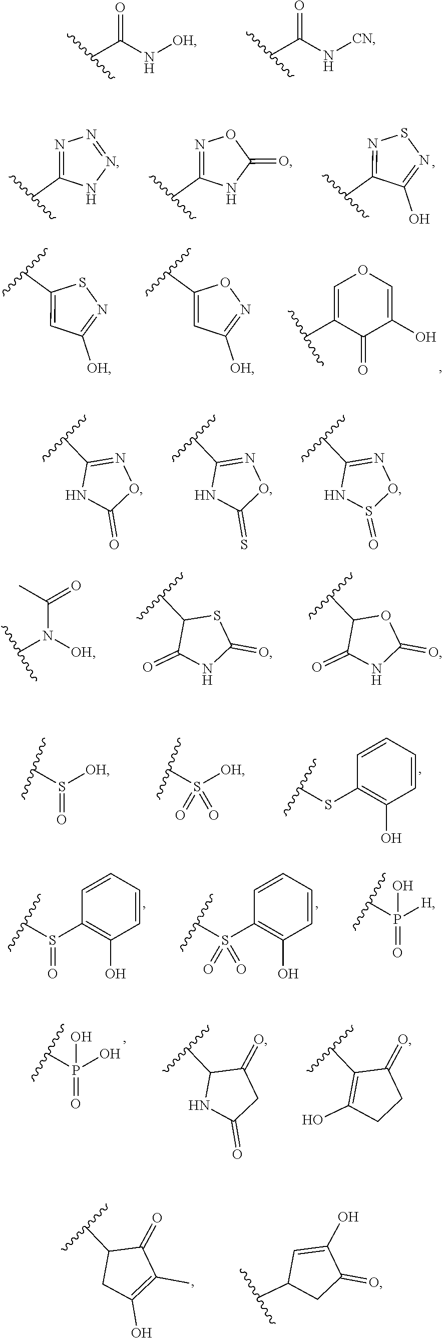

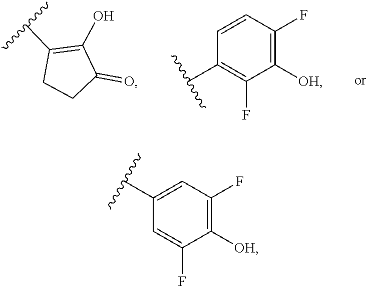 [SMILES: *C1=C(O)C(=O)CC1 ; *c1ccc(F)c(O)c1F ; *c1cc(F)c(O)c(F)c1]

wherein each hydrogen bound to a carbon atom is optionally replaced with methyl, ethyl, —CN, —CF₃, —OH, —OMe, —NH₂, or —NO₂, or a different halogen.

### Isomers/Stereoisomers.

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof In some situations, the compounds described herein possess one or more chiral centers and each center exists in the R configuration or S configuration. The compounds described herein include diastereomeric, enantiomeric, and epimeric forms as well as the corresponding mixtures thereof In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

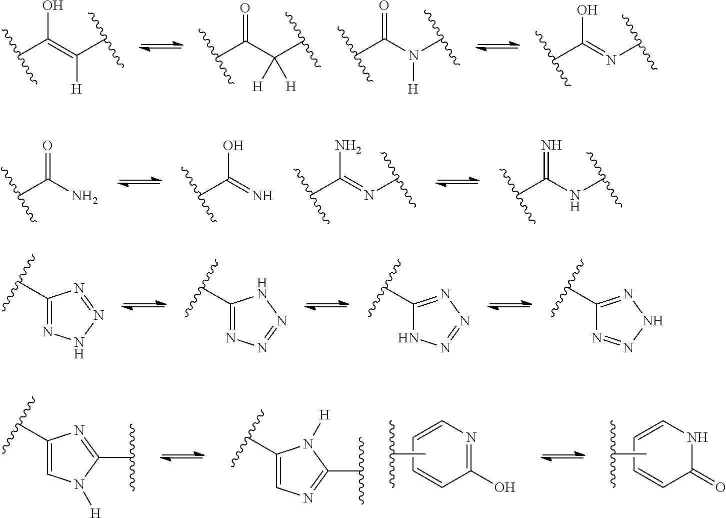

In some instances, the STAT5 inhibitory compounds disclosed herein exist in tautomeric forms. The structures of said compounds are illustrated in the one tautomeric form for clarity. The alternative tautomeric forms are expressly included in this disclosure.

### Labeled Compounds.

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Deuterated Compounds.

In certain embodiments, the abundance of ²H atoms in the compounds disclosed herein is enriched for some or all of the ¹H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

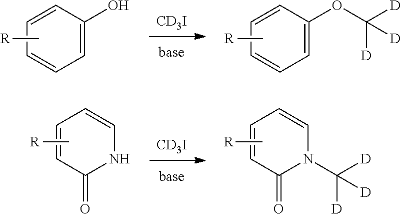

Deuterium-transfer reagents, such as lithium aluminum deuteride (LiAlD₄), are employed to transfer deuterium under reducing conditions to the reaction substrate. The use of LiAl₄ is illustrated, by way of example only, in the reaction schemes below.

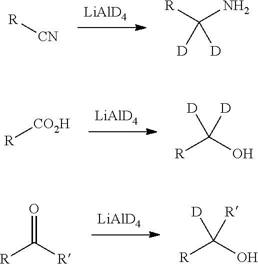

Deuterium gas and palladium catalyst are employed to reduce unsaturated carbon-carbon linkages and to perform a reductive substitution of aryl carbon-halogen bonds as illustrated, by way of example only, in the reaction schemes below.

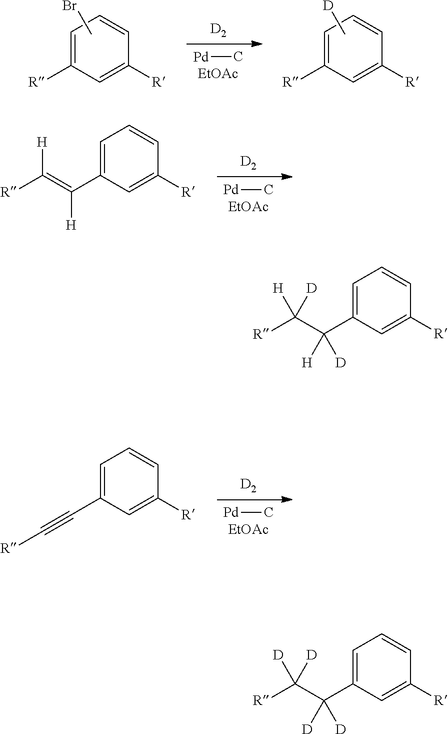

In some embodiments, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In some embodiments, the level of deuterium incorporation is determined by synthetic methods in which a deuterated synthetic building block is used as a starting material.

### Pharmaceutically Acceptable Salts.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates.

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Amorphous and Crystalline Forms.

The compounds described herein can exist in amorphous and/or crystalline forms, all of which are encompassed by the instant disclosure. In some embodiments, a herein described compound exists in an amorphous form. In some embodiments, a herein described compound exists in a crystalline form. One aspect of the present disclosure pertains to a crystalline polymorph of a compound described herein. In some embodiments, the crystalline polymorph is a stable polymorph of a described compound or a salt thereof.

The crystalline form of the described compounds can be identified by its unique solid state signature with respect to, for example, differential scanning calorimetry (DSC), X-ray powder diffraction (PXRD), and other solid state methods. Further characterization with respect to water or solvent content of the crystalline form can be gauged by any of the following methods for example, thermogravimetric analysis (TGA), DSC and the like. The crystalline polymorph can be prepared by any suitable method known in the art, for example, those described in K. J. Guillory, “Generation of Polymorphs, Hydrates, Solvates, and Amorphous Solids,” in: Polymorphism in Pharmaceutical Solids, ed. Harry G. Brittan, Vol. 95, Marcel Dekker, Inc:, New York, 1999, incorporated herein by reference in its entirety. In some embodiments, the crystalline polymorph is prepared by recrystallization. In some embodiments, the crystalline polymorph is a stable polymorph of a pharmaceutically acceptable salt of a compound described herein.

### Preparation of the Compounds.

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### III. Pharmaceutical Compositions.

In certain embodiments, the STAT5 inhibitory compound as described herein is administered as a pure chemical. In other embodiments, the STAT5 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one STAT5 inhibitory compound as described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described compound, or a pharmaceutically acceptable salt or solvate thereof, and a pharmaceutically acceptable excipient or carrier. In some embodiments, the disclosure provides a pharmaceutical composition comprising a compound of Formula (VI), (I), (II), (IIa), (IIb), (III), (IV), or (V), or a pharmaceutically acceptable salt or solvate thereof, and a pharmaceutically acceptable excipient or carrier.

In certain embodiments, the STAT5 inhibitory compound as described, such as a compound of Formula (VI), (I), (II), (IIa), (IIb), (III), (IV), or (V), is substantially pure, in that it contains less than about 5%, or less than about 1%, or less than about 0.1%, of other organic small molecules, such as unreacted intermediates or synthesis by-products that are created, for example, in one or more of the steps of a synthesis method.

The compounds and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The term parenteral as used herein includes subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. In certain embodiments, the compounds and formulations are administered orally. In certain embodiments, the compounds and formulations are administered topically.

In some embodiments, pharmaceutical compositions described herein are administered orally. Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)). In some embodiments, for solid dosage forms used in oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents, in the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

Compounds of the disclosure can also be administered via parenteral injection as liquid solution, which can include other chemical components, such as carriers, stabilizers, diluents, dispersing agents, suspending agents, thickening agents, preservatives, or excipients. Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water soluble form. For example, compositions described herein can be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used preservatives include chlorobutanol, m-cresol, benzyl alcohol, phenylethyl alcohol, phenol, methylparaben, or propylparaben. Commonly used buffers include histidine, acetate, phosphate, borate, or citrate. Commonly used tonicity adjustors include sodium chloride, mannitol and glycerin. The infusion solution may include 0 to 10% dextrose. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions, for example, a cyclodextrin or organic solvent. Organic solvents can include alcohols, for example, C₁-C₄ linear alkyl, C₃-C₄ branched alkyl, ethanol, ethylene glycol, glycerin, 2-hydroxypropanol, propylene glycol, maltitol, sorbitol, xylitol; substituted or unsubstituted aryl, and benzyl alcohol. Alternatively, the active ingredient can be in powder form for constitution with a suitable vehicle, e.g., sterile pyrogen-free water, before use.

The dose of the composition comprising at least one STAT5 inhibitory compound as described herein differ, depending upon the subject's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the subject, the type and severity of the subject's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject.

By way of example only, the dose of the compound described herein for methods of treating a disease as described herein is about 0.001 mg/kg to about 1 mg/kg body weight of the subject per day. In some embodiments, the dose of compound described herein for the described methods is about 0.001 mg to about 1000 mg per day for the subject being treated. In some embodiments, a compound described herein is administered to a subject at a daily dosage of from about 0.01 mg to about 500 mg, from about 0.01 mg to about 100 mg, or from about 0.01mg to about 50 mg.

### IV. Method of Treatment.

In one aspect, the disclosure provides a method of modulating signal transducer and activator of transcription proteins such as STAT5 and STAT3 in a subject in need thereof. In some embodiments, the methods comprise inhibiting STAT5 and/or STAT3 activities. In some embodiments, the method comprises administering to a subject a therapeutically effective amount a compound of Formula (VI), (I), (II), (IIa), (IIb), (III), (IV), or (V), or a pharmaceutically acceptable salt or solvate thereof. In some embodiments, the subject has cancer. In some embodiments, the cancer is a solid tumor or hematologic cancer.

Aberrant activation of STAT5 has been shown to contribute to malignant transformation and tumorigenesis. In particular, oncogenesis mediated by the aberrant activation of STAT5 is characterized in part by the transcriptional upregulation of genes that promote angiogenesis and tumor immune-tolerance. Therefore, modulating STAT5 signaling through the use of small-molecule inhibitors of STAT5 provides an effective and novel strategy for treating a wide variety of human tumors. STAT5-regulated genes include, but are not limited to, VEGF, Bcl.xL, matrix metalloproteinase 9, and c-Myc. In some embodiments, the present disclosure provides a method of decreasing the expression of VEGF, Bcl.xL, matrix metalloproteinase 9, or c-Myc in a cell, comprising contacting a compound of (VI), (I), (II), (IIa), (IIb), (III), (IV), or (V), or a pharmaceutically acceptable salt or solvate thereof with a cell.

In one aspect, the disclosure provides a method of treating cancer in a subject in need thereof. In some embodiments, the method comprises administering to a subject with cancer a therapeutically effective amount of a compound of Formula (VI), (I), (II), (IIa), (IIb), (III), (IV), or (V), or a pharmaceutically acceptable salt or solvate thereof. In some embodiments, the cancer is a solid tumor or hematologic cancer.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, colorectal cancer, gastric adenocarcinoma, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer.

In some embodiments, the subject is 5 to 75 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

Formation of transcriptionally active STAT5 can proceed through a phosphorylation-dimerization pathway, whereby STAT5 is first phosphorylated on a key tyrosine residue to provide phosphorylated STAT5 (pSTAT5), and the resulting phosphotyrosine residue binds to a Src-homology 2 (SH2) domain of another STAT5 or pSTAT5 protein. A pSTAT5 homodimer can then undergo nuclear transport and participate in direct DNA binding. In some embodiments, the present disclosure provides a method of inhibiting the formation of STAT5:pSTAT5 or pSTAT5:pSTAT5 hetero- or homodimers by contacting a cell with a compound of Formula (VI), (I), (II), (IIa), (IIb), (III), (IV), or (V). In some embodiments, the compound of Formula (VI), (I), (II), (IIa), (IIb), (III), (IV), or (V) binds to the SH2 domain of STAT5 or pSTAT5. In some embodiments, a compound described herein is an inhibitor of STAT dimerization, an inhibitor of a tyrosine kinase capable of phosphorylating STAT, an antagonist of SH2-pY interactions, an antagonist of STAT DNA binding, a tyrphostin inhibitor, an antagonist of STAT-dependent gene transactivation, an antagonist of IL-6 receptor activation, an antagonist of a cytokine that constitutively activates STAT, or an antagonist of a growth factor that constitutively activates STAT.

As used herein, the term “STAT5” can refer to a transcription factor encoded by the human STAT5a or STAT5b genes. The term is inclusive of splice isoforms or variants, as well as any non-human orthologs or homologs thereof.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### A: Synthesis of the Compounds.

The compounds of TABLE 1 and TABLE 2 have been synthesized according to organic synthesis techniques known to those skilled in this art, starting from commercially available chemicals and/or from compounds described in the chemical literature. The compounds of the disclosure and their syntheses are further illustrated by the following examples. A skilled person in the art would appreciate that other compounds of the disclosure, such as compounds of Formula (VI), (I), (II), (IIa), (IIb), (III), (IV), or (V), can be synthesized by similar approaches.

### Example A1: Synthesis of Compound 1116

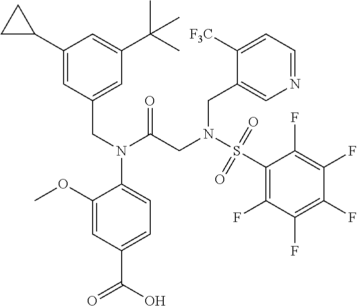 [SMILES: COc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F]

### Synthesis of Compound 1A.

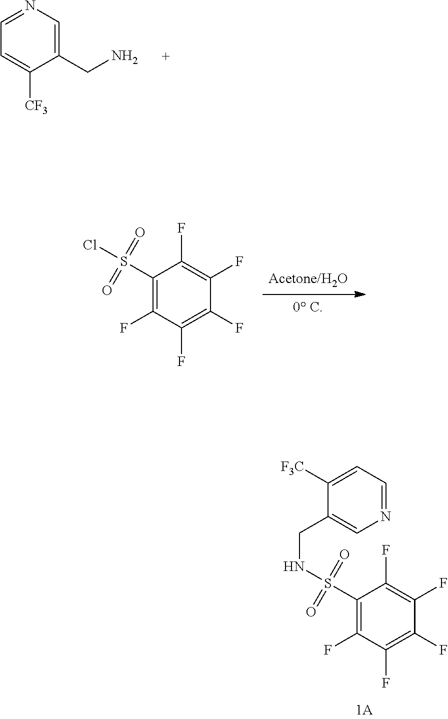 [SMILES: NCc1cnccc1C(F)(F)F ; O=S(=O)(Cl)c1c(F)c(F)c(F)c(F)c1F ; "0° C." ; "Acetone/H2O" ; O=S(=O)(NCc1cnccc1C(F)(F)F)c1c(F)c(F)c(F)c(F)c1F]

C-(4-Trifluoromethyl-pyridin-3-yl)methylamine hydrochloride (0.655 g, 3.08 mmol) was added to a solution of 2,3,4,5,6-pentafluorobenzenesulfonyl chloride (1.2 g, 4.50 mmol, 666.67 μL) and sodium bicarbonate (776.48 mg, 9.24 mmol, 359.48 μL) in acetone (20 mL) and water (5 mL) at 0° C. The resulting mixture was allowed to warm to room temperature, stirred for 1 hour, and purified by flash column chromatography to provide Compound 1A (1.1 g, 2.71 mmol, 87.89% yield).

### Synthesis of Compound 1B.

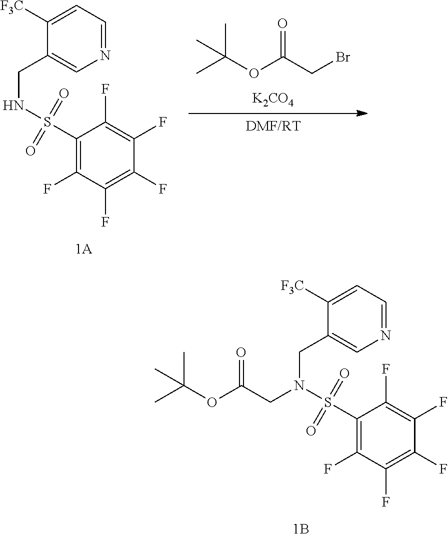

tert-Butyl 2-bromoacetate (792.22 mg, 4.06 mmol, 600.16 μL) was added to a solution of Compound 1A (1.1 g, 2.71 mmol) and potassium carbonate (748.46 mg, 5.42 mmol) in dimethylformamide (DMF) (25 mL) at 0° C. The reaction mixture was allowed to stir for 2 hours at room temperature (RT). The reaction mixture was diluted with ethyl acetate (EtOAc) (300 mL) and washed with water (3×200 mL). The organic layer was then dried with sodium sulfate, filtered, concentrated under vacuum, and the residue was purified by flash column chromatography to provide Compound 1B (660 mg, 1.27 mmol, 46.84% yield).

### Synthesis of Compound IC.

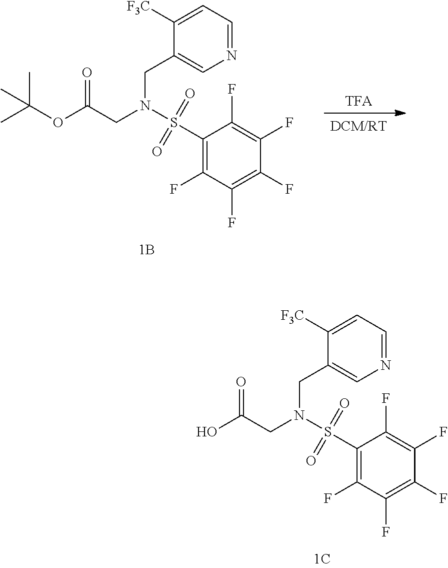 [SMILES: CC(C)(C)OC(=O)CN(Cc1cnccc1C(F)(F)F)S(=O)(=O)c1c(F)c(F)c(F)c(F)c1F ; "DCM/RT" ; "TFA" ; O=C(O)CN(Cc1cnccc1C(F)(F)F)S(=O)(=O)c1c(F)c(F)c(F)c(F)c1F]

Trifluoroacetic acid (2.98 g, 26.14 mmol, 2 mL) was added to a solution of Compound 1B (660 mg, 1.27 mmol) in dichloromethane (DCM) (2 mL) at room temperature, and the resulting mixture was stirred for 1 hour. The reaction mixture was concentrated by air stream and treated with 4M HCl in dioxane (2 mL) for 10 minutes. The mixture was concentrated, and the HCl treatment and concentration was repeated two more times to provide Compound 1C (580 mg, 1.25 mmol, 98.50% yield).

### Synthesis of Compound 1D.

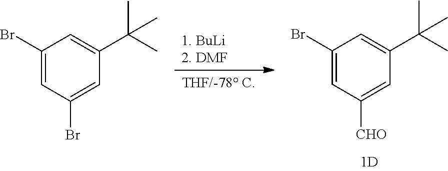

In a 250 ml flame/vacuum-dried round bottom flask, 1,3-dibromo-5-tert-butyl-benzene (5.21 g, 17.83 mmol) was dissolved in tetrahydrofuran (THF) (99.01 mL). n-Butyllithium (2.5 M, 7.49 mL) was then added dropwise at −78° C. to the resulting solution. DMF (7.82 g, 106.97 mmol, 8.28 mL) was added dropwise and then stirred for additional 15 minutes at −78° C. The reaction was then quenched by addition of a saturated aqueous solution of NH₄Cl (100 mL), the product was extracted with EtOAc (2×200 mL), and the combined organic phases were washed with a brine solution (100 mL). The combined organic phases were concentrated under vacuum, and the resulting residue was purified by flash column chromatography to provide compound 1D (3.37 g, 13.98 mmol, 78.3% yield).

### Synthesis of Compound 1E.

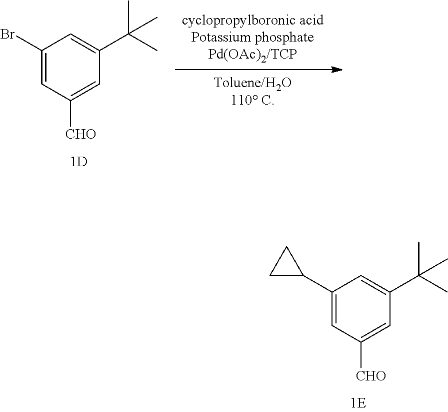

In a 250 ml flame/vacuum dried round bottom flask, Compound 1D (5.2 g, 21.57 mmol) was dissolved in toluene (97.05 mL). Cyclopropylboronic acid (2.04 g, 23.72 mmol), potassium phosphate (18.31 g, 86.26 mmol) and tricyclohexylphosphine (TCP) (604.76 mg, 2.16 mmol) were then added sequentially. After 10 minutes stirring under nitrogen gas flow, water (10.78 mL) was added to the suspension. Finally, palladium (II) acetate (242.08 mg, 1.08 mmol) was added, and the reaction was stirred at 110° C. for 16 hours. The reaction mixture was then diluted with water (100 mL), extracted with EtOAc (3×100 mL), and the combined organic phases were concentrated to dryness under vacuum. The resulting concentrate was purified by flash column chromatography to provide Compound 1E (3.53 g, 17.47 mmol, 81% yield).

### Synthesis of Compound 1F.

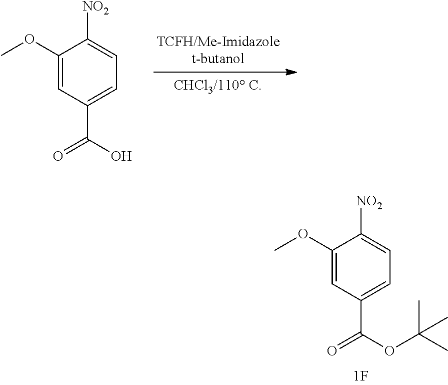 [SMILES: COc1cc(C(=O)O)ccc1[N+](=O)[O-] ; COc1cc(C(=O)OC(C)(C)C)ccc1[N+](=O)[O-]]

Tetramethylchloroformamidinium hexafluorophosphate (TCFH) (0.782 g, 2.79 mmol) and chloroform (15 mL) were added to a flame-dried microwave vial (20 mL) equipped with a stir bar. 3-methoxy-4-nitro-benzoic acid (0.5 g, 2.54 mmol) was then added to the mixture immediately thereafter, and the heterogeneous solution was allowed to stir for 5 mins. 1-Methylimidazole (624.67 mg, 7.61 mmol, 606.47 μL) was then added, and the vial was sealed and heated for 1 hour at 110° C. The reaction mixture was purified by flash column chromatography to afford Compound 1F (0.491 g, 1.94 mmol, 74% yield).

### Synthesis of Compound 1G.

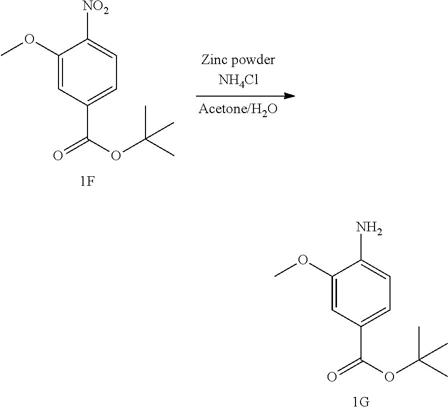 [SMILES: COc1cc(C(=O)OC(C)(C)C)ccc1[N+](=O)[O-] ; COc1cc(C(=O)OC(C)(C)C)ccc1N]

Compound 1F (1.37 g, 5.41 mmol), acetone (40 mL), and water (10 mL) was added to a round bottom flask. Zinc Powder (7.07 g, 108.19 mmol) and ammonium chloride (11.57 g, 216.39 mmol, 7.57 mL) were then added as single, separate portions. The reaction mixture was allowed to stir at room temperature for 7 hours, and then concentrated onto silica gel and purified by flash column chromatography to provide compound 1G (0.870g, 3.90 mmol, 72% yield).

### Synthesis of Compound 1H.

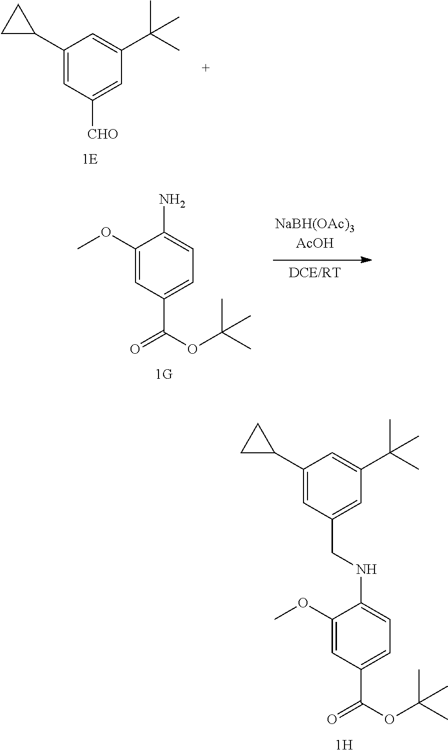 [SMILES: CC(C)(C)c1cc(C=O)cc(C2CC2)c1 ; COc1cc(C(=O)OC(C)(C)C)ccc1N ; COc1cc(C(=O)OC(C)(C)C)ccc1NCc1cc(C2CC2)cc(C(C)(C)C)c1]

Compound 1E (231 mg, 1.14 mmol) and dichloroethane (10 mL) was added to a round bottom flask, and the resulting solution was allowed to stir at room temperature for 2 minutes. Compound 1G (254.95 mg, 1.14 mmol) was subsequently added as a solid directly to the stirring aldehyde solution. After 2 minutes, sodium triacetoxyborohydride (363.03 mg, 1.71 mmol) was added to the heterogeneous solution, which immediately afforded a dark-yellow solution. After 2 minutes, acetic acid (468.00 mg, 7.79 mmol, 445.71 μL) was added, and the resulting mixture was allowed to stir for 3 hours. The crude was then purified by flash column chromatography to provide Compound 111 (444 mg, 1.08 mmol, 94.94% yield).

### Synthesis of Compound 1I.

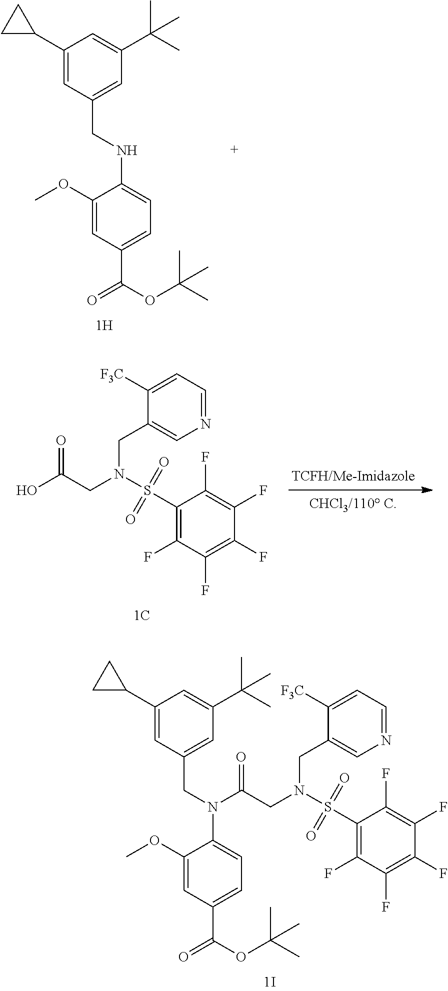 [SMILES: COc1cc(C(=O)OC(C)(C)C)ccc1NCc1cc(C2CC2)cc(C(C)(C)C)c1 ; O=C(O)CN(Cc1cnccc1C(F)(F)F)S(=O)(=O)c1c(F)c(F)c(F)c(F)c1F ; COc1cc(C(=O)OC(C)(C)C)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F]

Compound 1C (500 mg, 1.08 mmol) and chloroform (10.59 mL) were added to a flame-dried microwave vial (20 mL) equipped with a stir bar. Compound 111 (529.28 mg, 1.29 mmol) was added to the mixture immediately thereafter, and the resulting heterogeneous solution was allowed to stir for 5 minutes. 1-Methylimidazole (180 2.26 mmol) and TCFH (332.69 mg, 1.19 mmol) were then added, and the vial was sealed and heated for 1 hour at 110° C. The reaction was concentrated into a 20 mL scintillation vial and carried over to the next step without any purification.

### Synthesis of Compound 1J

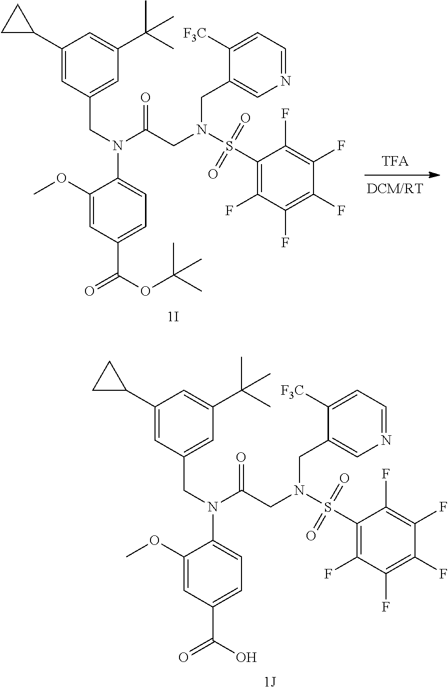 [SMILES: COc1cc(C(=O)OC(C)(C)C)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F ; COc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F]

DCM (3 mL) and TFA (3 mL) were added to a 20 mL scintillation vial containing crude Compound 1I (580 mg, 677.70 μmol). The resulting solution was allowed to stir for 60 minutes at room temperature, whereafter the solvents were removed by air steam, and the reaction mixture was purified by preparative HPLC to provide Compound 1J (0.532 g, 665 mmol, 98% yield).

### Example A2. Synthesis of Compound 1167

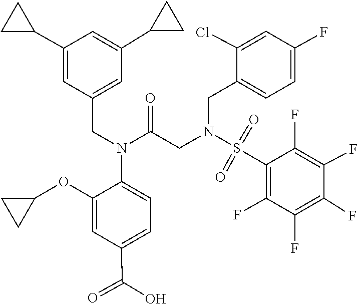 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1]

### Synthesis of Compound 2A.

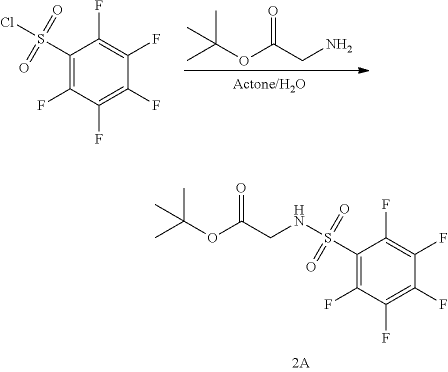

Glycine tert-butyl acetate hydrochloride (1 g, 5.97 mmol) was added to a solution of pentafluorobenzene sulfonyl chloride (2.39 g, 8.95 mmol) and sodium bicarbonate (1.50 g, 17.90 mmol, 696.05 μL) in acetone (47 mL) and water (15 mL) at 0° C. The reaction was stirred at 0° C. for 1 hour, and then stirred at room temperature for 3 hours. The reaction mixture was concentrated directly onto silica and purified by flash column chromatography provide Compound 2A (1.43 g, 3.96 mmol, 66% yield).

### Synthesis of Compound 2B.

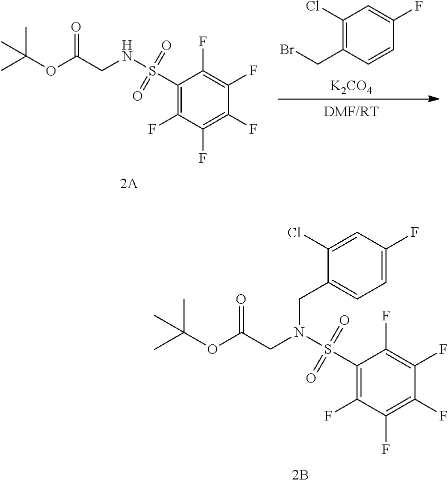

Compound 2A (890 mg, 2.46 mmol, in 12 mL DMF solution) was added dropwise to a solution of 1-(bromomethyl)-2-chloro-4-fluoro-benzene (658.60 mg, 2.95 mmol) and potassium carbonate (510.71 mg, 3.70 mmol) in DMF (12 mL), and the resulting mixture was stirred at room temperature for 2 hours. The product was extracted with EtOAc (150 mL), and the organic layer was washed with water (3×75 mL). The organic layer was concentrated under vacuum, and the residue was purified flash column chromatography to provide Compound 2B (0.897 g, 1.78 mmol, 72% yield).

### Synthesis of Compound 2C.

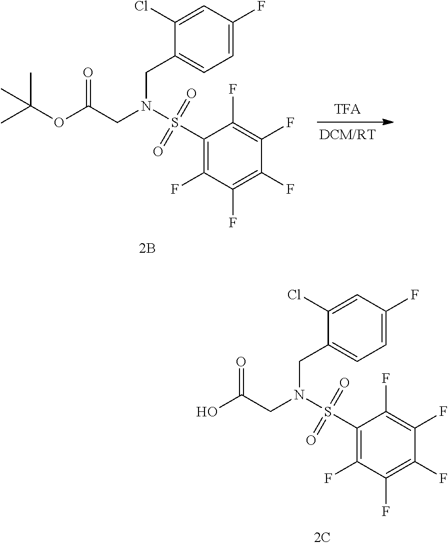 [SMILES: CC(C)(C)OC(=O)CN(Cc1ccc(F)cc1Cl)S(=O)(=O)c1c(F)c(F)c(F)c(F)c1F ; O=C(O)CN(Cc1ccc(F)cc1Cl)S(=O)(=O)c1c(F)c(F)c(F)c(F)c1F ; "TFA"]

DCM (3 mL) and TFA (3 mL) were added to a scintillation vial containing Compound 2B (893 mg, 1.77 mmol) at room temperature. The resulting solution was allowed to stir for 1 hour, whereafter the solvents were removed by air steam. The residual TFA was removed by redissolving the residue with 5 mL chloroform and concentrating the resulting solution via air stream. The redissolution and concentration was then repeated 3 times to provide Compound 2C (0.706 g, 1.58 mmol, 89% yield).

### Synthesis of Compound 2D.

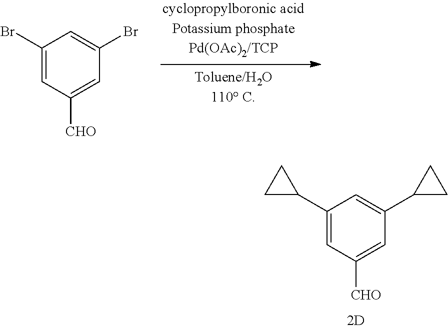 [SMILES: O=Cc1cc(Br)cc(Br)c1 ; O=Cc1cc(C2CC2)cc(C2CC2)c1]

To an oven-dried round bottom flask equipped with a stirbar, 3,5-dibromobenzaldehyde (3 g, 11.37 mmol), cyclopropylboronic acid (5.86 g, 68.20 mmol), tricyclohexylphosphine (318.77 mg, 1.14 mmol), potassium phosphate tribasic (4.19 g, 19.74 mmol), palladium acetate (127.60 mg, 568.37 μmol), toluene (20 mL), and H₂O (1.24 mL) were added, and the flask was placed in an oil bath heated to 100° C. and allowed to stir for 18 hours. The reaction mixture was concentrated under vacuum onto silica, and the residue was purified via flash column chromatography to provide Compound 2D (1.54 g, 8.26 mmol, 72% yield).

### Synthesis of Compound 2E.

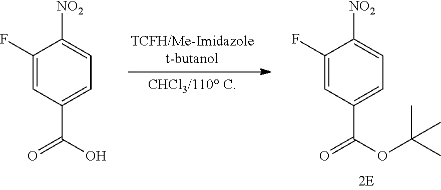 [SMILES: O=C(O)c1ccc([N+](=O)[O-])c(F)c1 ; CC(C)(C)OC(=O)c1ccc([N+](=O)[O-])c(F)c1]

In a 500 mL flame dried round bottom flask, 3-fluoro-4-nitro-benzoic acid (5 g, 27.01 mmol) was diluted in chloroform (130.32 mL) (ca.) to provide a suspension. 1-Methylimidazole (4.88 g, 59.42 mmol, 4.74 mL) and TCFH (8.34 g, 29.71 mmol) were then added to the suspension. t-BuOH was subsequently added (20.02 g, 270.11 mmol), and the flask was then transferred to an oil bath and stirred for 2 hours at 100° C. The reaction mixture is concentrated onto silica and purified by flash column chromatography to provide Compound 2E (6.07 g, 25.16 mmol, 93% yield).

### Synthesis of Compound 2F.

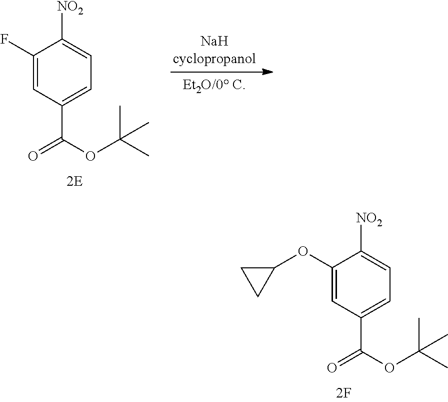 [SMILES: CC(C)(C)OC(=O)c1ccc([N+](=O)[O-])c(F)c1 ; CC(C)(C)OC(=O)c1ccc([N+](=O)[O-])c(OC2CC2)c1]

To a vial containing Compound 2E (200 mg, 829.14 μmol) was added Et₂O (9.12 mL), followed by the careful addition of sodium hydride (39.80 mg, 1.66 mmol) at 0° C. The reaction was allowed to equilibrate to room temperature, and then stirred for 3 hours at room temperature. The reaction mixture was concentrated onto silica and purified by flash column chromatography provide Compound 2F (0.137 g, 0.493 mmol, 59% yield).

### Synthesis of Compound 2G.

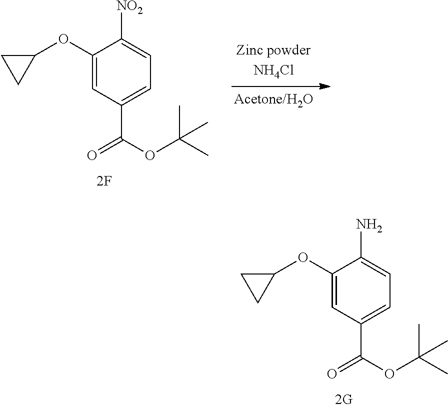 [SMILES: CC(C)(C)OC(=O)c1ccc([N+](=O)[O-])c(OC2CC2)c1 ; CC(C)(C)OC(=O)c1ccc(N)c(OC2CC2)c1]

Compound 2F (160 mg, 504.14 μmol) and ammonium chloride (1.07 g, 20.02 mmol) were diluted in acetone (8.43 mL) in a 20 ml scintillation vial, to provide a yellow solution. Water (1.65 mL), followed by zinc powder (662.01 mg, 10.12 mmol) were added, and the mixture was stirred for 2 hours at 25° C. The reaction was then filtered, concentrated onto silica, and purified by flash column chromatography to provide Compound 2G (0.125 g, 0.501 mmol, 99% yield).

### Synthesis of Compound 2H.

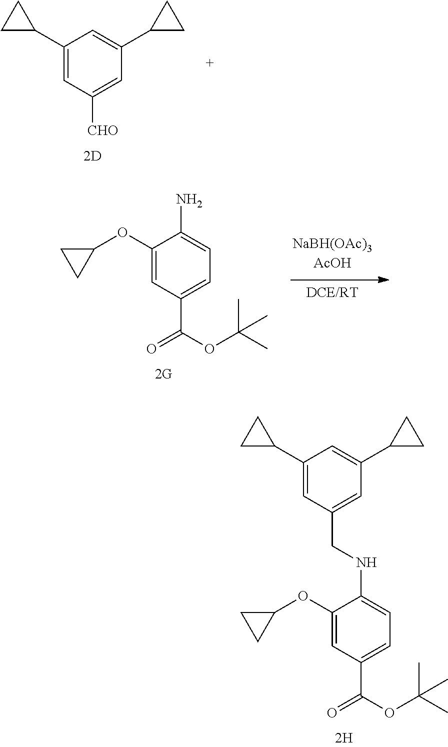 [SMILES: O=Cc1cc(C2CC2)cc(C2CC2)c1 ; CC(C)(C)OC(=O)c1ccc(N)c(OC2CC2)c1 ; CC(C)(C)OC(=O)c1ccc(NCc2cc(C3CC3)cc(C3CC3)c2)c(OC2CC2)c1]

To a solution of Compound 2D (40.01 mg, 214.80 μmol) and Compound 2G (51.2 mg, 204.57 μmol) in dichloroethane (DCE) (2.05 mL) was added glacial acetic acid (0.2 mL) followed by sodium triacetoxyborohydride (130.07 mg, 613.71 μmol) at room temperature. The reaction was allowed to stir for 3 hours at room temperature, and then concentrated onto silica and purified by flash column chromatography provide Compound 211 (65.7 mg, 156.7 μmol, 76% yield).

### Synthesis of Compound 2I.

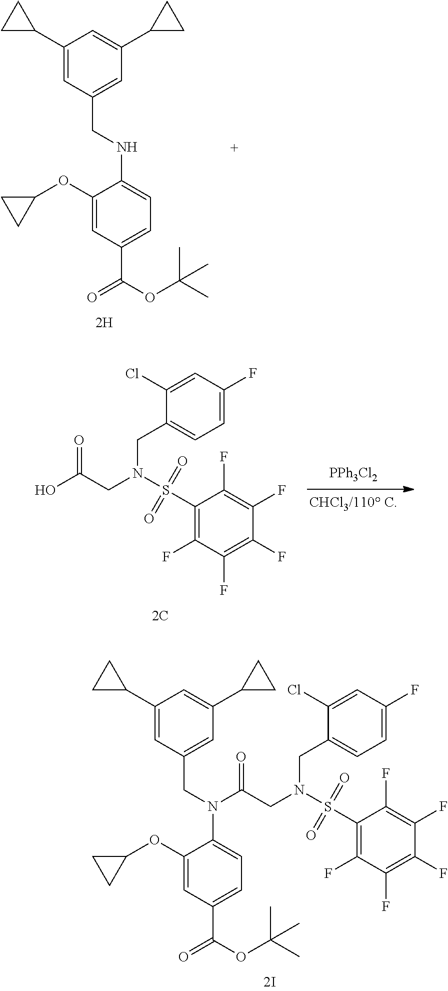 [SMILES: CC(C)(C)OC(=O)c1ccc(NCc2cc(C3CC3)cc(C3CC3)c2)c(OC2CC2)c1 ; O=C(O)CN(Cc1ccc(F)cc1Cl)S(=O)(=O)c1c(F)c(F)c(F)c(F)c1F ; CC(C)(C)OC(=O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1 ; "PPh3Cl2"]

Compound 2H (267 mg, 636.39 μmol) and chloroform (6.36 mL) was added to a flame-dried microwave vial (20 mL) equipped with a stir bar. Compound 2C (284.93 mg, 636.39 μmol) was added to the mixture at room temperature immediately thereafter, and the resulting heterogeneous solution was allowed to stir for 10 mins. Dichlorotriphenylphosphorane (508.89 mg, 1.53 mmol) was then added at room temperature. The vial was sealed and stirred in an oil bath for 2 hours at 110° C. The reaction was concentrated into a 20 mL scintillation vial and carried over to the next step without any purification.

### Synthesis of Compound 2J

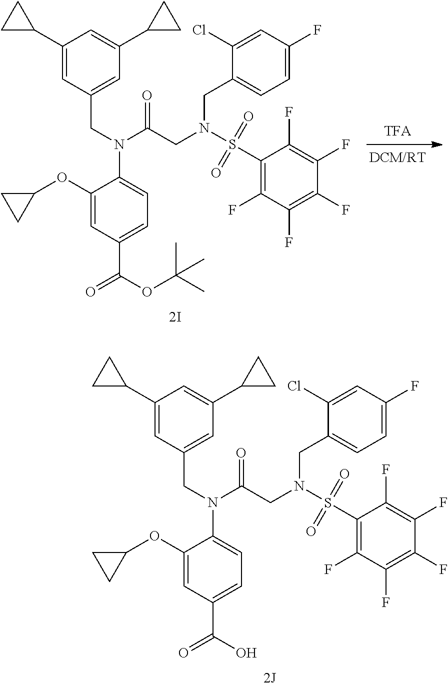 [SMILES: CC(C)(C)OC(=O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1 ; "TFA" ; O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1]

DCM (5 mL) and TFA (5 mL) were added to a scintillation vial containing crude Compound 2I (2 g, 2.35 mmol) at room temperature. The resulting solution was allowed to stir for 60 minutes, whereafter the solvents were removed by air stream. The reaction mixture was then purified by HPLC to provide Compound 2J (0.990 g, 1.25 mmol, 53% yield).

### Example A3. Synthesis of Compound 1252

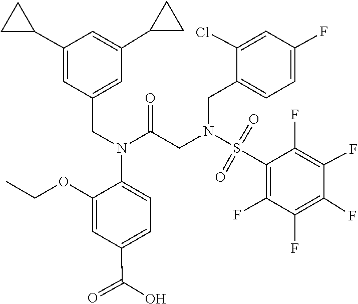 [SMILES: CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F]

### Synthesis of Compound 4A.

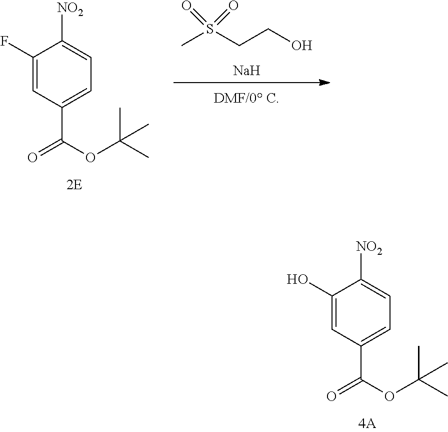

2-(Methylsulfonyl-ethanol) (154 mg, 1.24 mmol) was added to a stirring solution of Compound 2E (200 mg, 829.14 μmol) in DMF (1.26 mL), and the resulting mixture was cooled to 0° C. NaH (59 mg, 2.49 mmol) was then added, and the reaction was stirred for 3 hours. The reaction was then warmed to room temperature, quenched with a 1N HCl solution, and partitioned between ethyl acetate and brine. The organic layer was concentrated to dryness, and the residue was purified flash column chromatography to afford Compound 4A (198.5 mg, 827.6 μmol, 99% yield).

### Synthesis of Compound 4B.

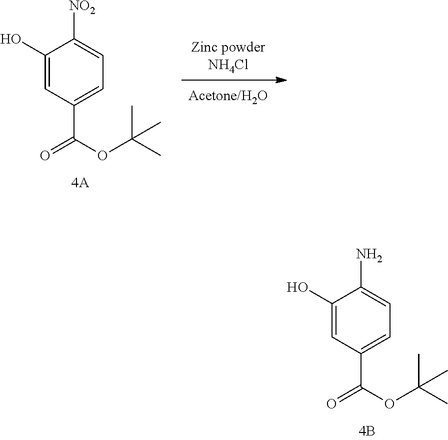 [SMILES: CC(C)(C)OC(=O)c1ccc([N+](=O)[O-])c(O)c1 ; "Zinc powderNH4Cl" ; CC(C)(C)OC(=O)c1ccc(N)c(O)c1]

Compound 4A (198 mg, 827.68 μmol), ammonium chloride (1.76 g, 32.87 mmol), acetone (12.18 mL), and water (2.38 mL) were combined in a 20 ml scintillation vial. Zinc powder (1.09 g, 16.62 mmol) was then added and the mixture was stirred for 1 hour at 25° C. The reaction mixture was filtered, concentrated, redissolved in MeOH, and K₂CO₃ was added. The resulting suspension was stirred for 20 minutes, filtrated, concentrated, and then purified by flash column chromatography to provide Compound 4B (145.4 mg, 694.8 μmol, 83% yield).

### Synthesis of Compound 4C.

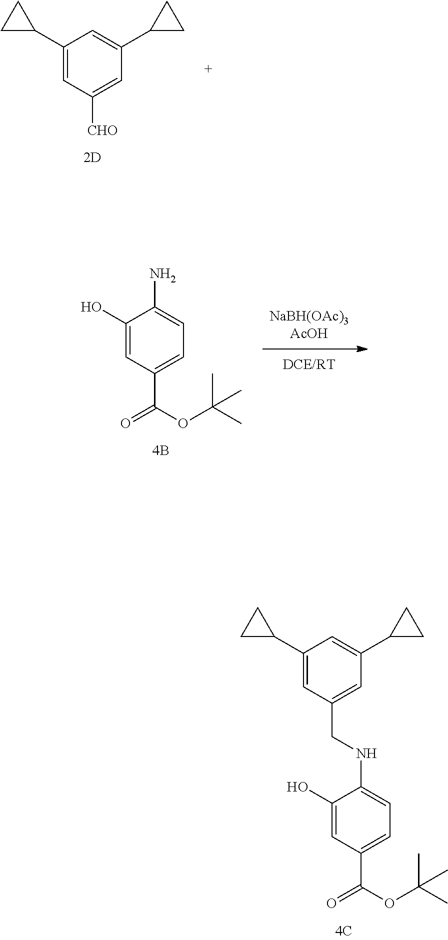 [SMILES: O=Cc1cc(C2CC2)cc(C2CC2)c1 ; CC(C)(C)OC(=O)c1ccc(N)c(O)c1 ; CC(C)(C)OC(=O)c1ccc(NCc2cc(C3CC3)cc(C3CC3)c2)c(O)c1 ; "NaBH(OAc)3AcOH"]

Compound 2D (380.08 mg, 2.04 mmol), Compound 4B (427 mg, 2.04 mmol), and DCE (19.71 mL) were combined in a flame dried 100 ml round bottom flask charged with molecular sieves (4 A). Acetic acid (735.27 mg, 12.24 mmol, 700.25 μL) was then added incrementally in small portions. Na(OAc)₃BH (1.08 g, 5.10 mmol) was added in a single portion, and the reaction was stirred at 25° C. for 4 hours. The solution was concentrated onto silica, and the residue was purified by flash column chromatography to provide Compound 4C (512.1 mg, 1.33 mmol, 66% yield).

### Synthesis of Compound 4D.

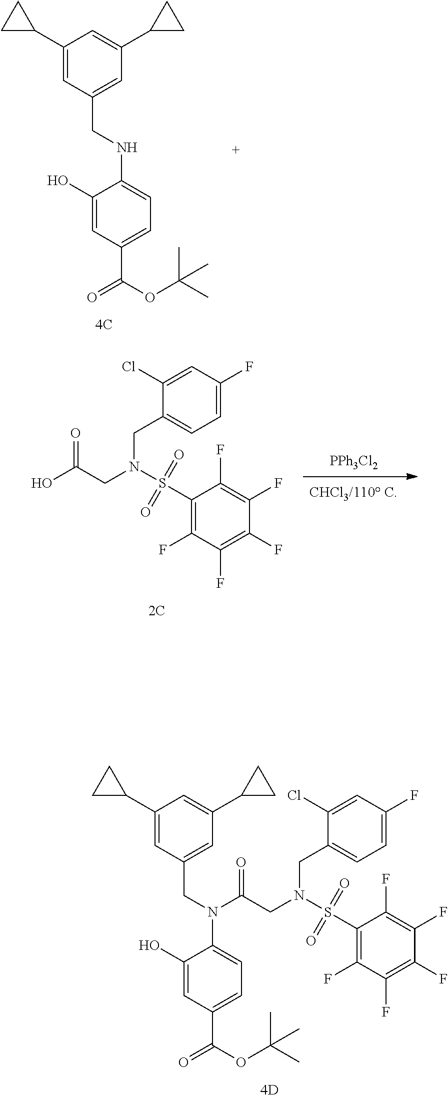 [SMILES: CC(C)(C)OC(=O)c1ccc(NCc2cc(C3CC3)cc(C3CC3)c2)c(O)c1 ; O=C(O)CN(Cc1ccc(F)cc1Cl)S(=O)(=O)c1c(F)c(F)c(F)c(F)c1F ; CC(C)(C)OC(=O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(O)c1 ; "PPh3Cl2"]

Compound 2C (579 mg, 1.29 mmol) was dissolved in anhydrous chloroform (12.72 mL) in a 20 mL microwave vial. Then, 1-methylimidazole (247.97 mg, 3.02 mmol, 240.75 μL) and TCFH (423.73 mg, 1.51 mmol) were added sequentially. Compound 4C (521 mg, 1.37 mmol) was then added, and the resulting solution was sealed and refluxed at 110° C. for 1 hour. The solution was concentrated, and the residue purified by flash column chromatography to provide Compound 4D (462.3 mg, 571.2 μmol, 41.6% yield).

### Synthesis of Compound 4E.

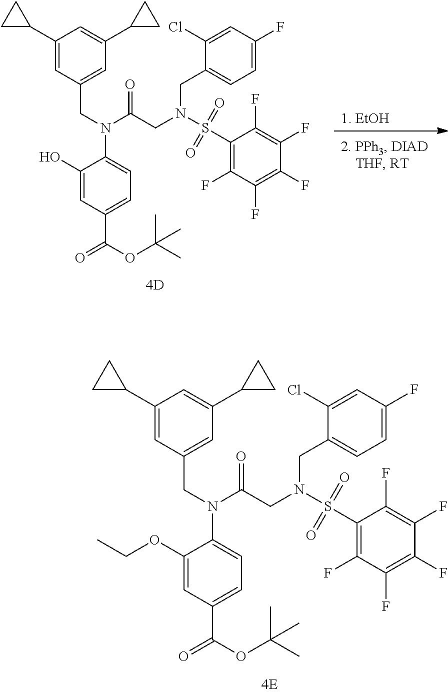 [SMILES: CC(C)(C)OC(=O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(O)c1 ; "1. EtOH" ; CCOc1cc(C(=O)OC(C)(C)C)ccc1N(Cc1cc(C2CC2)cc(C2CC2)c1)C(=O)CN(Cc1ccc(F)cc1Cl)S(=O)(=O)c1c(F)c(F)c(F)c(F)c1F]

Triphenylphosphine (14.30 mg, 54.53 μmol) and diisopropyl azodicarboxylate (DIAD) (13.76 mg, 54.53 μmol) were diluted with THF (357.79 μL) in a 1.5 mL centrifuge tube (150 μL) and stirred at 25° C. In separate tube, Compound 4D (29.42 mg, 36.36 μmol) and ethanol (3.35 mg, 72.71 μmol, 4.25 μL) were diluted with THF (357.79 μL). After complete dissolution, the solution of Compound 4D was added dropwise to the first solution, and the resulting mixture was stirred for 2 hours. The reaction was then concentrated, and the residue was purified by flash column chromatography to provide Compound 4E (30.0 mg, 35.83 μmol, 98.2% yield).

### Synthesis of Compound 4F.

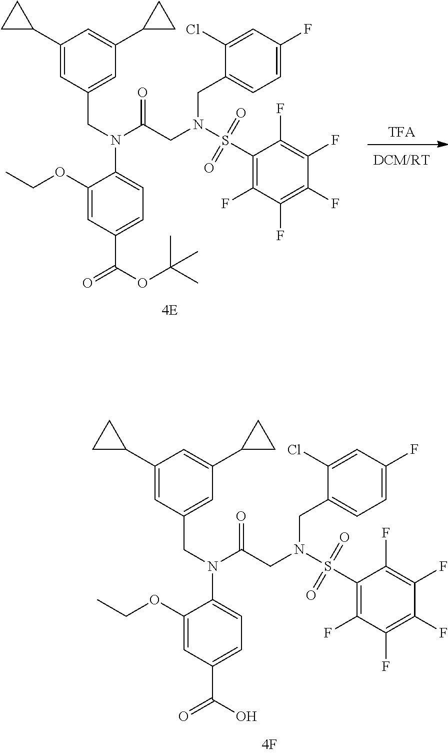 [SMILES: CCOc1cc(C(=O)OC(C)(C)C)ccc1N(Cc1cc(C2CC2)cc(C2CC2)c1)C(=O)CN(Cc1ccc(F)cc1Cl)S(=O)(=O)c1c(F)c(F)c(F)c(F)c1F ; CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F ; "TFA"]

In a 20 mL scintillation vial, Compound 4E (30 mg, 35.83 μmol) was dissolved in DCM (2 mL), and then diluted with trifluoroacetic acid (TFA) (2 mL). The resulting solution was stirred for 1 hour at 25° C., concentrated, and purified by HPLC to provide Compound 4F (12.9 mg, 16.51 μmol, 46% yield).

### Example A4—General Procedure F

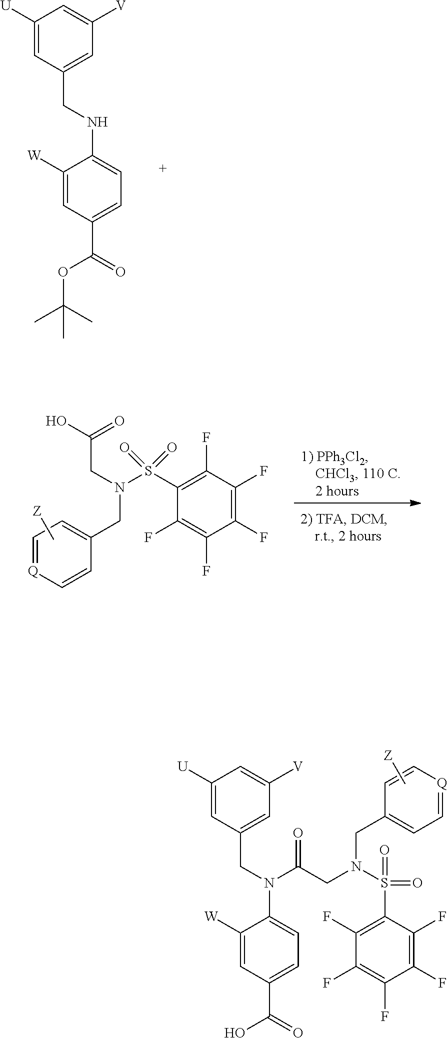

Under an inert atmosphere of nitrogen gas, a secondary aniline (1 eq.) and a functionalized glycine (1.2 eq.) were dissolved in anhydrous chloroform (0.1-0.25 M). The resulting mixture was stirred at room temperature for 10 minutes before neat dichlorotriphenylphosphorane (2.2-2.5 eq.) was added in one portion. The reaction was heated to and maintained at a temperature of 110° C. for 2 hours and subsequently cooled to room temperature. Once at ambient temperature, the solvent was evaporated off and the remaining residue re-suspended in a 2:1 (v/v) mixture of anhydrous dichloromethane (DCM) and trifluoroacetic acid (TFA). After 2 hours, the solvent was concentrated in vacuo and residual TFA co-distilled off with chloroform. The crude reaction mixture was purified by Prep-HPLC, running a mobile phase of 50% to 0% H₂O (0.1% FA) in ACN (0.1% FA) over 60 minutes, and the product containing fractions lyophilized to afford the desired product. Each U, V, W, and Z refers to a relevant substituent as described herein.

### Example A5—Synthesis of Compound 1272

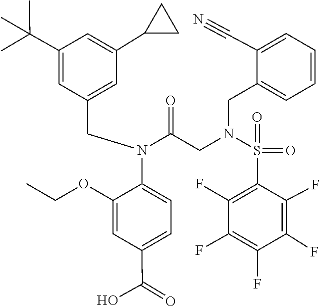 [SMILES: CCOc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F]

The title compound, 4-(N-(3-(tert-butyl)-5-cyclopropylbenzyl)-2-((N-(2-cyanobenzyl)-2,3,4,5,6-pentafluorophenyl)sulfonamido)acetamido)-3-ethoxybenzoic acid, was prepared according to the protocol described in general procedure F and isolated as a white powder (0.049 g, 69% yield). ¹H NMR (400 MHz, CDCl₃) δ 7.70 (d, J=7.9 Hz, 1H), 7.67-7.56 (m, 3H), 7.52 (d, J=1.6 Hz, 1H), 7.43 (t, J=7.6 Hz, 1H), 7.06 (d, J=8.1 Hz, 1H), 6.97 (s, 1H), 6.70 (s, 1H), 6.63 (s, 1H), 5.02 (d, J=15.5 Hz, 1H), 4.85-4.60 (m, 3H), 4.18 (d, J=17.1 Hz, 1H), 4.11-3.93 (m, 1H), 3.84-3.59 (m, 2H), 1.84 (ddd, J=13.5, 8.5, 5.1 Hz, 1H), 1.29 (t, J=7.0 Hz, 3H), 1.19 (s, 9H), 0.99-0.90 (m, 2H), 0.67-0.53 (m, 2H). ¹⁹F NMR (376 MHz, CDCl₃) δ −133.96-−134.17 (m, 2F), −146.14-−146.37 (m, 1F), −159.43-−159.66 (m, 2F). ESI-MS: measured m/z 768.1 [M−H]⁻. Purity by HPLC: 99.9% at 254 nm.

### Example A6—Synthesis of Compound 1273

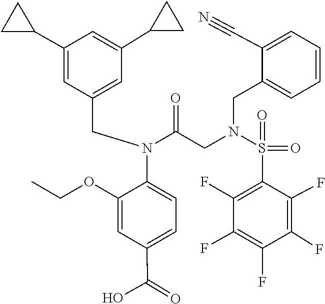 [SMILES: CCOc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F]

The title compound, 4-(2-((N-(2-cyanobenzyl)-2,3,4,5,6-pentafluorophenyl)sulfonamido)-N-(3,5-dicyclopropylbenzyl)acetamido)-3-ethoxybenzoic acid, was prepared according to the protocol described in general procedure F and isolated as a white powder (0.070 g, 38% yield). ¹H NMR (400 MHz, CDCl₃) δ 7.70 (d, J=7.8 Hz, 1H), 7.66-7.57 (m, 3H), 7.53 (d, J=1.6 Hz, 1H), 7.42 (td, J=7.7, 1.1 Hz, 1H), 7.08 (d, J=8.1 Hz, 1H), 6.63 (s, 1H), 6.53 (d, J=1.6 Hz, 2H), 5.01 (d, J=15.5 Hz, 1H), 4.81-4.59 (m, 3H), 4.18 (d, J=17.6 Hz, 1H), 4.04 (tt, J=15.0, 7.8 Hz, 1H), 3.79 (tt, J=14.2, 7.1 Hz, 1H), 3.69 (d, J=18.8 Hz, 1H), 1.78 (ddd, J=13.5, 8.5, 5.1 Hz, 2H), 1.31 (t, J=7.0 Hz, 3H), 0.96-0.84 (m, 4H), 0.63-0.49 (m, 4H). ¹⁹F NMR (376 MHz, CDCl₃) δ −133.85-−134.22 (m, 2F), -146.12-−146.58 (m, 1F), −159.45-−159.72 (m, 2F). ESI-MS: measured m/z 754.10 [M+H]⁺. Purity by HPLC: 98.8% at 254 nm.

### Example A7—Synthesis of Compound 1274

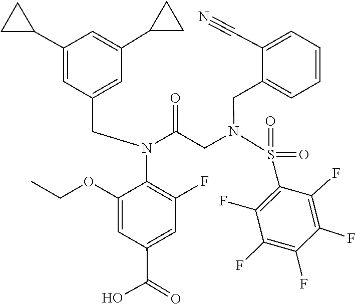 [SMILES: CCOc1cc(C(=O)O)cc(F)c1N(Cc1cc(C2CC2)cc(C2CC2)c1)C(=O)CN(Cc1ccccc1C#N)S(=O)(=O)c1c(F)c(F)c(F)c(F)c1F]

The title compound, 4-(2-((N-(2-cyanobenzyl)-2,3,4,5,6-pentafluorophenyl)sulfonamido)-N-(3,5-dicyclopropylbenzyl)acetamido)-3-ethoxy-5-fluorobenzoic acid, was prepared according to the protocol described in general procedure F and isolated as a white powder (0.029 g, 43% yield). ¹H NMR (400 MHz, CDCl₃) δ 7.67-7.55 (m, 3H), 7.44-7.37 (m, 1H), 7.33 (dd, J=9.2, 1.5 Hz, 1H), 7.27 (s, 1H), 6.60 (s, 1H), 6.49 (d, J=1.5 Hz, 2H), 4.97 (dd, J=19.3, 14.6 Hz, 2H), 4.78 (d, J=15.8 Hz, 1H), 4.38 (d, J=14.1 Hz, 1H), 4.15 (d, J=16.7 Hz, 1H), 4.07-3.93 (m, 1H), 3.78-3.58 (m, 2H), 1.80-1.67 (m, 2H), 1.26 (t, J=7.0 Hz, 3H), 0.95-0.77 (m, 4H), 0.63-0.41 (m, 4H). ¹⁹F NMR (376 MHz, CDCl₃) δ −115.59-−115.74 (m, 1F), −133.72-−134.43 (m, 2F), −145.91-−146.22 (m, 1F), −159.34-−159.63 (m, 2F). ESI-MS: measured m/z 772.2 [M+H]⁺. Purity by HPLC: 99.9% at 254 nm.

### Example A8—Synthesis of Compound 1275

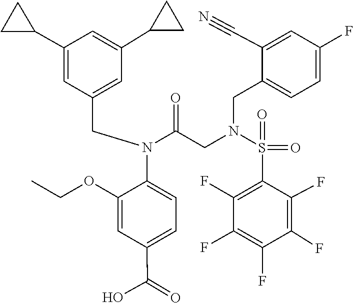 [SMILES: CCOc1cc(C(=O)O)ccc1N(Cc1cc(C2CC2)cc(C2CC2)c1)C(=O)CN(Cc1ccc(F)cc1C#N)S(=O)(=O)c1c(F)c(F)c(F)c(F)c1F]

The title compound, 4-(2-((N-(2-cyano-4-fluorobenzyl)-2,3,4,5,6-pentafluorophenyl)sulfonamido)-N-(3,5-dicyclopropylbenzyl)acetamido)-3-ethoxybenzoic acid, was prepared according to the protocol described in general procedure F and isolated as a white powder (0.037 g, 43% yield). ¹H NMR (400 MHz, CDCl₃) δ 7.74 (dd, J=9.5, 5.3 Hz, 1H), 7.63 (dd, J=8.1, 1.7 Hz, 1H), 7.55 (t, J=2.6 Hz, 1H), 7.37-7.29 (m, 2H), 7.11 (d, J=8.1 Hz, 1H), 6.63 (s, 1H), 6.53 (d, J=1.6 Hz, 2H), 4.97 (d, J=16.1 Hz, 1H), 4.77 (d, J=14.4 Hz, 1H), 4.65 (dd, J=13.9 Hz, 2H), 4.20 (d, J=20.7 Hz, 1H), 4.08 (dq, J=14.0, 7.0 Hz, 1H), 3.80 (dq, J=14.0, 7.0 Hz, 1H), 3.68 (d, J=16.0 Hz, 1H), 1.84-1.69 (m, 2H), 1.33 (t, J=7.0 Hz, 3H), 0.98-0.82 (m, 4H), 0.62-0.45 (m, 4H). ¹⁹F NMR (376 MHz, CDCl₃) δ −110.20 (m, 1F), −133.93-−134.12 (m, 2F), −145.91-−146.14 (m, 1F), −159.34-−159.60 (m, 2F). ESI-MS: measured m/z 772.1 [M+H]⁺. Purity by HPLC: 99.9% at 254 nm.

### Example A9—Synthesis of Compound 1276

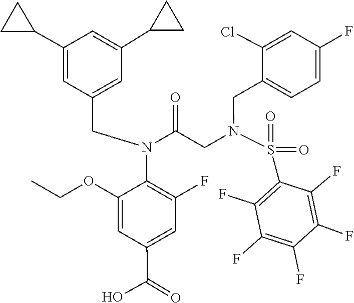 [SMILES: CCOc1cc(C(=O)O)cc(F)c1N(Cc1cc(C2CC2)cc(C2CC2)c1)C(=O)CN(Cc1ccc(F)cc1Cl)S(=O)(=O)c1c(F)c(F)c(F)c(F)c1F]

The title compound, 4-(2-((N-(2-chloro-4-fluorobenzyl)-2,3,4,5,6-pentafluorophenyl)sulfonamido)-N-(3,5-dicyclopropylbenzyl)acetamido)-3-ethoxy-5-fluorobenzoic acid, was prepared according to the protocol described in general procedure F and isolated as a white powder (0.022 g, 43% yield). ¹H NMR (400 MHz, CDC13) δ 7.46 (dd, J=8.6, 6.0 Hz, 1H), 7.37 (dd, J=9.2, 1.6 Hz, 1H), 7.30 (s, 1H), 7.08 (dd, J=8.4, 2.6 Hz, 1H), 7.01-6.91 (m, 1H), 6.62 (s, 1H), 6.48 (d, J=1.5 Hz, 2H), 4.89 (d, J=14.3 Hz, 2H), 4.66 (d, J=14.7 Hz, 1H), 4.39 (d, J=14.1 Hz, 1H), 4.19-3.95 (m, 2H), 3.73 (dq, J=9.3, 7.0 Hz, 1H), 3.60 (d, J=21.5 Hz, 1H), 1.74 (ddd, J=13.5, 8.4, 5.1 Hz, 2H), 1.28 (t, J=7.0 Hz, 3H), 0.95-0.80 (m, 4H), 0.61-0.42 (m, 4H). ¹⁹F NMR (376 MHz, CDCl₃) δ −110.91 (dd, J=14.0, 7.6 Hz, 1F), −115.62 (d, J=9.2 Hz, 1F), −133.94-−134.11 (m, 2F), −146.37-−146.56 (m, 1F), —159.59-−159.80 (m, 2F). ESI-MS: measured m/z 799.0 [M+1]⁺. Purity by HPLC: 99.9% at 254 nm.

### Example A10—Synthesis of Compound 1277

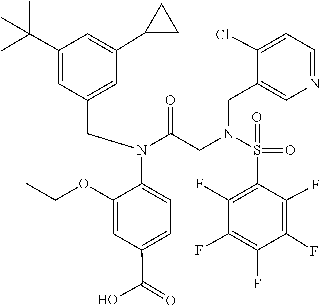 [SMILES: CCOc1cc(C(=O)O)ccc1N(Cc1cc(C2CC2)cc(C(C)(C)C)c1)C(=O)CN(Cc1cnccc1Cl)S(=O)(=O)c1c(F)c(F)c(F)c(F)c1F]

The title compound, 4-(N-(3-(tert-butyl)-5-cyclopropylbenzyl)-2-((N-((4-chloropyridin-3-yl)methyl)-2,3,4,5,6-pentafluorophenyl)sulfonamido)acetamido)-3-ethoxybenzoic acid, was prepared according to the protocol described in general procedure F and isolated as a white powder (0.005 g, 8% yield). ¹H NMR (400 MHz, CD3CN) δ 8.63 (s, 1H), 8.53 (s, 1H), 7.53-7.46 (m, 3H), 7.04-6.95 (m, 2H), 6.80 (d, J=2.0 Hz, 1H), 6.62 (s, 1H), 4.87 (d, J=15.5 Hz, 1H), 4.82-4.63 (m, 3H), 4.11-3.99 (m, 2H), 3.88-3.77 (m, 2H), 1.84 (tq, J=9.5, 4.9 Hz, 1H), 1.26 (t, J=6.9 Hz, 3H), 1.19 (s, 9H), 0.92 (dd, J=8.4, 2.5 Hz, 2H), 0.58 (dt, J=5.6, 3.4 Hz, 2H). ¹⁹F NMR (376 MHz, CD₃CN) δ −136.11-−136.30 (m, 2F), −148.68-−148.87 (m, 1F), −161.76-−162.00 (m, 2F). ESI-MS: measured m/z 780.3 [M+H]⁺. Purity by HPLC: 99.8% at 254 nm.

### Example A11—Synthesis of Compound 1278

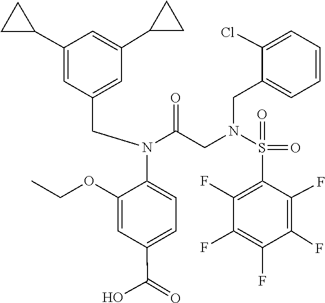 [SMILES: CCOc1cc(C(=O)O)ccc1N(Cc1cc(C2CC2)cc(C2CC2)c1)C(=O)CN(Cc1ccccc1Cl)S(=O)(=O)c1c(F)c(F)c(F)c(F)c1F]

The title compound, 4-(2-((N-(2-chlorobenzyl)-2,3,4,5,6-pentafluorophenyl)sulfonamido)-N-(3,5-dicyclopropylbenzyl)acetamido)-3-ethoxybenzoic acid, was prepared according to the protocol described in general procedure F and isolated as a white powder (0.031 g, 55% yield). ¹H NMR (400 MHz, CDCl₃) δ 7.60-7.51 (m, 2H), 7.48 (dd, J=5.5, 3.9 Hz, 1H), 7.36-7.31 (m, 1H), 7.28-7.19 (m, 2H), 6.82 (d, J=8.5 Hz, 1H), 6.65 (s, 1H), 6.54 (d, J=1.6 Hz, 2H), 4.92 (d, J=15.0 Hz, 1H), 4.86 (d, J=14.2 Hz, 1H), 4.73 (d, J=15.0 Hz, 1H), 4.45 (d, J=14.1 Hz, 1H), 4.13-3.96 (m, 2H), 3.87 (dq, J=14.0, 7.0 Hz, 1H), 3.67 (d, J=16.1 Hz, 1H), 1.79 (ddd, J=13.5, 8.5, 5.1 Hz, 2H), 1.33 (t, J=7.0 Hz, 3H), 0.96-0.83 (m, 4H), 0.62-0.52 (m, 4H). ¹⁹F NMR (376 MHz, CDCl₃) δ −134.00-−134.28 (m, 2F), −146.68-−146.96 (m, 1F), −159.71-−159.94 (m, 2F). ESI-MS: measured m/z 761.1 [M−H]⁻. Purity by HPLC: 99.9% at 254 nm.

### Example A12—Synthesis of Compound 1279

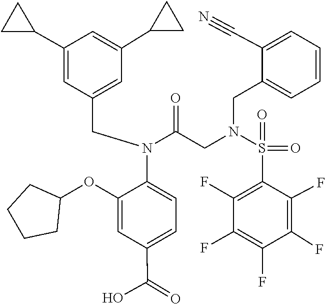 [SMILES: N#Cc1ccccc1CN(CC(=O)N(Cc1cc(C2CC2)cc(C2CC2)c1)c1ccc(C(=O)O)cc1OC1CCCC1)S(=O)(=O)c1c(F)c(F)c(F)c(F)c1F]

The title compound, 4-(2-((N-(2-cyanobenzyl)-2,3,4,5,6-pentafluorophenyl)sulfonamido)-N-(3,5-dicyclopropylbenzyl)acetamido)-3-(cyclopentyloxy)benzoic acid, was prepared according to the protocol described in general procedure F and isolated as a white powder (0.056 g, 46% yield). ¹H NMR (400 MHz, CDCl₃) δ 7.70 (d, J=7.8 Hz, 1H), 7.66-7.52 (m, 4H), 7.46-7.39 (m, 1H), 7.06 (d, J=8.0 Hz, 1H), 6.63 (s, 1H), 6.52 (d, J=1.6 Hz, 2H), 5.01 (d, J=15.5 Hz, 1H), 4.70 (m, J=16.6 Hz, 4H), 4.14 (d, J=17.1 Hz, 1H), 3.70 (d, J=17.6 Hz, 1H), 1.97-1.82 (m, 2H), 1.82-1.71 (m, 3H), 1.70-1.49 (m, 4H), 1.49-1.36 (m, 1H), 1.03-0.78 (m, 4H), 0.70-0.40 (m, 4H). ¹⁹F NMR (376 MHz, CDCl₃) δ −133.81-−134.36 (m, 2F), −146.16-−146.39 (m, 1F), −159.48-—159.79 (m, 2F). ESI-MS: measured m/z 794.1 [M+H]⁺. Purity by HPLC: 99.9% at 254 nm.

### Example A13—Synthesis of Compound 1280

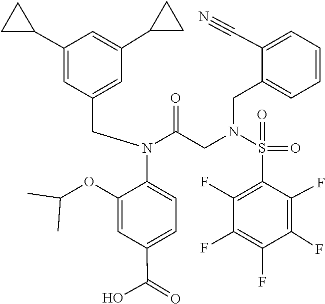 [SMILES: CC(C)Oc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F]

The title compound, 4-(2-((N-(2-cyanobenzyl)-2,3,4,5,6-pentafluorophenyl)sulfonamido)-N-(3,5-dicyclopropylbenzyl)acetamido)-3-isopropoxybenzoic acid, was prepared according to the protocol described in general procedure F and isolated as a white powder (0.038 g, 33% yield). ¹H NMR (400 MHz, CDCl₃) 6 7.71 (d, J=7.9 Hz, 1H), 7.66-7.50 (m, 4H), 7.42 (t, J=7.1 Hz, 1H), 7.07 (d, J=8.1 Hz, 1H), 6.62 (s, 1H), 6.55 (d, J=1.5 Hz, 2H), 5.03 (d, J=15.7 Hz, 1H), 4.79-4.60 (m, 3H), 4.60-4.42 (m, 1H), 4.20 (d, J=18.8 Hz, 1H), 3.65 (d, J=17.1 Hz, 1H), 1.78 (ddd, J=13.5, 8.4, 5.0 Hz, 2H), 1.28 (d, J=5.6 Hz, 3H), 1.09 (d, J=6.0 Hz, 3H), 0.95-0.82 (m, 4H), 0.63-0.45 (m, 4H). ¹⁹F NMR (376 MHz, CDCl₃) δ −133.68-−134.14 (m, 2F), −146.21-−146.40 (m, 1F), −159.11-−160.72 (m, 2F). ESI-MS: measured m/z 768.2 [M+H]⁺. Purity by HPLC: 97.1% at 254 nm.

### Example A14—Synthesis of Compound 1281

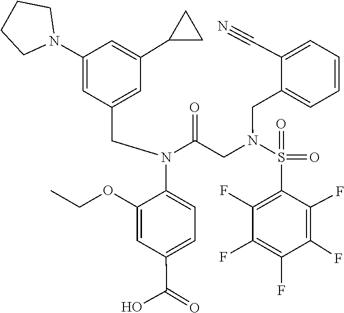 [SMILES: CCOc1cc(C(=O)O)ccc1N(Cc1cc(C2CC2)cc(N2CCCC2)c1)C(=O)CN(Cc1ccccc1C#N)S(=O)(=O)c1c(F)c(F)c(F)c(F)c1F]

The title compound, 4-(2-((N-(2-cyanobenzyl)-2,3,4,5,6-pentafluorophenyl)sulfonamido)-N-(3-cyclopropyl-5-(pyrrolidin-1-yl)benzyl)acetamido)-3-ethoxybenzoic acid, was prepared according to the protocol described in general procedure F and isolated as a white powder (0.048 g, 67% yield). ¹H NMR (400 MHz, CDCl₃) δ 7.68 (d, J=7.8 Hz, 1H), 7.65-7.52 (m, 4H), 7.41 (td, J=7.6, 1.0 Hz, 1H), 7.05 (d, J=8.0 Hz, 1H), 6.15 (s, 1H), 6.08 (s, 1H), 6.04 (s, 1H), 5.00 (d, J=15.6 Hz, 1H), 4.77 (dd, J=23.3, 14.8 Hz, 2H), 4.56 (d, J=13.9 Hz, 1H), 4.22-4.00 (m, 2H), 3.90-3.80 (m, 1H), 3.74 (d, J=17.1 Hz, 1H), 3.19 (t, J=6.5 Hz, 4H), 2.01-1.91 (m, 4H), 1.77 (ddd, J=13.5, 8.5, 5.1 Hz, 1H), 1.33 (t, J=6.9 Hz, 3H), 0.93-0.79 (m, 2H), 0.56 (p, J=9.5 Hz, 2H). ¹⁹F NMR (376 MHz, CDCl₃) δ −133.96-−134.16 (m, 2F), −146.29-−146.48 (m, 1F), −159.53-−159.77 (m, 2F). ESI-MS: measured m/z 781.1 [M−H]⁻. Purity by HPLC: 99.5% at 254 nm.

### Example A15—Synthesis of Compound 1141

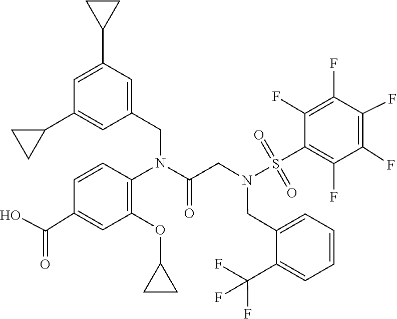 [SMILES: O=C(O)c1ccc(N(Cc2cc(C3CC3)cc(C3CC3)c2)C(=O)CN(Cc2ccccc2C(F)(F)F)S(=O)(=O)c2c(F)c(F)c(F)c(F)c2F)c(OC2CC2)c1]

The title compound, 4-(2-((N-(2-cyanobenzyl)-2,3,4,5,6-pentafluorophenyl)sulfonamido)-N-(3-cyclopropyl-5-(pyrrolidin-1-yl)benzyl)acetamido) ethoxybenzoic acid, was prepared according to the protocol described in general procedure F and isolated as a white powder (0.048 g, 67% yield). ¹H NMR (400 MHz, CDCl₃) δ 7.68 (d, J=7.8 Hz, 1H), 7.65-7.52 (m, 4H), 7.41 (td, J=7.6, 1.0 Hz, 1H), 7.05 (d, J=8.0 Hz, 1H), 6.15 (s, 1H), 6.08 (s, 1H), 6.04 (s, 1H), 5.00 (d, J=15.6 Hz, 1H), 4.77 (dd, J=23.3, 14.8 Hz, 2H), 4.56 (d, J=13.9 Hz, 1H), 4.22-4.00 (m, 2H), 3.90-3.80 (m, 1H), 3.74 (d, J=17.1 Hz, 1H), 3.19 (t, J=6.5 Hz, 4H), 2.01-1.91 (m, 4H), 1.77 (ddd, J=13.5, 8.5, 5.1 Hz, 1H), 1.33 (t, J=6.9 Hz, 3H), 0.93-0.79 (m, 2H), 0.56 (p, J=9.5 Hz, 2H). ¹⁹F NMR (376 MHz, CDCl₃) δ −133.96-−134.16 (m, 2F), −146.29-−146.48 (m, 1F), −159.53-−159.77 (m, 2F). ESI-MS: measured m/z 781.1 [M−H]⁻. Purity by HPLC: 99.5% at 254 nm.

### B: Biological Assays.

Suitable assays can be used to evaluate the efficacy and safety of the described novel STAT inhibitors. For example, considerations such as the potency, selectivity, stability, water-solubility, and bioavailability can be assessed by suitable in vitro and in vivo assays. Suitable assays include, but are not limited to, fluorescence polarization assay (for STAT inhibition), electrophoretic mobility shift assay (EMSA) (for STAT inhibition), western blot analysis (for STAT inhibition), surface plasmon resonance (SPR) studies (for binding affinity), mouse model-based blood brain barrier permeability, and Caco-2 cells permeability. Cell cultures can be used to evaluate the potency and selectivity of the compounds. For example, the potency of the compounds can be assessed using cell lines that harbor aberrant STAT proteins, such as human erythroleukemia K562 and MV-4-11 cells, breast carcinoma lines MDA-MB-231 and MDA-MB-468, androgen-insensitive human PC cell lines DU-145 and PC-3, and human lung cancer cells A549. The selectivity of the compounds can be assessed by cell culture cytotoxicity assays of non-target cells such as normal NIH 3T3 (3T3) cells, mouse thymus stromal epithelial cells, TE-71, Stat3-null mouse embryonic fibroblasts (−/−MEFs), NIH 3T3/v-Ras (v-Ras), normal human fibroblast (NHF) cells, and A2780S cells that do not harbor aberrantly active STAT3.

Several assay protocols and results are provided below for illustration purposes, and alternative assays can be used to evaluate the compounds. A skilled person in the art would appreciate that the disclosed compounds are potent STAT5 inhibitors with minimum off-target effects and superior stability and permeability.

### Example B1. MV4-11 Cell Cytotoxicity Assay

In some embodiments, the potency of the STAT5 inhibitors are evaluated by an in vitro assay such as MV4-11 Cell Cytotoxicity Assay. MV4-11 cells were grown in Iscove's Modified Dulbecco's Medium (IMDM) supplemented with 10% fetal bovine serum (FBS). 10,000 cells were plated per well in 96-well flat-bottom sterile culture plates with low-evaporation lids. After 24 h, inhibitors and a vehicle control (0.5% DMSO) were added and the cells were incubated for 72 h at 37° C. in 5% CO2. Inhibitors were examined in triplicate at a maximal concentration of 50 μM, followed by 1:2 dilutions in subsequent wells (25, 12.5, 6.25, 3.125, 1.5625, 0.78125, 0.390625, 0.195313 and 0.097656 μM). After 72 h, the wells were treated with CellTiter-Blue® (20 μL/well), and the plates were incubated using standard cell culture conditions for 1 hour. Fluorescence was measured at 560/590 nm. IC50 values were determined using non-linear regression analysis, and are provided in TABLE 3 and TABLE 4 below.

### Example B2. NHF Cell Cytotoxicity Assay

In some embodiments, the off-target effects of the compounds are evaluated in healthy human cells, such as in a normal human fibroblast (NHF) cell cytotoxicity assay.

Cell viability was examined following treatment at various concentrations of inhibitor (0.097656-50 μM) using a cell Titer-Blue cell viability assay. 1×10⁴ normal human fibroblast cells per well were plated in 96-well assay plates in culture medium. All cells are grown in DMEM, IMDM and RPMI-1640 were supplemented with 10% FBS. After 24 hours, test compounds and vehicle controls are added to appropriate wells so the final volume was 100 μl in each well. The cells are cultured for the desired test exposure period (72 hours) at 37° C. and 5% CO₂. The assay plates are removed from 37° C. incubator and 20 μL/well of CellTiter-Blue® Reagent is added. The plates are incubated using standard cell culture conditions for 1-4 hours and the plates are shaken for 10 seconds and record fluorescence at 560/590nm. IC₅₀ values were determined using non-linear regression analysis, and are provided in TABLE 3 and TABLE 4 below. For each sample well, value is normalized between the DMSO control and the highest concentration in case of plateau, and converted into a percentage. In the absence of plateau, minimum lecture is obtained from a different sample within the same experiment. For each concentration, the four replicates are averaged and standard deviation calculated. Data is fitted to a log(inhibitor) vs response curve with variable slope model using Microsoft Excel, obtaining IC₅₀ and Hill slope variables.

A person skilled in the art would appreciate that a higher IC50 value in this NHF assay can indicate lower off-target effects.

### Example B3. Reactivity Profiling with Glutathione (GSH)

In some embodiments, metabolic stability of the compounds are evaluated according to their reactivity profiles with GSH.

3.5 μL of 5 mM stocking solution of the inhibitors in DMSO was added to 697.5 μL of Iscove's Modified Dulbecco's Medium (IMDM) supplemented with 10% FBS and antibiotic antimycotic solution, with 5 mM glutathione to afford a final concentration of 25 μM inhibitor with 0.5% DMSO. The solution was then immediately placed in the sample tray at 25° C. Sample was analyzed at pre-defined intervals, typically every 1.5 hours, for up to four injections, by HPLC, included at time zero, without further pre-treatment. For each inhibitor, its peak is integrated at different time points and compared to the time zero injection in order to obtain a percentage remaining. Half-life is calculated according to a first order reaction kinetic taking into account those time points for which remaining percentage of inhibitor is above 40%, using the formula: t½=Ln(2)/k, where k is the slope of the linear plot of Ln[Inhibitor] vs time, according to the formula: Ln[A]=Ln[A]₀−kt, where [A] is the value resulting from the integration at each time point, [A]₀ the value at time zero, and t the time. For each inhibitor, both replicates are averaged and the resulting t½ reported. Selective reactivity against GSH in particular is confirmed by incubation of the inhibitor in the same solution without the presence of GSH, and single analysis after a time longer than the latest time point analyzed for the samples with GSH.

### Example B4. Parallel Artificial Membrane Permeability Assay (PAMPA)

In some embodiments, a PAMPA assay is used to determine the permeability of compounds of the present disclosure. The results of a PAMPA assay can correlate to a compound's permeability across a variety of barriers such as Caco-2 cells. The PAMPA assay can also be used to correlate the bioavailability of the compounds.

Stock solutions of positive controls (testosterone and methotrexate) were prepared in DMSO at the concentration of 10 mM, and further diluted with PBS (pH 7.4) to afford 10 μM solutions of the test compounds.

A 1.8% solution (w/v) of lecithin in dodecane was prepared and sonicated until complete dissolution was observed. 5 μL of the lecithin/dodecane mixture was then pipetted into each acceptor plate well (top compartment) of a 96-well filter plate with 0.45 μm pore size hydrophobic PDVF membrane, avoiding pipette tip contact with the membrane. Immediately after the application of the artificial membrane (within 10 minutes), 300 μL of PBS (pH 7.4) solution was added to each well of the acceptor plate. 300 μL of drug-containing solutions was then added to each well of the donor plate (bottom compartment) in triplicate. The acceptor plate was slowly placed into the donor plate, ensuring that the underside of the membrane maintained contact with the drug-containing solutions in all wells. The plate lid was replaced, and the solutions were incubated and rocked at 25° C., 60 rpm for 16 hours. After incubation, aliquots of 50 μL from each well of acceptor and donor plate were transferred into a 96-well plate. 200 μL of methanol containing 100 nM alprazolam, 200 nM labetalol and 2 μM ketoprofen was placed in each well. The plate lid was then replaced, and the plates were shaken at 750 rpm for 100 seconds. The samples were then centrifuged at 3,220 g for 20 minutes. The concentrations of the compound were determined by LC/MS/MS.

### Example B5. Exemplary Assay Data

In some embodiments, the activities and other properties of the disclosed exemplary compounds as determined by the above assays are shown in TABLE 3.

TABLE 3
Exemplary Assay Data

| | | | | Perme- | |
|---|---|---|---|---|---|
| | | | | ability | |
| | | | IC₅₀- | LogPe- | |
| | | | Pooled | PAMPA | T_{1/2}- |
| Compound | | IC₅₀-MV- | NHF | (4% | GSH |
| No. | Structure | 4-11 (μM) | (μM) | BSA) | (min) |
| 1008 | 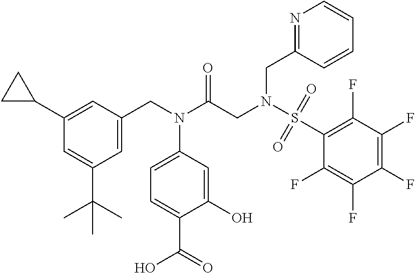 [SMILES: CC(C)(C)c1cc(CN(C(=O)CN(Cc2ccccn2)S(=O)(=O)c2c(F)c(F)c(F)c(F)c2F)c2ccc(C(=O)O)c(O)c2)cc(C2CC2)c1] | D | | | |
| 1009 | 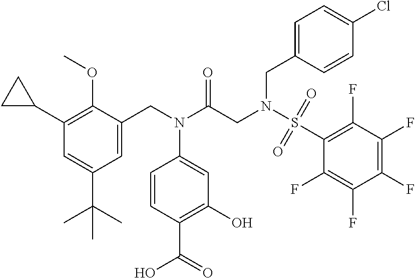 [SMILES: COc1c(CN(C(=O)CN(Cc2ccc(Cl)cc2)S(=O)(=O)c2c(F)c(F)c(F)c(F)c2F)c2ccc(C(=O)O)c(O)c2)cc(C(C)(C)C)cc1C1CC1] | D | | | |
| 1010 | 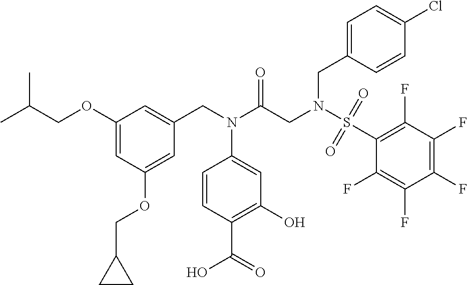 [SMILES: CC(C)COc1cc(CN(C(=O)CN(Cc2ccc(Cl)cc2)S(=O)(=O)c2c(F)c(F)c(F)c(F)c2F)c2ccc(C(=O)O)c(O)c2)cc(OCC2CC2)c1] | D | | | C |
| 1011 | 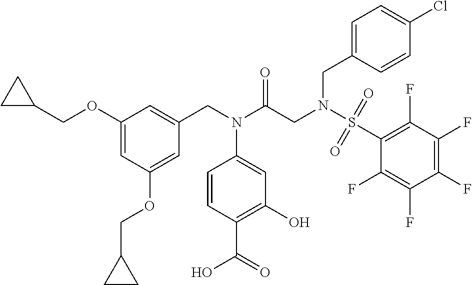 [SMILES: O=C(O)c1ccc(N(Cc2cc(OCC3CC3)cc(OCC3CC3)c2)C(=O)CN(Cc2ccc(Cl)cc2)S(=O)(=O)c2c(F)c(F)c(F)c(F)c2F)cc1O] | D | | | |
| 1013 | 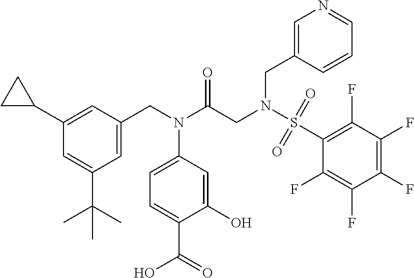 [SMILES: CC(C)(C)c1cc(CN(C(=O)CN(Cc2cccnc2)S(=O)(=O)c2c(F)c(F)c(F)c(F)c2F)c2ccc(C(=O)O)c(O)c2)cc(C2CC2)c1] | D | | | |
| 1015 | 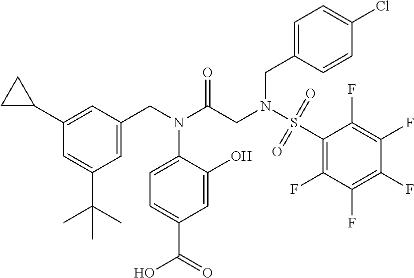 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)cc2O)cc(C2CC2)c1] | D | | | B |
| 1016 | 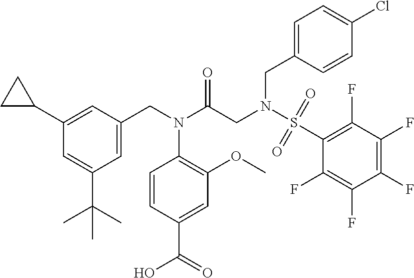 [SMILES: COc1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | B | C | | B |
| 1017 | 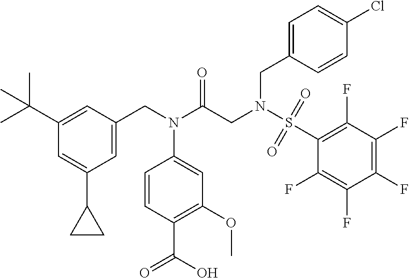 [SMILES: COc1cc(N(Cc2cc(C3CC3)cc(C(C)(C)C)c2)C(=O)CN(Cc2ccc(Cl)cc2)S(=O)(=O)c2c(F)c(F)c(F)c(F)c2F)ccc1C(=O)O] | B | | | B |
| 1018 | 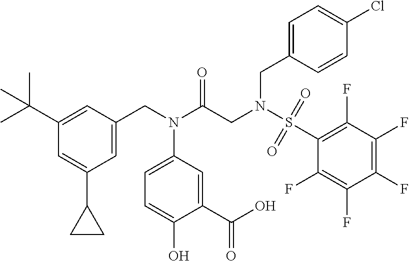 [SMILES: CC(C)(C)c1cc(CN(C(=O)CN(Cc2ccc(Cl)cc2)S(=O)(=O)c2c(F)c(F)c(F)c(F)c2F)c2ccc(O)c(C(=O)O)c2)cc(C2CC2)c1] | D | | | |
| 1019 | 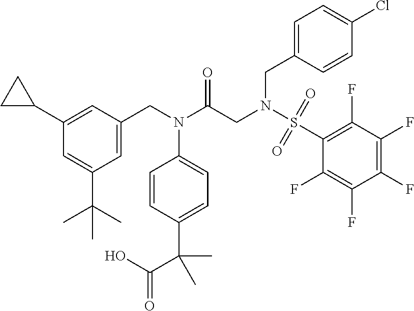 [SMILES: CC(C)(C)c1cc(CN(C(=O)CN(Cc2ccc(Cl)cc2)S(=O)(=O)c2c(F)c(F)c(F)c(F)c2F)c2ccc(C(C)(C)C(=O)O)cc2)cc(C2CC2)c1] | C | | | |
| 1020 | 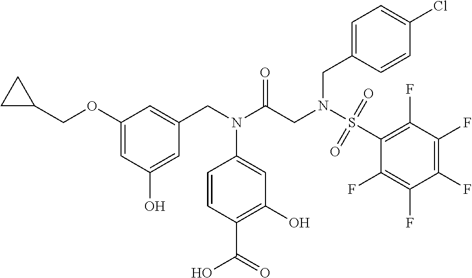 [SMILES: O=C(O)c1ccc(N(Cc2cc(O)cc(OCC3CC3)c2)C(=O)CN(Cc2ccc(Cl)cc2)S(=O)(=O)c2c(F)c(F)c(F)c(F)c2F)cc1O] | D | | | |
| 1021 | 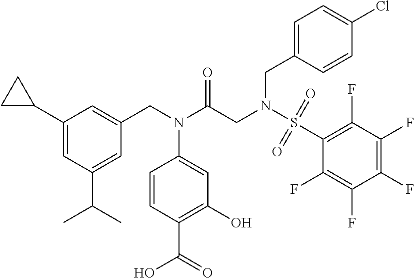 [SMILES: CC(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CC2)c1] | D | | | D |
| 1026 | 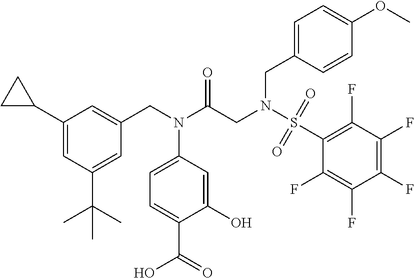 [SMILES: COc1ccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)cc1] | D | | | |
| 1027 | 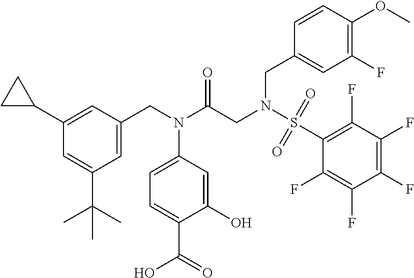 [SMILES: COc1ccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)cc1F] | D | | | |
| 1028 | 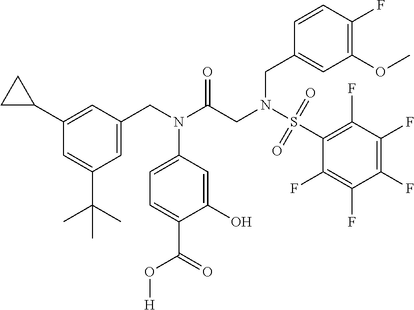 [SMILES: COc1cc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)ccc1F] | C | | | |
| 1029 | 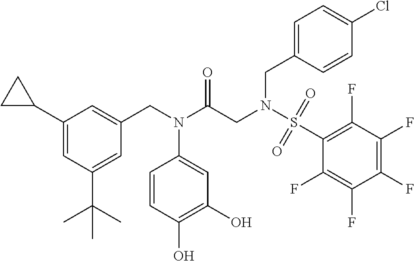 [SMILES: CC(C)(C)c1cc(CN(C(=O)CN(Cc2ccc(Cl)cc2)S(=O)(=O)c2c(F)c(F)c(F)c(F)c2F)c2ccc(O)c(O)c2)cc(C2CC2)c1] | D | | | |
| 1030 | 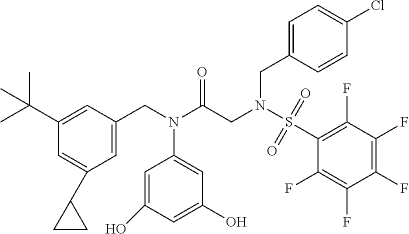 [SMILES: CC(C)(C)c1cc(CN(C(=O)CN(Cc2ccc(Cl)cc2)S(=O)(=O)c2c(F)c(F)c(F)c(F)c2F)c2cc(O)cc(O)c2)cc(C2CC2)c1] | D | | | |
| 1031 | 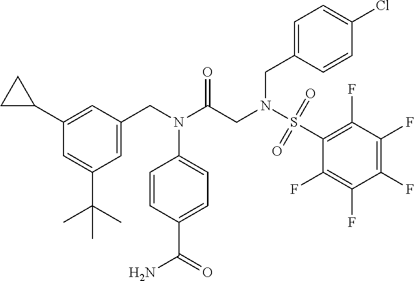 [SMILES: CC(C)(C)c1cc(CN(C(=O)CN(Cc2ccc(Cl)cc2)S(=O)(=O)c2c(F)c(F)c(F)c(F)c2F)c2ccc(C(N)=O)cc2)cc(C2CC2)c1] | C | | C | A |
| 1032 | 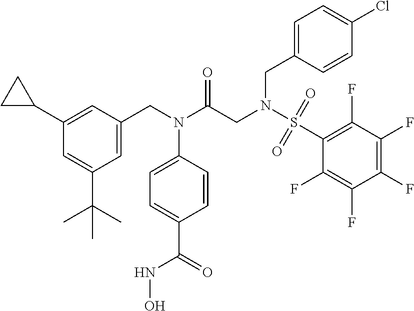 [SMILES: CC(C)(C)c1cc(CN(C(=O)CN(Cc2ccc(Cl)cc2)S(=O)(=O)c2c(F)c(F)c(F)c(F)c2F)c2ccc(C(=O)NO)cc2)cc(C2CC2)c1] | D | | | |
| 1033 | 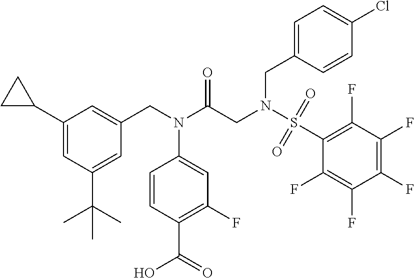 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(F)c2)cc(C2CC2)c1] | C | | A | D |
| 1034 | 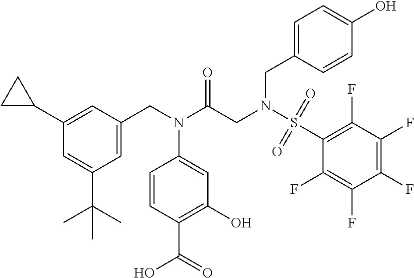 [SMILES: CC(C)(C)c1cc(CN(C(=O)CN(Cc2ccc(O)cc2)S(=O)(=O)c2c(F)c(F)c(F)c(F)c2F)c2ccc(C(=O)O)c(O)c2)cc(C2CC2)c1] | | | | |
| 1035 | 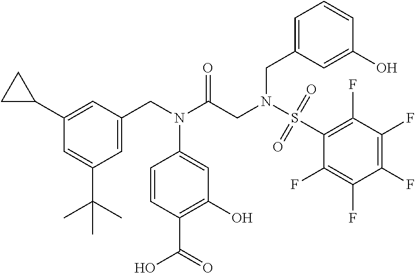 [SMILES: CC(C)(C)c1cc(CN(C(=O)CN(Cc2cccc(O)c2)S(=O)(=O)c2c(F)c(F)c(F)c(F)c2F)c2ccc(C(=O)O)c(O)c2)cc(C2CC2)c1] | D | | | |
| 1036 | 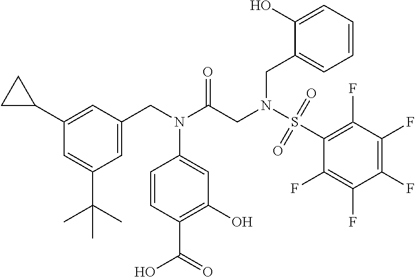 [SMILES: CC(C)(C)c1cc(CN(C(=O)CN(Cc2ccccc2O)S(=O)(=O)c2c(F)c(F)c(F)c(F)c2F)c2ccc(C(=O)O)c(O)c2)cc(C2CC2)c1] | C | | | |
| 1037 | 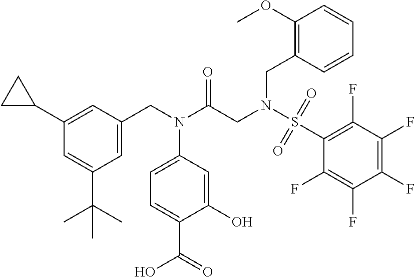 [SMILES: COc1ccccc1CN(CC(=O)N(Cc1cc(C2CC2)cc(C(C)(C)C)c1)c1ccc(C(=O)O)c(O)c1)S(=O)(=O)c1c(F)c(F)c(F)c(F)c1F] | C | | | C |
| 1038 | 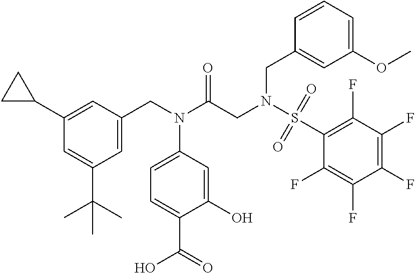 [SMILES: COc1cccc(CN(CC(=O)N(Cc2cc(C3CC3)cc(C(C)(C)C)c2)c2ccc(C(=O)O)c(O)c2)S(=O)(=O)c2c(F)c(F)c(F)c(F)c2F)c1] | D | | | |
| 1039 | 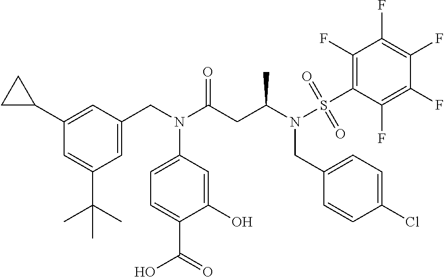 [SMILES: C[C@H](CC(=O)N(Cc1cc(C2CC2)cc(C(C)(C)C)c1)c1ccc(C(=O)O)c(O)c1)N(Cc1ccc(Cl)cc1)S(=O)(=O)c1c(F)c(F)c(F)c(F)c1F] | C | | | C |
| 1040 | 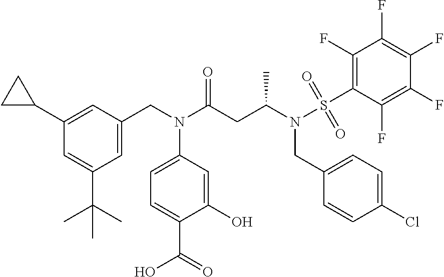 [SMILES: C[C@@H](CC(=O)N(Cc1cc(C2CC2)cc(C(C)(C)C)c1)c1ccc(C(=O)O)c(O)c1)N(Cc1ccc(Cl)cc1)S(=O)(=O)c1c(F)c(F)c(F)c(F)c1F] | D | | | B |
| 1041 | 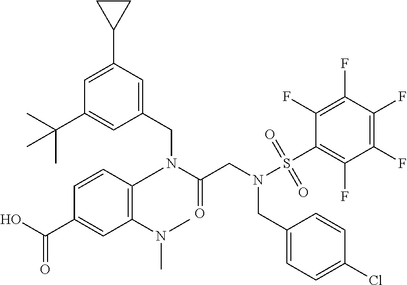 [SMILES: CN(C)c1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | B | C | C | A |
| 1042 | 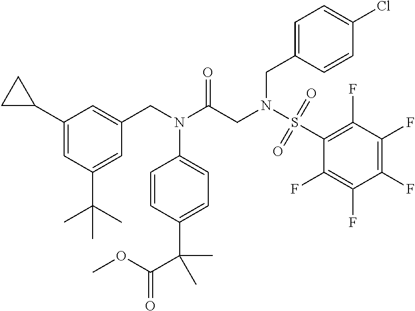 [SMILES: COC(=O)C(C)(C)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1] | D | | | |
| 1048 | 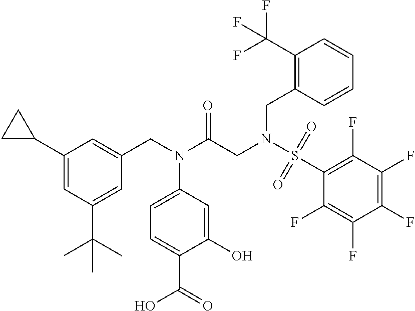 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)c(O)c2)cc(C2CC2)c1] | C | | | A |
| 1050 | 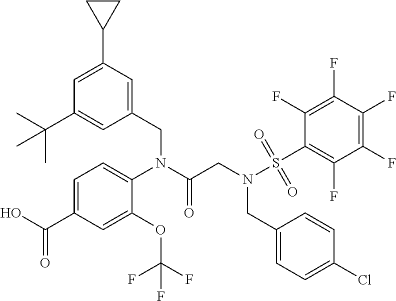 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)cc2OC(F)(F)F)cc(C2CC2)c1] | C | | | A |
| 1051 | 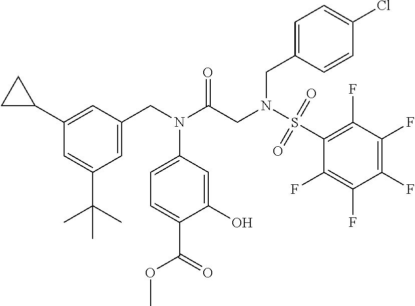 [SMILES: COC(=O)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1O] | D | | | |
| 1052 | 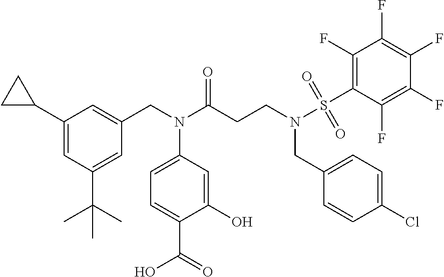 [SMILES: CC(C)(C)c1cc(CN(C(=O)CCN(Cc2ccc(Cl)cc2)S(=O)(=O)c2c(F)c(F)c(F)c(F)c2F)c2ccc(C(=O)O)c(O)c2)cc(C2CC2)c1] | D | | | C |
| 1054 | 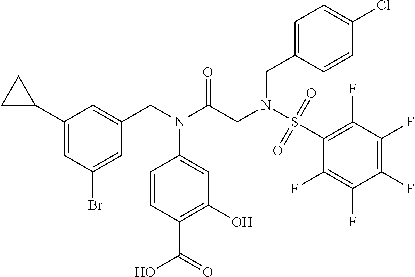 [SMILES: O=C(O)c1ccc(N(Cc2cc(Br)cc(C3CC3)c2)C(=O)CN(Cc2ccc(Cl)cc2)S(=O)(=O)c2c(F)c(F)c(F)c(F)c2F)cc1O] | D | | | D |
| 1055 | 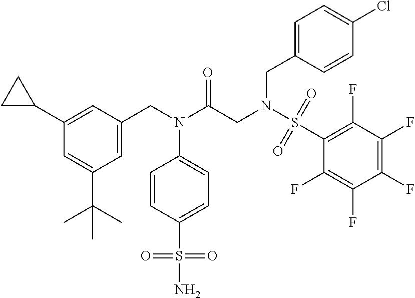 [SMILES: CC(C)(C)c1cc(CN(C(=O)CN(Cc2ccc(Cl)cc2)S(=O)(=O)c2c(F)c(F)c(F)c(F)c2F)c2ccc(S(N)(=O)=O)cc2)cc(C2CC2)c1] | C | | D | A |
| 1056 | 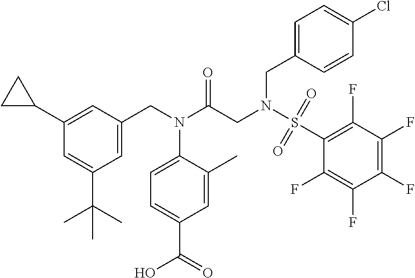 [SMILES: Cc1cc(C(=O)O)ccc1N(Cc1cc(C2CC2)cc(C(C)(C)C)c1)C(=O)CN(Cc1ccc(Cl)cc1)S(=O)(=O)c1c(F)c(F)c(F)c(F)c1F] | B | | | B |
| 1058 | 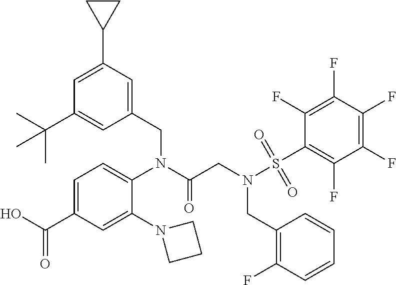 [SMILES: CC(C)(C)c1cc(CN(C(=O)CN(Cc2ccccc2F)S(=O)(=O)c2c(F)c(F)c(F)c(F)c2F)c2ccc(C(=O)O)cc2N2CCC2)cc(C2CC2)c1] | B | | | A |
| 1059 | 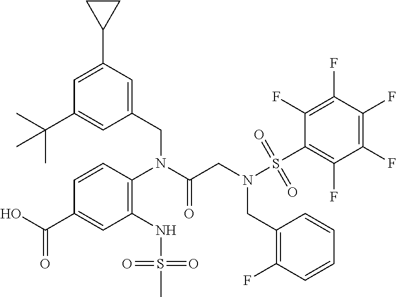 [SMILES: CC(C)(C)c1cc(CN(C(=O)CN(Cc2ccccc2F)S(=O)(=O)c2c(F)c(F)c(F)c(F)c2F)c2ccc(C(=O)O)cc2NS(C)(=O)=O)cc(C2CC2)c1] | D | | | |
| 1060 | 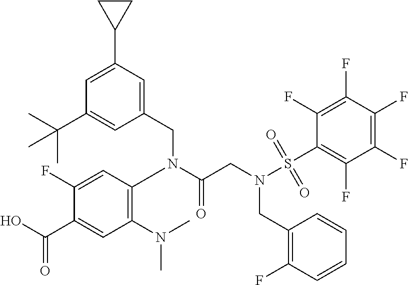 [SMILES: CN(C)c1cc(C(=O)O)c(F)cc1N(Cc1cc(C2CC2)cc(C(C)(C)C)c1)C(=O)CN(Cc1ccccc1F)S(=O)(=O)c1c(F)c(F)c(F)c(F)c1F] | A | | B | B |
| 1061 | 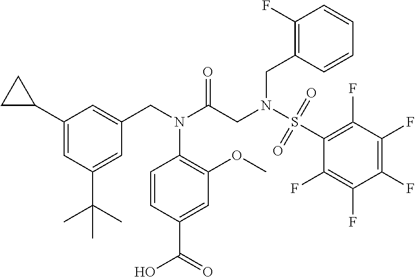 [SMILES: COc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | A | B | B | C |
| 1062 | 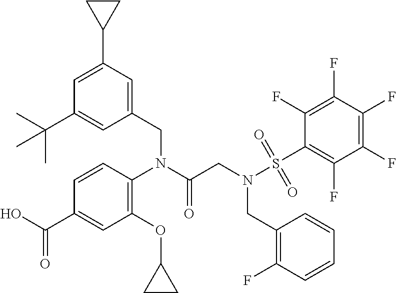 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CC2)cc(C2CC2)c1] | A | B | C | A |
| 1064 | 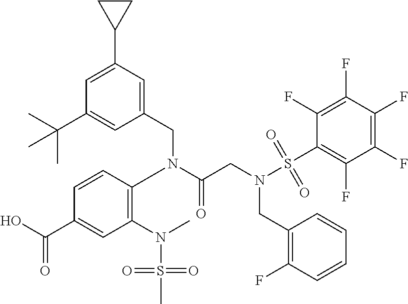 [SMILES: CN(c1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F)S(C)(=O)=O] | C | | A | B |
| 1065 | 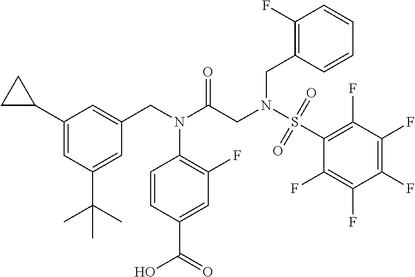 [SMILES: CC(C)(C)c1cc(CN(C(=O)CN(Cc2ccccc2F)S(=O)(=O)c2c(F)c(F)c(F)c(F)c2F)c2ccc(C(=O)O)cc2F)cc(C2CC2)c1] | B | | | D |
| 1066 | 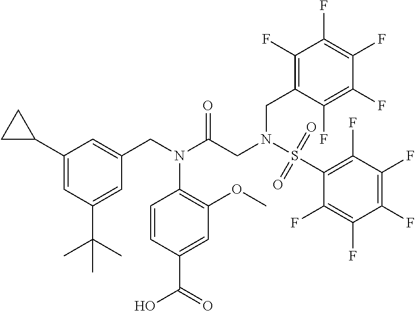 [SMILES: COc1cc(C(=O)O)ccc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | A | A | D | B |
| 1067 | 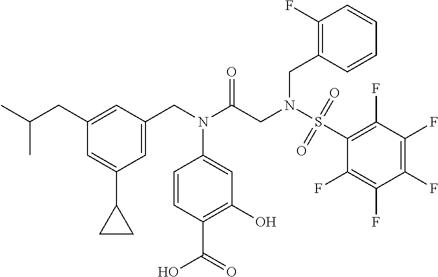 [SMILES: CC(C)Cc1cc(CN(C(=O)CN(Cc2ccccc2F)S(=O)(=O)c2c(F)c(F)c(F)c(F)c2F)c2ccc(C(=O)O)c(O)c2)cc(C2CC2)c1] | D | | | |
| 1069 | 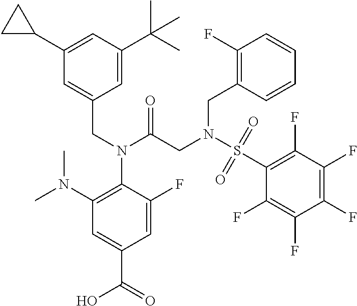 [SMILES: CN(C)c1cc(C(=O)O)cc(F)c1N(Cc1cc(C2CC2)cc(C(C)(C)C)c1)C(=O)CN(Cc1ccccc1F)S(=O)(=O)c1c(F)c(F)c(F)c(F)c1F] | B | | | |
| 1070 | 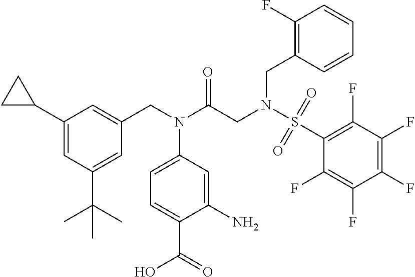 [SMILES: CC(C)(C)c1cc(CN(C(=O)CN(Cc2ccccc2F)S(=O)(=O)c2c(F)c(F)c(F)c(F)c2F)c2ccc(C(=O)O)c(N)c2)cc(C2CC2)c1] | B | | | D |
| 1071 | 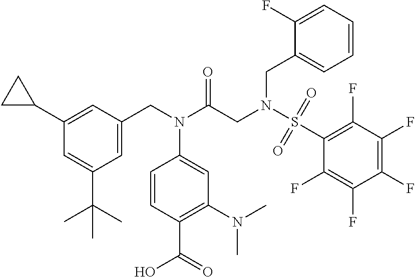 [SMILES: CN(C)c1cc(N(Cc2cc(C3CC3)cc(C(C)(C)C)c2)C(=O)CN(Cc2ccccc2F)S(=O)(=O)c2c(F)c(F)c(F)c(F)c2F)ccc1C(=O)O] | B | | | C |
| 1072 | 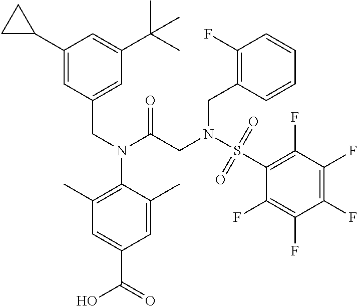 [SMILES: Cc1cc(C(=O)O)cc(C)c1N(Cc1cc(C2CC2)cc(C(C)(C)C)c1)C(=O)CN(Cc1ccccc1F)S(=O)(=O)c1c(F)c(F)c(F)c(F)c1F] | B | | B | B |
| 1073 | 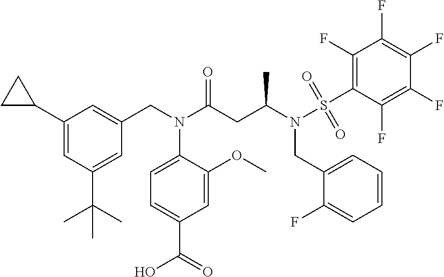 [SMILES: COc1cc(C(=O)O)ccc1N(Cc1cc(C2CC2)cc(C(C)(C)C)c1)C(=O)C[C@@H](C)N(Cc1ccccc1F)S(=O)(=O)c1c(F)c(F)c(F)c(F)c1F] | B | | | D |
| 1074 | 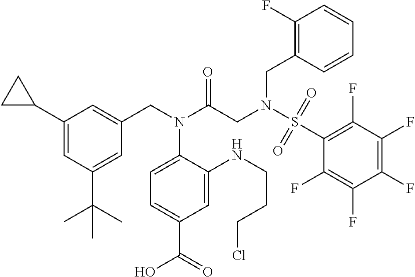 [SMILES: CC(C)(C)c1cc(CN(C(=O)CN(Cc2ccccc2F)S(=O)(=O)c2c(F)c(F)c(F)c(F)c2F)c2ccc(C(=O)O)cc2NCCCCl)cc(C2CC2)c1] | C | | | |
| 1075 | 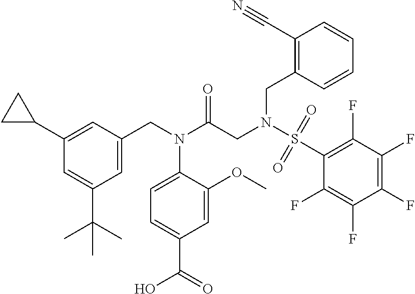 [SMILES: COc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | A | B | A | D |
| 1076 | 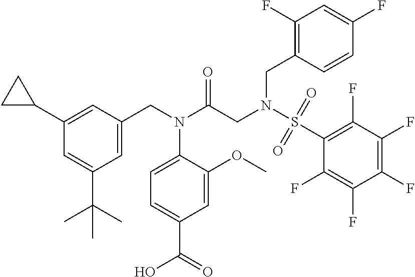 [SMILES: COc1cc(C(=O)O)ccc1N(Cc1cc(C2CC2)cc(C(C)(C)C)c1)C(=O)CN(Cc1ccc(F)cc1F)S(=O)(=O)c1c(F)c(F)c(F)c(F)c1F] | B | | | C |
| 1077 | 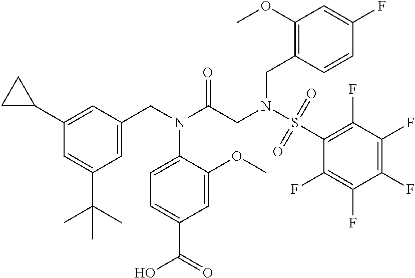 [SMILES: COc1cc(F)ccc1CN(CC(=O)N(Cc1cc(C2CC2)cc(C(C)(C)C)c1)c1ccc(C(=O)O)cc1OC)S(=O)(=O)c1c(F)c(F)c(F)c(F)c1F] | B | | | C |
| 1078 | 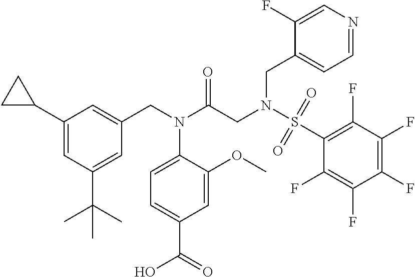 [SMILES: COc1cc(C(=O)O)ccc1N(Cc1cc(C2CC2)cc(C(C)(C)C)c1)C(=O)CN(Cc1ccncc1F)S(=O)(=O)c1c(F)c(F)c(F)c(F)c1F] | B | A | A | D |
| 1079 | 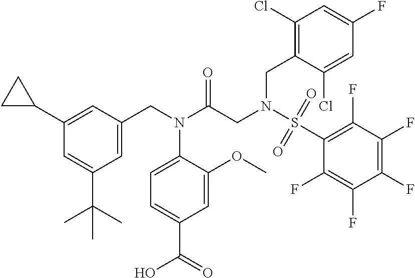 [SMILES: COc1cc(C(=O)O)ccc1N(Cc1cc(C2CC2)cc(C(C)(C)C)c1)C(=O)CN(Cc1c(Cl)cc(F)cc1Cl)S(=O)(=O)c1c(F)c(F)c(F)c(F)c1F] | A | | | B |
| 1080 | 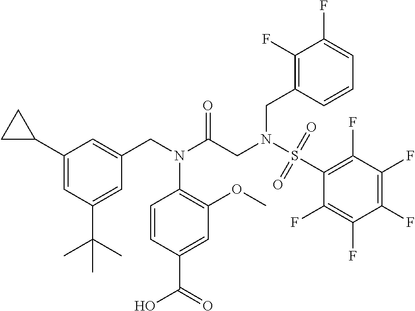 [SMILES: COc1cc(C(=O)O)ccc1N(Cc1cc(C2CC2)cc(C(C)(C)C)c1)C(=O)CN(Cc1cccc(F)c1F)S(=O)(=O)c1c(F)c(F)c(F)c(F)c1F] | A | | A | C |
| 1081 | 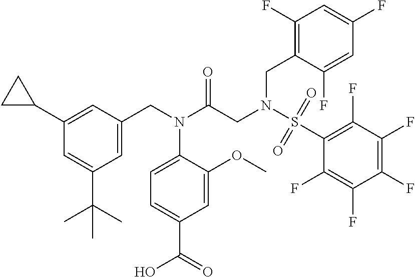 [SMILES: COc1cc(C(=O)O)ccc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | A | A | A | B |
| 1082 | 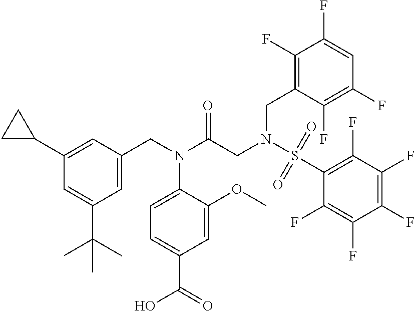 [SMILES: COc1cc(C(=O)O)ccc1N(Cc1cc(C2CC2)cc(C(C)(C)C)c1)C(=O)CN(Cc1c(F)c(F)cc(F)c1F)S(=O)(=O)c1c(F)c(F)c(F)c(F)c1F] | A | | | C |
| 1083 | 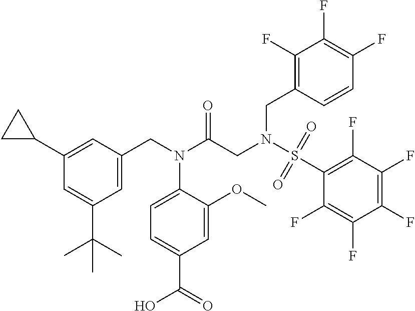 [SMILES: COc1cc(C(=O)O)ccc1N(Cc1cc(C2CC2)cc(C(C)(C)C)c1)C(=O)CN(Cc1ccc(F)c(F)c1F)S(=O)(=O)c1c(F)c(F)c(F)c(F)c1F] | A | | | B |
| 1084 | 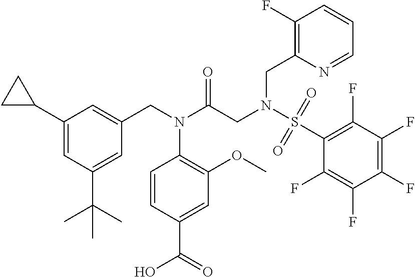 [SMILES: COc1cc(C(=O)O)ccc1N(Cc1cc(C2CC2)cc(C(C)(C)C)c1)C(=O)CN(Cc1ncccc1F)S(=O)(=O)c1c(F)c(F)c(F)c(F)c1F] | B | | A | D |
| 1085 | 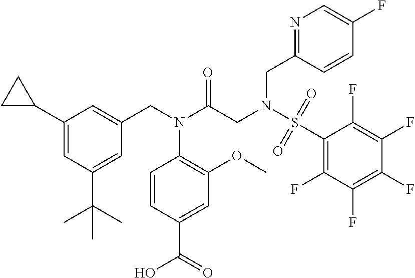 [SMILES: COc1cc(C(=O)O)ccc1N(Cc1cc(C2CC2)cc(C(C)(C)C)c1)C(=O)CN(Cc1ccc(F)cn1)S(=O)(=O)c1c(F)c(F)c(F)c(F)c1F] | B | | | D |
| 1086 | 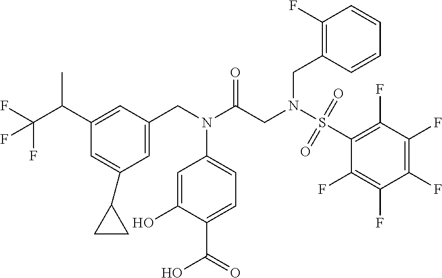 [SMILES: CC(c1cc(CN(C(=O)CN(Cc2ccccc2F)S(=O)(=O)c2c(F)c(F)c(F)c(F)c2F)c2ccc(C(=O)O)c(O)c2)cc(C2CC2)c1)C(F)(F)F] | C | | | C |
| 1087 | 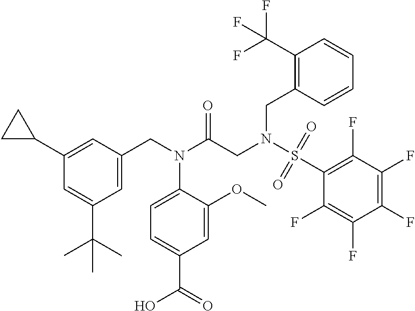 [SMILES: COc1cc(C(=O)O)ccc1N(Cc1cc(C2CC2)cc(C(C)(C)C)c1)C(=O)CN(Cc1ccccc1C(F)(F)F)S(=O)(=O)c1c(F)c(F)c(F)c(F)c1F] | A | C | A | B |
| 1088 | 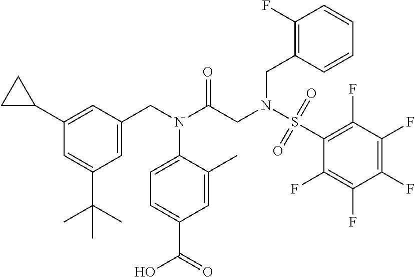 [SMILES: Cc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | A | | B | C |
| 1089 | 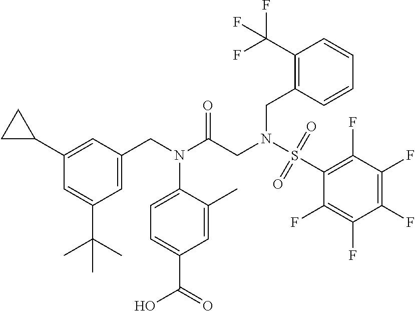 [SMILES: Cc1cc(C(=O)O)ccc1N(Cc1cc(C2CC2)cc(C(C)(C)C)c1)C(=O)CN(Cc1ccccc1C(F)(F)F)S(=O)(=O)c1c(F)c(F)c(F)c(F)c1F] | B | | | A |
| 1090 | 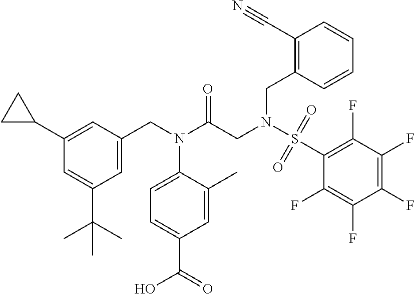 [SMILES: Cc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | B | | | C |
| 1091 | 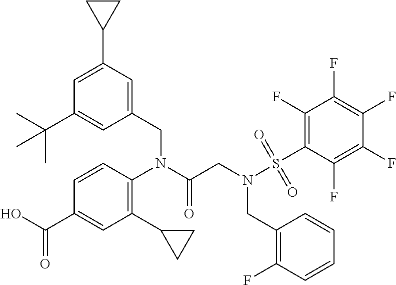 [SMILES: CC(C)(C)c1cc(CN(C(=O)CN(Cc2ccccc2F)S(=O)(=O)c2c(F)c(F)c(F)c(F)c2F)c2ccc(C(=O)O)cc2C2CC2)cc(C2CC2)c1] | A | A | B | B |
| 1092 | 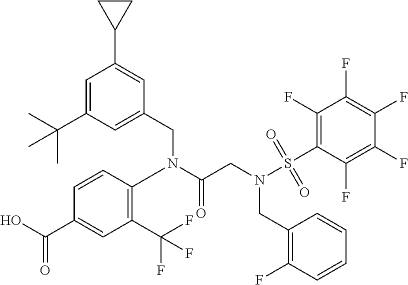 [SMILES: CC(C)(C)c1cc(CN(C(=O)CN(Cc2ccccc2F)S(=O)(=O)c2c(F)c(F)c(F)c(F)c2F)c2ccc(C(=O)O)cc2C(F)(F)F)cc(C2CC2)c1] | A | B | B | B |
| 1093 | 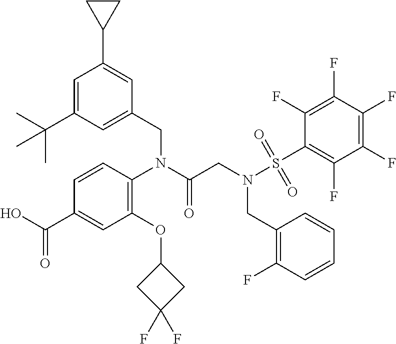 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CC(F)(F)C2)cc(C2CC2)c1] | B | | B | A |
| 1094 | 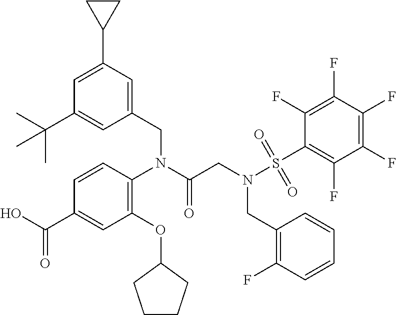 [SMILES: CC(C)(C)c1cc(CN(C(=O)CN(Cc2ccccc2F)S(=O)(=O)c2c(F)c(F)c(F)c(F)c2F)c2ccc(C(=O)O)cc2OC2CCCC2)cc(C2CC2)c1] | A | C | | A |
| 1095 | 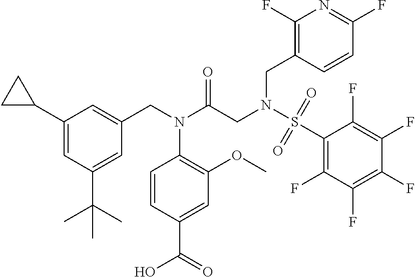 [SMILES: COc1cc(C(=O)O)ccc1N(Cc1cc(C2CC2)cc(C(C)(C)C)c1)C(=O)CN(Cc1ccc(F)nc1F)S(=O)(=O)c1c(F)c(F)c(F)c(F)c1F] | A | | | D |
| 1096 | 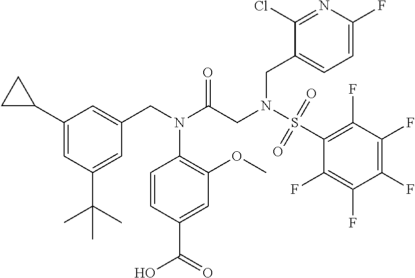 [SMILES: COc1cc(C(=O)O)ccc1N(Cc1cc(C2CC2)cc(C(C)(C)C)c1)C(=O)CN(Cc1ccc(F)nc1Cl)S(=O)(=O)c1c(F)c(F)c(F)c(F)c1F] | A | | | |
| 1097 | 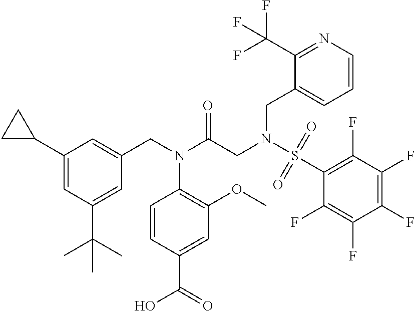 [SMILES: COc1cc(C(=O)O)ccc1N(Cc1cc(C2CC2)cc(C(C)(C)C)c1)C(=O)CN(Cc1cccnc1C(F)(F)F)S(=O)(=O)c1c(F)c(F)c(F)c(F)c1F] | B | | | B |
| 1098 | 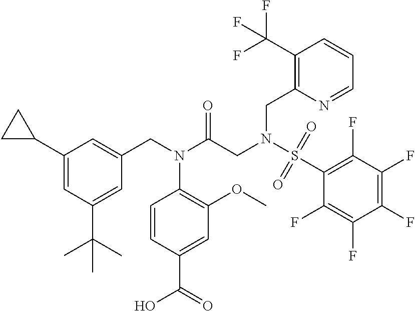 [SMILES: COc1cc(C(=O)O)ccc1N(Cc1cc(C2CC2)cc(C(C)(C)C)c1)C(=O)CN(Cc1ncccc1C(F)(F)F)S(=O)(=O)c1c(F)c(F)c(F)c(F)c1F] | B | C | A | C |
| 1099 | 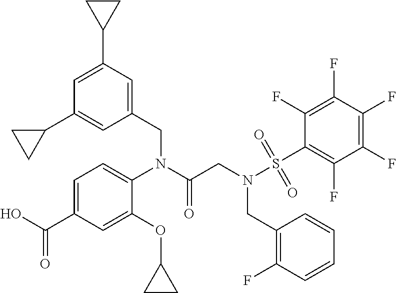 [SMILES: O=C(O)c1ccc(N(Cc2cc(C3CC3)cc(C3CC3)c2)C(=O)CN(Cc2ccccc2F)S(=O)(=O)c2c(F)c(F)c(F)c(F)c2F)c(OC2CC2)c1] | A | | | B |
| 1100 | 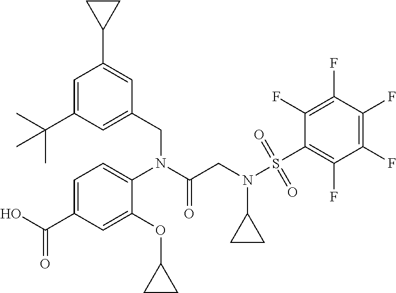 [SMILES: CC(C)(C)c1cc(CN(C(=O)CN(C2CC2)S(=O)(=O)c2c(F)c(F)c(F)c(F)c2F)c2ccc(C(=O)O)cc2OC2CC2)cc(C2CC2)c1] | B | | | D |
| 1101 | 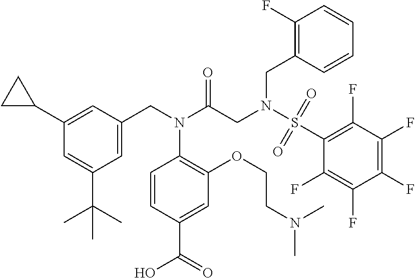 [SMILES: CN(C)CCOc1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | D | | | |
| 1105 | 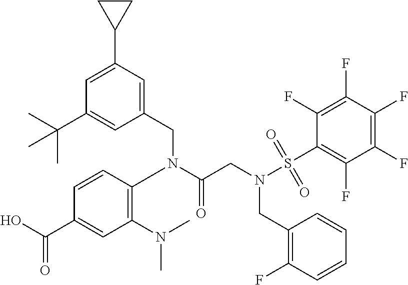 [SMILES: CN(C)c1cc(C(=O)O)ccc1N(Cc1cc(C2CC2)cc(C(C)(C)C)c1)C(=O)CN(Cc1ccccc1F)S(=O)(=O)c1c(F)c(F)c(F)c(F)c1F] | A | | | B |
| 1106 | 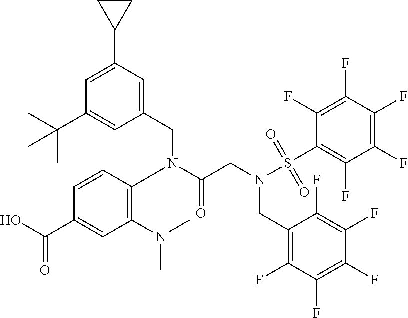 [SMILES: CN(C)c1cc(C(=O)O)ccc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | A | | | |
| 1107 | 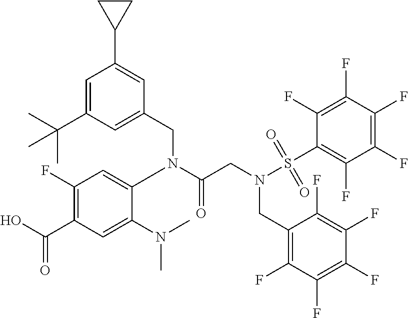 [SMILES: CN(C)c1cc(C(=O)O)c(F)cc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | A | | | |
| 1108 | 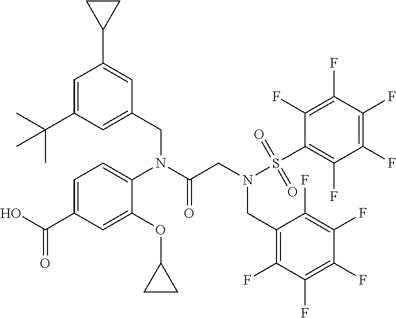 [SMILES: CC(C)(C)c1cc(CN(C(=O)CN(Cc2c(F)c(F)c(F)c(F)c2F)S(=O)(=O)c2c(F)c(F)c(F)c(F)c2F)c2ccc(C(=O)O)cc2OC2CC2)cc(C2CC2)c1] | A | A | | A |
| 1110 | 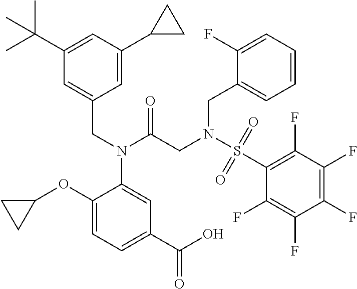 [SMILES: CC(C)(C)c1cc(CN(C(=O)CN(Cc2ccccc2F)S(=O)(=O)c2c(F)c(F)c(F)c(F)c2F)c2cc(C(=O)O)ccc2OC2CC2)cc(C2CC2)c1] | B | | B | C |
| 1111 | 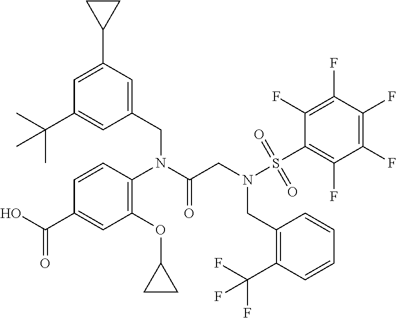 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)cc2OC2CC2)cc(C2CC2)c1] | A | D | B | A |
| 1112 | 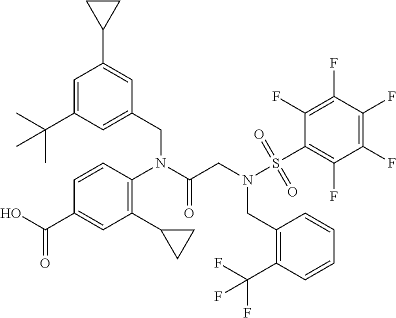 [SMILES: CC(C)(C)c1cc(CN(C(=O)CN(Cc2ccccc2C(F)(F)F)S(=O)(=O)c2c(F)c(F)c(F)c(F)c2F)c2ccc(C(=O)O)cc2C2CC2)cc(C2CC2)c1] | A | | | |
| 1113 | 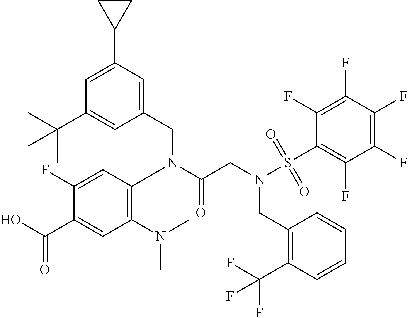 [SMILES: CN(C)c1cc(C(=O)O)c(F)cc1N(Cc1cc(C2CC2)cc(C(C)(C)C)c1)C(=O)CN(Cc1ccccc1C(F)(F)F)S(=O)(=O)c1c(F)c(F)c(F)c(F)c1F] | B | | | |
| 1114 | 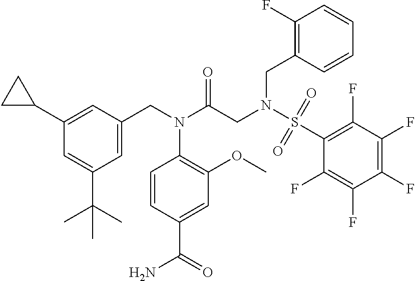 [SMILES: COc1cc(C(N)=O)ccc1N(Cc1cc(C2CC2)cc(C(C)(C)C)c1)C(=O)CN(Cc1ccccc1F)S(=O)(=O)c1c(F)c(F)c(F)c(F)c1F] | B | | C | |
| 1115 | 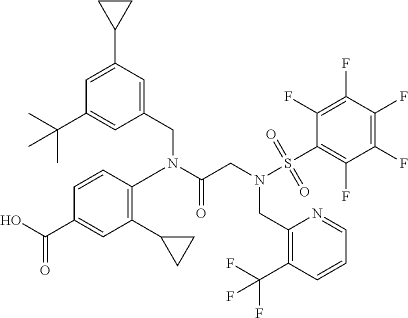 [SMILES: CC(C)(C)c1cc(CN(C(=O)CN(Cc2ncccc2C(F)(F)F)S(=O)(=O)c2c(F)c(F)c(F)c(F)c2F)c2ccc(C(=O)O)cc2C2CC2)cc(C2CC2)c1] | A | | | B |
| 1116 | 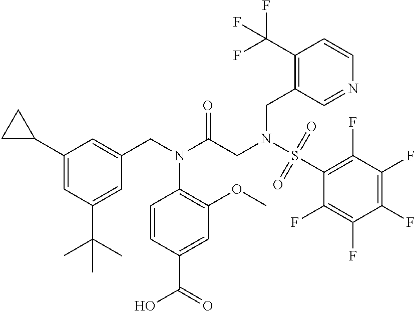 [SMILES: COc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | A | B | A | C |
| 1117 | 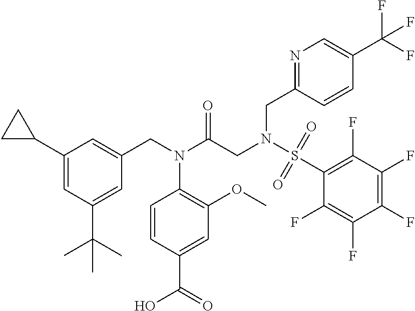 [SMILES: COc1cc(C(=O)O)ccc1N(Cc1cc(C2CC2)cc(C(C)(C)C)c1)C(=O)CN(Cc1ccc(C(F)(F)F)cn1)S(=O)(=O)c1c(F)c(F)c(F)c(F)c1F] | B | | A | C |
| 1118 | 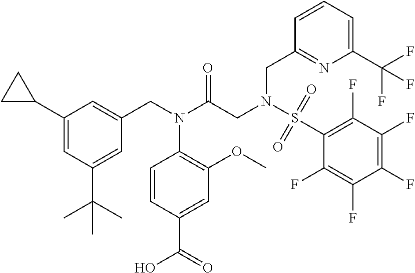 [SMILES: COc1cc(C(=O)O)ccc1N(Cc1cc(C2CC2)cc(C(C)(C)C)c1)C(=O)CN(Cc1cccc(C(F)(F)F)n1)S(=O)(=O)c1c(F)c(F)c(F)c(F)c1F] | B | | A | D |
| 1119 | 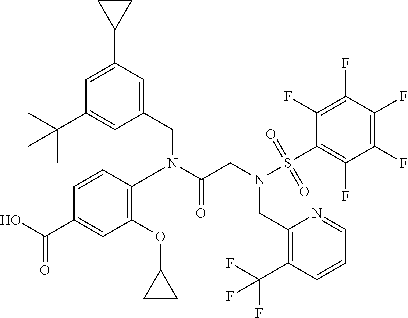 [SMILES: CC(C)(C)c1cc(CN(C(=O)CN(Cc2ncccc2C(F)(F)F)S(=O)(=O)c2c(F)c(F)c(F)c(F)c2F)c2ccc(C(=O)O)cc2OC2CC2)cc(C2CC2)c1] | B | D | A | B |
| 1120 | 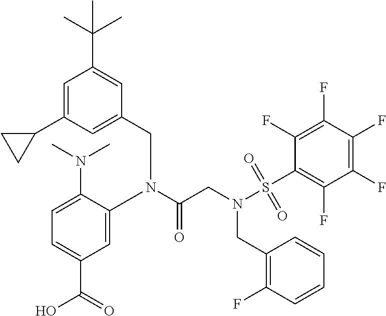 [SMILES: CN(C)c1ccc(C(=O)O)cc1N(Cc1cc(C2CC2)cc(C(C)(C)C)c1)C(=O)CN(Cc1ccccc1F)S(=O)(=O)c1c(F)c(F)c(F)c(F)c1F] | B | | B | |
| 1122 | 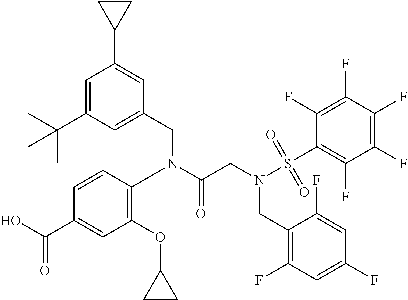 [SMILES: CC(C)(C)c1cc(CN(C(=O)CN(Cc2c(F)cc(F)cc2F)S(=O)(=O)c2c(F)c(F)c(F)c(F)c2F)c2ccc(C(=O)O)cc2OC2CC2)cc(C2CC2)c1] | A | B | A | A |
| 1123 | 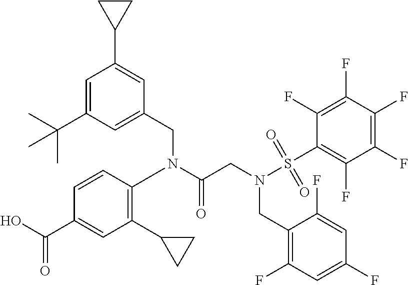 [SMILES: CC(C)(C)c1cc(CN(C(=O)CN(Cc2c(F)cc(F)cc2F)S(=O)(=O)c2c(F)c(F)c(F)c(F)c2F)c2ccc(C(=O)O)cc2C2CC2)cc(C2CC2)c1] | A | A | B | A |
| 1124 | 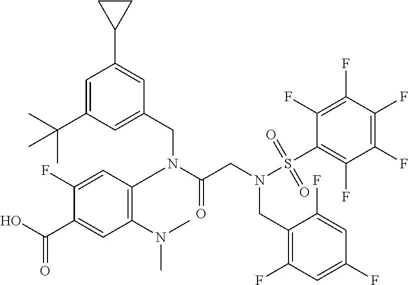 [SMILES: CN(C)c1cc(C(=O)O)c(F)cc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | A | | A | B |
| 1125 | 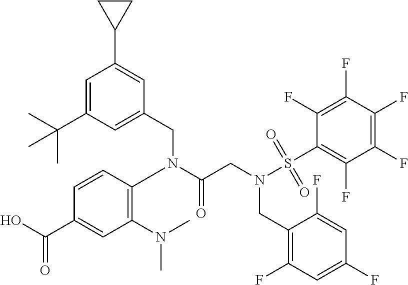 [SMILES: CN(C)c1cc(C(=O)O)ccc1N(Cc1cc(C2CC2)cc(C(C)(C)C)c1)C(=O)CN(Cc1c(F)cc(F)cc1F)S(=O)(=O)c1c(F)c(F)c(F)c(F)c1F] | B | | B | |
| 1126 | 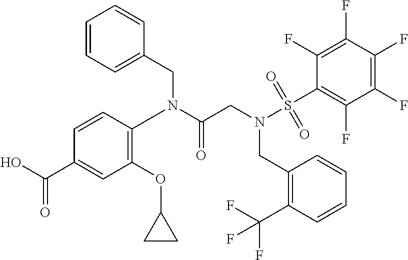 [SMILES: O=C(O)c1ccc(N(Cc2ccccc2)C(=O)CN(Cc2ccccc2C(F)(F)F)S(=O)(=O)c2c(F)c(F)c(F)c(F)c2F)c(OC2CC2)c1] | C | | | D |
| 1127 | 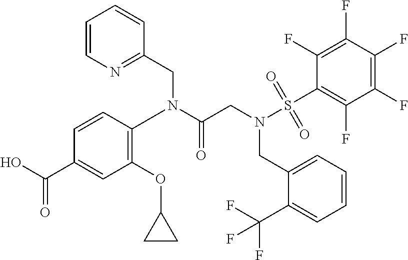 [SMILES: O=C(O)c1ccc(N(Cc2ccccn2)C(=O)CN(Cc2ccccc2C(F)(F)F)S(=O)(=O)c2c(F)c(F)c(F)c(F)c2F)c(OC2CC2)c1] | C | | | |
| 1128 | 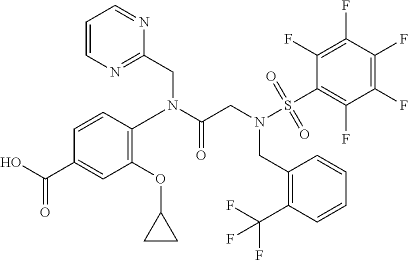 [SMILES: O=C(O)c1ccc(N(Cc2ncccn2)C(=O)CN(Cc2ccccc2C(F)(F)F)S(=O)(=O)c2c(F)c(F)c(F)c(F)c2F)c(OC2CC2)c1] | C | | B | D |
| 1129 | 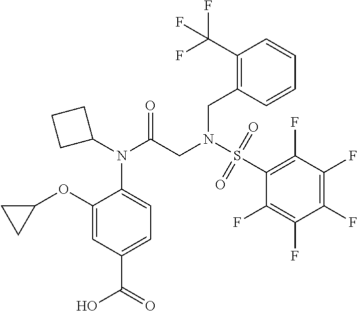 [SMILES: O=C(O)c1ccc(N(C(=O)CN(Cc2ccccc2C(F)(F)F)S(=O)(=O)c2c(F)c(F)c(F)c(F)c2F)C2CCC2)c(OC2CC2)c1] | D | | A | C |
| 1130 | 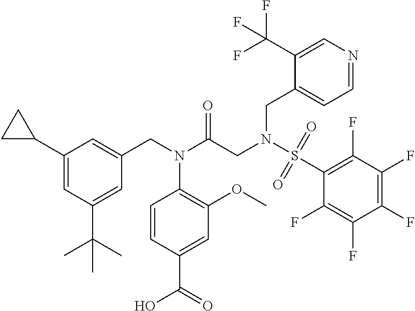 [SMILES: COc1cc(C(=O)O)ccc1N(Cc1cc(C2CC2)cc(C(C)(C)C)c1)C(=O)CN(Cc1ccncc1C(F)(F)F)S(=O)(=O)c1c(F)c(F)c(F)c(F)c1F] | B | | A | C |
| 1131 | 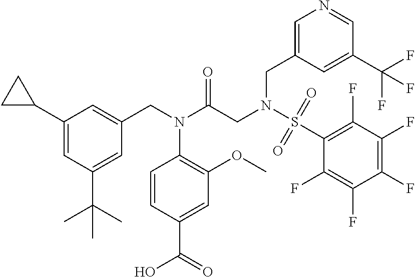 [SMILES: COc1cc(C(=O)O)ccc1N(Cc1cc(C2CC2)cc(C(C)(C)C)c1)C(=O)CN(Cc1cncc(C(F)(F)F)c1)S(=O)(=O)c1c(F)c(F)c(F)c(F)c1F] | B | | | |
| 1132 | 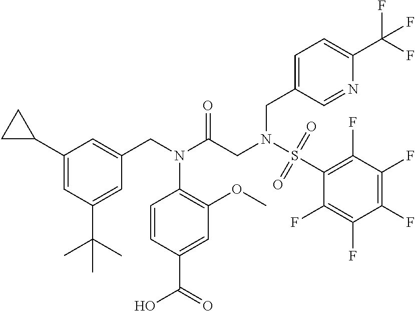 [SMILES: COc1cc(C(=O)O)ccc1N(Cc1cc(C2CC2)cc(C(C)(C)C)c1)C(=O)CN(Cc1ccc(C(F)(F)F)nc1)S(=O)(=O)c1c(F)c(F)c(F)c(F)c1F] | B | | | C |
| 1133 | 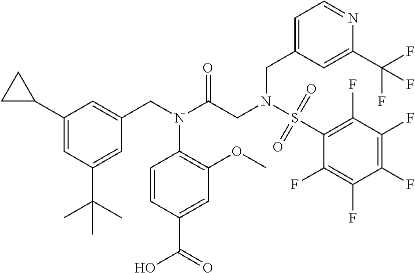 [SMILES: COc1cc(C(=O)O)ccc1N(Cc1cc(C2CC2)cc(C(C)(C)C)c1)C(=O)CN(Cc1ccnc(C(F)(F)F)c1)S(=O)(=O)c1c(F)c(F)c(F)c(F)c1F] | B | | | D |
| 1134 | 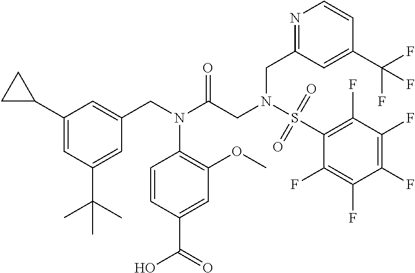 [SMILES: COc1cc(C(=O)O)ccc1N(Cc1cc(C2CC2)cc(C(C)(C)C)c1)C(=O)CN(Cc1cc(C(F)(F)F)ccn1)S(=O)(=O)c1c(F)c(F)c(F)c(F)c1F] | C | | | C |
| 1135 | 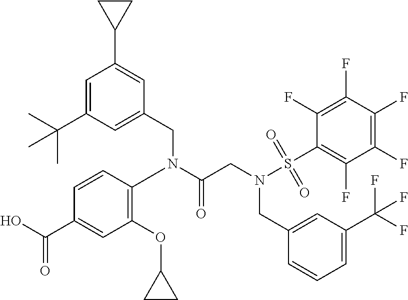 [SMILES: CC(C)(C)c1cc(CN(C(=O)CN(Cc2cccc(C(F)(F)F)c2)S(=O)(=O)c2c(F)c(F)c(F)c(F)c2F)c2ccc(C(=O)O)cc2OC2CC2)cc(C2CC2)c1] | B | | B | |
| 1136 | - 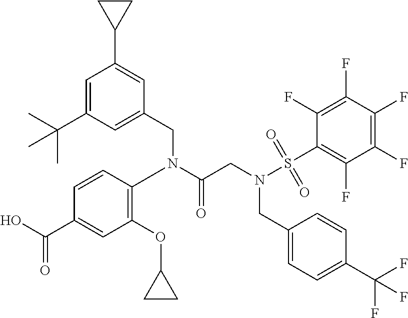 [SMILES: CC(C)(C)c1cc(CN(C(=O)CN(Cc2ccc(C(F)(F)F)cc2)S(=O)(=O)c2c(F)c(F)c(F)c(F)c2F)c2ccc(C(=O)O)cc2OC2CC2)cc(C2CC2)c1] | C | | B | A |
| 1137 | 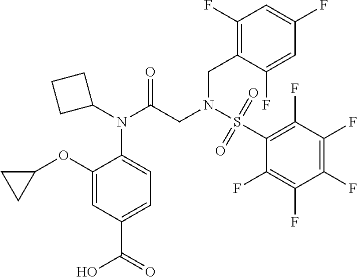 [SMILES: O=C(O)c1ccc(N(C(=O)CN(Cc2c(F)cc(F)cc2F)S(=O)(=O)c2c(F)c(F)c(F)c(F)c2F)C2CCC2)c(OC2CC2)c1] | C | | | D |
| 1138 | 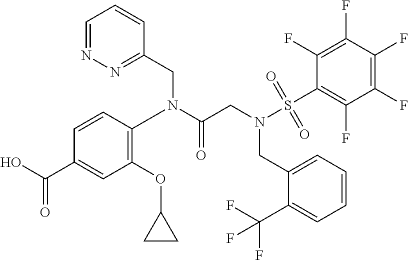 [SMILES: O=C(O)c1ccc(N(Cc2cccnn2)C(=O)CN(Cc2ccccc2C(F)(F)F)S(=O)(=O)c2c(F)c(F)c(F)c(F)c2F)c(OC2CC2)c1] | D | | | |
| 1139 | 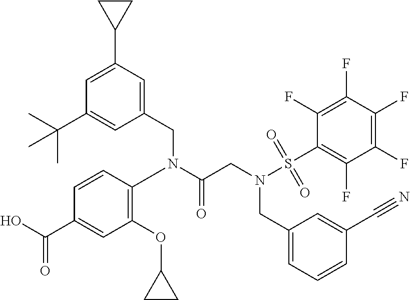 [SMILES: CC(C)(C)c1cc(CN(C(=O)CN(Cc2cccc(C#N)c2)S(=O)(=O)c2c(F)c(F)c(F)c(F)c2F)c2ccc(C(=O)O)cc2OC2CC2)cc(C2CC2)c1] | B | | | |
| 1140 | 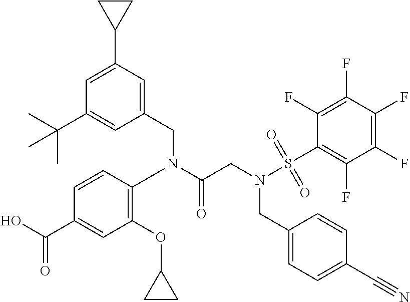 [SMILES: CC(C)(C)c1cc(CN(C(=O)CN(Cc2ccc(C#N)cc2)S(=O)(=O)c2c(F)c(F)c(F)c(F)c2F)c2ccc(C(=O)O)cc2OC2CC2)cc(C2CC2)c1] | B | | | |
| 1141 | 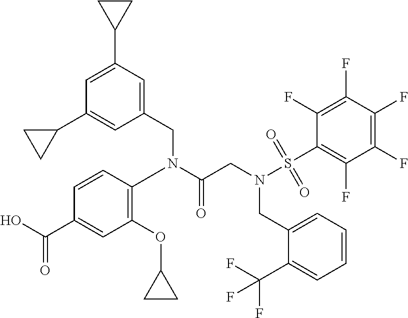 [SMILES: O=C(O)c1ccc(N(Cc2cc(C3CC3)cc(C3CC3)c2)C(=O)CN(Cc2ccccc2C(F)(F)F)S(=O)(=O)c2c(F)c(F)c(F)c(F)c2F)c(OC2CC2)c1] | A | C | | B |
| 1142 | 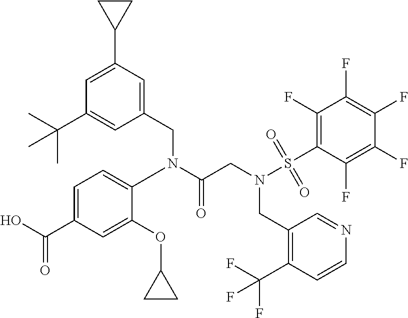 [SMILES: CC(C)(C)c1cc(CN(C(=O)CN(Cc2cnccc2C(F)(F)F)S(=O)(=O)c2c(F)c(F)c(F)c(F)c2F)c2ccc(C(=O)O)cc2OC2CC2)cc(C2CC2)c1] | A | C | A | B |
| 1143 | 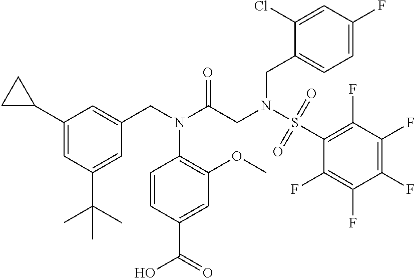 [SMILES: COc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | A | B | A | B |
| 1144 | 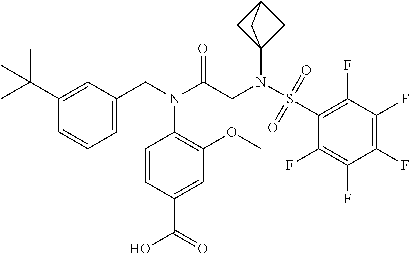 [SMILES: COc1cc(C(=O)O)ccc1N(Cc1cccc(C(C)(C)C)c1)C(=O)CN(C12CC(C1)C2)S(=O)(=O)c1c(F)c(F)c(F)c(F)c1F] | B | | | |
| 1145 | 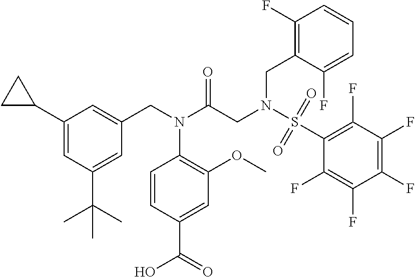 [SMILES: COc1cc(C(=O)O)ccc1N(Cc1cc(C2CC2)cc(C(C)(C)C)c1)C(=O)CN(Cc1c(F)cccc1F)S(=O)(=O)c1c(F)c(F)c(F)c(F)c1F] | A | A | | C |
| 1146 | 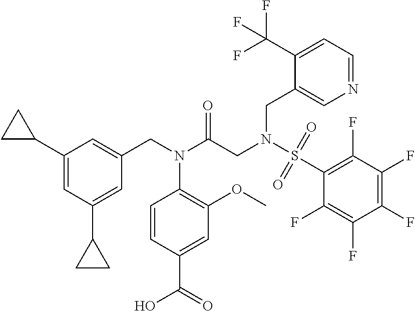 [SMILES: COc1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | B | A | | D |
| 1147 | 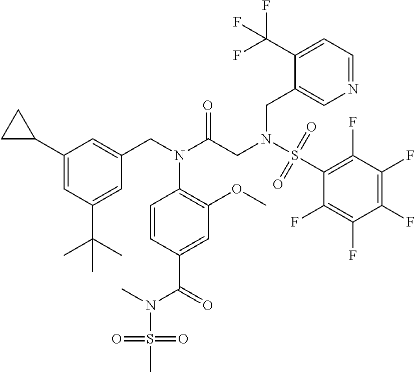 [SMILES: COc1cc(C(=O)N(C)S(C)(=O)=O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | A | | D | A |
| 1148 | 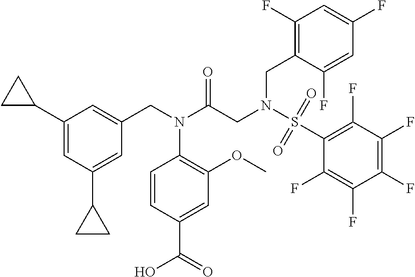 [SMILES: COc1cc(C(=O)O)ccc1N(Cc1cc(C2CC2)cc(C2CC2)c1)C(=O)CN(Cc1c(F)cc(F)cc1F)S(=O)(=O)c1c(F)c(F)c(F)c(F)c1F] | A | A | A | C |
| 1149 | 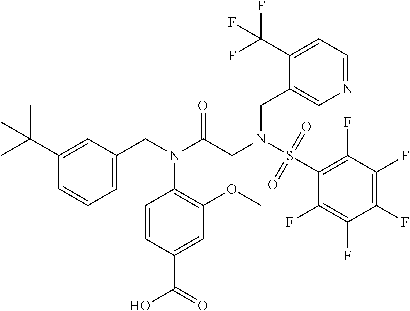 [SMILES: COc1cc(C(=O)O)ccc1N(Cc1cccc(C(C)(C)C)c1)C(=O)CN(Cc1cnccc1C(F)(F)F)S(=O)(=O)c1c(F)c(F)c(F)c(F)c1F] | B | | A | D |
| 1150 | 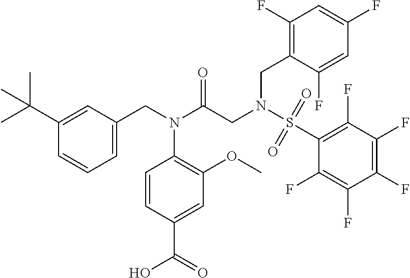 [SMILES: COc1cc(C(=O)O)ccc1N(Cc1cccc(C(C)(C)C)c1)C(=O)CN(Cc1c(F)cc(F)cc1F)S(=O)(=O)c1c(F)c(F)c(F)c(F)c1F] | A | | A | D |
| 1151 | 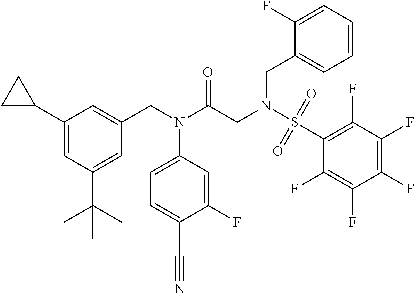 [SMILES: CC(C)(C)c1cc(CN(C(=O)CN(Cc2ccccc2F)S(=O)(=O)c2c(F)c(F)c(F)c(F)c2F)c2ccc(C#N)c(F)c2)cc(C2CC2)c1] | D | | | |
| 1152 | 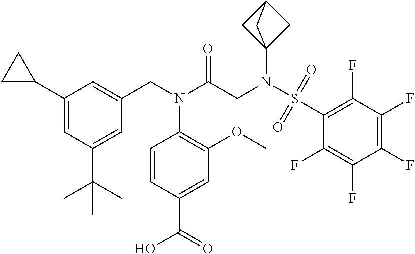 [SMILES: COc1cc(C(=O)O)ccc1N(Cc1cc(C2CC2)cc(C(C)(C)C)c1)C(=O)CN(C12CC(C1)C2)S(=O)(=O)c1c(F)c(F)c(F)c(F)c1F] | C | | | D |
| 1153 | 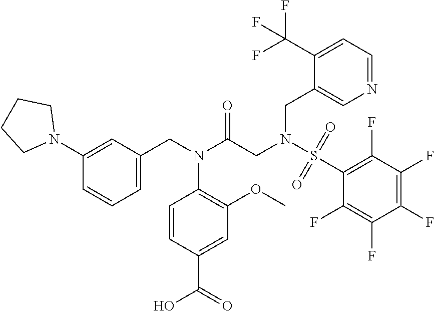 [SMILES: COc1cc(C(=O)O)ccc1N(Cc1cccc(N2CCCC2)c1)C(=O)CN(Cc1cnccc1C(F)(F)F)S(=O)(=O)c1c(F)c(F)c(F)c(F)c1F] | B | | A | D |
| 1154 | 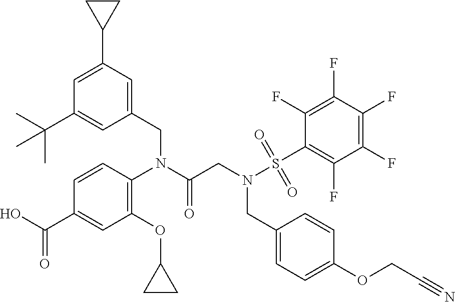 [SMILES: CC(C)(C)c1cc(CN(C(=O)CN(Cc2ccc(OCC#N)cc2)S(=O)(=O)c2c(F)c(F)c(F)c(F)c2F)c2ccc(C(=O)O)cc2OC2CC2)cc(C2CC2)c1] | | | | |
| 1155 | 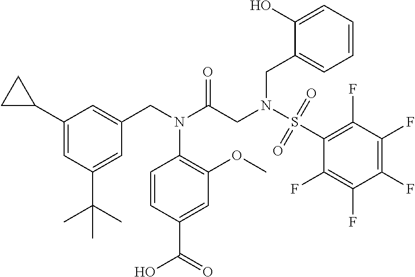 [SMILES: COc1cc(C(=O)O)ccc1N(Cc1cc(C2CC2)cc(C(C)(C)C)c1)C(=O)CN(Cc1ccccc1O)S(=O)(=O)c1c(F)c(F)c(F)c(F)c1F] | | | A | |
| 1156 | 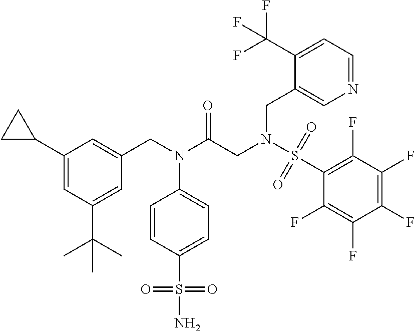 [SMILES: CC(C)(C)c1cc(CN(C(=O)CN(Cc2cnccc2C(F)(F)F)S(=O)(=O)c2c(F)c(F)c(F)c(F)c2F)c2ccc(S(N)(=O)=O)cc2)cc(C2CC2)c1] | | | | |
| 1157 | 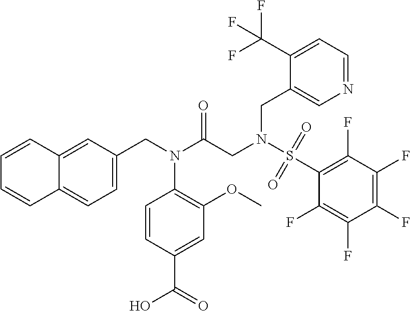 [SMILES: COc1cc(C(=O)O)ccc1N(Cc1ccc2ccccc2c1)C(=O)CN(Cc1cnccc1C(F)(F)F)S(=O)(=O)c1c(F)c(F)c(F)c(F)c1F] | C | | A | D |
| 1158 | 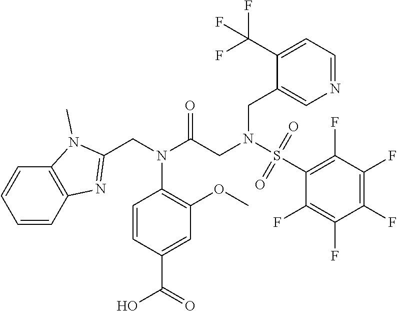 [SMILES: COc1cc(C(=O)O)ccc1N(Cc1nc2ccccc2n1C)C(=O)CN(Cc1cnccc1C(F)(F)F)S(=O)(=O)c1c(F)c(F)c(F)c(F)c1F] | D | | | |
| 1159 | 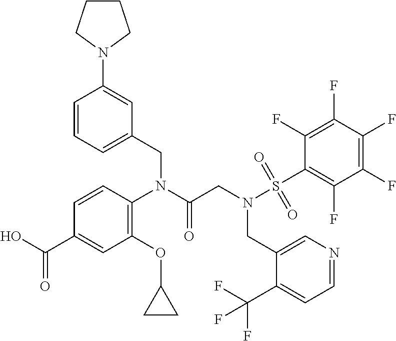 [SMILES: O=C(O)c1ccc(N(Cc2cccc(N3CCCC3)c2)C(=O)CN(Cc2cnccc2C(F)(F)F)S(=O)(=O)c2c(F)c(F)c(F)c(F)c2F)c(OC2CC2)c1] | | | A | D |
| 1160 | 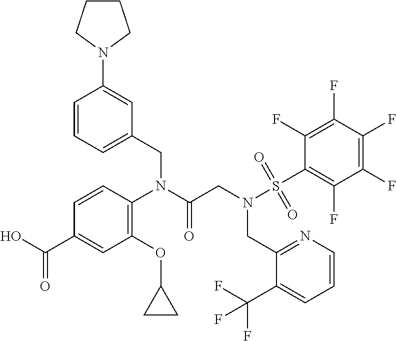 [SMILES: O=C(O)c1ccc(N(Cc2cccc(N3CCCC3)c2)C(=O)CN(Cc2ncccc2C(F)(F)F)S(=O)(=O)c2c(F)c(F)c(F)c(F)c2F)c(OC2CC2)c1] | | | | D |
| 1161 | 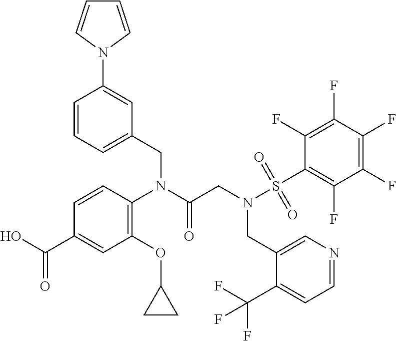 [SMILES: O=C(O)c1ccc(N(Cc2cccc(-n3cccc3)c2)C(=O)CN(Cc2cnccc2C(F)(F)F)S(=O)(=O)c2c(F)c(F)c(F)c(F)c2F)c(OC2CC2)c1] | B | | | D |
| 1162 | 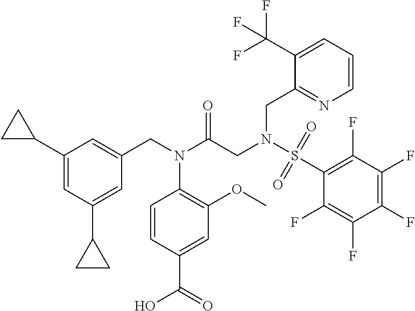 [SMILES: COc1cc(C(=O)O)ccc1N(Cc1cc(C2CC2)cc(C2CC2)c1)C(=O)CN(Cc1ncccc1C(F)(F)F)S(=O)(=O)c1c(F)c(F)c(F)c(F)c1F] | | B | | C |
| 1163 | 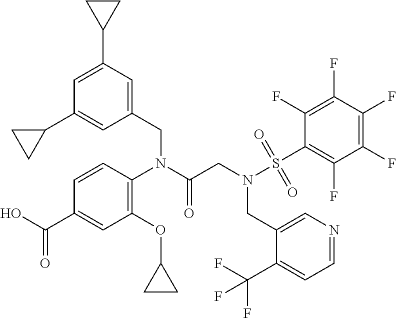 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(OC2CC2)c1] | A | | | C |
| 1164 | 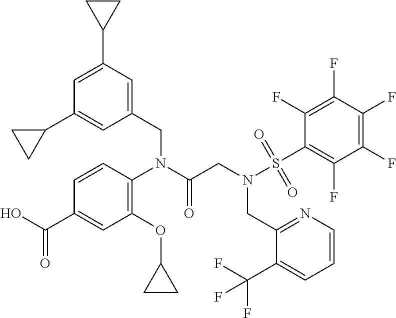 [SMILES: O=C(O)c1ccc(N(Cc2cc(C3CC3)cc(C3CC3)c2)C(=O)CN(Cc2ncccc2C(F)(F)F)S(=O)(=O)c2c(F)c(F)c(F)c(F)c2F)c(OC2CC2)c1] | B | C | | B |
| 1165 | 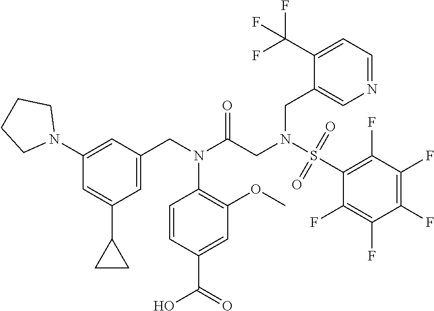 [SMILES: COc1cc(C(=O)O)ccc1N(Cc1cc(C2CC2)cc(N2CCCC2)c1)C(=O)CN(Cc1cnccc1C(F)(F)F)S(=O)(=O)c1c(F)c(F)c(F)c(F)c1F] | B | | | D |
| 1166 | 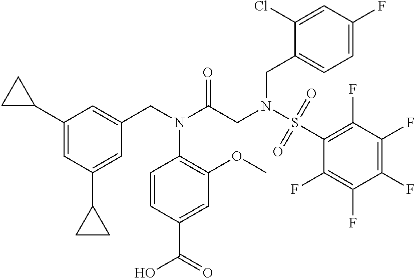 [SMILES: COc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | A | A | | C |
| 1167 | 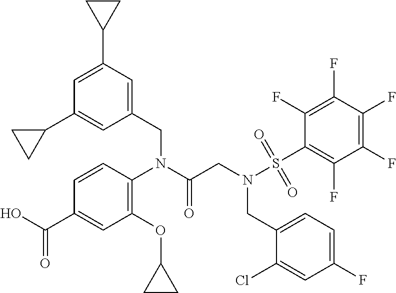 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CC2)c1] | A | B | | B |
| 1168 | 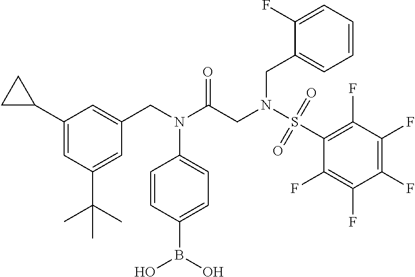 [SMILES: CC(C)(C)c1cc(CN(C(=O)CN(Cc2ccccc2F)S(=O)(=O)c2c(F)c(F)c(F)c(F)c2F)c2ccc(B(O)O)cc2)cc(C2CC2)c1] | | | | |
| 1169 | 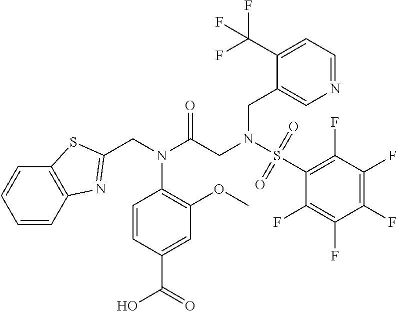 [SMILES: COc1cc(C(=O)O)ccc1N(Cc1nc2ccccc2s1)C(=O)CN(Cc1cnccc1C(F)(F)F)S(=O)(=O)c1c(F)c(F)c(F)c(F)c1F] | | | | |
| 1170 | 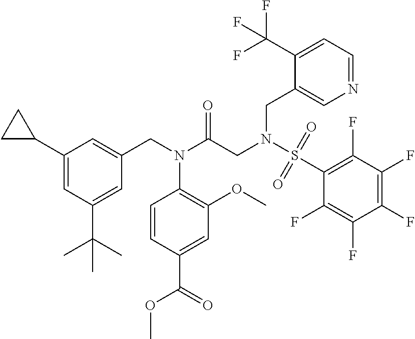 [SMILES: COC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | | | | |
| 1171 | 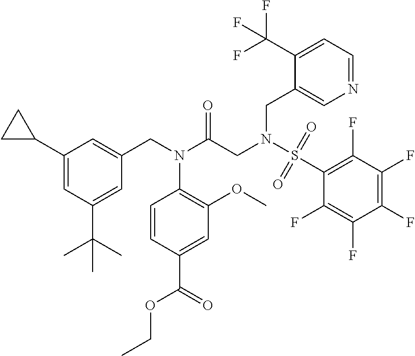 [SMILES: CCOC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | | | | |
| 1172 | 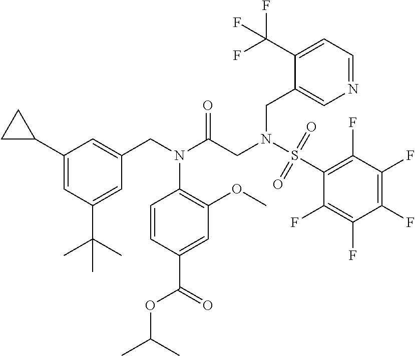 [SMILES: COc1cc(C(=O)OC(C)C)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | | | | A |
| 1173 | 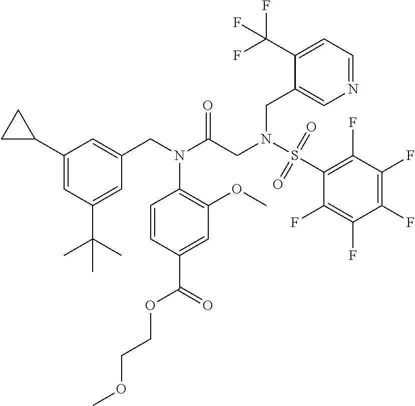 [SMILES: COCCOC(=O)c1ccc(N(Cc2cc(C3CC3)cc(C(C)(C)C)c2)C(=O)CN(Cc2cnccc2C(F)(F)F)S(=O)(=O)c2c(F)c(F)c(F)c(F)c2F)c(OC)c1] | | | | |
| 1174 | 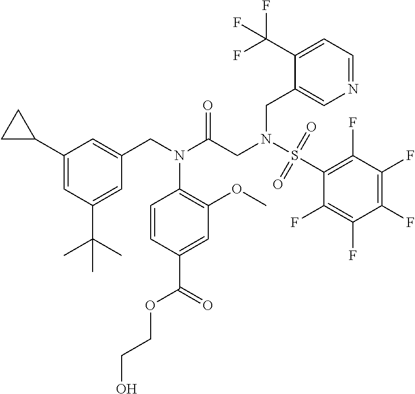 [SMILES: COc1cc(C(=O)OCCO)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | | | | |
| 1175 | 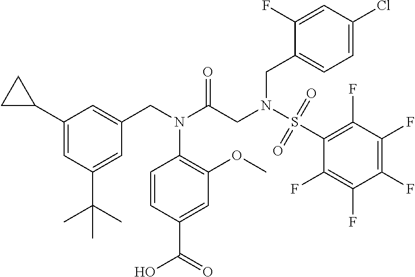 [SMILES: COc1cc(C(=O)O)ccc1N(Cc1cc(C2CC2)cc(C(C)(C)C)c1)C(=O)CN(Cc1ccc(Cl)cc1F)S(=O)(=O)c1c(F)c(F)c(F)c(F)c1F] | B | | | |
| 1176 | 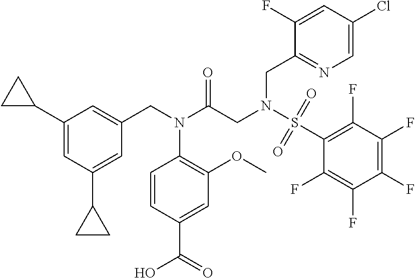 [SMILES: COc1cc(C(=O)O)ccc1N(Cc1cc(C2CC2)cc(C2CC2)c1)C(=O)CN(Cc1ncc(Cl)cc1F)S(=O)(=O)c1c(F)c(F)c(F)c(F)c1F] | A | | | C |
| 1177 | 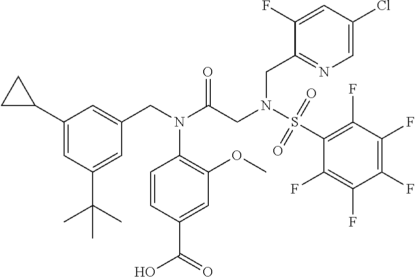 [SMILES: COc1cc(C(=O)O)ccc1N(Cc1cc(C2CC2)cc(C(C)(C)C)c1)C(=O)CN(Cc1ncc(Cl)cc1F)S(=O)(=O)c1c(F)c(F)c(F)c(F)c1F] | | | | D |
| 1178 | 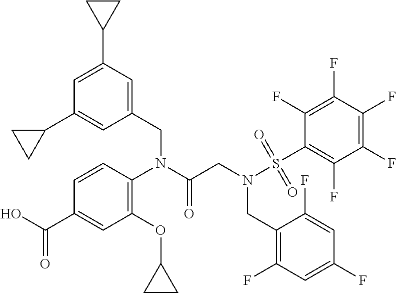 [SMILES: O=C(O)c1ccc(N(Cc2cc(C3CC3)cc(C3CC3)c2)C(=O)CN(Cc2c(F)cc(F)cc2F)S(=O)(=O)c2c(F)c(F)c(F)c(F)c2F)c(OC2CC2)c1] | A | B | | B |
| 1179 | 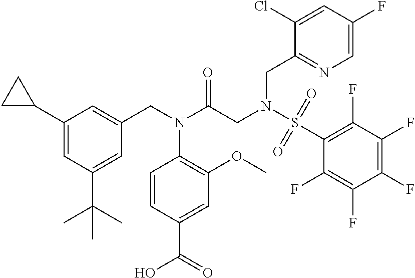 [SMILES: COc1cc(C(=O)O)ccc1N(Cc1cc(C2CC2)cc(C(C)(C)C)c1)C(=O)CN(Cc1ncc(F)cc1Cl)S(=O)(=O)c1c(F)c(F)c(F)c(F)c1F] | A | | | C |
| 1180 | 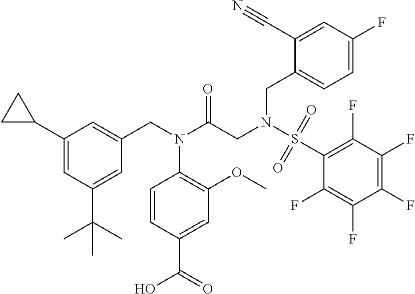 [SMILES: COc1cc(C(=O)O)ccc1N(Cc1cc(C2CC2)cc(C(C)(C)C)c1)C(=O)CN(Cc1ccc(F)cc1C#N)S(=O)(=O)c1c(F)c(F)c(F)c(F)c1F] | A | | | C |
| 1181 | 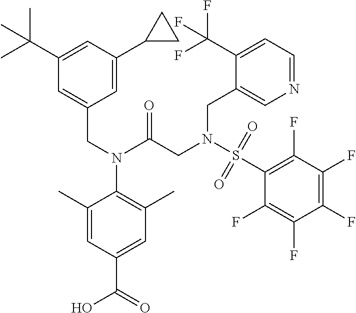 [SMILES: Cc1cc(C(=O)O)cc(C)c1N(Cc1cc(C2CC2)cc(C(C)(C)C)c1)C(=O)CN(Cc1cnccc1C(F)(F)F)S(=O)(=O)c1c(F)c(F)c(F)c(F)c1F] | B | | | |
| 1182 | 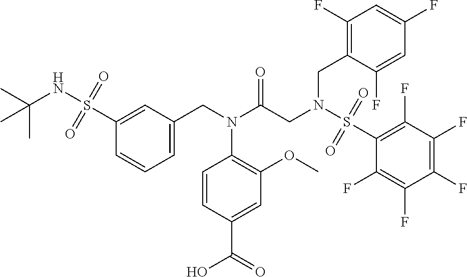 [SMILES: COc1cc(C(=O)O)ccc1N(Cc1cccc(S(=O)(=O)NC(C)(C)C)c1)C(=O)CN(Cc1c(F)cc(F)cc1F)S(=O)(=O)c1c(F)c(F)c(F)c(F)c1F] | | | | |
| 1183 | 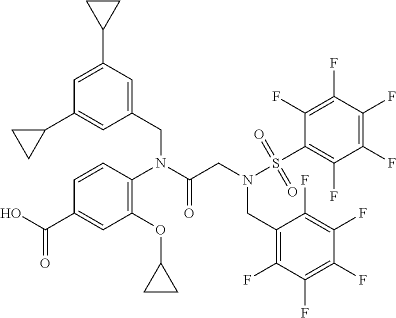 [SMILES: O=C(O)c1ccc(N(Cc2cc(C3CC3)cc(C3CC3)c2)C(=O)CN(Cc2c(F)c(F)c(F)c(F)c2F)S(=O)(=O)c2c(F)c(F)c(F)c(F)c2F)c(OC2CC2)c1] | A | A | | A |
| 1186 | 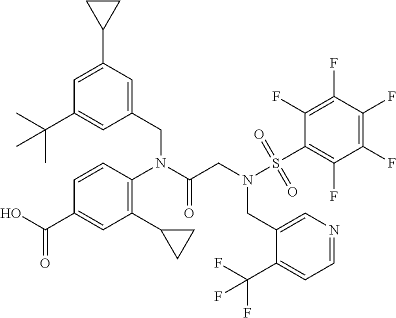 [SMILES: CC(C)(C)c1cc(CN(C(=O)CN(Cc2cnccc2C(F)(F)F)S(=O)(=O)c2c(F)c(F)c(F)c(F)c2F)c2ccc(C(=O)O)cc2C2CC2)cc(C2CC2)c1] | A | A | | C |
| 1187 | 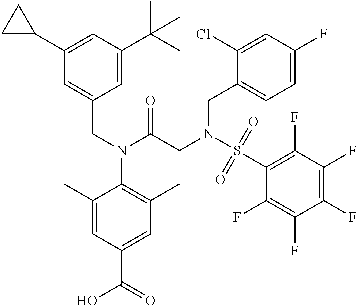 [SMILES: Cc1cc(C(=O)O)cc(C)c1N(Cc1cc(C2CC2)cc(C(C)(C)C)c1)C(=O)CN(Cc1ccc(F)cc1Cl)S(=O)(=O)c1c(F)c(F)c(F)c(F)c1F] | B | | | |
| 1188 | 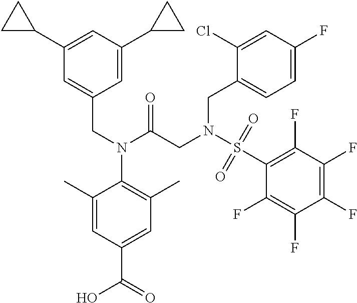 [SMILES: Cc1cc(C(=O)O)cc(C)c1N(Cc1cc(C2CC2)cc(C2CC2)c1)C(=O)CN(Cc1ccc(F)cc1Cl)S(=O)(=O)c1c(F)c(F)c(F)c(F)c1F] | B | A | | |
| 1189 | 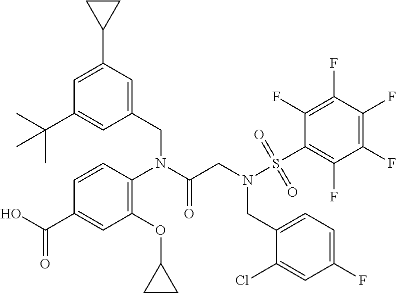 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2OC2CC2)cc(C2CC2)c1] | A | B | | A |
| 1190 | 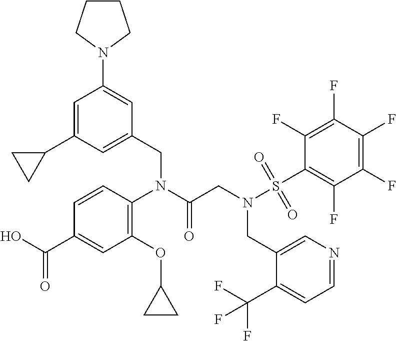 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(OC2CC2)c1] | | A | | C |
| 1191 | 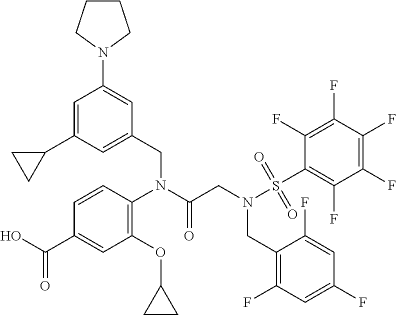 [SMILES: O=C(O)c1ccc(N(Cc2cc(C3CC3)cc(N3CCCC3)c2)C(=O)CN(Cc2c(F)cc(F)cc2F)S(=O)(=O)c2c(F)c(F)c(F)c(F)c2F)c(OC2CC2)c1] | B | A | | A |
| 1192 | 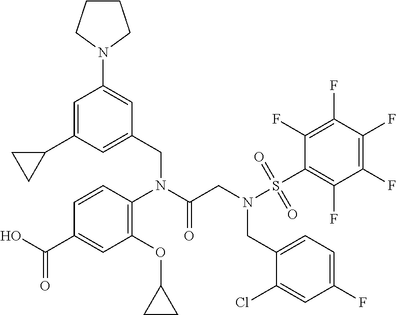 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(OC2CC2)c1] | B | B | | A |
| 1193 | 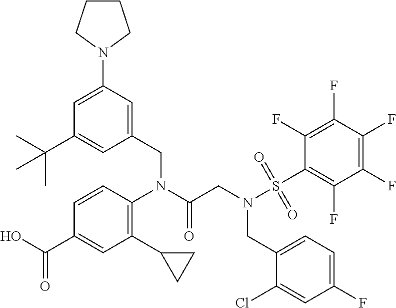 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2C2CC2)cc(N2CCCC2)c1] | A | B | | A |
| 1194 | 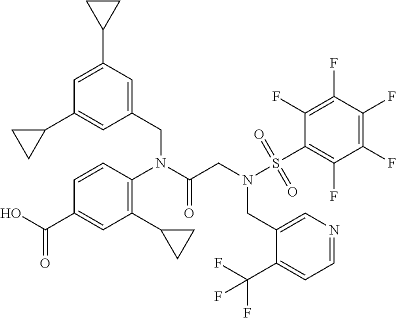 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(C2CC2)c1] | A | A | | D |
| 1195 | 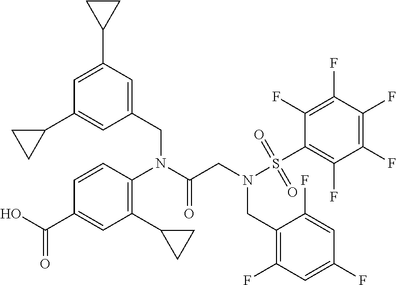 [SMILES: O=C(O)c1ccc(N(Cc2cc(C3CC3)cc(C3CC3)c2)C(=O)CN(Cc2c(F)cc(F)cc2F)S(=O)(=O)c2c(F)c(F)c(F)c(F)c2F)c(C2CC2)c1] | A | A | | C |
| 1196 | . 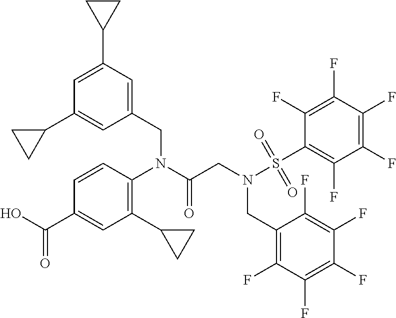 [SMILES: O=C(O)c1ccc(N(Cc2cc(C3CC3)cc(C3CC3)c2)C(=O)CN(Cc2c(F)c(F)c(F)c(F)c2F)S(=O)(=O)c2c(F)c(F)c(F)c(F)c2F)c(C2CC2)c1] | A | A | | A |
| 1197 | 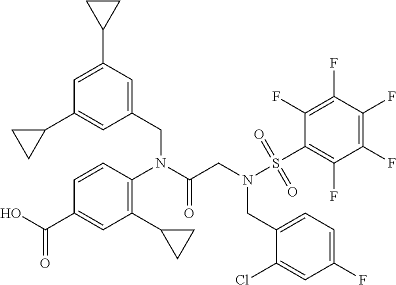 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(C2CC2)c1] | A | B | | B |
| 1198 | 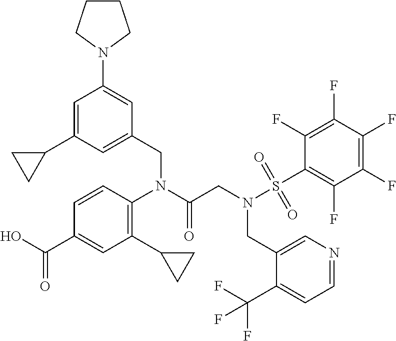 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(C2CC2)c1] | | | | |
| 1199 | 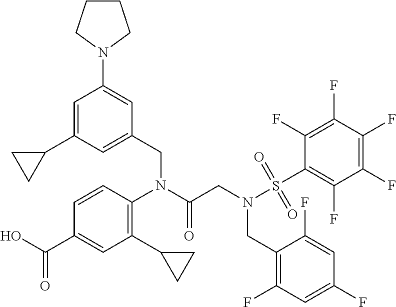 [SMILES: O=C(O)c1ccc(N(Cc2cc(C3CC3)cc(N3CCCC3)c2)C(=O)CN(Cc2c(F)cc(F)cc2F)S(=O)(=O)c2c(F)c(F)c(F)c(F)c2F)c(C2CC2)c1] | A | A | | A |
| 1200 | 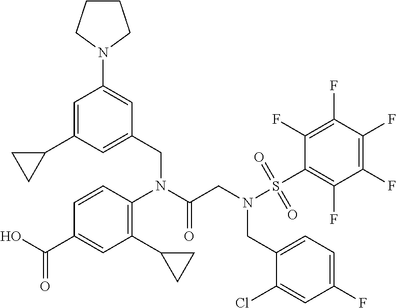 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(C2CC2)c1] | A | A | | A |
| 1201 | 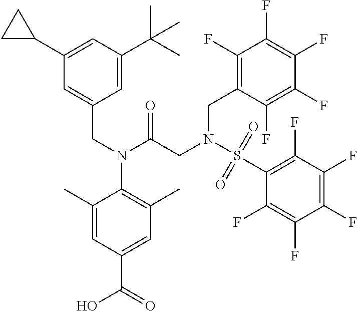 [SMILES: Cc1cc(C(=O)O)cc(C)c1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F] | B | | | A |
| 1202 | 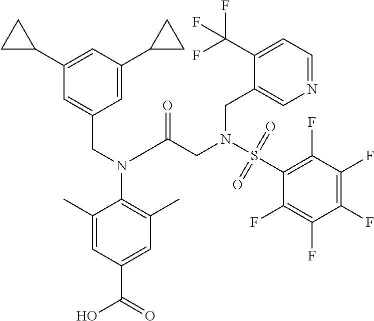 [SMILES: Cc1cc(C(=O)O)cc(C)c1N(Cc1cc(C2CC2)cc(C2CC2)c1)C(=O)CN(Cc1cnccc1C(F)(F)F)S(=O)(=O)c1c(F)c(F)c(F)c(F)c1F] | B | | | |
| 1203 | 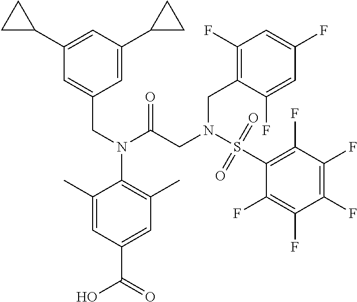 [SMILES: Cc1cc(C(=O)O)cc(C)c1N(Cc1cc(C2CC2)cc(C2CC2)c1)C(=O)CN(Cc1c(F)cc(F)cc1F)S(=O)(=O)c1c(F)c(F)c(F)c(F)c1F] | A | A | | B |
| 1204 | 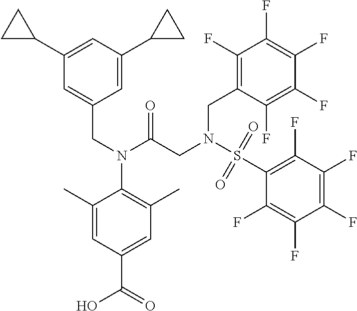 [SMILES: Cc1cc(C(=O)O)cc(C)c1N(Cc1cc(C2CC2)cc(C2CC2)c1)C(=O)CN(Cc1c(F)c(F)c(F)c(F)c1F)S(=O)(=O)c1c(F)c(F)c(F)c(F)c1F] | B | A | | |
| 1205 | 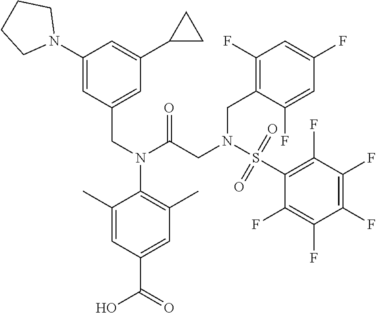 [SMILES: Cc1cc(C(=O)O)cc(C)c1N(Cc1cc(C2CC2)cc(N2CCCC2)c1)C(=O)CN(Cc1c(F)cc(F)cc1F)S(=O)(=O)c1c(F)c(F)c(F)c(F)c1F] | B | | | |
| 1206 | 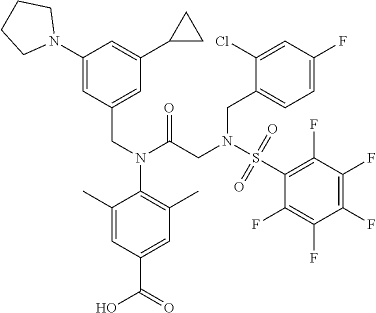 [SMILES: Cc1cc(C(=O)O)cc(C)c1N(Cc1cc(C2CC2)cc(N2CCCC2)c1)C(=O)CN(Cc1ccc(F)cc1Cl)S(=O)(=O)c1c(F)c(F)c(F)c(F)c1F] | B | | | |
| 1207 | 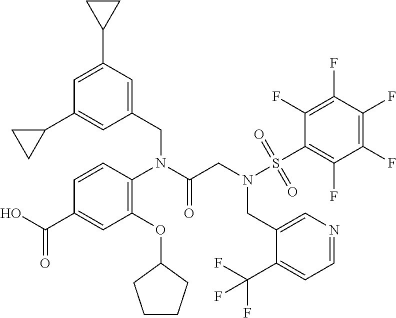 [SMILES: O=C(O)c1ccc(N(Cc2cc(C3CC3)cc(C3CC3)c2)C(=O)CN(Cc2cnccc2C(F)(F)F)S(=O)(=O)c2c(F)c(F)c(F)c(F)c2F)c(OC2CCCC2)c1] | A | B | | B |
| 1208 | 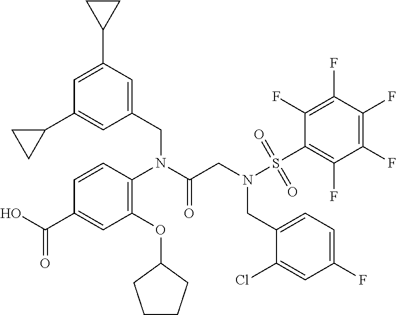 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCCC2)c1] | A | B | | A |
| 1209 | 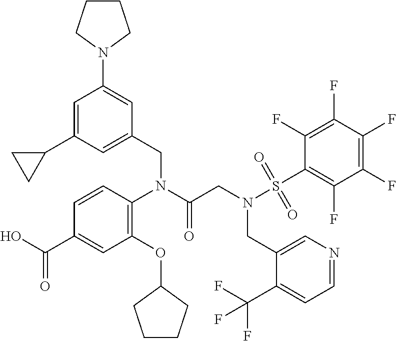 [SMILES: O=C(O)c1ccc(N(Cc2cc(C3CC3)cc(N3CCCC3)c2)C(=O)CN(Cc2cnccc2C(F)(F)F)S(=O)(=O)c2c(F)c(F)c(F)c(F)c2F)c(OC2CCCC2)c1] | A | B | | A |
| 1210 | 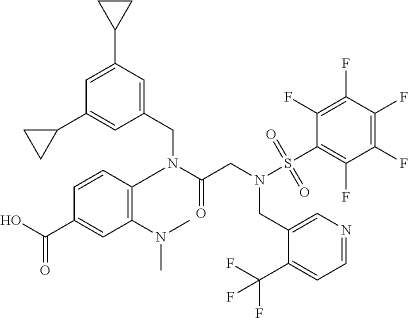 [SMILES: CN(C)c1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | A | A | | D |
| 1211 | 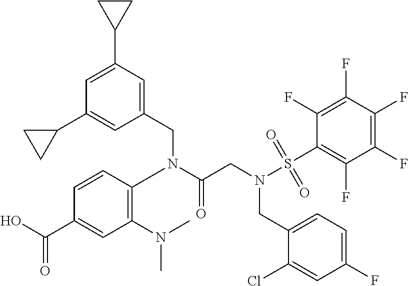 [SMILES: CN(C)c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | B | B | | B |
| 1212 | 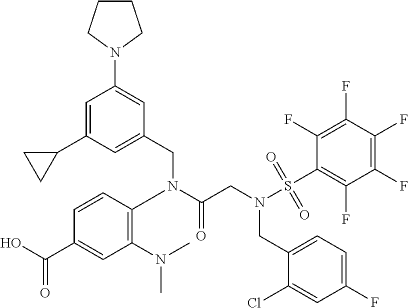 [SMILES: CN(C)c1cc(C(=O)O)ccc1N(Cc1cc(C2CC2)cc(N2CCCC2)c1)C(=O)CN(Cc1ccc(F)cc1Cl)S(=O)(=O)c1c(F)c(F)c(F)c(F)c1F] | B | B | | |
| 1213 | 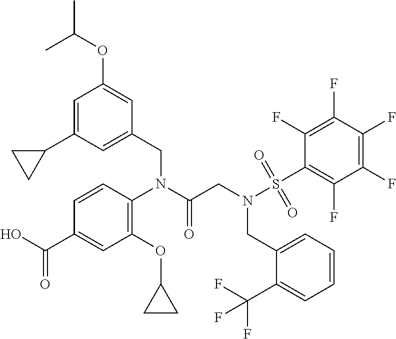 [SMILES: CC(C)Oc1cc(CN(C(=O)CN(Cc2ccccc2C(F)(F)F)S(=O)(=O)c2c(F)c(F)c(F)c(F)c2F)c2ccc(C(=O)O)cc2OC2CC2)cc(C2CC2)c1] | A | B | | C |
| 1214 | 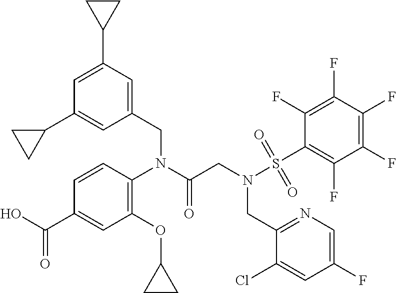 [SMILES: O=C(O)c1ccc(N(Cc2cc(C3CC3)cc(C3CC3)c2)C(=O)CN(Cc2ncc(F)cc2Cl)S(=O)(=O)c2c(F)c(F)c(F)c(F)c2F)c(OC2CC2)c1] | A | A | | B |
| 1215 | 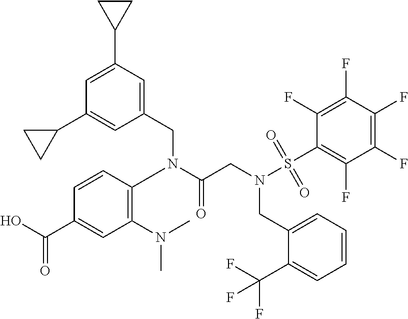 [SMILES: CN(C)c1cc(C(=O)O)ccc1N(Cc1cc(C2CC2)cc(C2CC2)c1)C(=O)CN(Cc1ccccc1C(F)(F)F)S(=O)(=O)c1c(F)c(F)c(F)c(F)c1F] | A | C | | B |
| 1216 | 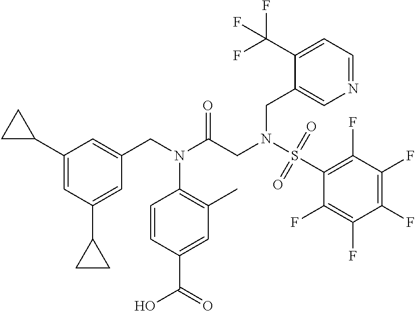 [SMILES: Cc1cc(C(=O)O)ccc1N(Cc1cc(C2CC2)cc(C2CC2)c1)C(=O)CN(Cc1cnccc1C(F)(F)F)S(=O)(=O)c1c(F)c(F)c(F)c(F)c1F] | A | A | | D |
| 1217 | 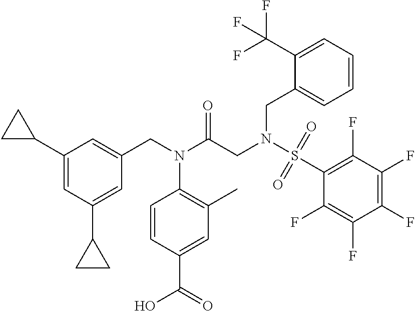 [SMILES: Cc1cc(C(=O)O)ccc1N(Cc1cc(C2CC2)cc(C2CC2)c1)C(=O)CN(Cc1ccccc1C(F)(F)F)S(=O)(=O)c1c(F)c(F)c(F)c(F)c1F] | A | C | | B |
| 1218 | 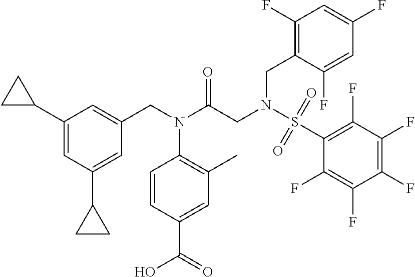 [SMILES: Cc1cc(C(=O)O)ccc1N(Cc1cc(C2CC2)cc(C2CC2)c1)C(=O)CN(Cc1c(F)cc(F)cc1F)S(=O)(=O)c1c(F)c(F)c(F)c(F)c1F] | A | A | | D |
| 1219 | 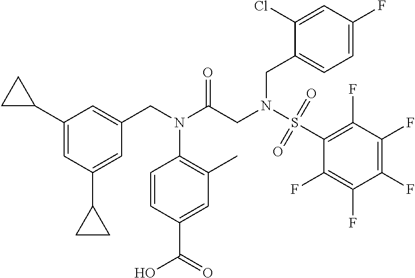 [SMILES: Cc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | A | A | | C |
| 1220 | 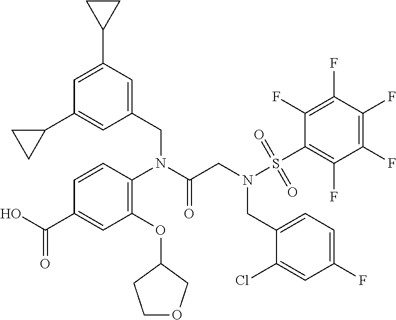 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCOC2)c1] | A | A | | C |
| 1221 | 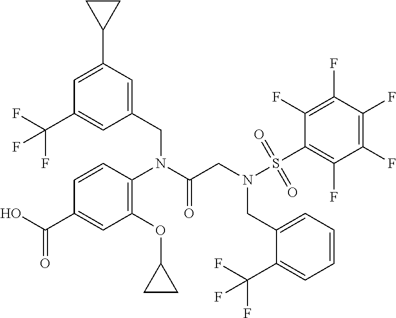 [SMILES: O=C(O)c1ccc(N(Cc2cc(C3CC3)cc(C(F)(F)F)c2)C(=O)CN(Cc2ccccc2C(F)(F)F)S(=O)(=O)c2c(F)c(F)c(F)c(F)c2F)c(OC2CC2)c1] | B | C | | A |
| 1222 | 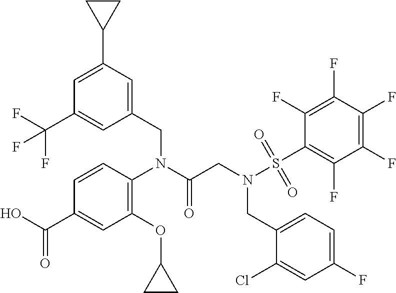 [SMILES: O=C(O)c1ccc(N(Cc2cc(C3CC3)cc(C(F)(F)F)c2)C(=O)CN(Cc2ccc(F)cc2Cl)S(=O)(=O)c2c(F)c(F)c(F)c(F)c2F)c(OC2CC2)c1] | A | B | | B |
| 1223 | 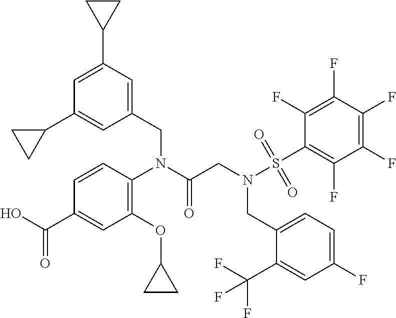 [SMILES: O=C(O)c1ccc(N(Cc2cc(C3CC3)cc(C3CC3)c2)C(=O)CN(Cc2ccc(F)cc2C(F)(F)F)S(=O)(=O)c2c(F)c(F)c(F)c(F)c2F)c(OC2CC2)c1] | | C | | B |
| 1224 | 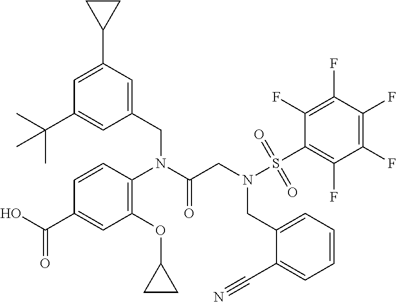 [SMILES: CC(C)(C)c1cc(CN(C(=O)CN(Cc2ccccc2C#N)S(=O)(=O)c2c(F)c(F)c(F)c(F)c2F)c2ccc(C(=O)O)cc2OC2CC2)cc(C2CC2)c1] | | A | | |
| 1225 | 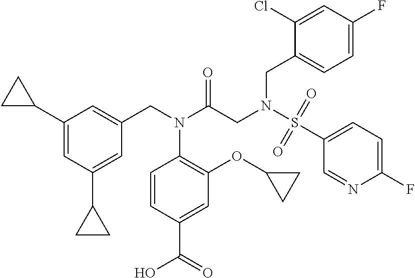 [SMILES: O=C(O)c1ccc(N(Cc2cc(C3CC3)cc(C3CC3)c2)C(=O)CN(Cc2ccc(F)cc2Cl)S(=O)(=O)c2ccc(F)nc2)c(OC2CC2)c1] | D | | | A |
| 1226 | 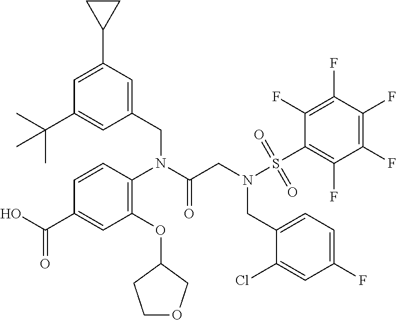 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2OC2CCOC2)cc(C2CC2)c1] | A | A | | C |
| 1227 | 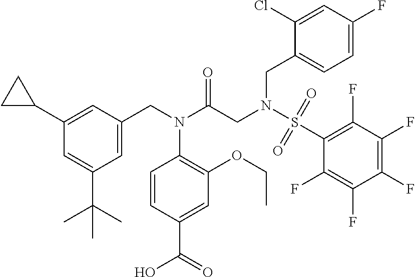 [SMILES: CCOc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | A | B | | B |
| 1228 | 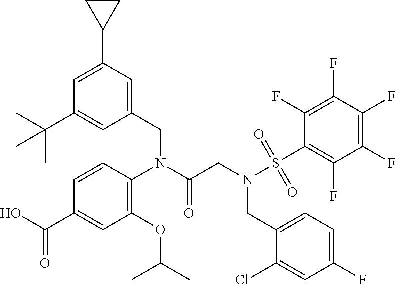 [SMILES: CC(C)Oc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | A | C | | A |
| 1229 | 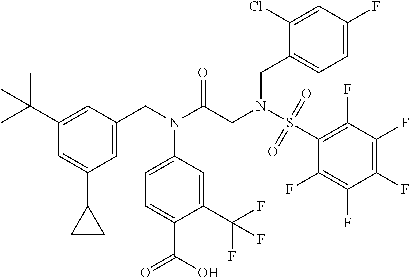 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)c(C(F)(F)F)c2)cc(C2CC2)c1] | C | | | |
| 1230 | . 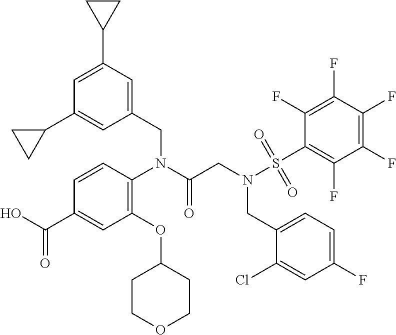 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCOCC2)c1] | | A | | C |
| 1231 | 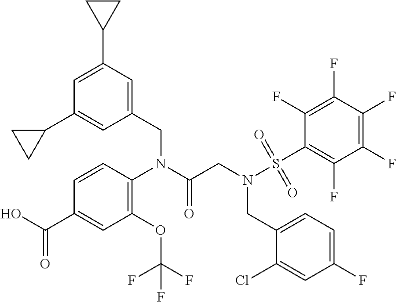 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC(F)(F)F)c1] | A | B | | B |
| 1232 | 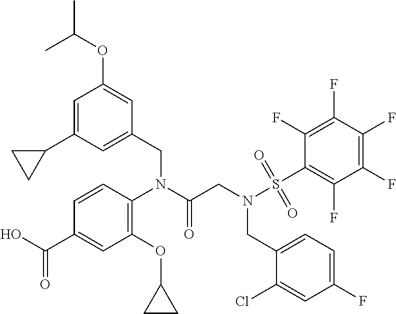 [SMILES: CC(C)Oc1cc(CN(C(=O)CN(Cc2ccc(F)cc2Cl)S(=O)(=O)c2c(F)c(F)c(F)c(F)c2F)c2ccc(C(=O)O)cc2OC2CC2)cc(C2CC2)c1] | A | B | | B |
| 1234 | 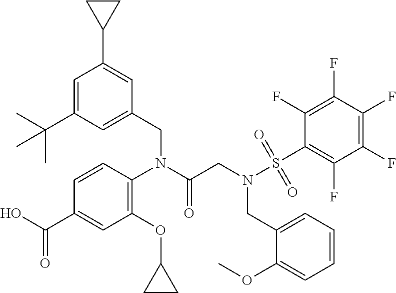 [SMILES: COc1ccccc1CN(CC(=O)N(Cc1cc(C2CC2)cc(C(C)(C)C)c1)c1ccc(C(=O)O)cc1OC1CC1)S(=O)(=O)c1c(F)c(F)c(F)c(F)c1F] | B | | | B |
| 1235 | 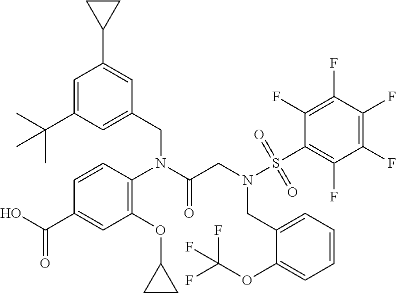 [SMILES: CC(C)(C)c1cc(CN(C(=O)CN(Cc2ccccc2OC(F)(F)F)S(=O)(=O)c2c(F)c(F)c(F)c(F)c2F)c2ccc(C(=O)O)cc2OC2CC2)cc(C2CC2)c1] | B | | | A |
| 1236 | 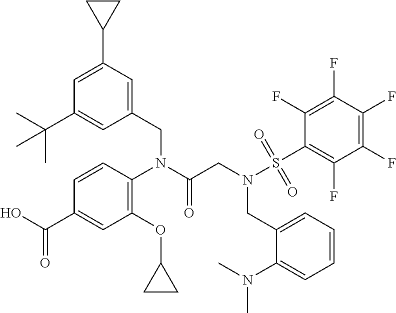 [SMILES: CN(C)c1ccccc1CN(CC(=O)N(Cc1cc(C2CC2)cc(C(C)(C)C)c1)c1ccc(C(=O)O)cc1OC1CC1)S(=O)(=O)c1c(F)c(F)c(F)c(F)c1F] | B | | | A |
| 1237 | 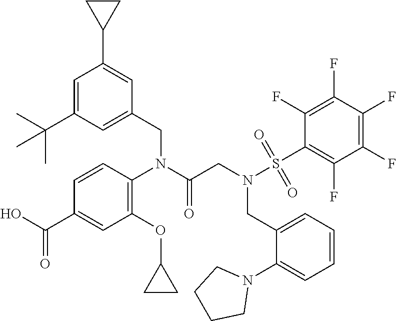 [SMILES: CC(C)(C)c1cc(CN(C(=O)CN(Cc2ccccc2N2CCCC2)S(=O)(=O)c2c(F)c(F)c(F)c(F)c2F)c2ccc(C(=O)O)cc2OC2CC2)cc(C2CC2)c1] | B | | | A |
| 1238 | 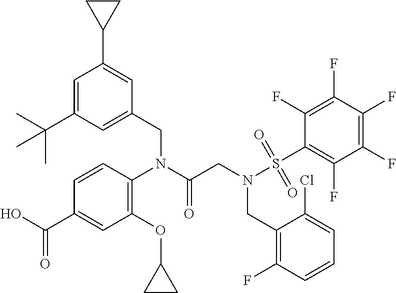 [SMILES: CC(C)(C)c1cc(CN(C(=O)CN(Cc2c(F)cccc2Cl)S(=O)(=O)c2c(F)c(F)c(F)c(F)c2F)c2ccc(C(=O)O)cc2OC2CC2)cc(C2CC2)c1] | A | B | | A |
| 1239 | 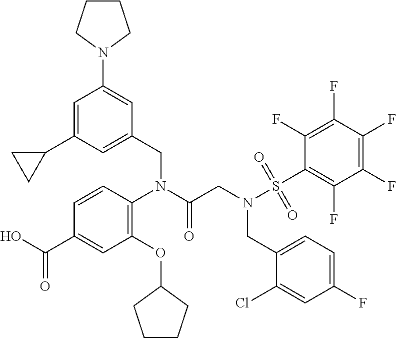 [SMILES: O=C(O)c1ccc(N(Cc2cc(C3CC3)cc(N3CCCC3)c2)C(=O)CN(Cc2ccc(F)cc2Cl)S(=O)(=O)c2c(F)c(F)c(F)c(F)c2F)c(OC2CCCC2)c1] | A | C | | |
| 1240 | 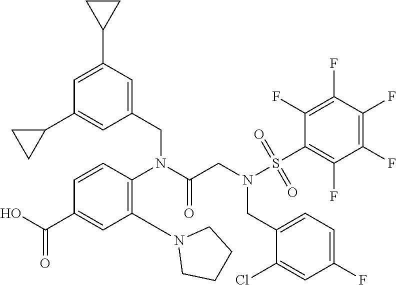 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCCC2)c1] | A | B | | |
| 1241 | 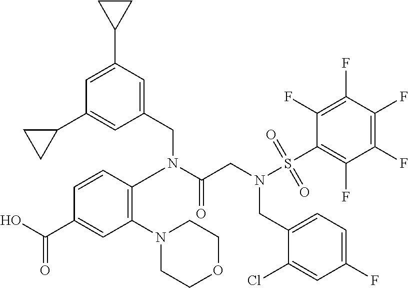 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCOCC2)c1] | A | A | | B |
| 1242 | 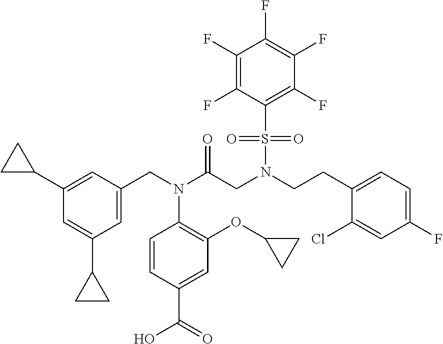 [SMILES: O=C(O)c1ccc(N(Cc2cc(C3CC3)cc(C3CC3)c2)C(=O)CN(CCc2ccc(F)cc2Cl)S(=O)(=O)c2c(F)c(F)c(F)c(F)c2F)c(OC2CC2)c1] | B | B | | D |
| 1243 | 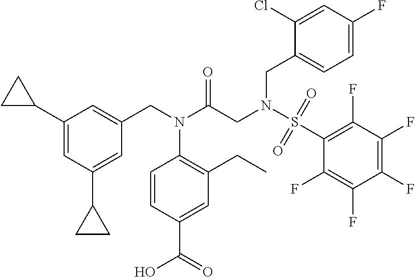 [SMILES: CCc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | B | | |
| 1244 | 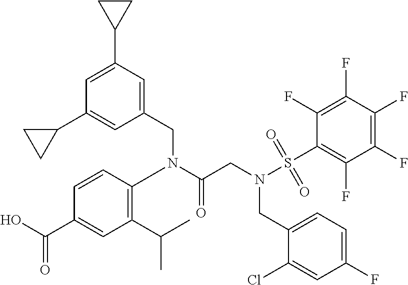 [SMILES: CC(C)c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | B | | |
| 1245 | 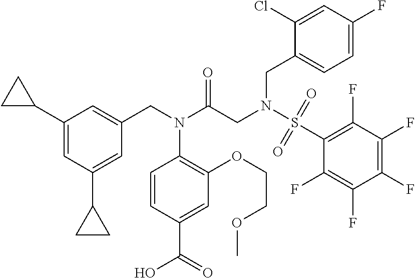 [SMILES: COCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | A | | C |
| 1246 | 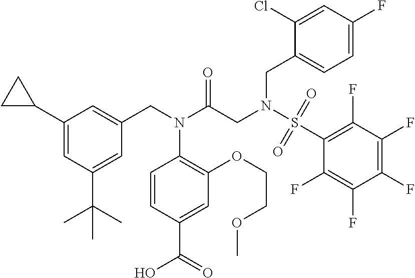 [SMILES: COCCOc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | A | B | | C |
| 1247 | 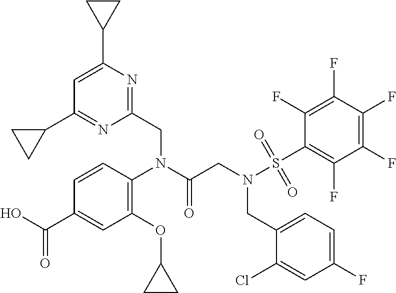 [SMILES: O=C(O)c1ccc(N(Cc2nc(C3CC3)cc(C3CC3)n2)C(=O)CN(Cc2ccc(F)cc2Cl)S(=O)(=O)c2c(F)c(F)c(F)c(F)c2F)c(OC2CC2)c1] | | B | | C |
| 1248 | 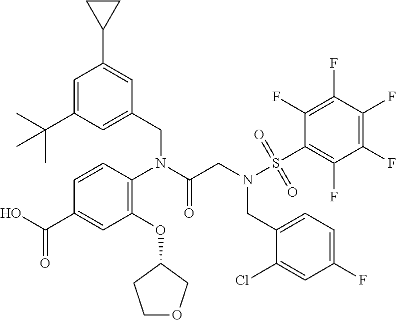 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2O[C@H]2CCOC2)cc(C2CC2)c1] | | A | | C |
| 1249 | 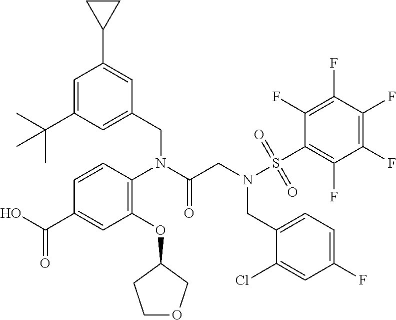 [SMILES: CC(C)(C)c1cc(CN(C(=O)CN(Cc2ccc(F)cc2Cl)S(=O)(=O)c2c(F)c(F)c(F)c(F)c2F)c2ccc(C(=O)O)cc2O[C@@H]2CCOC2)cc(C2CC2)c1] | | B | | C |
| 1250 | 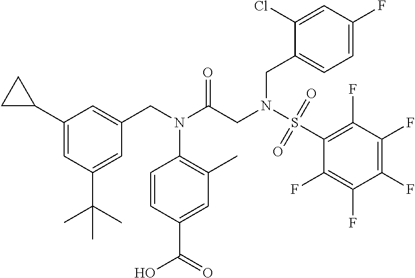 [SMILES: Cc1cc(C(=O)O)ccc1N(Cc1cc(C2CC2)cc(C(C)(C)C)c1)C(=O)CN(Cc1ccc(F)cc1Cl)S(=O)(=O)c1c(F)c(F)c(F)c(F)c1F] | A | A | | |
| 1251 | 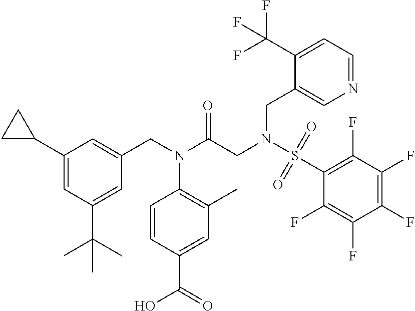 [SMILES: Cc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | A | A | | D |
| 1252 | 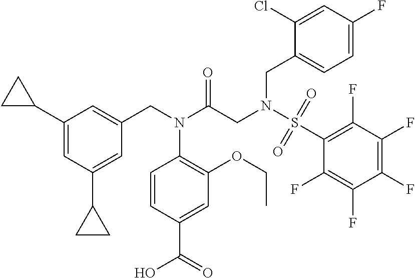 [SMILES: CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | A | A | | C |
| 1253 | 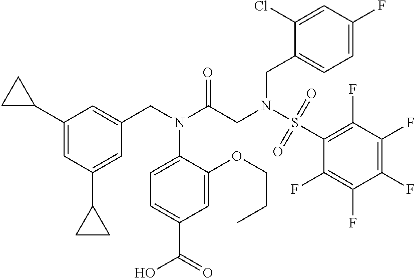 [SMILES: CCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | | | |
| 1254 | 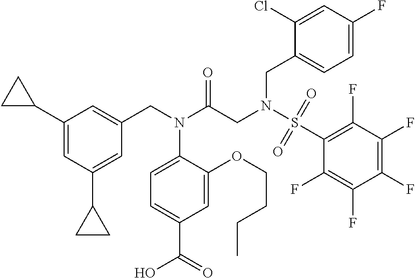 [SMILES: CCCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | A | C | | A |
| 1255 | 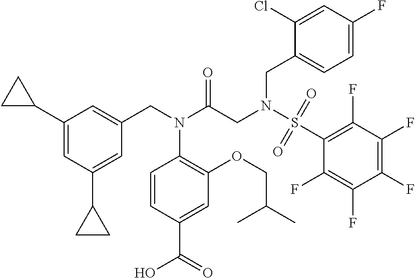 [SMILES: CC(C)COc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | B | | A |
| 1256 | 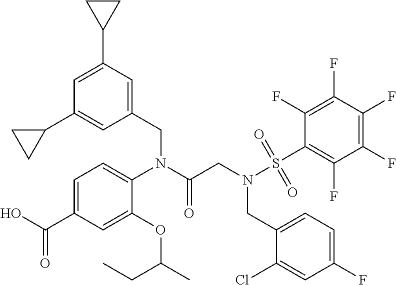 [SMILES: CCC(C)Oc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | | | |
| 1257 | 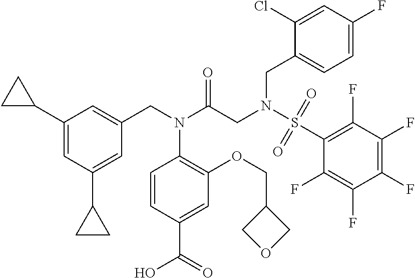 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OCC2COC2)c1] | | B | | D |
| 1258 | 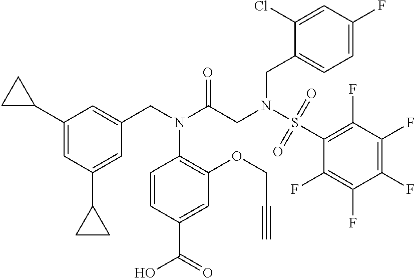 [SMILES: C#CCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | A | B | | D |
| 1260 | 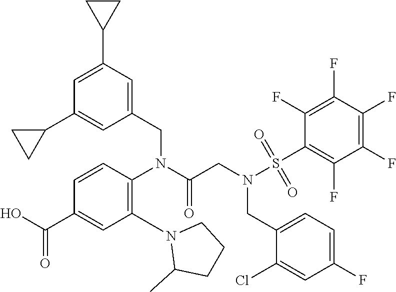 [SMILES: CC1CCCN1c1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | | | |
| 1261 | 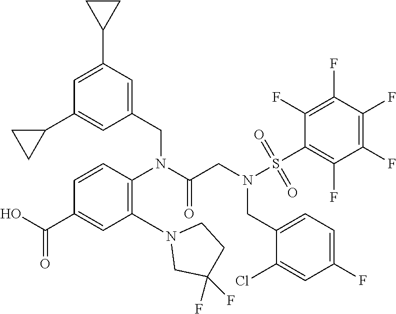 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCC(F)(F)C2)c1] | | | | |
| 1262 | 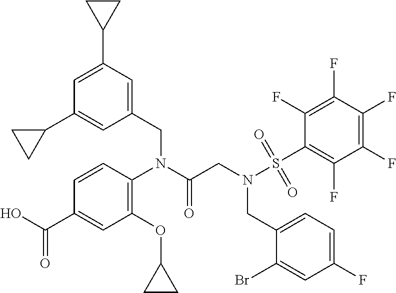 [SMILES: O=C(O)c1ccc(N(Cc2cc(C3CC3)cc(C3CC3)c2)C(=O)CN(Cc2ccc(F)cc2Br)S(=O)(=O)c2c(F)c(F)c(F)c(F)c2F)c(OC2CC2)c1] | | | | |
| 1263 | 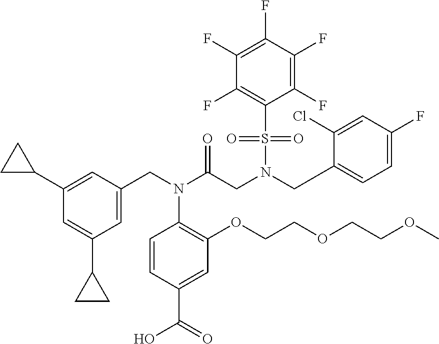 [SMILES: COCCOCCOc1cc(C(=O)O)ccc1N(Cc1cc(C2CC2)cc(C2CC2)c1)C(=O)CN(Cc1ccc(F)cc1Cl)S(=O)(=O)c1c(F)c(F)c(F)c(F)c1F] | | | | |
| 1264 | 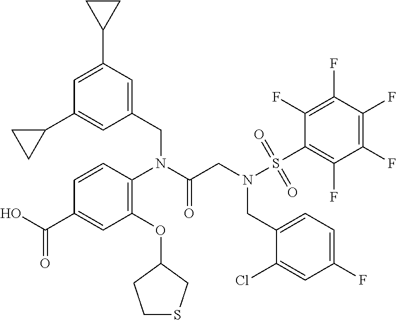 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OC2CCSC2)c1] | | B | | |
| 1267 | 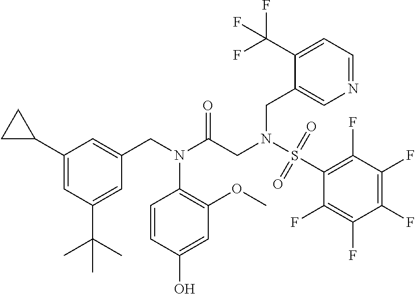 [SMILES: COc1cc(O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | | | | A |
| 1268 | 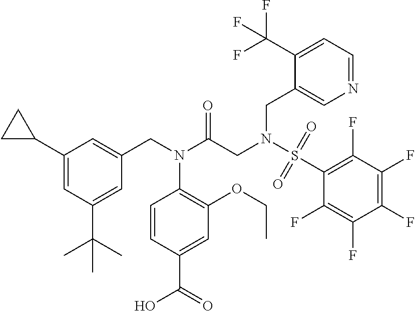 [SMILES: CCOc1cc(C(=O)O)ccc1N(Cc1cc(C2CC2)cc(C(C)(C)C)c1)C(=O)CN(Cc1cnccc1C(F)(F)F)S(=O)(=O)c1c(F)c(F)c(F)c(F)c1F] | | | | |
| 1270 | 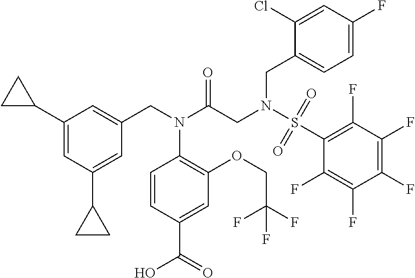 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(OCC(F)(F)F)c1] | | | | |
| 1271 | 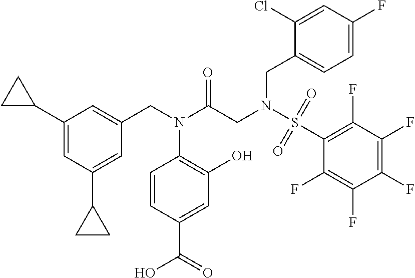 [SMILES: O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(O)c1] | | | | |
| 1272 | 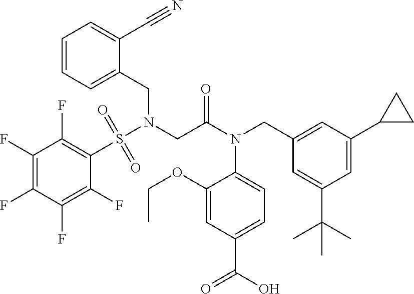 [SMILES: CCOc1cc(C(=O)O)ccc1N(Cc1cc(C2CC2)cc(C(C)(C)C)c1)C(=O)CN(Cc1ccccc1C#N)S(=O)(=O)c1c(F)c(F)c(F)c(F)c1F] | A | B | | C |
| 1273 | 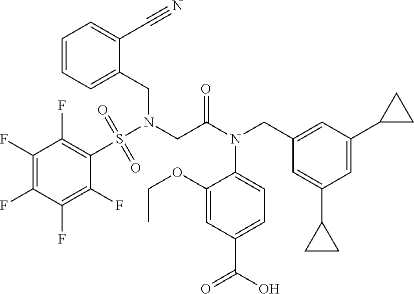 [SMILES: CCOc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F] | A | B | A | D |
| 1274 | 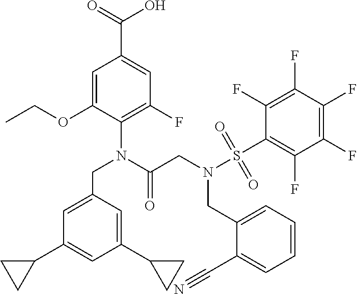 [SMILES: CCOc1cc(C(=O)O)cc(F)c1N(Cc1cc(C2CC2)cc(C2CC2)c1)C(=O)CN(Cc1ccccc1C#N)S(=O)(=O)c1c(F)c(F)c(F)c(F)c1F] | A | B | A | C |
| 1275 | 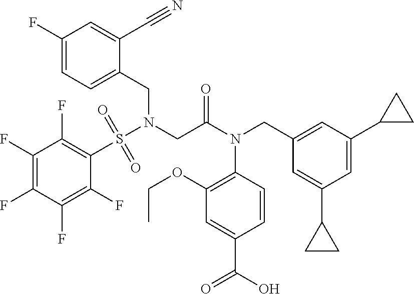 [SMILES: CCOc1cc(C(=O)O)ccc1N(Cc1cc(C2CC2)cc(C2CC2)c1)C(=O)CN(Cc1ccc(F)cc1C#N)S(=O)(=O)c1c(F)c(F)c(F)c(F)c1F] | A | A | | |
| 1276 | 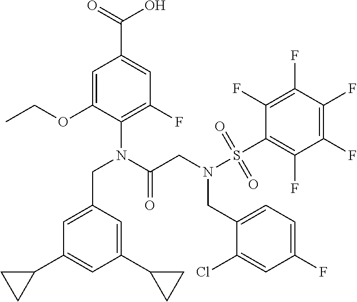 [SMILES: CCOc1cc(C(=O)O)cc(F)c1N(Cc1cc(C2CC2)cc(C2CC2)c1)C(=O)CN(Cc1ccc(F)cc1Cl)S(=O)(=O)c1c(F)c(F)c(F)c(F)c1F] | A | B | | B |
| 1277 | 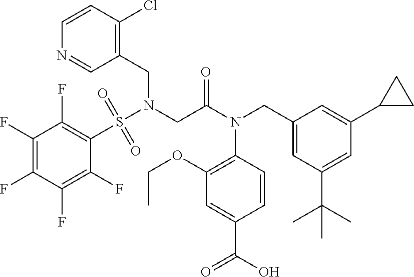 [SMILES: CCOc1cc(C(=O)O)ccc1N(Cc1cc(C2CC2)cc(C(C)(C)C)c1)C(=O)CN(Cc1cnccc1Cl)S(=O)(=O)c1c(F)c(F)c(F)c(F)c1F] | A | A | | D |
| 1278 | 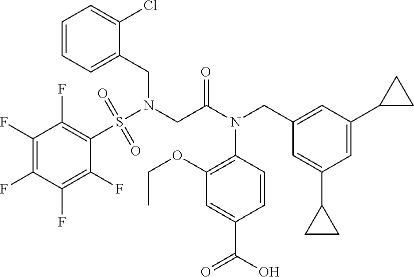 [SMILES: CCOc1cc(C(=O)O)ccc1N(Cc1cc(C2CC2)cc(C2CC2)c1)C(=O)CN(Cc1ccccc1Cl)S(=O)(=O)c1c(F)c(F)c(F)c(F)c1F] | A | B | | C |
| 1279 | 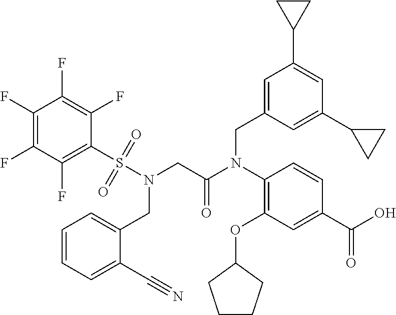 [SMILES: N#Cc1ccccc1CN(CC(=O)N(Cc1cc(C2CC2)cc(C2CC2)c1)c1ccc(C(=O)O)cc1OC1CCCC1)S(=O)(=O)c1c(F)c(F)c(F)c(F)c1F] | A | B | | B |
| 1280 | 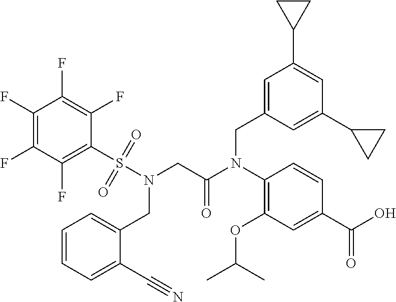 [SMILES: CC(C)Oc1cc(C(=O)O)ccc1N(Cc1cc(C2CC2)cc(C2CC2)c1)C(=O)CN(Cc1ccccc1C#N)S(=O)(=O)c1c(F)c(F)c(F)c(F)c1F] | A | B | | C |
| 1281 | 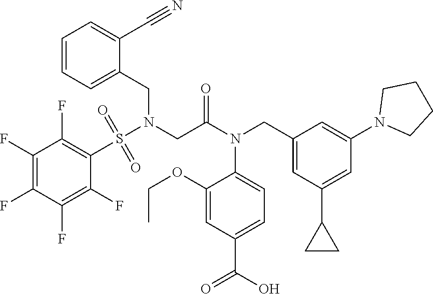 [SMILES: CCOc1cc(C(=O)O)ccc1N(Cc1cc(C2CC2)cc(N2CCCC2)c1)C(=O)CN(Cc1ccccc1C#N)S(=O)(=O)c1c(F)c(F)c(F)c(F)c1F] | A | B | A | C |

Data are designated within the following ranges:

IC50 MV-4-11: 0.00001<A<0.316≤B<1≤C<3.16≤D<100 (μM)

IC50—Pooled NHF: 0.001<A<10≤B<20≤C<30≤D<100 (μM)

Permeability −Log Pe—PAMPA (4% BSA): 0.1<A<5.5≤B<6≤C<6.5≤D<10

t1/2 GSH HPLC: 0.1<D<142≤C<247≤B<554≤A<100,000 (minutes)

In some embodiments, the activities and other properties of the disclosed exemplary compounds as determined by the above assays are shown in Table 4.

TABLE 4
Exemplary Assay Data

| | | IC₅₀- | Permeability | |
|---|---|---|---|---|
| | | MV-4- | LogPe- | T_{l/2}- |
| Compound | | 11 | PAMPA | GSH |
| No. | Structure | (μM) | (4% BSA) | (min) |
| 2001 | 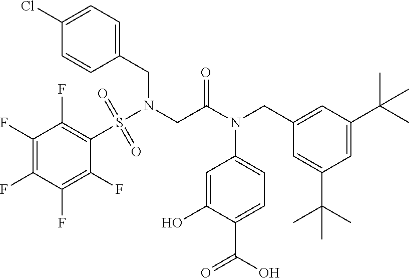 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C(C)(C)C)c1] | D | C | B |
| 2002 | 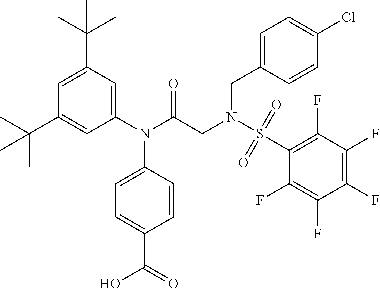 [SMILES: CC(C)(C)c1cc(N(C(=O)CN(Cc2ccc(Cl)cc2)S(=O)(=O)c2c(F)c(F)c(F)c(F)c2F)c2ccc(C(=O)O)cc2)cc(C(C)(C)C)c1] | B | | |
| 2003 | 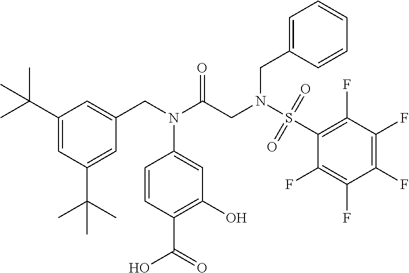 [SMILES: CC(C)(C)c1cc(CN(C(=O)CN(Cc2ccccc2)S(=O)(=O)c2c(F)c(F)c(F)c(F)c2F)c2ccc(C(=O)O)c(O)c2)cc(C(C)(C)C)c1] | C | | |
| 2004 | 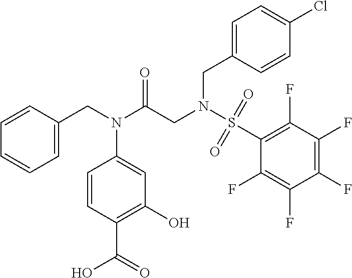 [SMILES: O=C(O)c1ccc(N(Cc2ccccc2)C(=O)CN(Cc2ccc(Cl)cc2)S(=O)(=O)c2c(F)c(F)c(F)c(F)c2F)cc1O] | D | | D |
| 2005 | 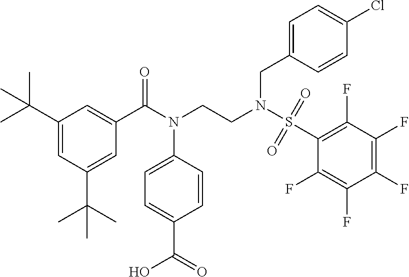 [SMILES: CC(C)(C)c1cc(C(=O)N(CCN(Cc2ccc(Cl)cc2)S(=O)(=O)c2c(F)c(F)c(F)c(F)c2F)c2ccc(C(=O)O)cc2)cc(C(C)(C)C)c1] | B | | |
| 2006 | 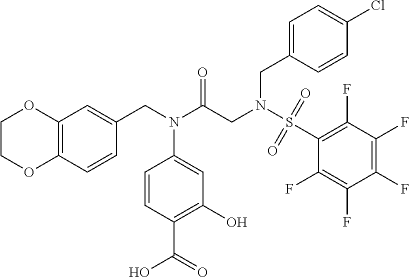 [SMILES: O=C(O)c1ccc(N(Cc2ccc3c(c2)OCCO3)C(=O)CN(Cc2ccc(Cl)cc2)S(=O)(=O)c2c(F)c(F)c(F)c(F)c2F)cc1O] | D | | |
| 2007 | 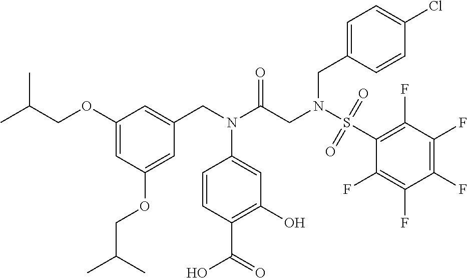 [SMILES: CC(C)COc1cc(CN(C(=O)CN(Cc2ccc(Cl)cc2)S(=O)(=O)c2c(F)c(F)c(F)c(F)c2F)c2ccc(C(=O)O)c(O)c2)cc(OCC(C)C)c1] | D | | |
| 2008 | 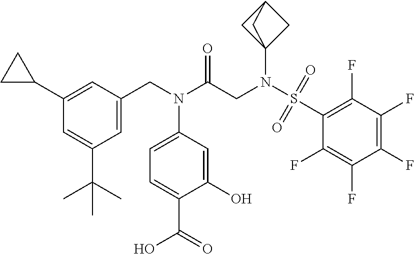 [SMILES: CC(C)(C)c1cc(CN(C(=O)CN(C23CC(C2)C3)S(=O)(=O)c2c(F)c(F)c(F)c(F)c2F)c2ccc(C(=O)O)c(O)c2)cc(C2CC2)c1] | D | | |
| 2009 | 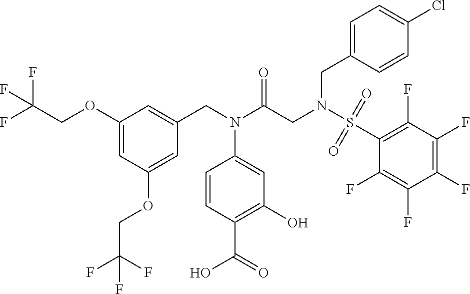 [SMILES: O=C(O)c1ccc(N(Cc2cc(OCC(F)(F)F)cc(OCC(F)(F)F)c2)C(=O)CN(Cc2ccc(Cl)cc2)S(=O)(=O)c2c(F)c(F)c(F)c(F)c2F)cc1O] | D | | |
| 2010 | 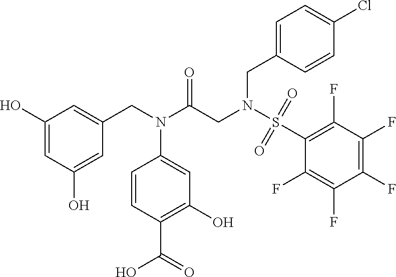 [SMILES: O=C(O)c1ccc(N(Cc2cc(O)cc(O)c2)C(=O)CN(Cc2ccc(Cl)cc2)S(=O)(=O)c2c(F)c(F)c(F)c(F)c2F)cc1O] | D | | |
| 2011 | 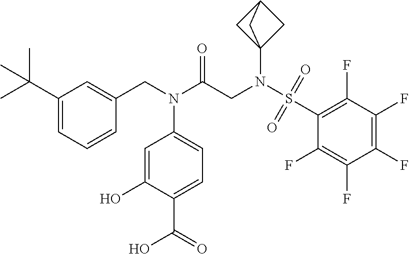 [SMILES: CC(C)(C)c1cccc(CN(C(=O)CN(C23CC(C2)C3)S(=O)(=O)c2c(F)c(F)c(F)c(F)c2F)c2ccc(C(=O)O)c(O)c2)c1] | C | | |
| 2012 | 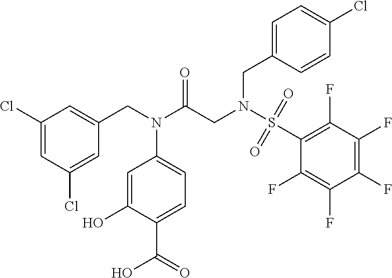 [SMILES: O=C(O)c1ccc(N(Cc2cc(Cl)cc(Cl)c2)C(=O)CN(Cc2ccc(Cl)cc2)S(=O)(=O)c2c(F)c(F)c(F)c(F)c2F)cc1O] | D | | |
| 2013 | 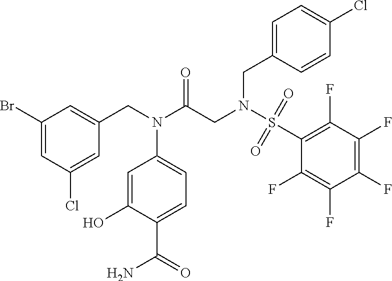 [SMILES: NC(=O)c1ccc(N(Cc2cc(Cl)cc(Br)c2)C(=O)CN(Cc2ccc(Cl)cc2)S(=O)(=O)c2c(F)c(F)c(F)c(F)c2F)cc1O] | D | | D |
| 2014 | 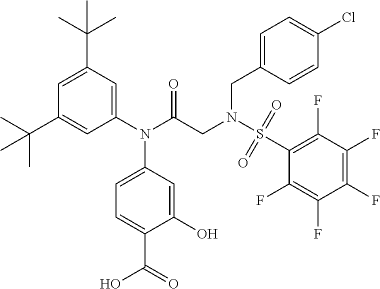 [SMILES: CC(C)(C)c1cc(N(C(=O)CN(Cc2ccc(Cl)cc2)S(=O)(=O)c2c(F)c(F)c(F)c(F)c2F)c2ccc(C(=O)O)c(O)c2)cc(C(C)(C)C)c1] | C | | |
| 2015 | 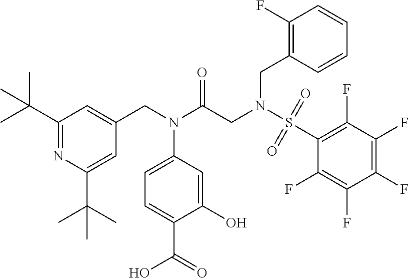 [SMILES: CC(C)(C)c1cc(CN(C(=O)CN(Cc2ccccc2F)S(=O)(=O)c2c(F)c(F)c(F)c(F)c2F)c2ccc(C(=O)O)c(O)c2)cc(C(C)(C)C)n1] | D | C | C |
| 2016 | 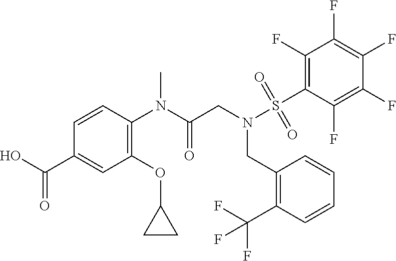 [SMILES: CN(C(=O)CN(Cc1ccccc1C(F)(F)F)S(=O)(=O)c1c(F)c(F)c(F)c(F)c1F)c1ccc(C(=O)O)cc1OC1CC1] | C | B | D |
| 2017 | 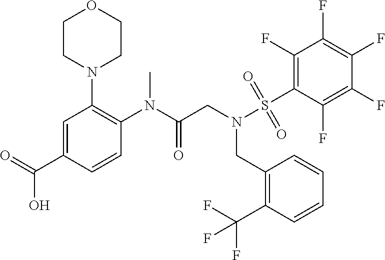 [SMILES: CN(C(=O)CN(Cc1ccccc1C(F)(F)F)S(=O)(=O)c1c(F)c(F)c(F)c(F)c1F)c1ccc(C(=O)O)cc1N1CCOCC1] | C | | D |
| 2018 | 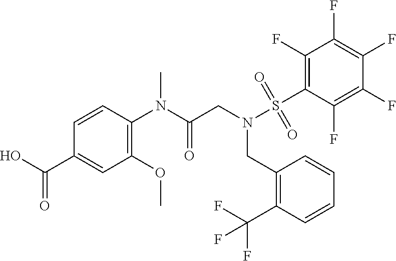 [SMILES: COc1cc(C(=O)O)ccc1N(C)C(=O)CN(Cc1ccccc1C(F)(F)F)S(=O)(=O)c1c(F)c(F)c(F)c(F)c1F] | C | | |
| 2020 | 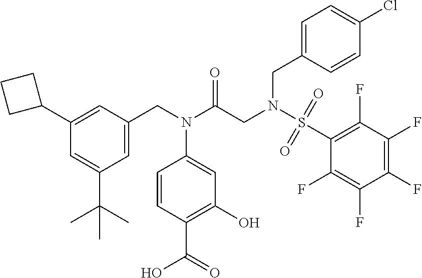 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CCC2)c1] | D | | B |
| 2022 | 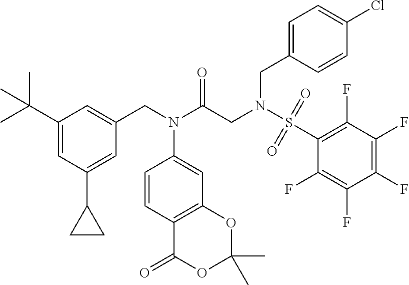 [SMILES: CC1(C)OC(=O)c2ccc(N(Cc3cc(C4CC4)cc(C(C)(C)C)c3)C(=O)CN(Cc3ccc(Cl)cc3)S(=O)(=O)c3c(F)c(F)c(F)c(F)c3F)cc2O1] | D | | |

Data are designated within the following ranges:

IC50 MV-4-11: 0.00001<A<0.316≤B<1≤C<3.16≤D<100 (μM)

Permeability −Log Pe—PAMPA (4% BSA): 0.1<A<5.5<B≤6≤C<6.5≤D<100

t1/2 GSH HPLC: 0.1<D<142≤C<247≤B<554≤A<100,000 (minutes)

We claim:

1. A compound of Formula (I), or a pharmaceutically acceptable salt or solvate thereof: 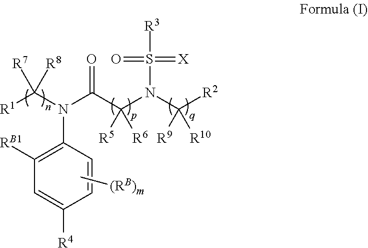
wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰ , taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
provided that p is 1 and q is 1;
each of R^{B} and R^{B1} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl;
each of n and q is independently 0, 1, 2, or 3;
p is 1, 2, or 3; and
m is 0, 1, 2, or 3.

2. The compound of claim 1, wherein each R⁶ is independently selected from H, F, —CN, substituted or unsubstituted C₁-C₆ alkyl, substituted or unsubstituted C₁-C₆haloalkyl, substituted or unsubstituted C₁-C₆heteroalkyl, and substituted or unsubstituted C₁-C₆ alkoxy.

3. The compound of claim 1, wherein each R⁶ is independently selected from H, F, methyl, ethyl, propyl, iso-propyl, n-butyl, iso-butyl, sec-butyl, t-butyl, —CF₃, —CH₂CF₃, —CH₂CH₂F, —OCF₃, —OH, —OCH₃, —OC₂CH₃, —OC₂OMe, and —OC₂C₂OH.

4. The compound of claim 1, wherein each R⁶ is H.

5. The compound of claim 1, wherein X is O.

6. The compound of claim 1, wherein p is 1; n is 0 or 1; and q is 0 or 1.

7. The compound of claim 1, wherein the compound is

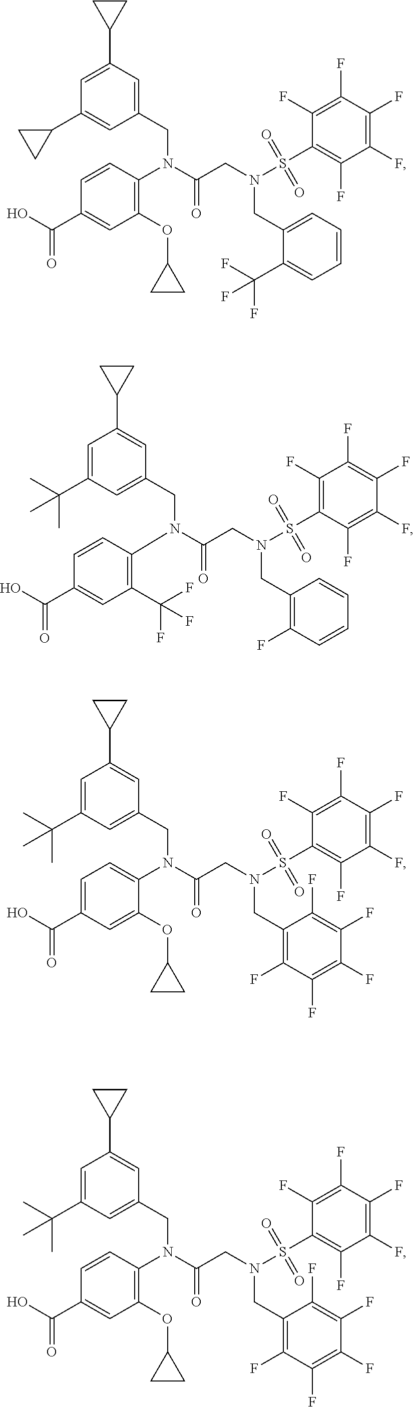 [SMILES: O=C(O)c1ccc(N(Cc2cc(C3CC3)cc(C3CC3)c2)C(=O)CN(Cc2ccccc2C(F)(F)F)S(=O)(=O)c2c(F)c(F)c(F)c(F)c2F)c(OC2CC2)c1 ; CC(C)(C)c1cc(CN(C(=O)CN(Cc2ccccc2F)S(=O)(=O)c2c(F)c(F)c(F)c(F)c2F)c2ccc(C(=O)O)cc2C(F)(F)F)cc(C2CC2)c1 ; CC(C)(C)c1cc(CN(C(=O)CN(Cc2c(F)c(F)c(F)c(F)c2F)S(=O)(=O)c2c(F)c(F)c(F)c(F)c2F)c2ccc(C(=O)O)cc2OC2CC2)cc(C2CC2)c1 ; CC(C)(C)c1cc(CN(C(=O)CN(Cc2c(F)c(F)c(F)c(F)c2F)S(=O)(=O)c2c(F)c(F)c(F)c(F)c2F)c2ccc(C(=O)O)cc2OC2CC2)cc(C2CC2)c1]

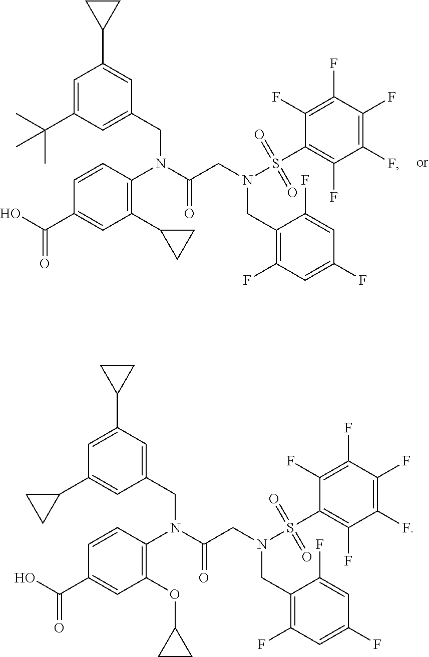 [SMILES: CC(C)(C)c1cc(CN(C(=O)CN(Cc2c(F)cc(F)cc2F)S(=O)(=O)c2c(F)c(F)c(F)c(F)c2F)c2ccc(C(=O)O)cc2C2CC2)cc(C2CC2)c1 ; O=C(O)c1ccc(N(Cc2cc(C3CC3)cc(C3CC3)c2)C(=O)CN(Cc2c(F)cc(F)cc2F)S(=O)(=O)c2c(F)c(F)c(F)c(F)c2F)c(OC2CC2)c1]

8. The compound of claim 1, wherein the compound has a structure of Formula (II), or a pharmaceutically acceptable salt or solvate thereof: 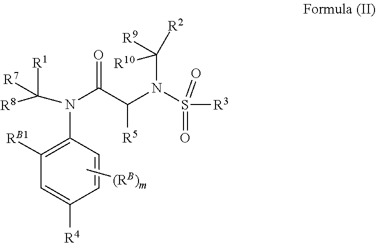
wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

9. The compound of claim 1 or 8 , wherein the compound has a structure of Formula (IIa), or a pharmaceutically acceptable salt or solvate thereof: 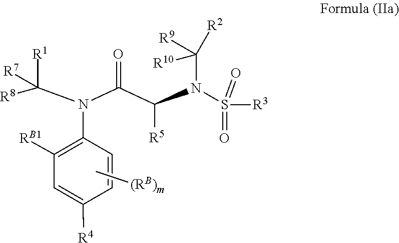
wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰ , taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C1-C6heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

10. The compound of claim 1 or 8, wherein the compound has a structure of Formula (IIb), or a pharmaceutically acceptable salt or solvate thereof: 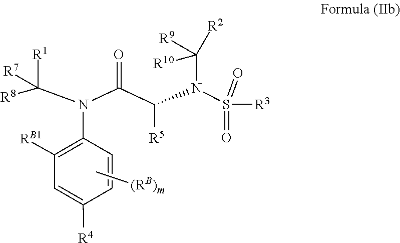
wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁷ and R⁸, taken together with the carbon to which they are attached form a substituted or unsubstituted 3, 4, 5, or 6-membered ring;
R⁵ is selected from hydrogen, F, —CN, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
wherein each of R⁹ and R¹⁰ is independently selected from the group consisting of H, F, substituted or unsubstituted C₁-C₆ alkyl, substituted or unsubstituted C₁-C₆ haloalkyl, and substituted or unsubstituted C₁-C₆ heteroalkyl, or R⁹ and R¹⁰, taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring, and R¹⁰ is selected from the group consisting of H, F, substituted or unsubstituted C₁-C₆ alkyl, substituted or unsubstituted C₁-C₆ haloalkyl, and substituted or unsubstituted C₁-C₆heteroalkyl, wherein the alkyl, haloalkyl or heteroalkyl is optionally substituted with hydroxy, amino, or methoxy;
each of R^{B} and R^{B1} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

11. The compound of claim 1, wherein each of R⁵ is independently selected from the group consisting of H, F, —OR¹¹, —SR¹¹, -N(R¹¹)₂, substituted or unsubstituted C₁-C₆ alkyl, substituted or unsubstituted C₁-C₆ haloalkyl, substituted or unsubstituted C₁-C₆heteroalkyl, substituted or unsubstituted C₃-C₈ cycloalkyl, and substituted or unsubstituted C₃-C₇ heterocycloalkyl.

12. The compound of claim 1, wherein each of R⁷, R⁸, R⁹, and R¹⁰ is independently selected from the group consisting of H, amino, F, substituted or unsubstituted C₁-C₆alkoxy, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl, and heteroalkyl is optionally substituted with hydroxy, amino, or methoxy.

13. The compound of claim 1, wherein each of R⁷, R⁸, R⁹, and R¹⁰ is H.

14. The compound of claim 1, wherein the compound has a structure of Formula (III), or a pharmaceutically acceptable salt or solvate thereof: 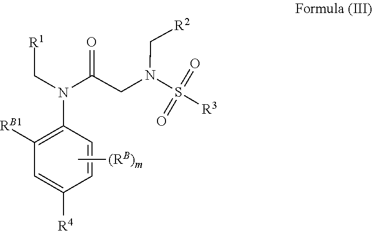
wherein,
R¹ is substituted or unsubstituted phenyl, substituted or unsubstituted C₃-C₈ cycloalkyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{B} and R^{B1} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl; and
m is 0, 1, 2, or 3.

15. The compound of claim 1, wherein R^{B1} is halogen.

16. The compound of claim 15, wherein R^{B1} is F or Cl.

17. The compound of claim 1, wherein R^{B1} is a linear or branched, substituted or unsubstituted C₁-C₆ alkyl.

18. The compound of claim 17, wherein R^{B1} is methyl, ethyl, propyl, iso-propyl, n-butyl, iso-butyl, sec-butyl, t-butyl, linear or branched pentyl, linear or branched hexyl, —CF₃, —C₂NH₂, —CH₂CF₃, —CH₂CHNH₂, —CH₂C₂F, —CH₂OH, or —CH₂C₂OH.

19. The compound of claim 1, wherein R^{B1} is substituted or unsubstituted —C₀₋₃ alkylene-C₃₋₈ cycloalkyl, or substituted or unsubstituted —C₀₋₃ alkylene-C₃₋₇ heterocycloalkyl.

20. The compound of claims 19, wherein R^{B1} is C₃₋₆ cycloalkyl, —CH₂-C₃₋₆cycloalkyl, —(C₂)₂—C₃₋₆ cycloalkyl, —(C₂)₃—C₃₋₆ cycloalkyl, C₃₋₅ heterocycloalkyl, —CH₂—C₃₋₅ heterocycloalkyl, —(C₂)₂—C₃₋₅ heterocycloalkyl, or —(C₂)₃—C₃₋₅ heterocycloalkyl, wherein the cycloalkyl and heterocycloalkyl is substituted or unsubstituted.

21. The compound of claim 18, wherein the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

22. The compound of claim 19, wherein R^{B1} is

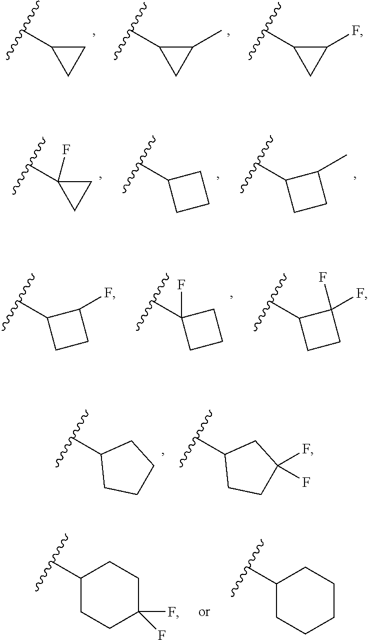

23. The compound of claim 19, wherein R^{B1} is

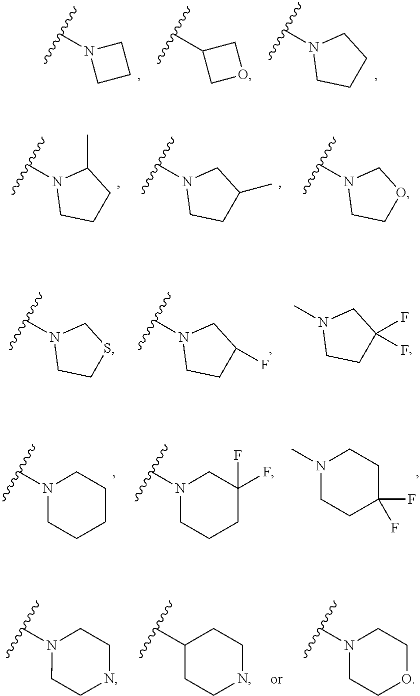

24. The compound of claim 1, wherein R^{B1} is —OR¹¹.

25. The compound of claim 24, wherein R^{B1} is OH.

26. The compound of claim 24, wherein R^{B1} is substituted or unsubstituted —O—C₁-C₆ alkyl.

27. The compound of claim 26, wherein R^{B1} is

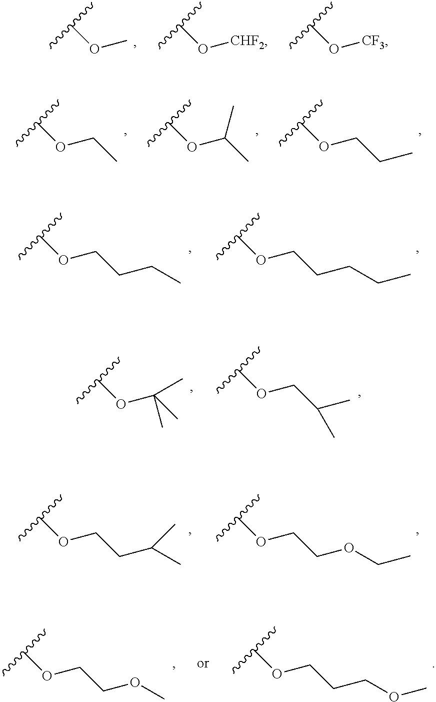

28. The compound of claim 1, wherein R^{B1} is substituted or unsubstituted —O—C₀₋₆ alkylene-C₃₋₈ cycloalkyl, or substituted or unsubstituted —O—C₀₋₆ alkylene-C₃₋₇ heterocycloalkyl.

29. The compound of claim 28, wherein R^{B1} is

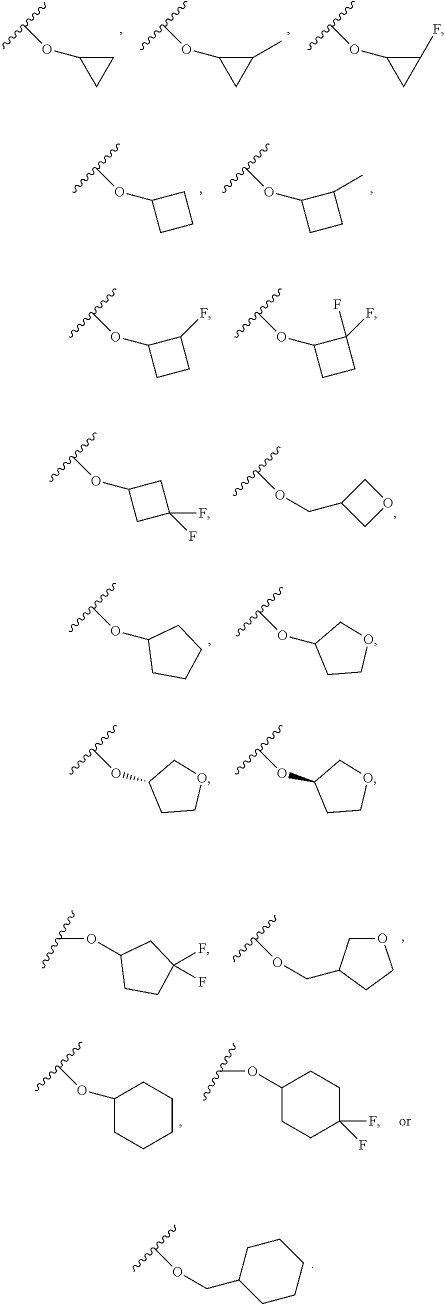

30. The compound of claim 1, wherein R^{B1} is —N(R¹¹)₂.

31. The compound of claim 30, wherein R^{B1} is —N(CH₃)₂, —NHCH₃, —N(C₂CH₃)₂, —NHCH₂CH₃, or —N(C₂C₂CH₃)₂.

32. The compound of claim 1, wherein R^{B1} is —NR¹¹S(═O)₂R¹¹.

33. The compound of claim 32, wherein R^{B1} is —NCH₃S(═O)₂CH₃.

34. The compound of claims 1, wherein m is 0 or 1.

35. The compound of claim 1, wherein each R^{B} is independently halogen, D, —CN, —OR¹¹, —SR¹¹, —N(R¹¹)₂, —NR¹¹S(═O)₂R¹¹, substituted or unsubstituted C₁-C₆ alkyl, substituted or unsubstituted —C₀₋₃ alkylene-C₃₋₆ cycloalkyl, or substituted or unsubstituted —C₀₋₃ alkylene-C₃₋₅ heterocycloalkyl.

36. The compound of claim 35, wherein each R^{B} is independently a halogen selected from F and Cl.

37. The compound of claim 35, wherein each R^{B} is independently linear or branched, substituted or unsubstituted C₁-C₆ alkyl.

38. The compound of claim 37, wherein each C₁-C₆ alkyl is independently methyl, ethyl, propyl, iso-propyl, n-butyl, iso-butyl, sec-butyl, t-butyl, linear or branched pentyl, linear or branched hexyl, —CF₃, —CH₂NH₂, —CH₂CF₃, —CH₂CHNH₂, —CH₂C₂F, —CH₂OH, or —CH₂C₂OH.

39. The compound of claim 35, wherein each of R^{B} is independently substituted or unsubstituted —C₀₋₆ alkylene-C₃₋₈ cycloalkyl, or substituted or unsubstituted —C₀₋₆ alkylene-C₃₋₇ heterocycloalkyl.

40. The compound of claim 39, wherein each of R^{B} is independently C₃₋₆ cycloalkyl, —CH₂—C₃₋₆ cycloalkyl, —(C₂)₂—C₃₋₆ cycloalkyl, —(C₂)₃—C₃₋₆ cycloalkyl, C₃₋₅ heterocycloalkyl, —C₂—C₃₋₅ heterocycloalkyl, —(C₂)₂—C₃₋₅ heterocycloalkyl, or —(C₂)₃—C₃-sheterocycloalkyl, wherein the cycloalkyl and heterocycloalkyl is substituted or unsubstituted.

41. The compound of claim 39, wherein the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

42. The compound of claim 39, wherein each of the cycloalkyl is independently

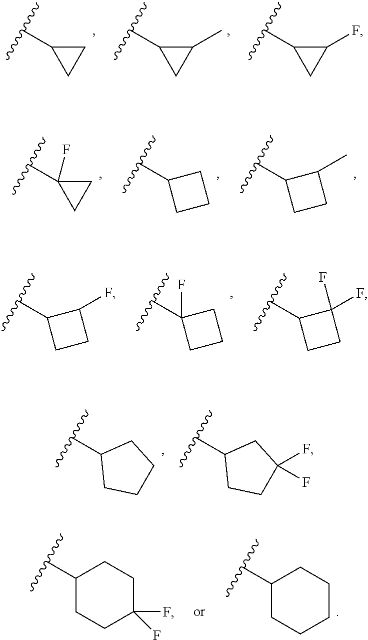

43. The compound of claim 39, wherein each of the heterocycloalkyl is independently

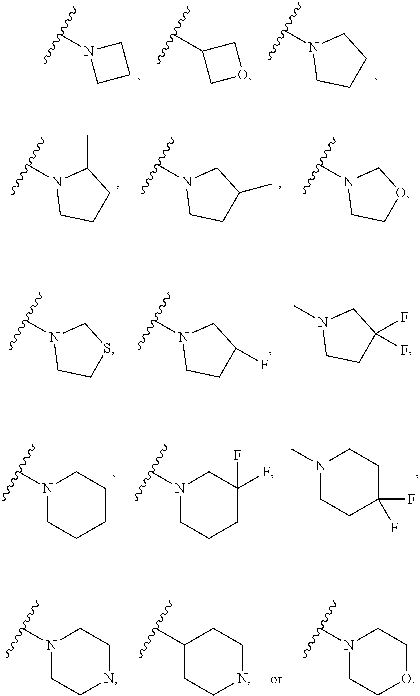

44. The compound of claim 35, wherein each R^{B} is independently —OR¹¹.

45. The compound of claim 44, wherein each —OR¹¹ is independently OH, —O—C₁-C₆ alkyl, —O—C₁-C₆ haloalkyl, —O—C₁-C₆ heteroalkyl, —O—C₀₋₆ alkylene-C₃₋₈ cycloalkyl, or —O—C₀₋₆ alkylene-C₃₋₇ heterocycloalkyl, wherein the alkyl, haloalkyl, heteroalkyl, cycloalkyl, and heterocycloalkyl is substituted or unsubstituted.

46. The compound of claim 44, wherein each R^{B} is independently

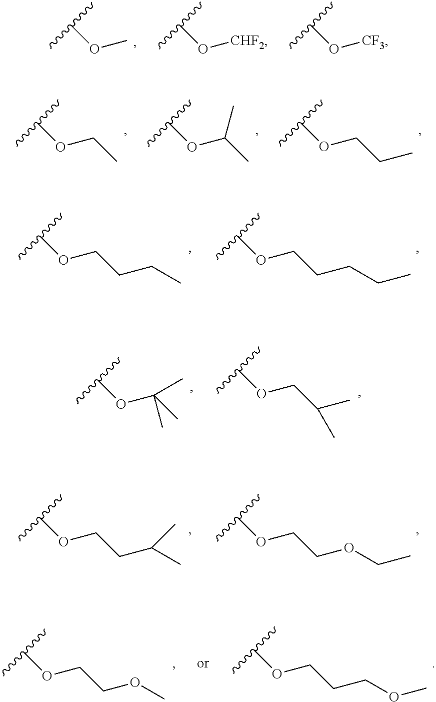

47. The compound of claim 44, wherein each R^{B} is independently

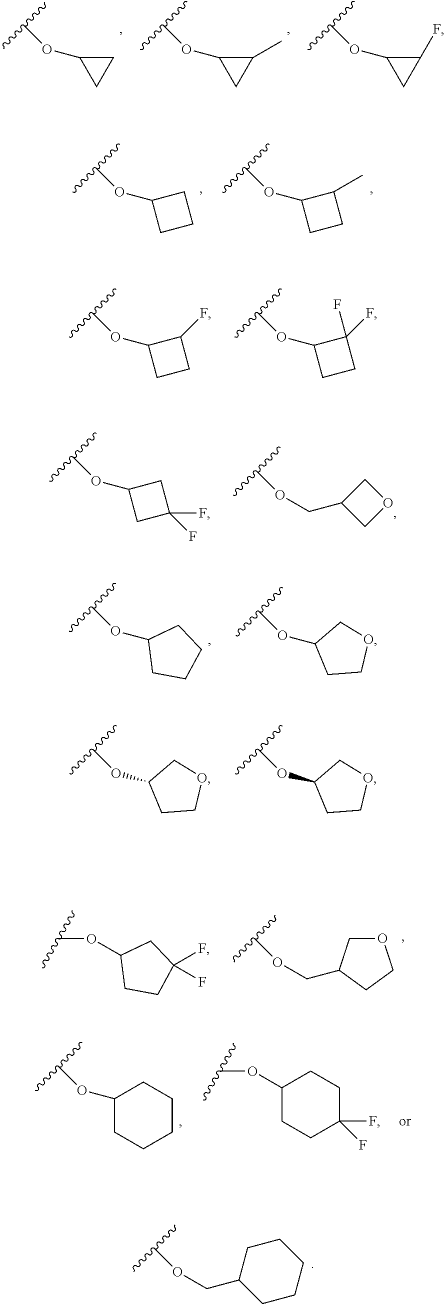

48. The compound of claim 35, wherein each R^{B} is independently —N(R¹¹)₂.

49. The compound of claim 48, wherein each R^{B} is independently —N(CH₃)₂, —NHCH₃, —N(C₂CH₃)₂, —NHC₂CH₃, or —N(C₂C₂CH₃)₂.

50. The compound of claim 35, wherein at least one R^{B} is —NR¹¹S(═O)₂R¹¹.

51. The compound of claim 50, wherein the -NR¹¹S(═O)2R¹¹ is —NCH₃S(═O)₂CH₃.

52. The compound of claim 1, wherein R⁴ is COOH or an isostere thereof.

53. The compound of claim 1, wherein R⁴ is SO₂H.

54. The compound of claim 1, wherein R⁴ is —OR¹¹.

55. The compound of claim 54, wherein R⁴ is

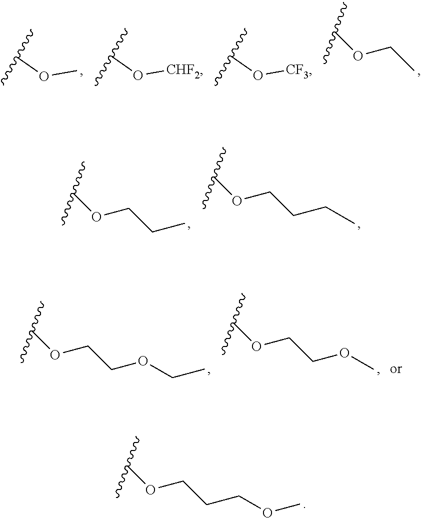

56. The compound of claim 1, wherein

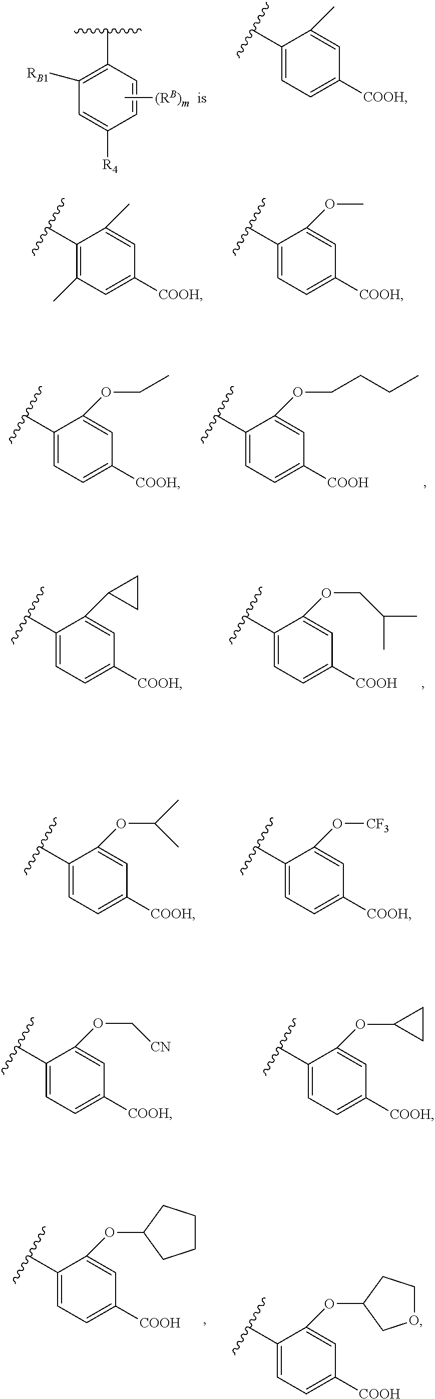

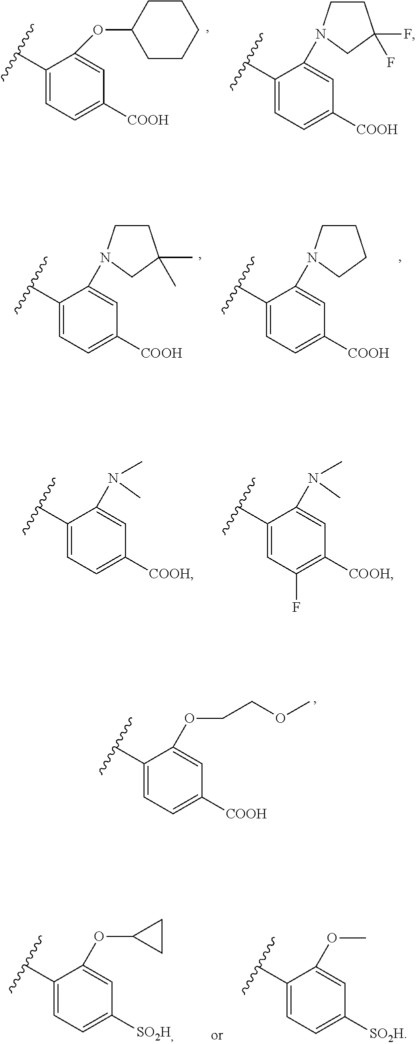

57. The compound of claim 1, wherein R¹ is substituted or unsubstituted phenyl.

58. The compound of claim 57, wherein R¹ is substituted phenyl, and wherein the phenyl is substituted with 1 to 5 substituents independently selected from halogen, D, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇heterocycloalkyl.

59. The compound of claim 58, wherein R¹ is substituted phenyl, and wherein the phenyl is substituted with F or Cl.

60. The compound of claim 58, wherein R¹ is substituted phenyl, wherein the phenyl is substituted with —O—C₁-C₆ alkyl, and wherein the alkyl is substituted or unsubstituted.

61. The compound of claim 58, wherein R¹ is substituted phenyl, and wherein the phenyl is substituted with one or two C₁-C₆ alkyl, and wherein the alkyl is linear or branched, substituted or unsubstituted.

62. The compound of claim 58, wherein R¹ is substituted phenyl, and wherein the phenyl is substituted with one or two C₃₋₈ cycloalkyl, and wherein the cycloalkyl is substituted or unsubstituted.

63. The compound of claim 58, wherein R¹ is

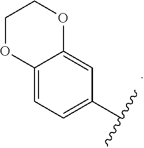 [SMILES: *c1ccc2c(c1)OCCO2]

64. The compound of claim 58, wherein R¹ is

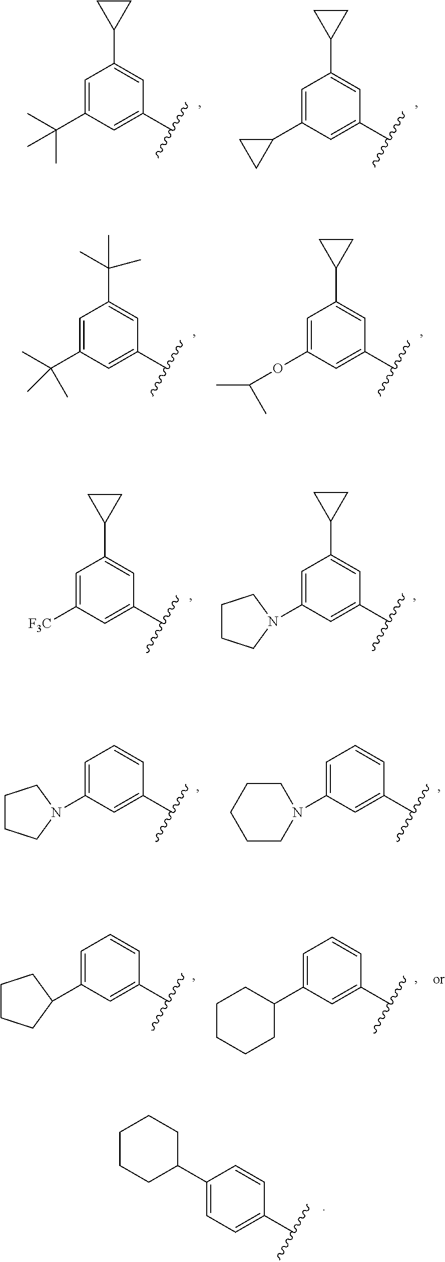

65. The compound of claim 64, wherein R¹ is

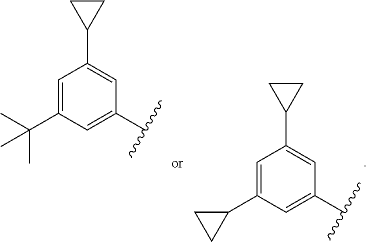 [SMILES: *c1cc(C2CC2)cc(C(C)(C)C)c1 ; *c1cc(C2CC2)cc(C2CC2)c1]

66. The compound of claim 58, wherein Ris naphthyl.

67. The compound claim 1, wherein R¹ is substituted or unsubstituted C₃-C₈ cycloalkyl.

68. The compound of claim 67, wherein R¹ is

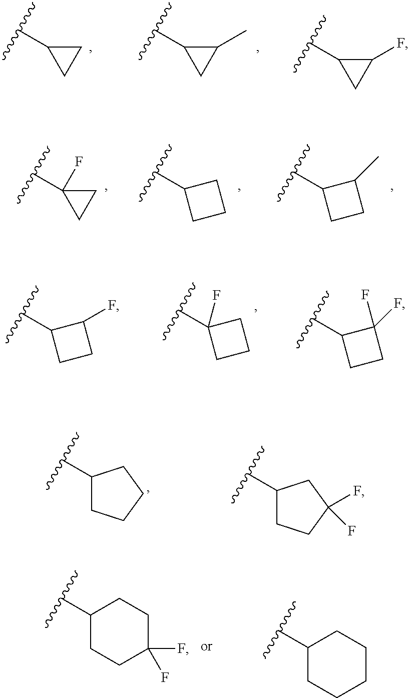

69. The compound of claim 1, wherein R¹ is substituted or unsubstituted monocyclic heteroaryl containing 1, 2, or 3 nitrogen(s).

70. The compound of claim 69, wherein R¹ is substituted or unsubstituted pyridinyl, pyridazinyl, or pyrimidinyl.

71. The compound of claim 1, wherein R¹ is substituted or unsubstituted bicyclic heteroaryl comprising 1 to 2 N.

72. The compound of claim 71, wherein R¹ is

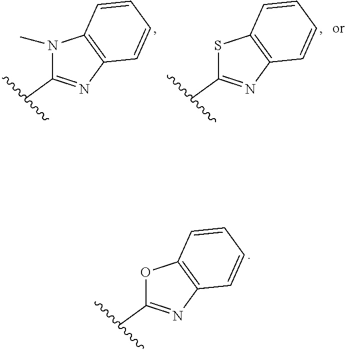 [SMILES: *c1nc2ccccc2n1C ; *c1nc2ccccc2s1 ; *c1nc2ccccc2o1]

73. A compound of Formula (IV), or a pharmaceutically acceptable salt or solvate thereof: 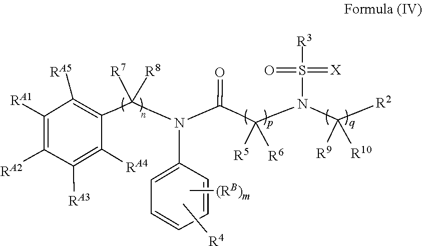
wherein,
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R⁷ and R⁸ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆heteroalkyl, wherein the alkyl is optionally substituted with hydroxy, amino, or methoxy, or R⁷ and R⁸, taken together with the carbon to which they are attached form a substituted or unsubstituted 3, 4, 5, or 6-membered ring;
each of R⁵ and R⁶ is independently selected from hydrogen, F, —CN, —OR¹¹, —SR¹¹, —N(R¹¹)₂, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl, or R⁵ and R⁶, taken together form an oxo, oxime, or with the carbon to which they are attached form a substituted or unsubstituted spirocyclic 3, 4, 5, or 6-membered ring,
wherein each of R⁹ and R¹⁰ is independently selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, or R⁹ and R¹⁰ , taken together with the carbon to which they are attached form a substituted or unsubstituted 3, 4, 5, or 6-membered ring; or
R⁵ and R⁹, taken together with the intervening atoms to which they are attached form a 4, 5 or 6-membered ring,
wherein R⁶ is selected from hydrogen, F, —CN, —OR¹¹, —SR¹¹, —N(R¹¹)₂, —C(═O)R¹¹, —C(═O)R¹¹, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₈ cycloalkyl, and substituted or unsubstituted C₃-C₇ heterocycloalkyl,
wherein R¹⁰ is selected from the group consisting of H, F, amino, —OR¹¹, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl or heteroalkyl is optionally substituted with hydroxy, amino, or methoxy,
provided that p is 1 and q is 1;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆ haloalkyl, substituted or unsubstituted —C₀₋₆ alkylene-C₃₋₈ cycloalkyl, and substituted or unsubstituted —C₀₋₆ alkylene-C₃₋₇ heterocycloalkyl, wherein
at least one of R^{A1} and R^{A3} is substituted or unsubstituted —C₃-C₈ cycloalkyl, substituted or unsubstituted —C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
X is O, NR¹¹, or absent;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or C₃₋₇ heterocycloalkyl;
each of n and q is independently 0, 1, 2, or 3;
p is 1, 2, or 3; and
m is 0, 1, 2, 3, or 4,
provided that when (i) 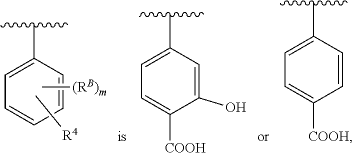
(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other is hydrogen or unsubstituted t-butyl,
then R^{A4} and R^{A5} are not the same.

74. The compound of claim 73, wherein the compound is

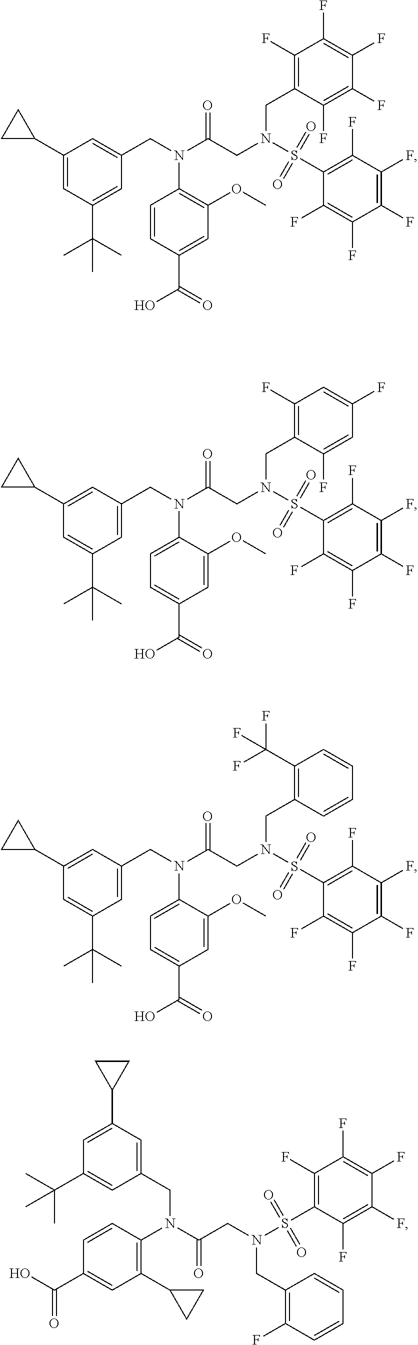 [SMILES: COc1cc(C(=O)O)ccc1N(Cc1cc(C2CC2)cc(C(C)(C)C)c1)C(=O)CN(Cc1c(F)c(F)c(F)c(F)c1F)S(=O)(=O)c1c(F)c(F)c(F)c(F)c1F ; COc1cc(C(=O)O)ccc1N(Cc1cc(C2CC2)cc(C(C)(C)C)c1)C(=O)CN(Cc1c(F)cc(F)cc1F)S(=O)(=O)c1c(F)c(F)c(F)c(F)c1F ; COc1cc(C(=O)O)ccc1N(Cc1cc(C2CC2)cc(C(C)(C)C)c1)C(=O)CN(Cc1ccccc1C(F)(F)F)S(=O)(=O)c1c(F)c(F)c(F)c(F)c1F ; CC(C)(C)c1cc(CN(C(=O)CN(Cc2ccccc2F)S(=O)(=O)c2c(F)c(F)c(F)c(F)c2F)c2ccc(C(=O)O)cc2C2CC2)cc(C2CC2)c1]

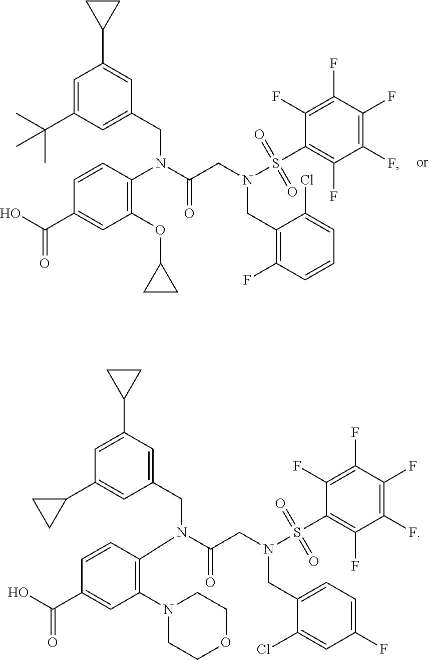 [SMILES: CC(C)(C)c1cc(CN(C(=O)CN(Cc2c(F)cccc2Cl)S(=O)(=O)c2c(F)c(F)c(F)c(F)c2F)c2ccc(C(=O)O)cc2OC2CC2)cc(C2CC2)c1 ; O=C(O)c1ccc(N(Cc2cc(C3CC3)cc(C3CC3)c2)C(=O)CN(Cc2ccc(F)cc2Cl)S(=O)(=O)c2c(F)c(F)c(F)c(F)c2F)c(N2CCOCC2)c1]

75. The compound of claim 73, wherein each of R⁵ and R⁶ is independently selected from the group consisting of H, F, —OR¹¹, —SR¹¹, —N(R¹¹)₂, substituted or unsubstituted C₁-C₆ alkyl, substituted or unsubstituted C₁-C₆haloalkyl, substituted or unsubstituted C₁-C₆heteroalkyl, substituted or unsubstituted C₃-C₈ cycloalkyl, and substituted or unsubstituted C₃-C₇ heterocycloalkyl.

76. The compound of claim 73, wherein each of R⁷, R⁸, R⁹, and R¹⁰ is independently selected from the group consisting of H, amino, F, substituted or unsubstituted C₁-C₆alkoxy, substituted or unsubstituted mono-C₁-C₆ alkylamino, substituted or unsubstituted di-C₁-C₆ alkylamino, substituted or unsubstituted C₁-C₆ alkyl, substituted or unsubstituted C₁-C₆ haloalkyl, and substituted or unsubstituted C₁-C₆ heteroalkyl, wherein the alkyl, haloalkyl, and heteroalkyl is optionally substituted with hydroxy, amino, or methoxy.

77. The compound of claim 76, wherein each of R⁷, R⁸, R⁹, and R¹⁰ is H.

78. The compound of claim 73, wherein X is 0.

79. The compound of claim 73, wherein the compound has a structure of Formula (V), or a pharmaceutically acceptable salt or solvate thereof: 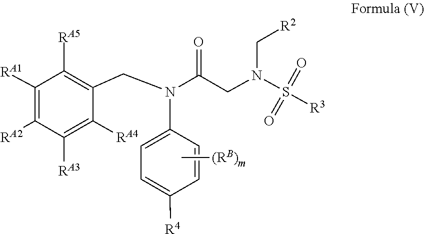
wherein,
R² is substituted or unsubstituted C₃-C₈ cycloalkyl, substituted or unsubstituted C₃-C₇ heterocycloalkyl, substituted or unsubstituted phenyl, substituted or unsubstituted naphthyl, or substituted or unsubstituted mono- or bi-cyclic heteroaryl, wherein the mono- or bi-cyclic heteroaryl contains 1 to 4 heteroatoms selected from O, N, and S;
R³ is pentafluorophenyl, or substituted or unsubstituted 5 or 6 membered heteroaryl;
R⁴ is —OR¹¹, —C₀₋₆ alkylene-R⁴¹, sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof, wherein the alkylene is substituted or unsubstituted and wherein R⁴¹ is sulfonic acid, sulfinic acid, tetrazole, acyl-sulfonamide, C(O)N(R¹¹)₂, C(O)OR¹¹, S(O)₂N(R¹¹)₂, or carboxylic acid or an isostere thereof;
each of R^{A1}, R^{A2}, R^{A3}, R^{A4}, and R^{A5} is independently selected from hydrogen, halogen, —CN, —NO₂, —OR¹¹, —N(R¹¹)₂, substituted or unsubstituted C₁-C₆ alkyl, substituted or unsubstituted C₁-C₆haloalkyl, substituted or unsubstituted —C₀₋₆ alkylene-C₃₋₈ cycloalkyl, and substituted or unsubstituted —C₀₋₆ alkylene-C₃₋₇ heterocycloalkyl,
wherein at least one of R^{A1} and R^{A3} is substituted or unsubstituted —C₃-C₈ cycloalkyl, substituted or unsubstituted 1'C₃-C₇ heterocycloalkyl, substituted or unsubstituted —O—C₀₋₆ alkylene-C₃₋₈ cycloalkyl, or substituted or unsubstituted —O—C₀₋₆ alkylene-C₃₋₇ heterocycloalkyl;
each of R^{B} is independently halogen, D, —CN, —NO₂, —OR¹¹, —SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl;
each R¹¹ is independently H, substituted or unsubstituted C₁-C₆ alkyl, substituted or unsubstituted C₁-C₆haloalkyl, substituted or unsubstituted C₁-C₆heteroalkyl, substituted or
m is 0, 1, 2, 3, or 4,
provided that when (i) 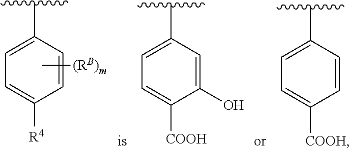
(ii) each of n, p, and q is 1, (iii) R² is phenyl substituted with 1-5 halogens, and (iv) R^{A1} and R^{A3} are both C₃-C₅ unsubstituted cycloalkyl, or one of R^{A1} and R^{A3} is C₃-C₅ unsubstituted cycloalkyl and the other of R^{A1} and R^{A3} is hydrogen or unsubstituted t-butyl,
then R^{A4} and R^{A5} are not the same.

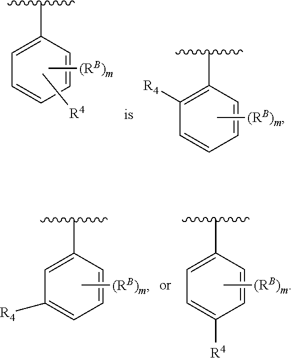

80. The compound of claim 73, wherein

81. The compound of claim 73, wherein m is 0, 1, or 2.

82. The compound of claim 73, wherein R⁴ is COOH.

83. The compound of claim 73, wherein R⁴ is SO₂H.

84. The compound of claim 73, wherein R⁴ is —OR¹¹.

85. The compound of claim 84, wherein R⁴ is

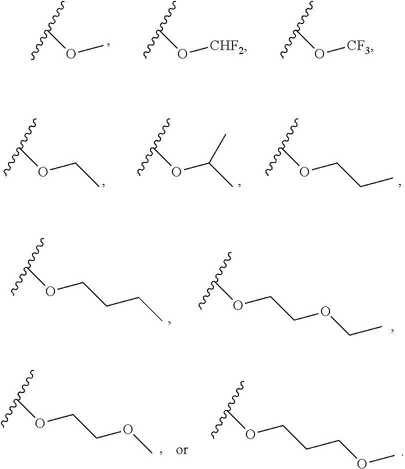

86. The compound of claim 73, wherein each R^{B} is independently halogen, D, —CN, —OR¹¹, ——SR¹¹, —N(R¹¹)₂, —NR¹¹S(═O)₂R¹¹, substituted or unsubstituted C₁-C₆ alkyl, substituted or unsubstituted —C₀₋₆ alkylene-C₃₋₈ cycloalkyl, or substituted or unsubstituted —C₀₋₆ alkylene-C₃₋₇ heterocycloalkyl.

87. The compound of claim 86, wherein at least one R^{B} is a halogen selected from F and Cl.

88. The compound of claim 86, wherein at least one R^{B} is a linear or branched, substituted or unsubstituted C₁-C₆ alkyl.

89. The compound of claim 88, wherein each C₁-C₆ alkyl is independently methyl, ethyl, propyl, iso-propyl, n-butyl, iso-butyl, sec-butyl, t-butyl, linear or branched pentyl, linear or branched hexyl, —CF₃, —CH₂NH₂, —CH₂CF₃, —CH₂CHNH₂, —CH₂C₂F, —CH₂OH, or —CH₂C₂OH.

90. The compound of claim 86, wherein at least one R^{B} is C₃₋₆ cycloalkyl, —CH₂-C₃₋₆cycloalkyl, —(C₂)₂—C₃₋₆ cycloalkyl, —(C₂)₃—C₃₋₆ cycloalkyl, C₃₋₅ heterocycloalkyl, —CH₂—C₃₋₅ heterocycloalkyl, —(C₂)₂—C₃₋₅ heterocycloalkyl, or —(C₂)₃—C₃₋₅heterocycloalkyl, wherein the cycloalkyl and heterocycloalkyl is substituted or unsubstituted.

91. The compound of claim 86, wherein the cycloalkyl or heterocycloalkyl is cyclopropyl, cyclobutyl, cyclopentyl, or cyclohexyl, wherein the cyclopropyl, cyclobutyl, cyclopentyl, and cyclohexyl is optionally substituted, and wherein 0 to 2 of the ring carbon atoms are optionally and independently replaced by nitrogen, oxygen and sulfur.

92. The compound of claim 86, wherein each of the cycloalkyl is independently

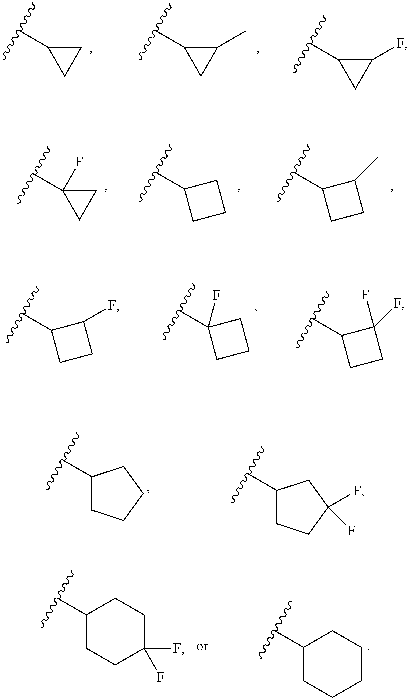

93. The compound of claim 86, wherein each of the heterocycloalkyl is independently

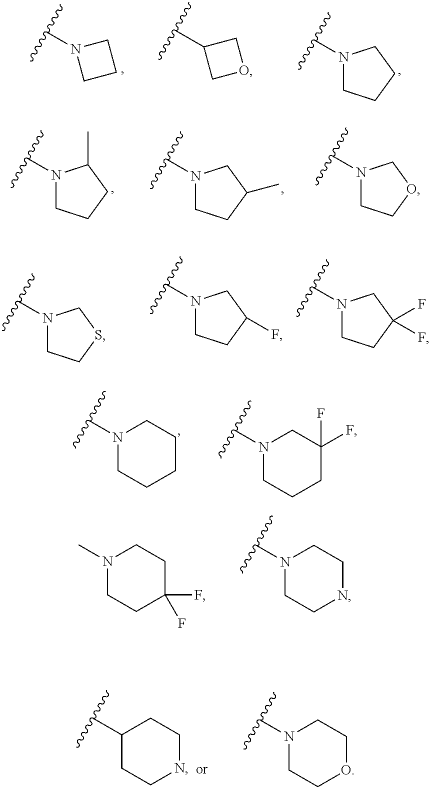

94. The compound of claim 86, wherein at least one R^{B} is —OR¹¹.

95. The compound of claim 94, wherein each -OR¹¹ is independently OH, —O—C₁-C₆ alkyl, —O—C₁-C₆ haloalkyl, —O—C₁-C₆ heteroalkyl, —O—C₀₋₆ alkylene-C₃₋₈ cycloalkyl, or —O—C₀₋₆ alkylene-C₃₋₇ heterocycloalkyl, wherein the alkyl, haloalkyl, heteroalkyl, cycloalkyl, and heterocycloalkyl is substituted or unsubstituted.

96. The compound of claim 95, wherein at least one R^{B} is

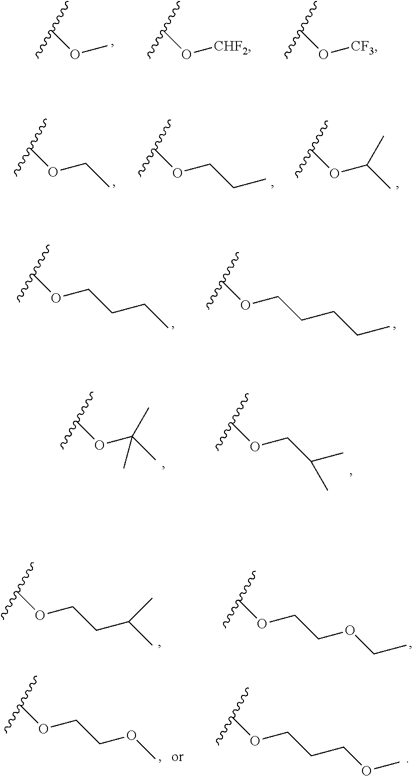

97. The compound of claim 95, wherein at least one R^{B} is OH.

98. The compound of claim 95, wherein at least one R^{B} is

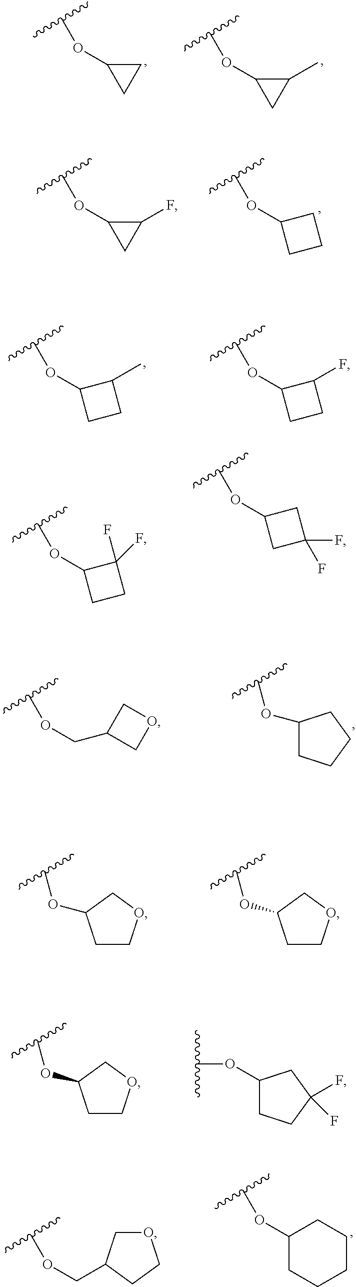

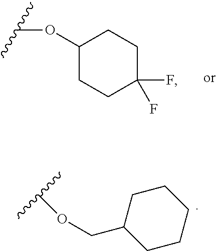 [SMILES: *OC1CCC(F)(F)CC1 ; *OCC1CCCCC1]

99. The compound of claim 86, wherein at least one R^{B} is —N(R¹¹)₂.

100. The compound of claim 99, wherein at least one R^{B} is —N(CH₃)₂, —NHCH₃, —N(C₂CH₃)₂, —NHC₂CH₃, or —N(C₂C₂CH₃)₂.

101. The compound of claim 86, wherein at least one R^{B} is —NR¹¹S(O)₂R¹¹, wherein each R¹¹ is independently H or C₁-C₃ alkyl.

102. The compound of claim 101, wherein the —NR¹¹S(═O)₂R¹¹ is —NCH₃S(═O)₂CH₃.

103. The compound of claim 73, wherein

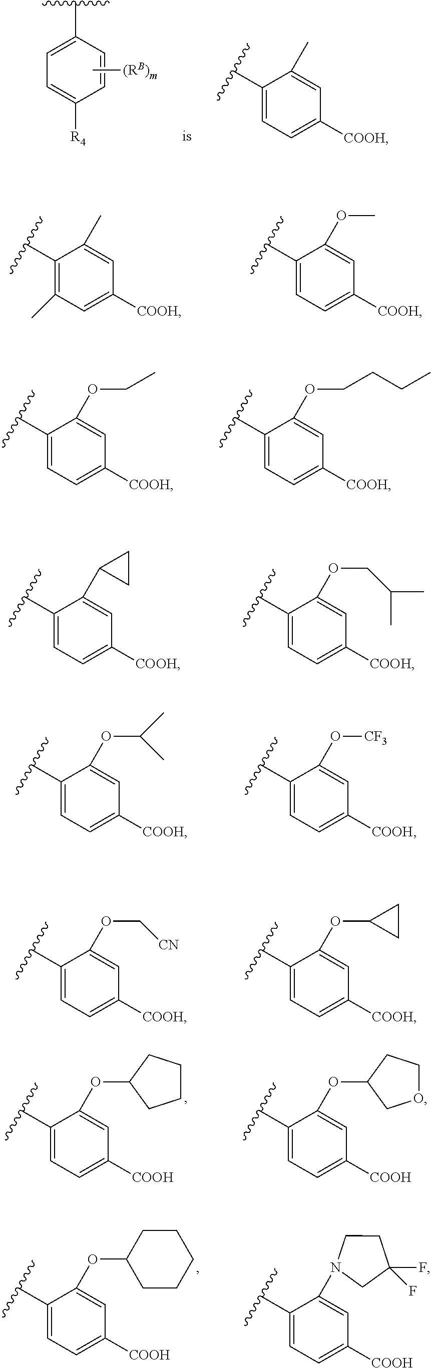

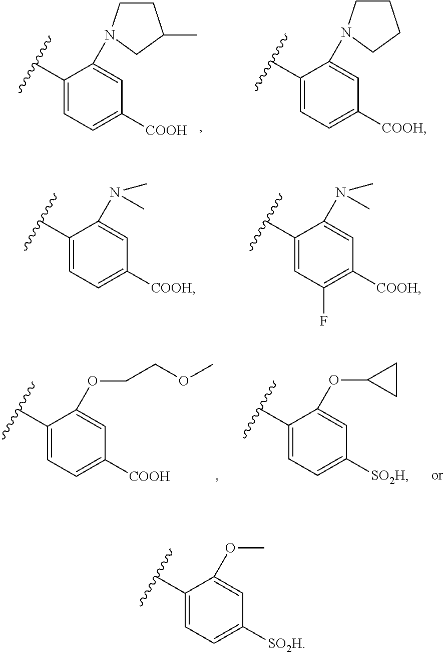

104. The compound of claim 73, wherein

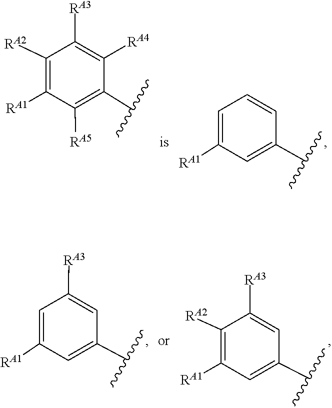

105. The compound of claim 104, wherein at least one of R^{A1} and R^{A3} is substituted or unsubstituted —C₃-C₆ cycloalkyl.

106. The compound of claim 105, wherein at least one of R^{A1} and R^{A3} is

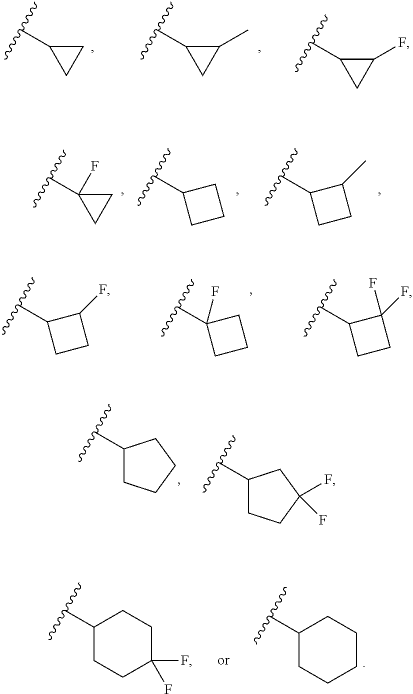

107. The compound of claim 104, wherein at least one of R^{A1} and R^{A3} is substituted or unsubstituted —C₃-C₅ heterocycloalkyl.

108. The compound of claim 107, wherein at least one of R^{A1} and R^{A3} is

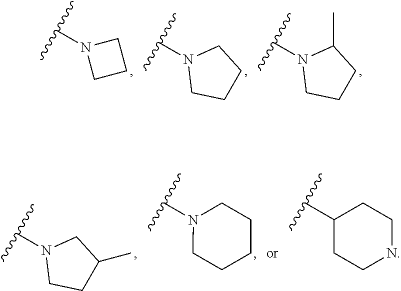

109. The compound of claim 104, wherein at least one of R^{A1} and R^{A3} is substituted or unsubstituted —O—C₀₋₃ alkylene-C₃₋₆ cycloalkyl, or substituted or unsubstituted —O—C₀₋₃ alkylene-C₃₋₅ heterocycloalkyl.

110. The compound of claim 73, wherein each of R^{A2}, R^{A3}, R^{A4}, and R^{A5} is independently halogen, C₁-C₆ alkyl, —C₀₋₃ alkylene-C₃₋₆ cycloalkyl, —C₀₋₃ alkylene-C₃₋₅ heterocycloalkyl, —O—C₁-C₆ alkyl, -0—C₀₋₃ alkylene-C₃₋₆ cycloalkyl, or —O—C₀₋₃ alkylene-C₃₋₅ heterocycloalkyl, and wherein the alkyl, cycloalkyl, and heterocycloalkyl is substituted or unsubstituted.

111. The compound of claim 110, wherein each of R^{A2}, R^{A3}, R^{A4}, and R^{A5} is independently F, Cl, methyl, ethyl, propyl, iso-propyl, n-butyl, iso-butyl, sec-butyl, t-butyl, —CF₃, —CH₂CF₃, —C₂C₂F, —OCF₃, —OH, −OCH₃, —OC₂CH₃, —OC₂₀Me, —OC₂C₂OH, —OC(CH₃)₃, —OC₂C₂OCH₃,

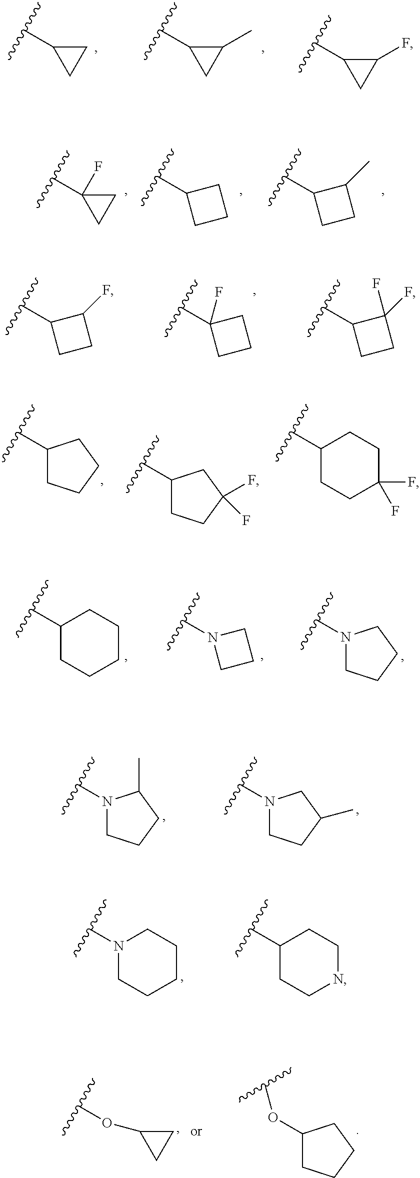

112. The compound of claim 73, wherein

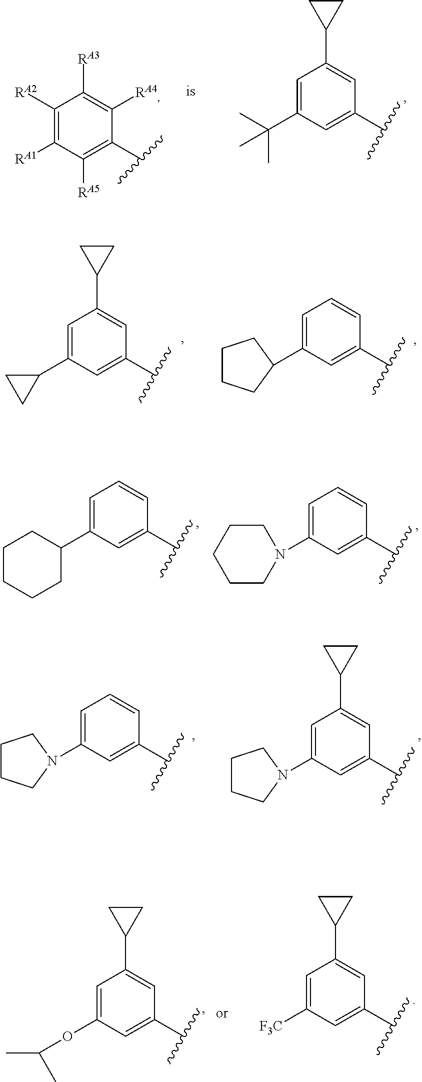

113. The compound of claim 1 or 73, wherein R² is phenyl or substituted phenyl.

114. The compound of claim 113, wherein R² is phenyl substituted with 1 to 5 R^{c}, and wherein each R^{c} is independently D, halogen, —OR¹¹, —SR¹¹, —N(R¹¹)₂, —CN, —NO₂, substituted or unsubstituted C₁-C₆ alkyl, substituted or unsubstituted C₁-C₆ haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇ heterocycloalkyl.

115. The compound of claim 114, wherein R² is phenyl substituted with 1 to 5 R^{c}, and wherein each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —CH₂C₂F, —OCF₃, —OH, —OCH₃, —OC₂CH₃, —OC₂OMe, —OC₂C₂OH, —OC(CH₃)₃, —OC₂C₂OCH₃,

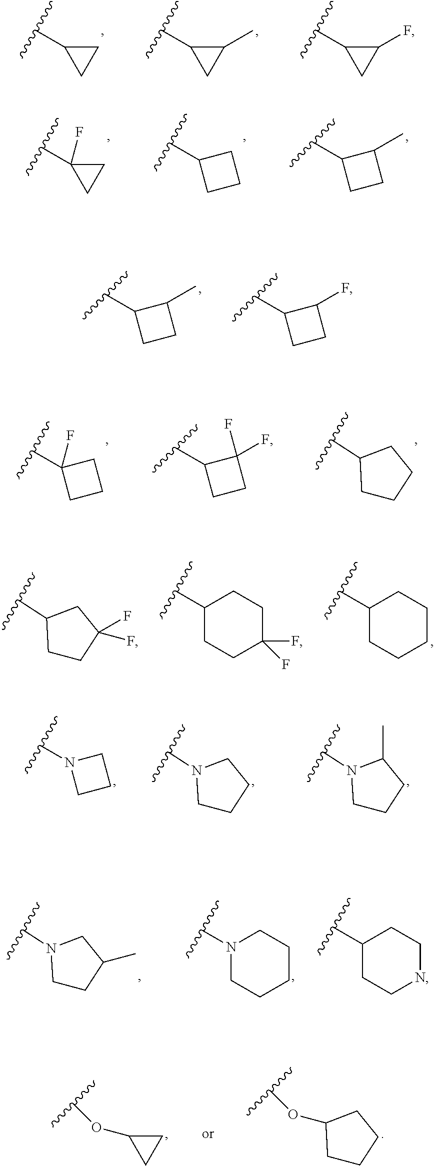

116. The compound of claim 114, wherein R² is

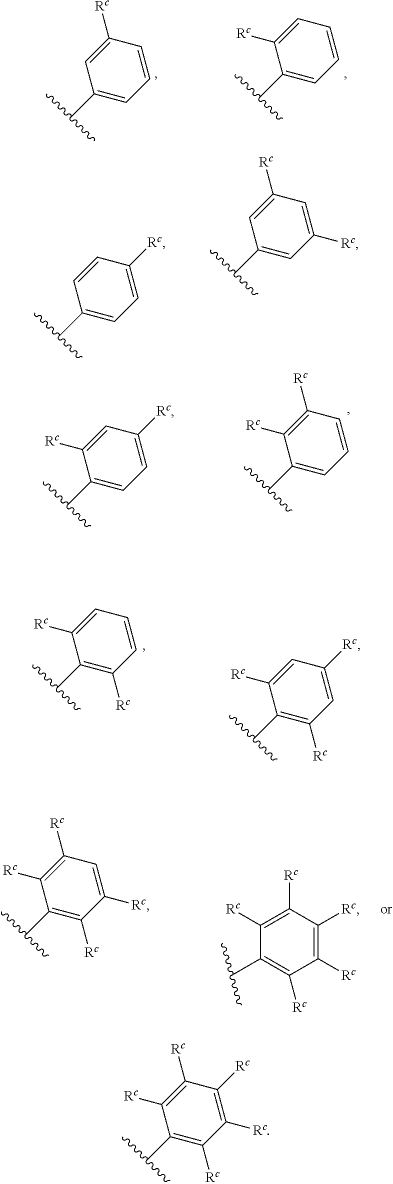

117. The compound of claims 116, wherein R² is

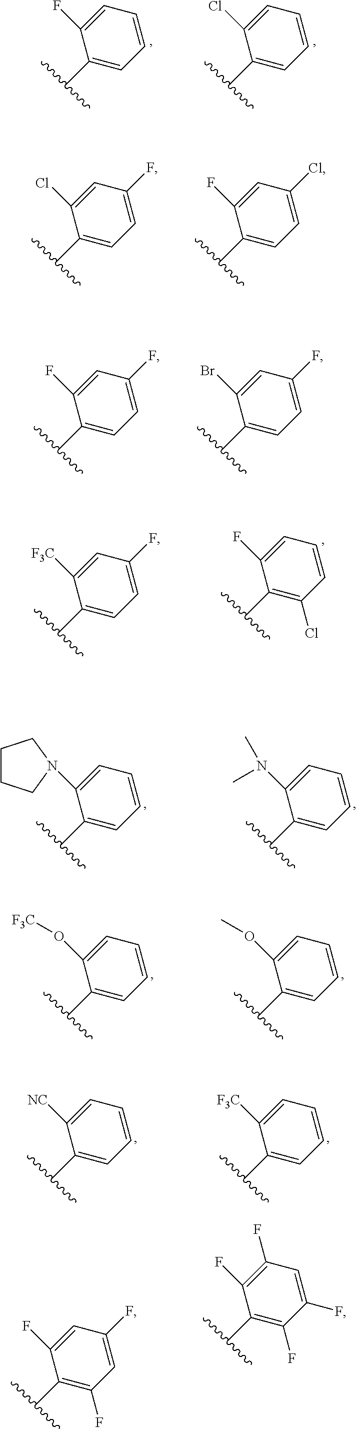

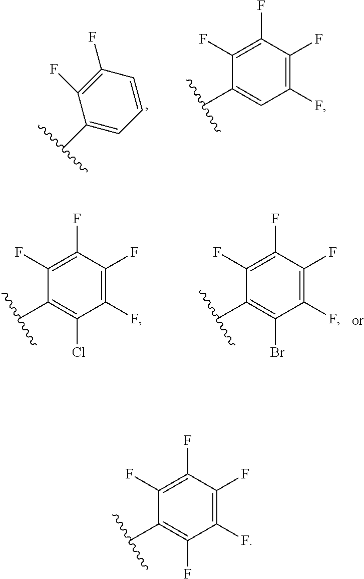 [SMILES: *c1cccc(F)c1F ; *c1cc(F)c(F)c(F)c1F ; *c1c(F)c(F)c(F)c(F)c1Cl ; *c1c(F)c(F)c(F)c(F)c1Br ; *c1c(F)c(F)c(F)c(F)c1F]

118. The compound of claim 1 or 73, wherein R² is substituted or unsubstituted 5-membered or 6-membered monocyclic heteroaryl.

119. The compound of claim 118, wherein R² is pyridinyl, pyridazinyl, pyrimidinyl, triazinyl, wherein the pyridinyl, pyridazinyl, pyrimidinyl, or triazinyl is substituted with 1 to 4 R^{c}, and wherein each R^{c} is independently D, halogen, —OR¹¹, —SR¹¹, —N(R¹¹)₂, —CN, —NO₂, substituted or unsubstituted C₁-C₆ alkyl, substituted or unsubstituted C₁-C₆haloalkyl, substituted or unsubstituted C₁-C₆ heteroalkyl, substituted or unsubstituted —C₀₋₆ alkylene-C₃₋₈ cycloalkyl, or substituted or unsubstituted —C₀₋₆ alkylene-C₃₋₇heterocycloalkyl.

120. The compound of claim 119, wherein each R^{c} is independently D, F, Cl, Br, —CN, OH, methyl, ethyl, propyl, iso-propyl, n-butyl, iso-butyl, sec-butyl, t-butyl, —CF₃, —CH₂CF₃, —C₂C₂F, —OCF₃, —OH, —OCH₃, —OC₂CH₃, —OC₂OMe, —OC₂C₂OH, —OC(CH₃)₃, —OC₂C₂OCH₃,

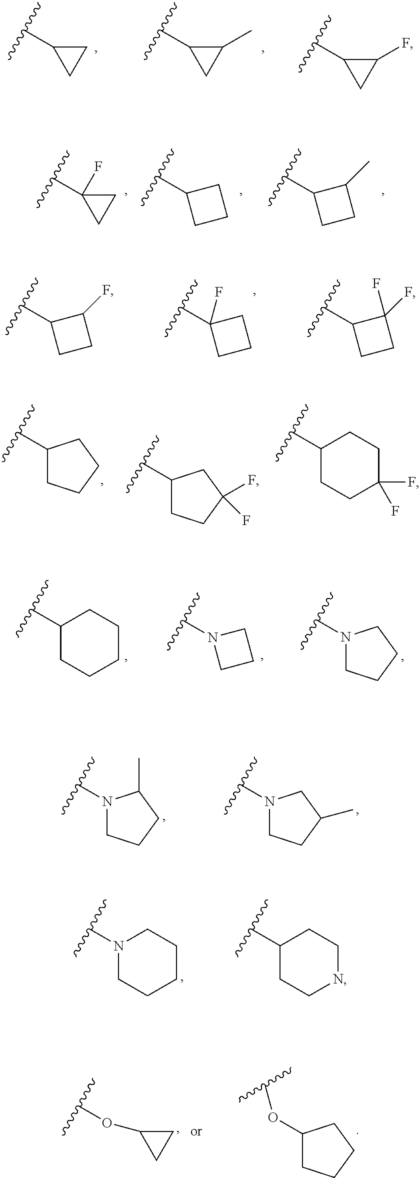

121. The compound of claim 116, wherein R² is

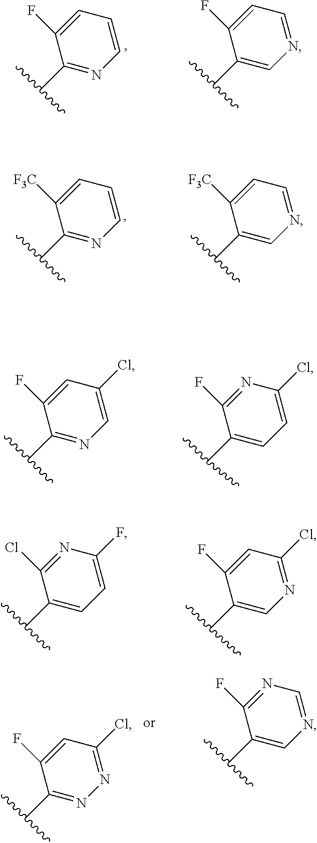

122. The compound of claim 1 or 73, wherein R² is substituted or unsubstituted 5-6, 6-6, or 6-5 fused bicyclic heteroaryl containing 1-3 hetero ring atoms selected from O, N and S.

123. The compound of claim 1 or 73, wherein R³ is substituted heteroaryl.

124. The compound of claim 123, wherein R³ is pentafluorophenyl.

125. A compound or a pharmaceutically acceptable salt or solvate thereof, wherein the compound is selected from a compound of Table 1 or Table 2.

126. A compound or a pharmaceutically acceptable salt or solvate thereof, wherein the compound is

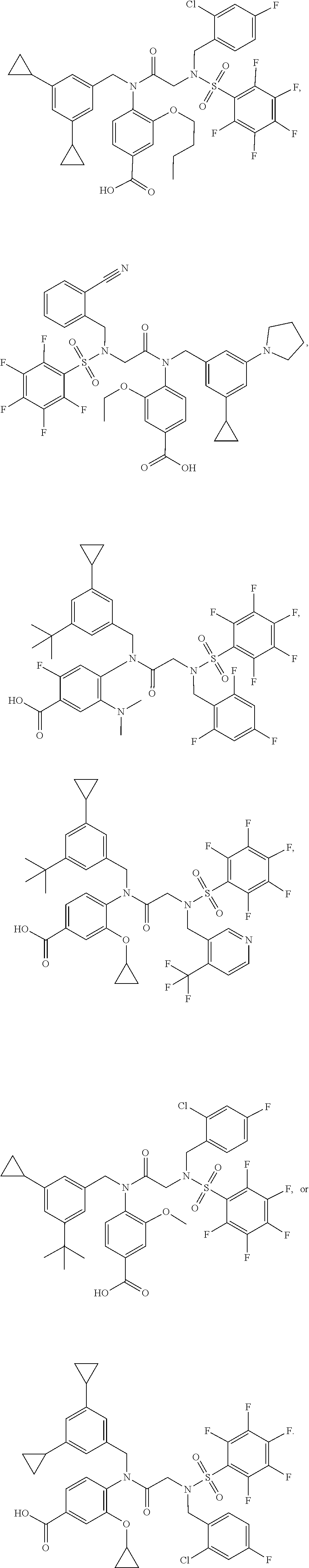

127. A pharmaceutical composition comprising a compound of any one of claims 1 to 125, a pharmaceutically acceptable salt, solvate, ester, hydrate, or polymorph thereof, and a pharmaceutically acceptable excipient or carrier.

128. A method of modulating signal transducer and activator of transcription 5a and 5b (STAT5) proteins in a subject in need thereof, comprising administering to a subject a therapeutically effective amount of a compound of any one of claims 1 to 125, or a pharmaceutically acceptable salt or solvate thereof.

129. The method of claim 128, wherein the subject has cancer.

130. A method of treating cancer in a subject in need thereof, comprising administering to a subject with cancer a therapeutically effective amount of a compound of any one of claims 1 to 125, or a pharmaceutically acceptable salt or solvate thereof

131. The method of claim 130, wherein the cancer is a solid tumor or hematologic cancer.

132. The method of claim 130, wherein the cancer is breast cancer, head and neck squamous cell carcinoma, non-small cell lung cancer, hepatocellular cancer, colorectal cancer, gastric adenocarcinoma, melanoma, or advanced cancer.
